(19) United States
(12) Patent Application Publication
(10) Pub. No.: US 2025/0042900 A1
(43) Pub. Date: Feb. 6, 2025

(54) INDOLIZINE COMPOUNDS FOR THE TREATMENT OF MENTAL DISORDERS OR INFLAMMATION

(71) Applicant: TACTOGEN INC, Palo Alto, CA (US)

(72) Inventor: Matthew J. Baggott, Redwood City, CA (US)

(73) Assignee: TACTOGEN INC, Palo Alto, CA (US)

(21) Appl. No.: 18/893,750

(22) Filed: Sep. 23, 2024

Related U.S. Application Data
(63) Continuation of application No. PCT/US2023/016297, filed on Mar. 24, 2023.
(60) Provisional application No. 63/323,410, filed on Mar. 24, 2022.
(60) Provisional application No. 63/323,411, filed on Mar. 24, 2022.
(60) Provisional application No. 63/323,412, filed on Mar. 24, 2022.

(51) Int. Cl. C07D 471/04; A61K 31/437

(52) U.S. Cl. CPC C07D 471/04; A61K 31/437

(57) ABSTRACT

The present invention discloses compounds, compositions, methods for modulating central nervous system activity, methods for treating inflammatory or metabolic disorders, and methods for treating central nervous system disorders, such as depression, anxiety, and trauma- and stressor-related disorders, comprising the indolizines having the structures disclosed herein.

### CROSS-REFERENCE TO RELATED APPLICATIONS

This application is a continuation of International Patent Application No. PCT/US2023/016297, filed in the U.S. Receiving Office on Mar. 24, 2023, which claims the benefit of U.S. Provisional Application 63/323,412 filed on Mar. 24, 2022; U.S. Provisional Application 63/323,410 filed on Mar. 24, 2022; and U.S. Provisional Application 63/323,411 filed on Mar. 24, 2022. The entirety of each of these applications is hereby incorporated by reference for all purposes.

### FIELD OF THE INVENTION

The present invention is in the area of pharmaceutically active indolizine compounds and compositions for modulating central nervous system activity, treating central nervous system disorders, or treating inflammation.

### BACKGROUND

Central nervous system (CNS) related health problems are a common challenge in society. An estimated 20.6% of U.S. adults (51.5 million people) experienced mental illness in 2019. This includes major depression (7.8% or 19.4 million people), anxiety disorders (19.1% or 48 million people), and posttraumatic stress disorder (PTSD) (3.6% or 9 million people). In addition to mental health challenges, there are other CNS disorders that cause substantial suffering and decreased quality of life. These include traumatic brain injury (TBI) (an estimated 12% of adults or 30 million people), dementias, and headache disorders (such as migraine, which affects about 15% of the general population or 47 million people). As the global population ages, many age-related CNS disorders are projected to become more common. For example, 6.2 million people aged 65 and older in the U.S. have Alzheimer's dementia and this population is expected to grow to 12.7 million by 2050.

There is a need for improved treatment of CNS disorders. Many patients fail to benefit adequately from available treatments. In addition, many available pharmacological treatments must be taken for weeks or months before the individual experiences therapeutic benefits. Because of these and other considerations, fewer than half of U.S. adults with mental illness (44.8%) received treatment in 2019.

A number of potential new experimental treatments are under investigation. These include novel compounds that modulate the functioning of the monoamine neurotransmitters, dopamine, norepinephrine, and serotonin. Dopamine is involved in learning, incentives, and the initiation of motor movements. Norepinephrine is important for attention and cardiovascular functioning.

Serotonin is incompletely understood but appears to adjust the stability of the individual's response to changing environmental conditions. As such, serotonin has been linked to mood, anxiety, and appetite.

New experimental treatment compounds include serotonin receptor agonists. Serotonin receptors have seven families and many receptors are able to stimulate multiple signaling pathways within a cell, which can make it complicated to predict therapeutic effects. Serotonin receptor types that have received recent attention for their therapeutic potential include 5-HT_{2A}, 5-HT_{2C}, 5-HT₆, 5-HT_{1A}, and 5-HT_{1B} receptors.

One group of experimental therapeutic compounds are 5-HT_{2A} receptor agonists. These are being investigated as tools for producing rapid therapeutic improvement in CNS disorders including depression, anxiety, and substance use disorders. Many, such as psilocybin and 5-methoxy-N,N-dimethyltryptamine (5-MeO-DMT), produce dramatic psychedelic effects resembling mystical experiences that may contribute to these therapeutic effects. These compounds also produce labile mood and often invoke acute anxiety, which makes close monitoring of patients necessary. There is accordingly a need for 5-HT_{2A} agonists that produce either minimal mood changes or reliably positive ones.

Indeed, another group of putative 5-HT_{2A} agonists, such as 6-methoxy-N,N-dimethyltryptamine (6-MeO-DMT) and 7-fluoro-N,N-dimethyltryptamine (7-F-DMT) appear to produce therapeutic changes in animal models of depression without producing psychedelic effects (Dunlap et al. 2020. Journal of medicinal chemistry, 63(3), pp. 1142-1155). Both psychedelic and non-psychedelic 5-HT_{2A} agonists may be useful in migraine, cluster headaches, and other headache disorders.

The therapeutic mechanisms of 5-HT_{2A} agonists are incompletely understood but may involve increased neuroplasticity (Ly et al. 2018. Cell reports, 23(11), pp. 3170-3182), suggesting potential benefits in TBI, neurological disorders, and conditions where behavior change or learning is desired.

Another potential therapeutic mechanism of 5-HT_{2A} agonists involves decreases in inflammation (e g., Flanagan, et al. 2019. Life sci., 236, 116790). Conditions that may benefit from improved anti-inflammatory treatment include rheumatoid and other forms of arthritis (such as enthesitis-related juvenile idiopathic arthritis, blau syndrome, and juvenile idiopathic arthritis), psoriasis, Crohn's disease, inflammatory bowel syndrome, ulcerative colitis, and ankylosing spondylitis. Inflammation has long been recognized to induce symptoms of depression (Lee & Giuliani. 2019. Frontiers in immunology, 10, 1696). Inflammatory processes have also been implicated in psychotic disorders (Borovcanin et al. 2012. J. Psych. Res., 46(11), 1421-1426) and bipolar disorders (Hamdani, Tamouza, & Leboyer. 2012. Front. Biosci. (Elite Ed.), 4, 2170-2182).

Patients who have depression or another CNS disorder often also exhibit chronic inflammation, although the direction of causality is still being elucidated. Inflammation has long been recognized to induce symptoms of depression (Lee & Giuliani. 2019. Frontiers in immunology, 10, 1696. doi:10.3389/fimmu.2019.01696). Inflammatory processes have also been implicated in psychotic disorders (Borovcanin et al. 2012. J. Psych. Res., 46(11), 1421-1426. doi: 10.1016/j.jpsychires.2012.08.016) and bipolar disorders (Hamdani, Tamouza, & Leboyer. 2012.

Front. Biosci. (Elite Ed.), 4, 2170-2182. doi:10.2741/534). 5-HT_{2A} agonists are also often 5-HT_{2B} agonists. This is undesirable because chronic stimulation of 5-HT_{2B} receptors causes cardiac valvulopathy (Rothman et al. 2000. Circulation, 102(23), pp. 2836-2841). There is therefore a need for serotonin agonists that have decreased ability to stimulate 5-HT_{2B} receptors.

5-HT_{2C} receptors are closely related to 5-HT_{2A} receptors, but have a different distribution in the brain and body. Compounds that stimulate 5-HT_{2C} receptors have been proposed as treatments for psychiatric disorders as well as other disorders such as sexual dysfunction, obesity, and urinary incontinence. Lorcaserin (Belviq) is a high affinity 5-HT_{2C} agonist that, until recently, was FDA-approved for use in conjunction with weight loss programs. The withdrawal of this medicine from the market because of increased risk of cancer highlights the need for safer serotonergic therapeutics that can stimulate 5-HT_{2C} receptors or otherwise aid weight loss.

5-HT₆ receptors are primarily located in the brain and preclinical research has suggested 6-HT₆ ligands may have potential for treating mood and anxiety disorders, cognitive impairments, and obsessive-compulsive disorder (Karila et al. Journal of medicinal chemistry, 58(20), pp. 7901-7912; Chaumont-Dubel et al. Neuropharmacology, 172, p.107839).

5-HT_{1A} receptor agonists modulate the functioning of dopamine and norepinephrine and decrease blood pressure and heart rate via a central mechanism. Drugs that are 5-HT_{1A} agonists have value for treating anxiety and depression. For example, buspirone (Buspar, Namanspin) is approved for anxiety disorders and may also be useful for treating hypoactive sexual desire disorder (HSDD). Studies in rats indicate that 5-HT_{1A} stimulation induces oxytocin release, which contributes to the social effects of 3,4-methylenedioxymethamphetamine (MDMA) (Thompson et al. 2007. Neuroscience, 146(2), pp. 509-514). Compounds (or compound combinations) that include 5-HT_{1A} stimulation in their pharmacological profile are therefore expected to have therapeutic benefits in comparison to those that do not.

Compounds that stimulate 5-HT_{1B} receptors alter the release of neurotransmitters such as dopamine, serotonin, GABA, acetylcholine, and glutamate and can modulate stress sensitivity, mood, anxiety, and aggression. 5-HT_{1B/1D} agonists such as sumatriptan (Imitrex) and zolmitriptan (Zomig) have been approved for treatment of headache disorders and the relative therapeutic contributions of 5-HT_{1B} and 5-HT_{1D} are incompletely understood. Although treatments for headache disorders are available, there is ongoing need for treatments with less risk of drug-drug interactions and with fewer adverse events (including idiosyncratic, teratogenic, and cognitive ones). 5-HT_{1B} agonists have also been reported to have anti-inflammatory in addition to their antinociceptive effects. Studies in mice suggest 5-HT_{1B} stimulation on dopamine-containing neurons in the central striatum contributes to social effects of MDMA (Heifets et al. 2019. Science translational medicine, 11(522)). Preclinical studies also suggest 5-HT_{1B} agonists may have antidepressant effects. More broadly, there is evidence that stimulating 5-HT_{1B} receptors can provide benefits to stress response, affect, and addiction (e.g., Fontaine et al. 2021. Neuropsychopharmacology, pp. 1-11). As with 5-HT_{1A} receptors, compounds (or compound combinations) that include 5-HT_{1B} stimulation in their pharmacological profile are therefore expected to have therapeutic benefits in comparison to those that do not.

Another group of experimental compounds interact with brain monoamine transporters to increase extracellular concentrations of the three monoamine neurotransmitters. Some compounds increase extracellular concentrations of these molecules by inhibiting reuptake of neurotransmitters, while others induce release of neurotransmitters.

Despite the ongoing research on potential new drugs to treat mental disorders, CNS disorders, and related gastrointestinal and inflammatory disorders, the enormous burden of disease caused by these disorders remains a global serious and systemic problem. In many cases, available drugs and treatments provide incomplete relief, present risks of drug-drug interactions, or cause side effects or adverse events (including idiosyncratic, teratogenic, and cognitive ones). New drugs and treatments are required to improve personal well-being, mental health, and physical health that are dependent on the alteration of neurotransmitter levels and performance.

It is therefore an object of the present invention to provide advantageous compounds and their use and manufacture for the treatment of mental disorders and/or inflammation in hosts, typically humans, in need thereof. Additional objects are to provide compounds with an efficient onset to be used in a clinical setting such as counseling or a home setting, which open the patient to empathy, sympathy and acceptance. A further object is to provide effective treatments for a range of CNS disorders.

### SUMMARY OF THE INVENTION

The present invention provides advantageous indolizine compounds and their pharmaceutically acceptable salts and salt mixtures thereof, pharmaceutical compositions, and methods to treat mental, inflammatory, and metabolic disorders. An indolizine compound of the present invention can be used for mental enhancement or to treat a mental disorder, inflammation, or metabolic disorder, comprising administering an effective amount of the compound to a host, typically a human, in need thereof. In some embodiments, the indolizine compounds or compositions described herein interacts with a serotonergic binding site and can exhibit entactogenic properties when administered in an effective amount to a host, typically a human, in need thereof. Thus, a compound described herein can be used as an effective agent for modulating CNS activity and treating CNS disorders described herein.

In certain aspects a compound or pharmaceutical composition of the present invention can be used as fast acting, more effective agent for modulating CNS activity and treating CNS disorders than currently available therapies. In certain embodiments a compound of the present invention is a fast-acting CNS modulator. In other embodiments a compound of the present invention modulates inflammation and can be used to treat inflammatory or metabolic disorders. For example, in certain aspects a compound is provided that interacts with 5-HT_{2A} and modulates CNS and/or inflammatory activity.

In certain aspects a compound of Formula I is provided, wherein the compound of Formula I is selected from

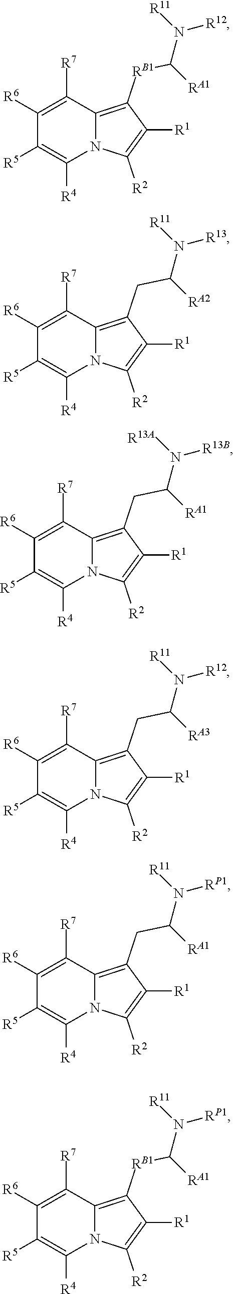

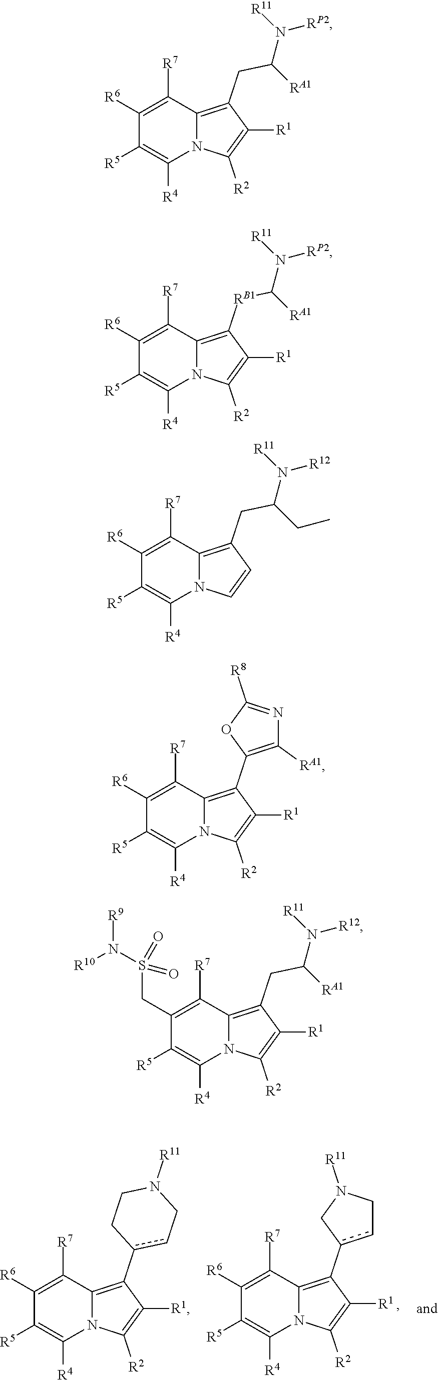

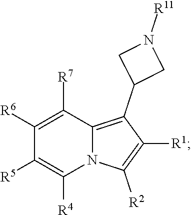

or a pharmaceutically acceptable salt or salt mixture, isotopic derivative, or prodrug thereof; wherein:
[symbol] is a single or double bond;
R^{A1} is hydrogen, —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R^{A2} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R^{A3} is —CH₂X, —CHX₂, —CX₃, —CH₂CH₂X, —CH₂ CHX₂, —CH₂CX₃, —CH₂OH, or —CH₂CH₂OH;
R^{B1} is

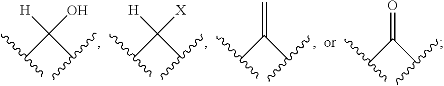

R¹, R², R⁴, R⁵ R⁶, R⁷, and R⁸ are independently selected from the group consisting of hydrogen, halogen, alkyl, haloalkyl, —OP(O)(OR⁹)₂, —SR⁹, —NR⁹R¹⁰, —NR^{P1}R¹⁰, —NR^{P2}R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
in certain embodiments R¹ is hydrogen;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹¹ is hydrogen, —(C₁-C₆)alkyl, —CH₂CH₂X, —CH₂ CHX₂, —CH₂CX₃, —CH₂CH₂OH, or hydroxy;
R¹³ is —(C₁-C₆)alkyl, —CH₂CH₂X, —CH₂ CHX₂, —CH₂CX₃, —CH₂CH₂OH, or hydroxy;
R^{13A} is —(C₁-C₆)alkyl, —CH₂CH₂X, —CH₂ CHX₂, —CH₂CX₃, or —CH₂CH₂OH;
R^{13B} is —(C₃-C₆)alkyl, —CH₂CH₂X, —CH₂ CHX₂, —CH₂CX₃, —CH₂CH₂OH, or hydroxy;
R^{13C} is alkyl, haloalkyl, —OP(O)(OR⁹)₂, —SR⁹, —NR⁹R¹⁰, —OR⁹, -alkyl-OP(O)(OR⁹)₂, -alkyl-SR⁹, -alkyl-NR⁹R¹⁰, or -alkyl-OR⁹;
R¹¹ is alkyl, haloalkyl, —NR⁹R¹⁰, or —OR⁹;
R^{P1} is selected at each instance from the group consisting of —C(O)R^{13C}, -alkyl-OC(O)R^{13C} and -alkyl-C(O)R^{13C};
R^{P2} is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R⁸ and —S(O)₂R¹⁸; and
R¹⁸ is selected from the group consisting of hydrogen, alkyl, aryl, arylalkyl, cycloalkyl, heterocycle, heteroaryl, and heteroarylalkyl.

In typical embodiments R^{P2} is attached through the C-terminus of the amino acid or peptide. For example, when R^{P2} is unsubstituted alanine

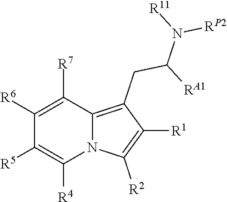

is

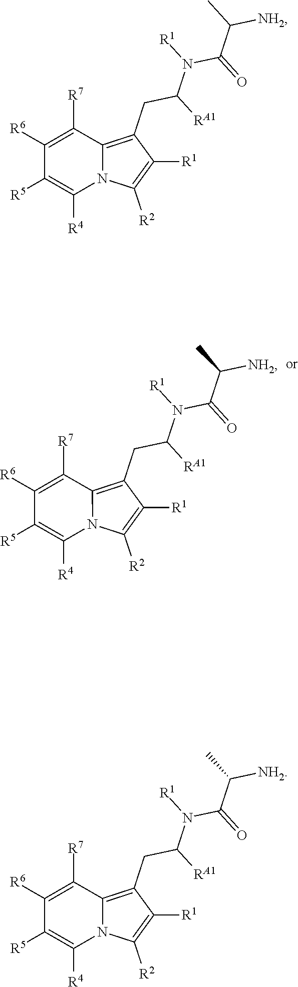

When R^{P2} is a peptide, the peptide is typically attached through the C-terminus and each amino acid is connected through an amide bond. For example, non-limiting examples of

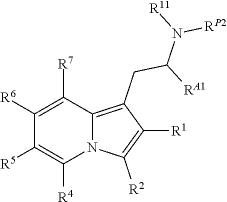

wherein R^{P2} is alanine-glutamine-glycine include

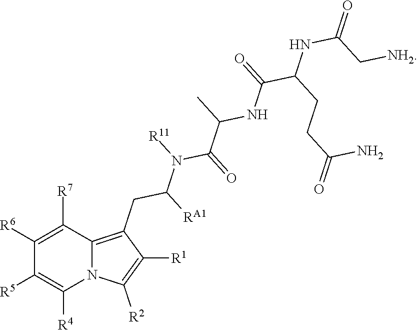

In certain embodiments the compound of the present invention is selected from:

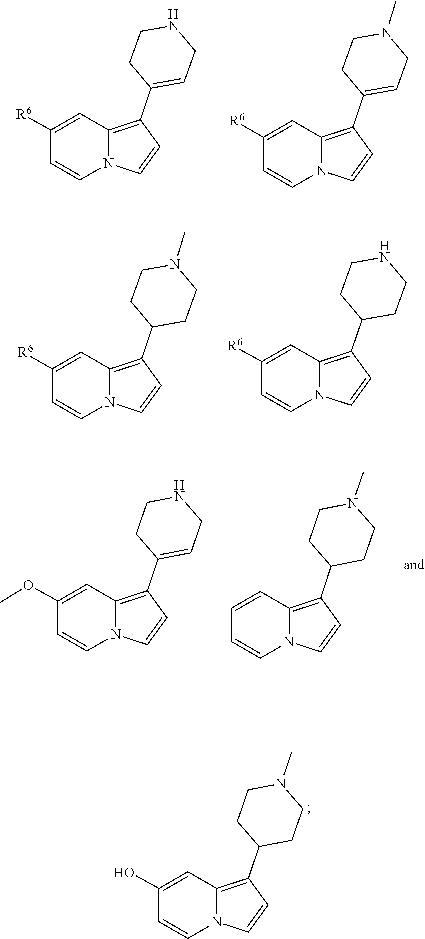

or a pharmaceutically acceptable salt or salt mixture, isotopic derivative, or prodrug thereof.

In certain embodiments the compound of the present invention is selected from:

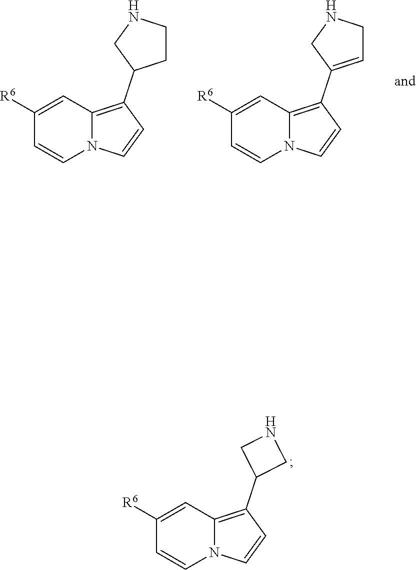

or a pharmaceutically acceptable salt or salt mixture, isotopic derivative, or prodrug thereof.

In certain embodiments the compound of the present invention is selected from:

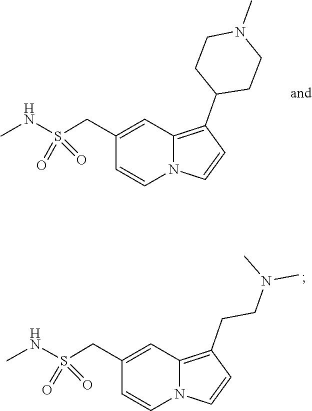

or a pharmaceutically acceptable salt or salt mixture, or isotopic derivative.

In other aspects a compound is provided of Formula:

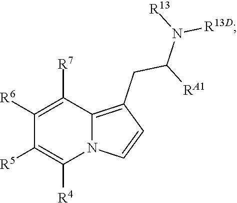

or a pharmaceutically acceptable salt or salt mixture, isotopic derivative, or prodrug thereof, wherein
R^{13D} is —(C₁-C₆)alkyl, —CH₂CH₂X, —CH₂ CHX₂, —CH₂CX₃, or —CH₂CH₂OH;
and all other variables are as defined herein.

In certain aspects of the present invention a compound described herein is used to treat an inflammatory disorder and/or metabolic disorder that has been linked to excessive inflammation in mammals. For example, in certain embodiments a compound of the present invention is used to treat a disorder selected from arthritis, psoriasis, Crohn's disease, inflammatory bowel syndrome, ulcerative colitis, and ankylosing spondylitis. In certain embodiments a compound described herein can decrease peripheral inflammation while treating a CNS disorder. In certain embodiments a compound described herein can treat an inflammatory or metabolic disorder, while minimizing direct CNS effects.

In other aspects of the present invention a compound described herein is used to treat a CNS disorder that has been linked to inadequate functioning of brain systems containing 5-HT_{2A} receptors in mammals. In certain embodiments a compound described herein can be administered in an effective amount to treat a mental disorder described herein or to provide mental enhancement to a human patient in need of thereof. In certain embodiments a compound described herein can be used to treat a host such as a human in need thereof as a milder therapeutic than MDMA and which is faster acting than typical selective serotonin reuptake inhibitors (SSRIs). This enhances the patient experience and encourages the needed medical therapy. In certain embodiments a compound described herein increases empathy, sympathy, openness and/or acceptance of oneself and others. This compound can be taken, if necessary, as part of one or more therapeutic counseling sessions, or when necessary, episodically, or even consistently, as prescribed by a healthcare provider. In some embodiments, a compound of the present invention acts within a reasonable waiting time in a clinic and lasts for one, two, or several hours or otherwise in a time sufficient to complete the therapy session and then diminishes in effect sufficiently for the patient to leave the clinic and resume normal activities. In other embodiments, the compound of the present invention is administered in a periodic or consistent dosage, including a daily dosage in a similar manner to an anti-depressant drug, to enhance self-acceptance, acceptance of others and a general feeling of peace and comfort with surroundings and events.

In certain embodiments a compound of the present invention is used to treat a migraine, headache, or cluster headache. Non-limiting examples of migraines include migraine without aura, migraine with aura, chronic migraine, abdominal migraine, acephalgic migraine, silent migraine, migraine with brainstem aura, hemiplegic migraine, retinal migraine, and status migrainosus.

In certain embodiments a compound of the present invention is used to treat or prevent seizures. Non-limiting examples of seizures include focal aware seizures, focal impaired awareness seizures, bilateral tonic-clonic seizures, absence seizures, atyptical absence seizures, tonic-clonic seizures, atonic seizures, clonic seizures, tonic seizures, myoclonic seizures, gelastic seizures, and dacrystic seizures. In certain embodiments a compound of the present invention is used to treat epilepsy.

In certain aspects the compound of Formula I is selected from:

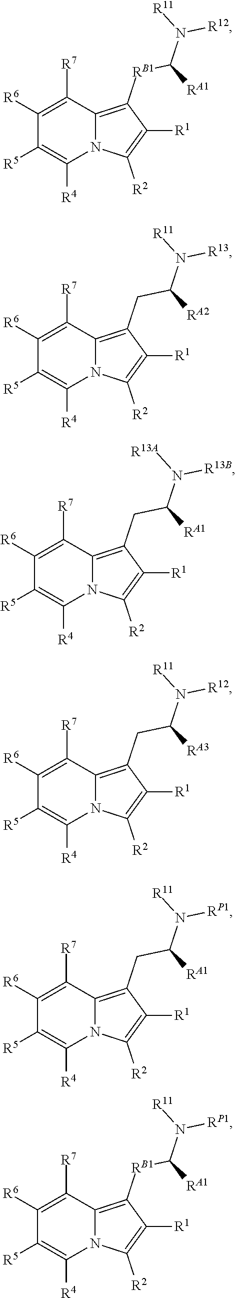

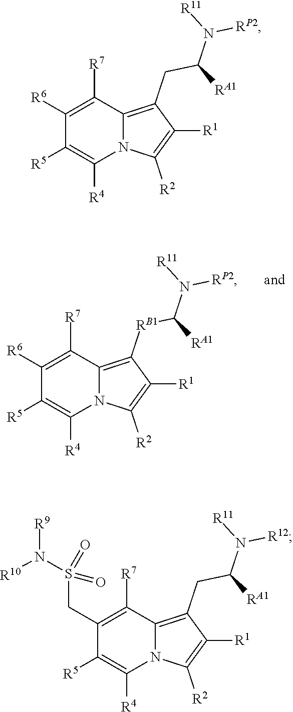

or a pharmaceutically acceptable salt or salt mixture, isotopic derivative, or prodrug thereof.

In certain aspects the compound of Formula I is selected from:

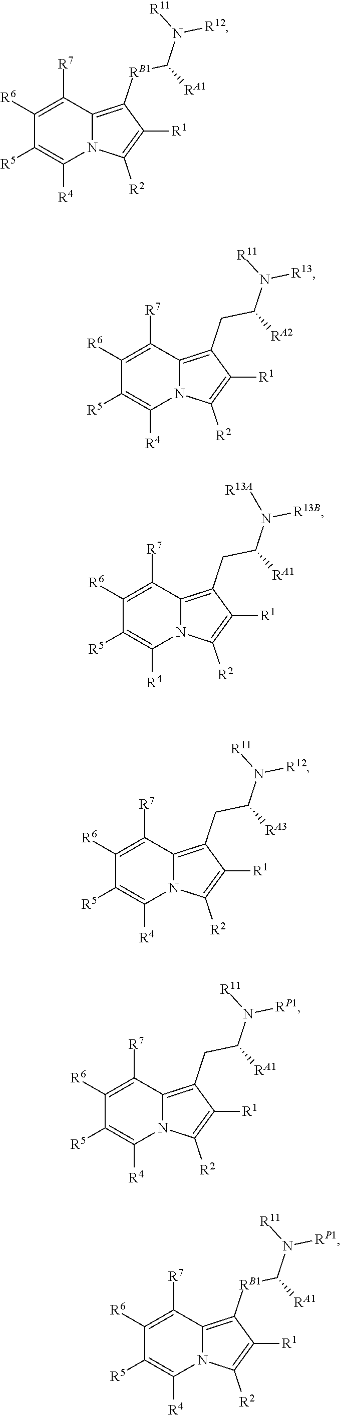

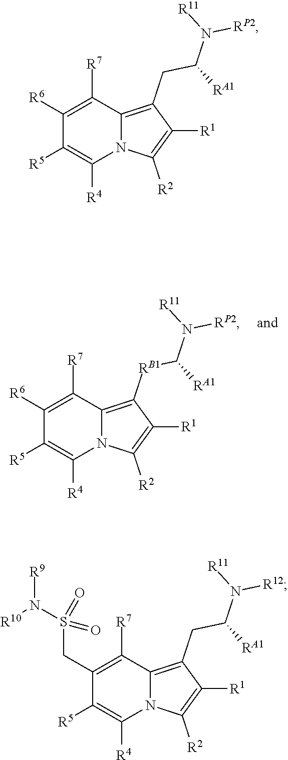

or a pharmaceutically acceptable salt or salt mixture, isotopic derivative, or prodrug thereof.

The R- or S-enantiomers of the present invention can exist in isolated form or mixed in such a way that one enantiomer is present in a greater amount than the other, referred to herein as an enantiomerically enriched mixture. An enantiomerically enriched mixture is a mixture that contains one enantiomer in a greater amount than the other. The term enantiomerically enriched mixture includes either the mixture enriched with the R-enantiomer or enriched with the S-enantiomer. Unless context clearly indicates otherwise, the term “enantiomerically enriched mixture” can be understood to mean “enantiomerically enriched mixture of the R- or S-enantiomer.” An enantiomerically enriched mixture of an S-enantiomer contains at least 55% of the S-enantiomer, and, typically at least about 60%, 65%, 70%, 75%, 80%, 85%, 90%, 95% of the S-enantiomer. An enantiomerically enriched mixture of an R-enantiomer contains at least 55% of the R-enantiomer, and typically at least about 60%, 65%, 70%, 75%, 80%, 85%, 90%, 95% of the R-enantiomer. The specific ratio of S or R enantiomer can be selected for the need of the patient according to the health care specialist to balance the desired effect. Typically, in the present application, the chiral carbon referred to in the term “enantiomerically enriched” is that carbon alpha to the amine in the provided structures. In typical embodiments enantiomerically enriched mixture or R-enantiomer has less than 98% R-enantiomer. In other embodiments the enantiomerically enriched mixture or R-enantiomer has less than or equal to 95% R-enantiomer. In typical embodiments enantiomerically enriched mixture or S-enantiomer has less than 98% S-enantiomer. In other embodiments the enantiomerically enriched mixture or R-enantiomer has less than or equal to 95% S-enantiomer. Thus, the term enantiomerically enriched mixture as used herein typically does not include either a racemic mixture or a pure enantiomer.

In other aspects of the present invention a method of treating a CNS disorder, providing mental enhancement, treating an inflammatory disorder, treating headaches or migraines, or treating a metabolic disorder is provided comprising administering a compound of Formula II to a patient in need thereof:

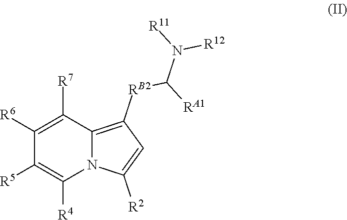

or a pharmaceutically acceptable salt or salt mixture, isotopic derivative, or prodrug thereof; wherein
R^{B2} is

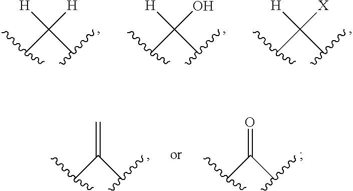

and wherein all other variables are as defined herein.

In certain embodiments a compound of Formula I or II is a fast-acting CNS agent. For example in certain embodiments an effective amount of a fast-acting CNS agent of Formula

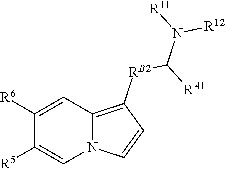

is administered to a patient in need thereof to treat a CNS disorder, wherein the fast-acting CNS agent has activity with a faster onset than other known CNS agents such as MDMA.

In other embodiments a compound of Formula I or II is potent 5-HT_{2A} ligand which modulates inflammation or metabolism. For example in certain embodiments an effective amount of a 5-HT_{2A} ligand of Formula

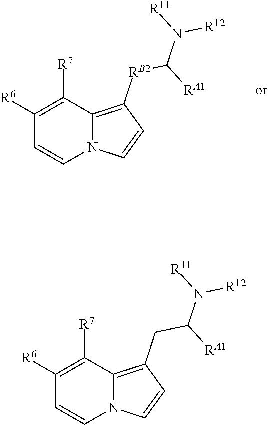

is administered to a patient in need thereof to treat an inflammatory disorder or a metabolic disorder.

In certain embodiments a compound of the present invention has improved neuroplasticity when compared to a known psychoactive agents such as tryptamine.

Non-limiting examples of compounds of the present invention include:

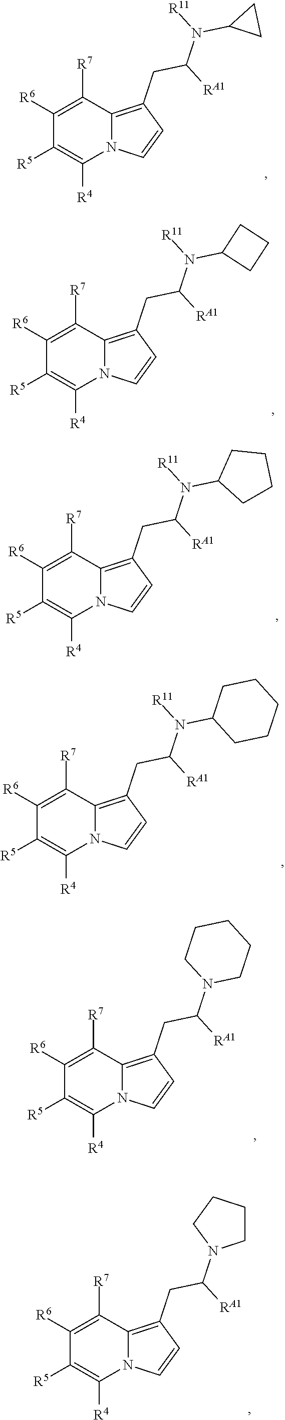

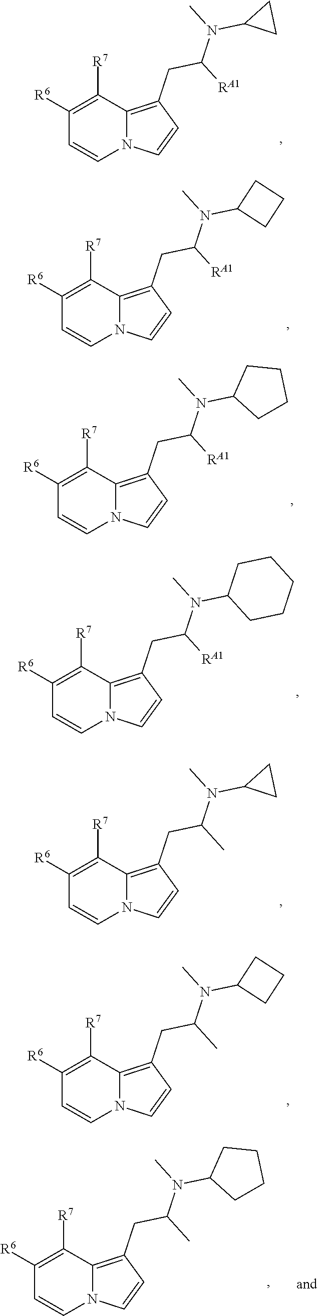

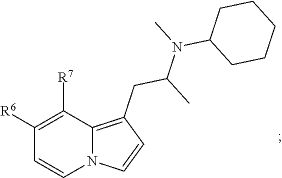

or a pharmaceutically acceptable salt or salt mixture thereof.

Additional non-limiting examples of compounds of the present invention include:

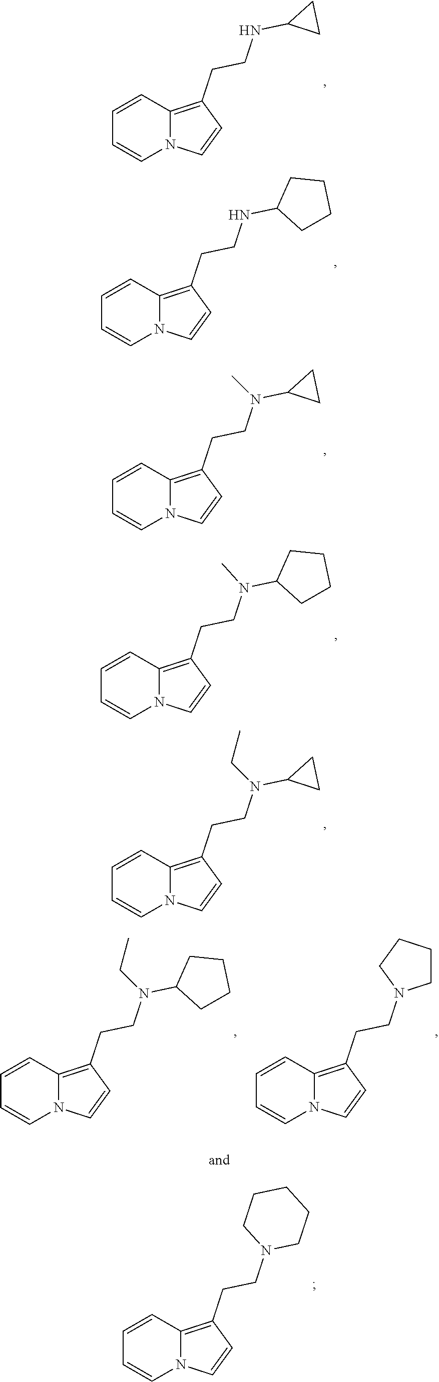

or a pharmaceutically acceptable salt or salt mixture thereof. In some embodiments, the indolizine compound of the current invention, as a racemic mixture, enantiomerically enriched mixture or pure enantiomer has a duration of acute therapeutic effects that is less than that of MDMA (reported to be 4.2 hours with a standard deviation of 1.3 hours after 75 or 125 mg MDMA by Vizeli & Liechti. 2017. Journal of Psychopharmacology, 31(5), 576-588). This can be desirable for reducing the costs and resources needed for pharmacotherapy sessions. In other embodiments, the indolizine compound of the current invention, as a racemic mixture, enantiomerically enriched mixture or pure enantiomer has a duration of acute therapeutic effects that is greater than that of MDMA. This avoids the need for re-administration of the entactogen, which produces nonlinear increases in plasma concentrations and greater unwanted effects.

In some embodiments, the indolizine compound of the current invention, as a racemic mixture, enantiomerically enriched mixture or pure enantiomer produces acute cardiovascular effects that are less than those of MDMA. MDMA produces acute tachycardia and hypertension, which requires safety monitoring and may limit its use in those with preexisting cardiovascular disease (Vizeli & Liechti. 2017. Journal of Psychopharmacology, 31(5), 576-588; MDMA Investigator's Brochure, 13th Edition: Mar. 22, 2021).

In some embodiments a compound of the present invention has favorable pharmacokinetic properties for administration to a mammal, for example a human. These properties can include having more reproducible and less variable pharmacokinetic properties than MDMA. In certain embodiments, a compound of the present invention has a less variable maximum plasma concentration (Cₘₐₓ) than MDMA. In certain embodiments, a compound of the present invention has a less variable area-under-the-concentration-versus-time-curve (AUC) than MDMA. An additional potential beneficial property of a compound of the present invention is reduced inhibition of CYP enzymes compared to MDMA. Inhibition of such enzymes can cause unwanted toxic drug-drug interactions. In certain embodiments, a compound of the present invention does not inhibit or shows minimal inhibition of cytochrome p450 isozyme 2D6 (CYP2D6). In certain embodiments, a compound of the present invention shows less potent inhibition of CYP2D6 than MDMA.

In further embodiments, an indolizine compound of the current invention is a direct 5-HT_{2A} agonist. In yet further embodiments, an indolizine compound of the current invention is a 5-HT releaser. 5-HT_{2A} agonists increase neuroplasticity and decrease inflammation and are currently being investigated for a variety of indications, including for treating chronic pain, headache, depression, anxiety, and substance use disorders. Most substances that are 5-HT_{2A} agonists have significant side effects that are often undesirable in a therapeutic context. For example, psilocybin often produces labile mood with frequent anxiety, derealization, and depersonalization, which are signs and symptoms that limit clinical use. In some aspects of the present invention, an indolizine compound releases 5-HT and is a 5-HT_{2A} agonist while displaying greatly decreased side effects compared to psilocybin, LSD, DMT, 5-MeO-DMT, and other clinically used 5-HT_{2A} agonists.

In further embodiments, an indolizine compound of the current invention is a 5-HT_{1B} agonist. 5-HT_{1B} agonists are being investigated as drugs for producing beneficial modulations in stress sensitivity, inflammation, sociability, mood, anxiety, and aggression. In yet further embodiments, an indolizine compound of the current invention is a 5-HT_{1B} agonist and 5-HT₆ agonist. In further embodiments, an indolizine compound of the current invention that is a 5-HT_{1B} and 5-HT₆ agonist can be used for treating mood and anxiety disorders, stress disorders, and obsessive-compulsive disorder.

In some embodiments, an enantiomerically enriched mixture of the S-enantiomer or pure enantiomer of Formula I increases the serotonin-receptor-dependent actions that contribute to therapeutic effects and minimizes adverse dopaminergic effects that can contribute to unwanted properties like addictive liability when administered to a host in need thereof, for example a mammal, including a human, relative to the racemic form.

In some embodiments, an enantiomerically enriched mixture of the R-enantiomer or pure enantiomer of Formula I increases the serotonin-receptor-dependent actions that contribute to therapeutic effects and minimizes adverse dopaminergic effects that can contribute to unwanted properties like addictive liability when administered to a host in need thereof, for example a mammal, including a human, relative to the racemic form.

In further embodiments, pharmaceutical compositions are disclosed which comprise a compound of Formula I as either racemic, as pure enantiomers, or in an enantiomerically enriched mixture, and which may be in association with another active agent, in a pharmaceutically acceptable composition that has a carrier, diluent, or excipient. The pharmaceutical compositions of the present invention may in certain embodiments include a salt mixture, wherein a salt mixture may comprise 1, 2 or more different pharmaceutically acceptable salts together to form a single composition. In some embodiments, enantiomers are mixed that each has a different salt or wherein there is a ratio of salts, as in Adderall, for example, which is a mixture of a racemate of amphetamine as an aspartate salt, racemate of amphetamine as a sulfate salt, and D-amphetamine as a saccharate salt and D-amphetamine as a sulfate salt. These kinds of mixtures of racemic, enantiomerically enriched and pure compounds can provide advantageous results.

The invention includes methods for modulating the activity of the CNS of a host in need thereof, such as a human, by administering an effective amount of a compound or composition of the invention. Examples are methods for treating a variety of CNS disorders, as generally listed herein, that have been linked to inadequate functioning of serotonergic neurotransmission in mammals, using a compound or composition of the invention. The invention also includes methods of improving CNS functioning such as reducing neuroticism or psychological defensiveness or increasing creativity, decision-making ability, or openness to experience in a human by administering an effective amount of a compound or composition of the invention.

Specifically, the invention includes methods to treat a neurological or psychiatric central nervous system disorder as further described herein, including a mental disorder, or to provide a mental enhancement, with a compound described herein, or a pharmaceutically acceptable salt or salt mixture thereof.

The invention also includes methods for treating disorders associated with inflammation or metabolic disorders host in need thereof, such as a human, by administering an effective amount of a compound or composition of the invention. For example, in certain embodiments a method is provided to treat a disorder selected from arthritis, psoriasis, Crohn's disease, inflammatory bowel syndrome, ulcerative colitis, and ankylosing spondylitis.

Additionally, the invention includes a method of treating a patient with primary or secondary headaches, comprising administering an effective amount of a compound, pure enantiomer, or enantiomerically enriched mixture of a compound of the present invention.

The present invention thus includes at least the following aspects:
(i) A compound of Formula I or a pharmaceutically acceptable salt or salt mixture, isotopic derivative, or prodrug thereof,
(ii) An enantiomerically enriched mixture of a compound of Formula I, or a pharmaceutically acceptable salt or salt mixture, isotopic derivative, or prodrug thereof, (iii) An enantiomerically pure compound of Formula I or a pharmaceutically acceptable salt or salt mixture, isotopic derivative, or prodrug thereof;
(iv) A pharmaceutical composition comprising an effective patient-treating amount of a compound of (i), (ii) or (iii) in a pharmaceutically acceptable carrier or diluent for any of the uses described herein;
(v) The pharmaceutically acceptable composition of (iv) in a solid or liquid, systemic, oral, topical or parenteral dosage form;
(vii) A method for treating any neurological or psychological CNS disorder comprising administering an effective amount of a compound of (i), (ii) or (iii) or a compound of Formula II or a pharmaceutically acceptable salt, isotopic derivative, or prodrug thereof, as described herein, to a patient, typically a human, in need thereof,
(viii) A method for treating an inflammatory disorder or metabolic disorder comprising administering an effective amount of a compound of (i), (ii) or (iii) or a compound of Formula II or a pharmaceutically acceptable salt, isotopic derivative, or prodrug thereof, as described herein, to a patient, typically a human, in need thereof,
(ix) A compound of (i), (ii) or (iii) or a compound of Formula II or a pharmaceutically acceptable salt, salt mixture, isotopic derivative, or prodrug thereof, for use to treat any disorder as described herein in an effective amount as further described herein;
(x) A compound of (i), (ii) or (iii) or a compound of Formula II for use in the manufacture of a medicament for the treatment of any of the disorders described herein;
(xi) Use of a compound of (i), (ii) or (iii) or a compound of Formula II or a pharmaceutically acceptable salt, salt mixture, isotopic derivative, or prodrug thereof, to treat any disorder as described herein in an effective amount as further described herein; and
(xii) Processes for the preparation of therapeutic products that contain an effective amount of a compound of (i), (ii) or (iii) or a compound of Formula II or a pharmaceutically acceptable salt or salt mixtures, isotopic derivatives, or prodrugs or compositions thereof, as described herein.

### BRIEF DESCRIPTION OF THE FIGURES

FIG. 1 is a concentration-response curve for compound 26-5 HCl when tested in the Dopamine Release Assay in rat synaptosomes as described in Example 12. The X-axis depicts the concentration (μM) of the compound, and the Y-axis depicts the normalized response.

FIG. 2 is a concentration-response curve for compound 27-1 HCl when tested in the Dopamine Release Assay in rat synaptosomes as described in Example 12. The X-axis depicts the concentration (μM) of the compound, and the Y-axis depicts the normalized response.

FIG. 3 is a concentration-response curve for compound 25-14 HCl when tested in the Dopamine Release Assay in rat synaptosomes as described in Example 12. The X-axis depicts the concentration (μM) of the compound, and the Y-axis depicts the normalized response.

FIG. 4 is a concentration-response curve for compound 28-12 oxalate when tested against the HTR2A (5-hydroxytryptamine (serotonin) receptor 2A) in the in vitro assay described in Example 15. The X-axis depicts the concentration (μM) of the compound, and the Y-axis depicts the normalized response.

FIG. 5 provides non-limiting examples of indolizine compounds of the present invention.

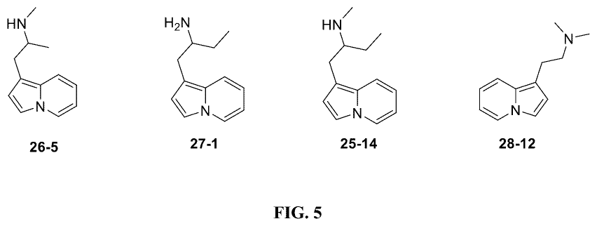

### Definitions

When introducing elements of the present invention or the typical embodiments thereof, the articles “a,” “an,” “the,” and “said” are intended to mean that there are one or more of the elements. The terms “comprising,” “including,” and “having” are intended to be inclusive and not exclusive (i.e., there may be other elements in addition to the recited elements). Thus, the terms “including,” “may include,” and “include,” as used herein mean, and are used interchangeably with, the phrase “including but not limited to.”

Where a range of values is provided, it is understood that the upper and lower limit, and each intervening value between the upper and lower limit of the range is encompassed within the embodiments.

Unless defined otherwise, all technical and scientific terms herein have the meaning as commonly understood by one of ordinary skill in the art to which this invention belongs. In the event there is a plurality of definitions for a term herein, those in this section prevail unless stated otherwise. Further definitions that may assist the reader to understand the disclosed embodiments are as follows, and such definitions may be used to interpret the defined terms, when those terms are used herein. However, the examples given in the definitions are generally non-exhaustive and must not be construed as limiting the invention. It also will be understood that a substituent should comply with chemical bonding rules and steric compatibility constraints in relation to the particular molecule to which it is attached.

A compound of the invention may contain one or more chiral centers and/or double bonds and therefore, may exist as stereoisomers, such as double-bond isomers (i.e., geometric isomers), enantiomers, or diastereomers. Accordingly, the chemical structures depicted herein independently encompass all possible enantiomers and stereoisomers of the illustrated compounds including the stereoisomerically pure form (for example, geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer. An enantiomerically enriched mixture of an R-enantiomer contains at least 55% of the R-enantiomer, and typically at least about 60%, 65%, 70%, 75%, 80%, 85%, 90% or 95% of the R-enantiomer. The specific ratio of S or R enantiomer can be selected for the need of the patient according to the health care specialist to balance the desired effect. In certain embodiments, as indicated by context, the term enantiomerically enriched does not include a pure enantiomer. The term enantiomerically enriched mixture as used in this application does not include a racemic mixture and does not include a pure isomer. Notwithstanding, it should be understood that any compound described herein in enantiomerically enriched form can be used as a pure isomer if it achieves the goal of any of the specifically itemized methods of treatment described herein.

“Composition of the invention” refers to at least one compound of the invention and a pharmaceutically acceptable vehicle, with which the compound is administered to a patient. When administered to a patient, the compounds of the invention are administered in isolated form, which means separated from a synthetic organic reaction mixture.

“Alkyl” is a branched, straight chain, or cyclic saturated hydrocarbon group. In one non-limiting embodiment, the alkyl group contains from 1 to 12 carbon atoms or more typically from 1 to 6 carbon atoms, from 1 to 4 carbon atoms, or from 1 to 3 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having 1, 2, 3, 4, 5, or 6 carbon atoms and is intended to mean that each of these is described as an independent species. For example, the term C₁-C₄ alkyl as used herein indicates a straight or branched alkyl group having 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. Unless otherwise indicated, the term alkyl includes cycloalkyl or carbocycle.

“Alkenyl” is a linear or branched hydrocarbon group having one or more carbon-carbon double bonds that may occur at a stable point along the chain. The specified ranges as used herein indicate an alkenyl group having each member of the range described as an independent species, as described above for the alkyl moiety. In one non-limiting embodiment, the alkenyl contains from 2 to 12 carbon atoms, more typically from 2 to 6 carbon atoms or from 2 to 4 carbon atoms.

In certain embodiments the alkenyl is C₂, C₂-C₃, C₂-C₄, C₂-C₅, or C₂-C₆. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. The term “Alkenyl” also encompasses cycloalkyl or carbocyclic groups possessing at least one point of unsaturation.

“Alkynyl” is a branched or straight chain hydrocarbon group having one or more carbon-carbon triple bonds that may occur at any stable point along the chain. The specified ranges as used herein indicate an alkynyl group having each member of the range described as an independent species, as described above for the alkyl moiety. In one non-limiting embodiment, the alkynyl contains from 2 to 12 carbon atoms, more typically from 2 to 6 carbon atoms or from 2 to 4 carbon atoms. In certain embodiments the alkynyl is C₂, C₂-C₃, C₂-C₄, C₂-C₅, or C₂-C₆. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. The term “Alkynyl” also encompasses cycloalkyl or carbocyclic groups possessing at least one point of triple bond unsaturation.

“Halo” and “Halogen” is independently fluorine, chlorine, bromine or iodine.

“Haloalkyl” is a branched or straight-chain alkyl group substituted with 1 or more halo atoms described above, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

As used herein, “aryl” refers to a radical of a monocyclic or polycyclic (e.g., bicyclic or tricyclic) 4n+2 aromatic ring system (e.g., having 6, 10, or 14 π L electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more cycloalkyl or heterocycle groups wherein the radical or point of attachment is on the aryl ring, and in such instances, the number of carbon atoms continue to designate the number of carbon atoms in the aryl ring system. The one or more fused cycloalkyl or heterocycle groups can be a 4 to 7-membered saturated or partially unsaturated cycloalkyl or heterocycle groups.

“Arylalkyl” refers to an alkyl group as defined herein substituted with an aryl group as defined herein. Typical arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀), more preferably, an arylalkyl group is (C₆-C₂₀) arylalkyl, e.g., the alkanyl, alkenyl or alkynyl moiety of the arylalkyl group is (C₁-C₈) and the aryl moiety is (C₆-C₁₂).

“Heteroalkyl” refers to an alkyl group, in which one or more of the carbon atoms (and any associated hydrogen atoms) are each independently replaced with the same or different heteroatomic groups. Typical heteroatomic groups include —O—, —S—, —O—O—, —S—S—, —O—S—, —NR′, ═N—N═, —N═N—, —N═N—NR′, —PH—, —P(O)₂—, —O—P(O)—, —S(O)—, —S(O)₂—, —SnH₂—and the like, wherein R′ is hydrogen, alkyl, substituted alkyl, cycloalkyl, substituted cycloalkyl, aryl or substituted aryl.

The term “heterocycle” denotes saturated and partially saturated heteroatom-containing ring radicals, wherein there are 1, 2, 3, or 4 heteroatoms independently selected from nitrogen, sulfur, boron, silicon, and oxygen. Heterocyclic rings may comprise monocyclic 3-10 membered rings, as well as 5-16 membered bicyclic ring systems (which can include bridged, fused, and spiro-fused bicyclic ring systems). For example, a partially unsaturated condensed heterocyclic group containing 1 to 5 nitrogen atoms, for example, indoline or isoindoline; a partially unsaturated condensed heterocyclic group containing 1 to 2 oxygen atoms and 1 to 3 nitrogen atoms; a partially unsaturated condensed heterocyclic group containing 1 to 2 sulfur atoms and 1 to 3 nitrogen atoms; and a saturated condensed heterocyclic group containing 1 to 2 oxygen or sulfur atoms. The term “heterocycle” does not include rings containing —O—O—, —O—S— or —S—S— portions. Examples of saturated heterocycle groups include saturated 3- to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ-benzo[d]isothiazol-6-yl, dihydropyranyl, dihydrofuryl and dihydrothiazolyl.

“Heterocycle” also includes groups wherein the heterocyclic radical is fused/condensed with an aryl or carbocycle radical, wherein the point of attachment is the heterocycle ring.

“Heterocycle” also includes groups wherein the heterocyclic radical is substituted with an oxo group (i.e.

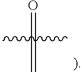 [SMILES: *=O]

The term “heterocycle” also includes “bicyclic heterocycle”. The term “bicyclic heterocycle” denotes a heterocycle as defined herein wherein there is one bridged, fused, or spirocyclic portion of the heterocycle. The bridged, fused, or spirocyclic portion of the heterocycle can be a carbocycle, heterocycle, or aryl group as long as a stable molecule results. Unless excluded by context the term “heterocycle” includes bicyclic heterocycles. Bicyclic heterocycle includes groups wherein the fused heterocycle is substituted with an oxo group. Non-limiting examples of bicyclic heterocycles include:

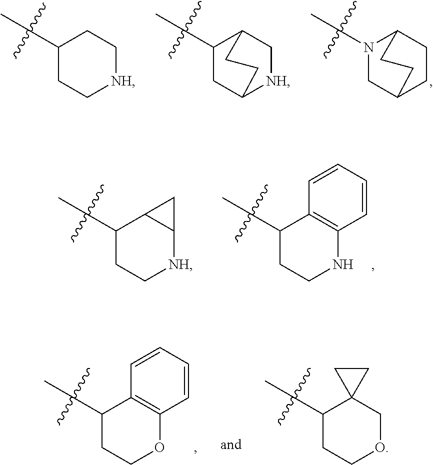

“Heterocyclealkyl” refers to either an alkyl group as defined herein substituted with a heterocycle group as defined herein.

The term “heteroaryl” denotes stable aromatic ring systems that contain 1, 2, 3, or 4 heteroatoms independently selected from O, N, and S, wherein the ring nitrogen and sulfur atom(s) are optionally oxidized, and nitrogen atom(s) are optionally quarternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5- oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. In certain embodiments the “heteroaryl” group is a 8, 9, or 10 membered bicyclic ring system. Examples of 8, 9, or 10 membered bicyclic heteroaryl groups include benzofurazanyl, benzothiophenyl, benzothiazolyl, benzoxazolyl, quinazolinyl, quinoxalinyl, naphthyridinyl, quinolinyl, isoquinolinyl, benzofuranyl, indolyl, indazolyl, and benzotriazolyl.

“Heteroarylalkyl” refers to either an alkyl group as defined herein substituted with a heteroaryl group as defined herein.

As used herein, “carbocyclic”, “carbocycle” or “cycloalkyl” includes a saturated or partially unsaturated (i.e., not aromatic) group containing all carbon ring atoms and from 3 to 14 ring carbon atoms (“C₃₋₁₄ cycloalkyl”) and zero heteroatoms in the non-aromatic ring system. In some embodiments, a cycloalkyl group has 3 to 10 ring carbon atoms (“C₃₋₁₀ cycloalkyl”). In some embodiments, a cycloalkyl group has 3 to 9 ring carbon atoms (“C₃₋₉ cycloalkyl”). In some embodiments, a cycloalkyl group has 3 to 8 ring carbon atoms (“C₃₋₈ cycloalkyl”). In some embodiments, a cycloalkyl group has 3 to 7 ring carbon atoms (“C₃₋₇ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cyclononyl (C₉), cyclononenyl (C₉), cyclodecyl (C₁₀), cyclodecenyl (C₁₀), and the like. As the foregoing examples illustrate, in certain embodiments, the cycloalkyl group can be saturated or can contain one or more carbon-carbon double bonds. The term “cycloalkyl” also includes ring systems wherein the cycloalkyl ring, as defined above, is fused with one heterocycle, aryl or heteroaryl ring wherein the point of attachment is on the cycloalkyl ring, and in such instances, the number of carbons continue to designate the number of carbons in the carbocyclic ring system.

The term “CNS disorder” as used herein refers to either a neurological condition (one that is typically treated by a neurologist) or a psychiatric condition (one that is typically treated by a psychiatrist). Neurological disorders are typically those affecting the structure, biochemistry or normal electrical functioning of the brain, spinal cord or other nerves. Psychiatric conditions are more typically thought of as mental disorders, which are primarily abnormalities of thought, feeling or behavior that cause significant distress or impairment of personal functioning. Thus, a disclosed compound can be used in an effective amount to improve neurological or psychiatric functioning in a patient in need thereof. Neurological indications include, but are not limited to improved neuroplasticity, including treatment of stroke, brain trauma, dementia, and neurodegenerative diseases. A compound of the current invention can be considered a psychoplastogen, that is, a small molecule that is able to induce rapid neuroplasticity. For example, in certain embodiments, the disclosed compound or composition can be used to improve stuttering and other dyspraxias or to treat Parkinson's disease or schizophrenia.

The term “neurological disease or disorder” includes Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that a disclosed compound will allow individuals to effectively contemplate actual or possible experiences that would normally be upsetting or even overwhelming. This includes individuals with fatal illness planning their last days and the disposition of their estate. This also includes couples discussing difficulties in their relationship and how to address them. This also includes individuals who wish to more effectively plan their career.

The term “inadequate functioning of neurotransmission” is used synonymously with a CNS disorder that adversely affects normal healthy neurotransmission.

Examples of isotopes that can be incorporated into a compound of the invention include isotopes of hydrogen, carbon, nitrogen, oxygen, fluorine and chlorine such as ²H, ³H, ¹¹C, ^{13C}, ¹⁴C, ¹³N ¹⁵N, ¹⁷O, ¹⁸O, ¹⁸F, ³⁶Cl, and respectively. In some non-limiting embodiments, an isotopically labelled compound can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. An isotopically labeled compound of this invention and a prodrug thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (3H) may be used anywhere in described structures that achieves the desired result. Alternatively, or in addition, isotopes of carbon, for example, ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is at least 60, 70, 80, 90, 95 or 99% or more enriched in an isotope at any location of interest. In some non-limiting embodiments, deuterium is at least 80, 90, 95 or 99% enriched at a desired location. Unless indicated to the contrary, the deuteration is at least 80% at the selected location. Deuteration can occur at any replaceable hydrogen that provides the desired results.

In some non-limiting embodiments, the substitution of a hydrogen atom for a deuterium atom can be provided in a compound or composition described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). A compound of the invention also includes an isotopically labeled compound where one or more atoms have an atomic mass different from the atomic mass conventionally found in nature. Examples of isotopes that may be incorporated into a compound of the invention include ²H, ³H, ¹³C, ¹⁴C, ¹³N, ¹⁵N, ¹⁸O, ¹⁷O, ³¹P, ³²P, ³⁵S, ¹⁸F, and ³⁶Cl.

An alkyl group on the nitrogen of Formula I of the invention is subject to enzymatic removal. The N-alkyl may be prepared with a deuterated reagent that replaces one, two, any, or all of the hydrogens on the N-alkyl group, which creates a higher activation energy for bond cleavage and a slower formation of the desalkyl metabolite. In general, when deuterium is substituted for a hydrogen at a location of metabolism in the compound, a more stable compound will result.

Several compounds of the present invention have a chiral center and thus exists as enantiomers that may be more appropriate for some applications. Accordingly, the present disclosure also includes stereoisomers of a compound described herein, where applicable, either individually or admixed in any proportions. Stereoisomers may include enantiomers, diastereomers, racemic mixtures, and combinations thereof.

“Stereoisomers” includes enantiomers, diastereomers, the components of racemic mixtures, and combinations thereof. Stereoisomers can be prepared or separated as described herein or by using other methods.

Such stereoisomers can be prepared and separated using conventional techniques, either by reacting enantiomeric starting materials, or by separating isomers of a compound disclosed herein.

“Agonist” refers to a modulator that binds to a receptor or enzyme and activates the receptor to produce a biological response. In some embodiments, “agonist” includes full agonists or partial agonists.

“Antagonism” refers to the inactivation of a receptor or enzyme by a modulator, or antagonist. Antagonism of a receptor, for example, is when a molecule binds to the receptor and does not allow activity to occur.

“IC₅₀” refers to the concentration of a substance (for example, a compound or a drug) that is required for 50% inhibition of a biological process. For example, IC₅₀ refers to the half maximal (50%) inhibitory concentration (IC) of a substance as determined in a suitable assay. Similarly, EC₅₀ refers to the concentration of a substance that provokes a response halfway between the baseline activity and maximum response. In some instances, an IC₅₀ or EC₅₀ is determined in an in vitro assay system. In some embodiments as used herein, IC₅₀ (or EC₅₀) refers to the concentration of a modulator that is required for 50% inhibition (or excitation) of a receptor, for example, 5HT_{1B}.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (for example, positive allosteric modulator) of a G protein-coupled receptor (for example, 5-HT_{1B}) are modulators of the receptor.

“Neuroplasticity” refers to the ability of the brain to change its structure and/or function throughout a subject's life. Examples of the changes to the brain include, but are not limited to, the ability to adapt or respond to internal and/or external stimuli, such as due to an injury, and the ability to produce new neurites, dendritic spines, and synapses.

“Subject,” as used herein, refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. The subject can be, for example, a child, such as an adolescent, or an adult.

“Treating” or “treatment” of a disease, as used in context, includes (i) inhibiting the disease, i.e., arresting or reducing the development or progression of the disease or its clinical symptoms; or (ii) relieving the disease, i.e., causing regression of the disease or its clinical symptoms. Inhibiting the disease, for example, would include prophylaxis. Hence, one of skill in the art will understand that a therapeutic amount necessary to effect treatment for purposes of this invention will, for example, be an amount that provides for objective indicia of improvement in patients having clinically diagnosable symptoms. Other such measurements, benefits, and surrogate or clinical endpoints, whether alone or in combination, would be understood to those of ordinary skill.

“Therapeutic effect” means the responses(s) in a host after treatment that is judged to be desirable or beneficial. Hence, depending on the CNS disorder to be treated, or improvement in CNS functioning sought, those responses shall differ, but would be readily understood by those of ordinary skill.

“Thio” means the radical —SH.

“Amino acid” refers the structural units of proteins. The twenty amino acids encoded by the genetic code are called “standard amino acids.” These amino acids have the structure H₂N—CHR—COOH, where R is a side chain specific to the amino acids. Standard amino acids are Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, and Valine. In addition to the twenty standard amino acids, there are two additional amino acids called selenocysteine and pyrroline. “Non-standard amino acids” are additional amino acids that are not typically incorporated into proteins. These include the sulfur-containing Taurine and the neurotransmitter Gamma-aminobutyric acid (GABA). Other examples are Lanthionine, 2-Aminoisobutyric acid, Dehydroalanine, Carnitine, Ornithine, and Citrulline. Amino acids can be present in two stereoisometric forms, called “D” and “L.” The D and L form of any amino acid have identical physical properties and chemical reactivities, but rotate the plane of plane-polarized light equally but in opposite directions and react at different rates with asymmetric reagents. Many enzymes acting upon amino acids have asymmetric binding sites and thus can discriminate between the D and L forms. Unless otherwise specified, discussion of any amino acid is intended to refer to all isomers. It is to be understood that amino acids may be modified to mask hydrogen bond donors and improve absorption, following the approach of Barlow et al. (2020, ACS Chemical Biology, 15(8), 2070-2078), wherein polarity is approximately preserved by adding structures with hydrogen bond acceptors to mask the donors.

TABLE I
Amino Acid Abbreviations

| Abbreviation | Amino acid name |
|---|---|
| 3Hyp | 3-Hydroxyproline |
| 4Abu | 4-Aminobutyric acid, piperidinic acid |
| 4Hyp | 4-Hydroxyproline |
| Aad | 2-Aminoadipic acid |
| Abu | 2-Aminobutyric acid |
| Acp | 6-Aminocaproic acid |
| Ahe | 2-Aminoheptanoic acid |
| aHyl | allo-Hydroxylysine |
| Aib | 2-Aminoisobutyric acid |
| aIle | allo-Isoleucine |
| Ala | Alanine |
| Apm | 2-Aminopimelic acid |
| Arg | Arginine |
| Asn | Asparagine |
| Asp | Aspartic acid |
| Asx | Aspartic acid or Asparagine |
| bAad | 3-Aminoadipic acid |
| bAib | 3-Aminoisobutyric acid |
| bAla | beta-Alanine, beta-Aminoproprionic acid |
| Cys | Cysteine |
| Dbu | 2,4-Diaminobutyric acid |
| Des | Desmosine |
| Dpm | 2,2′-Diaminopimelic acid |
| Dpr | 2,3-Diaminoproprionic acid |
| EtAsn | N-Ethylasparagine |
| EtGly | N-Ethylglycine |
| Gln | Glutamine |
| Glu | Glutamic acid |
| Glx | Glutamic acid or Glutamine |
| Gly | Glycine |
| His | Histidine |
| Hyl | Hydroxylysine |
| Ide | Isodesmosine |
| Ile | Isoleucine |
| Leu | Leucine |
| Lys | Lysine |
| MeGly | N-Methylglycine, sarcosine |
| MeIle | N-Methylisoleucine |
| MeLys | 6-N-Methyllysine |
| Met | Methionine |
| MeVal | N-Methylvaline |
| Nle | Norleucine |
| Nva | Norvaline |
| Orn | Ornithine |
| Phe | Phenylalanine |
| Pro | Proline |
| Pyl | Pyrrolysine |
| Sec | Selenocysteine |
| Ser | Serine |
| Thr | Threonine |
| Trp | Tryptophan |
| Tyr | Tyrosine |
| Val | Valine |
| Xaa | Any amino acid |
| Xle | Leucine or Isoleucine |

In certain embodiments “Pharmaceutically acceptable” means approved or approvable by a regulatory agency of the Federal or a state government or listed in the U.S. Pharmacopoeia or other generally recognized pharmacopoeia for use in animals, particularly mammals, and more particularly humans.

In certain embodiments “Pharmaceutically acceptable salt” refers to a salt of a compound of the invention, which is pharmaceutically acceptable and possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc, and the like. (See Berge et al. (1977) “Pharmaceutical Salts,” J. Pharm. Sci. 66:1-19.)

In certain embodiments “Pharmaceutically acceptable vehicle” or “pharmaceutically acceptable carrier,” refers to a diluent, adjuvant, excipient or carrier with which a compound of the invention is administered. This term includes a 0.01-0.1M and preferably 0.05M phosphate buffer, or in another embodiment 0.8% saline. Additionally, pharmaceutically acceptable carriers may be in other embodiments aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments, the carrier can be a) 10% PEG 400 (v/v)+30% (v/v) HPPCD, 50% w/v+60% (v/v) Sterile Water for Injection or b) 0.1% (v/v) Tween 80+0.5% (w/v) Carboxymethylcellulose in water.

In certain embodiments “Prodrug” refers to a derivative of a drug molecule that requires a transformation within the body to release a desired active drug. Prodrugs are frequently (though not necessarily) pharmacologically less active or inactive until converted to the parent drug. The prodrug will contain an “active” component, e.g., an indolizine-based drug, and a prodrug moiety. Removal of some or all of the prodrug moiety will convert the prodrug from a less active form to the desired active drug. This is done in the body by a chemical or biological reaction. In some cases, the moiety or chemicals formed from it may also have beneficial effects, including increasing therapeutic effects, decreasing undesirable side effects, or otherwise altering the pharmacokinetics or pharmacodynamics of the active drug. When the chemical formed from the prodrug moiety has beneficial effects that contribute to the overall beneficial effects of administering the prodrug, then the formed chemical is considered a “codrug.”

Types of prodrugs contemplated to be within the scope and spirit of the invention therefore include compounds that are transformed in various organs or locations in the body (e.g., liver, kidney, G.I., lung, tissue) to release the active compound. For example, liver prodrugs will include active compounds conjugated with a polymer or chemical moiety that is not released until acted upon by liver cytochrome enzymes; CYP metabolism includes dealkylation, dehydrogenation, reduction, hydrolysis, oxidation, and the breakdown of aromatic rings. Kidney prodrugs will include active compounds conjugated to L-gamma-glutamyl or N-acetyl-L-gamma glutamic moieties so that they are metabolized by gamma-glutamyl transpeptidase before they are bioactive; alternatively, they may be conjugated to alkylglucoside moieties to create glycosylation-based prodrugs. Digestive or G.I. prodrugs will include those where an active compound is, e.g., formulated into microspheres or nanospheres that do not degrade until the spheres are subjected to an acidic pH; formulated with an amide that will resist biochemical degradation until colonic pH is achieved; or conjugated with a linear polysaccharide such as pectin that will delay activation until the combination reaches the bacteria in the colon. Besides these exemplary prodrug forms, many others will be known to those of ordinary skill. Non-limiting examples of prodrugs are described in US20220324889A1 and WO2023283364.

“Substituted” refers to a group in which one or more hydrogen atoms are each independently replaced with the same or different substituent(s). In typical embodiments when not excluded by context optional substituents are selected from halogen, —R¹⁴, ═O, —OR¹⁴, —SR¹⁴, ═S, —NR¹⁴R¹⁵, —CH₂X, —CHX₂, —CX₃, —CN, —NO₂, —S(O)₂R¹⁴, —OS(O)₂R¹⁴, —P(O)(OR¹⁴)(OR¹⁵), —C(O)R¹⁴, —C(S)R¹⁴, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵, wherein each R¹⁴, R¹⁵, and R¹⁶, is independently hydrogen, alkyl, aryl, arylalkyl, cycloalkyl, heterocycle, heteroaryl, heteroarylalkyl, —C(O)R¹⁸ or —S(O)₂R¹⁸; and R¹⁸ is independently hydrogen, alkyl, aryl, arylalkyl, cycloalkyl, heterocycle, heteroaryl, or heteroarylalkyl.

### DETAILED DESCRIPTION OF THE INVENTION

Among the various aspects of the present invention are compounds, compositions, methods for modulation of CNS activity, and methods for treatment of CNS disorders, such as posttraumatic stress and adjustment disorders, comprising the indolizines disclosed herein. Methods to treat headaches, migraines, inflammatory disorders, and metabolic disorders are also provided.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

### EMBODIMENTS OF THE PRESENT INVENTION

### Embodiments of “Alkyl”

In certain embodiments “alkyl” is a C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

Non-limiting examples of “alkyl” include: methyl, ethyl, propyl, and isopropyl.

Additional non-limiting examples of “alkyl” include: butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of “Haloalkyl”

In certain embodiments “haloalkyl” is C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has two carbons and one halogen.

In certain embodiments “haloalkyl” has two carbons and two halogens.

In certain embodiments “haloalkyl” has two carbons and three halogens.

In certain embodiments “haloalkyl” has two carbons and four halogens.

In certain embodiments “haloalkyl” has two carbons and five halogens.

In certain embodiments “haloalkyl” has three carbons.

In certain embodiments “haloalkyl” has three carbons and one halogen.

In certain embodiments “haloalkyl” has three carbons and two halogens.

In certain embodiments “haloalkyl” has three carbons and three halogens.

In certain embodiments “haloalkyl” has three carbons and four halogens.

In certain embodiments “haloalkyl” has three carbons and five halogens.

In certain embodiments “haloalkyl” has three carbons and six halogens.

In certain embodiments “haloalkyl” has three carbons and seven halogens.

In certain embodiments “haloalkyl” has four carbons.

In certain embodiments “haloalkyl” has five carbons.

Non-limiting examples of “haloalkyl” include:

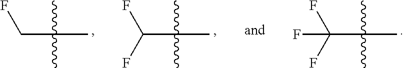

Additional non-limiting examples of “haloalkyl” include:

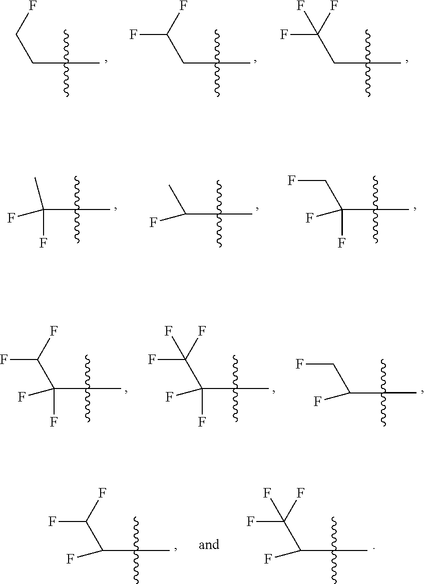

Additional non-limiting examples of “haloalkyl” include:

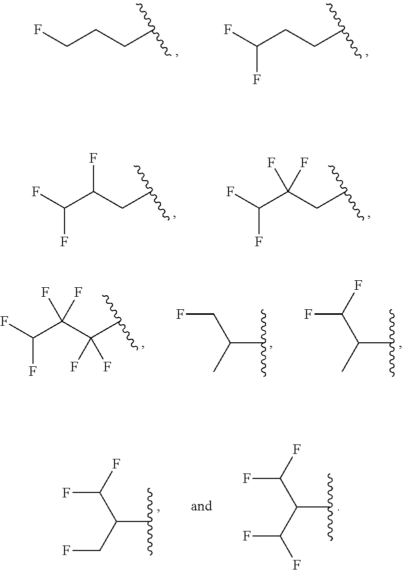

Additional non-limiting examples of “haloalkyl” include:

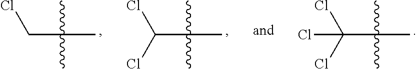

Additional non-limiting examples of “haloalkyl” include:

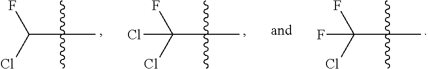

### Embodiments of “Aryl”

In certain embodiments “aryl” is a 6 carbon aromatic group (phenyl).

In certain embodiments “aryl” is a 10 carbon aromatic group (napthyl).

In certain embodiments “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example,

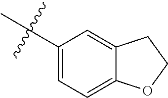 [SMILES: *c1ccc2c(c1)CCO2]

is an “aryl” group.

However,

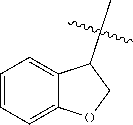 [SMILES: *C1COc2ccccc21]

is a “heterocycle” group.

In certain embodiments “aryl” is a 6 carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example,

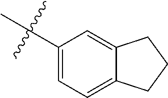 [SMILES: *c1ccc2c(c1)CCC2]

is an “aryl” group.

However,

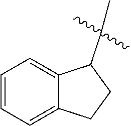 [SMILES: *C1CCc2ccccc21]

is a “cycloalkyl” group.

### Embodiments of “Heteroaryl”

In certain embodiments “heteroaryl” is a 5 membered aromatic group containing 1, 2, 3, or 4 nitrogen atoms.

Non-limiting examples of 5 membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5 membered “heteroaryl” groups include:

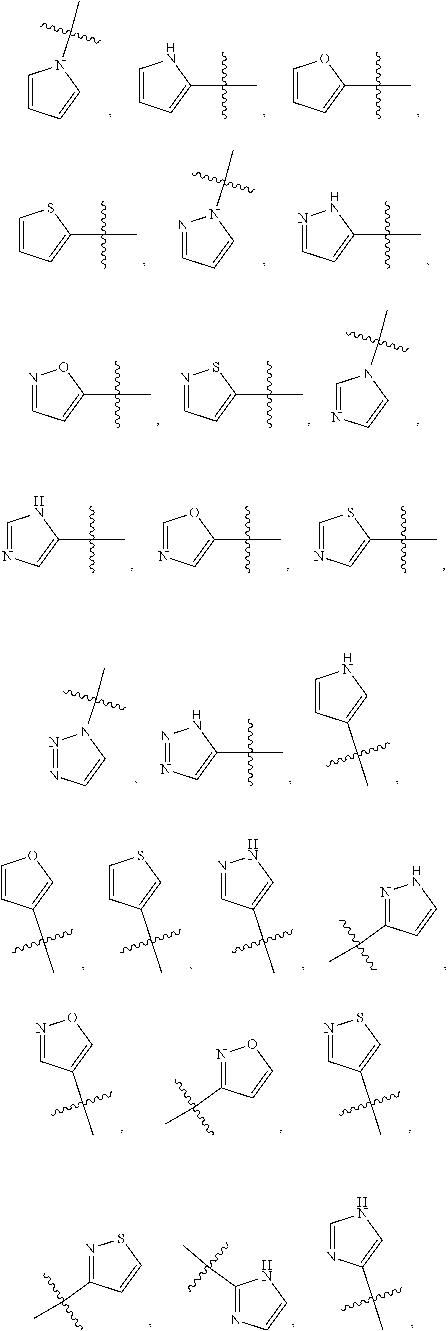

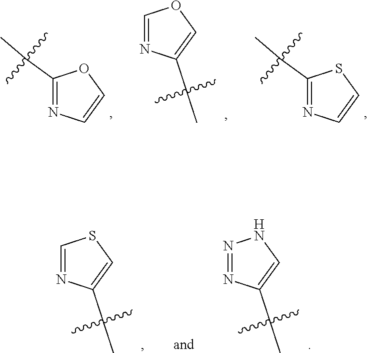 [SMILES: *c1ncco1 ; *c1cocn1 ; *c1nccs1 ; *c1cscn1 ; *c1c[nH]nn1]

In certain embodiments “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

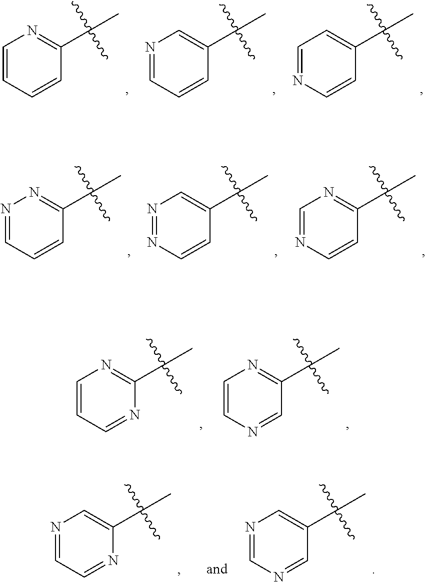

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

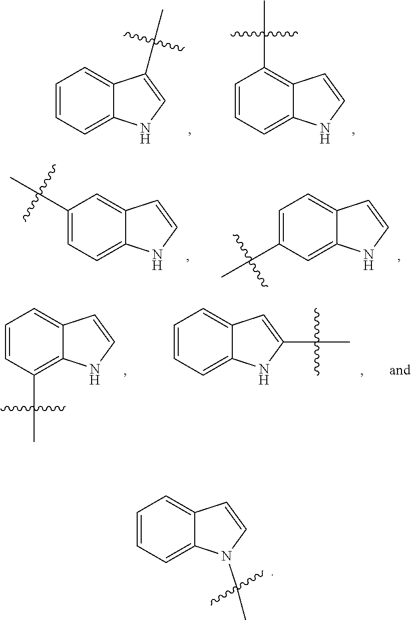

Additional non-limiting examples of “heteroaryl” groups that are bicyclic include:

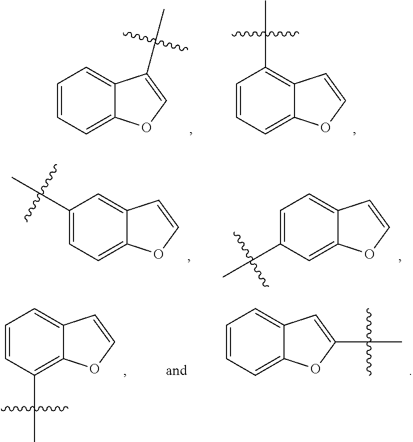 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

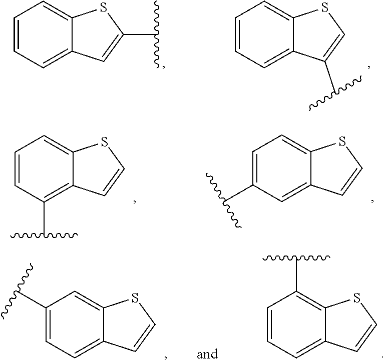 [SMILES: *c1cc2ccccc2s1 ; *c1csc2ccccc12 ; *c1cccc2sccc12 ; *c1ccc2sccc2c1 ; *c1ccc2ccsc2c1 ; *c1cccc2ccsc12]

Additional non-limiting examples of “heteroaryl” groups that are bicyclic include:

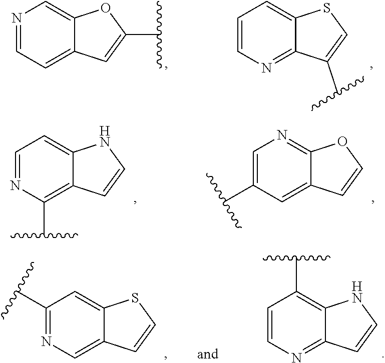 [SMILES: *c1cc2ccncc2o1 ; *c1csc2cccnc12 ; *c1nccc2[nH]ccc12 ; *c1cnc2occc2c1 ; *c1cc2sccc2cn1 ; *c1ccnc2cc[nH]c12]

Additional non-limiting examples of “heteroaryl” groups that are bicyclic include:

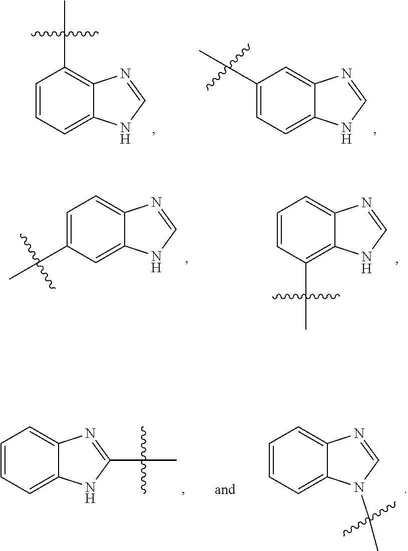 [SMILES: *c1cccc2[nH]cnc12 ; *c1ccc2[nH]cnc2c1 ; *c1ccc2nc[nH]c2c1 ; *c1cccc2nc[nH]c12 ; *c1nc2ccccc2[nH]1 ; *n1cnc2ccccc21]

In certain embodiments “heteroaryl” is a 10 membered bicyclic aromatic group containing 1 or 2 nitrogens.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

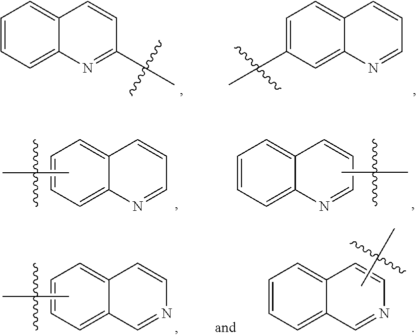

Additional non-limiting examples of “heteroaryl” groups that are bicyclic include:

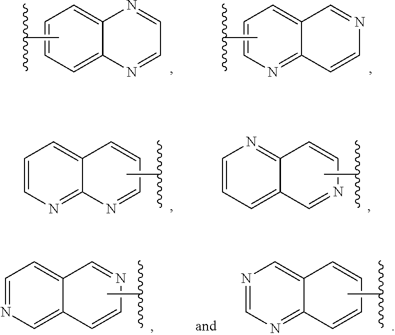

### Embodiments of “Cycloalkyl”

In certain embodiments “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In certain embodiments “cycloalkyl” has three carbons.

In certain embodiments “cycloalkyl” has four carbons.

In certain embodiments “cycloalkyl” has five carbons.

In certain embodiments “cycloalkyl” has six carbons.

In certain embodiments “cycloalkyl” has seven carbons.

In certain embodiments “cycloalkyl” has eight carbons.

In certain embodiments “cycloalkyl” has nine carbons.

In certain embodiments “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

Additional non-limiting examples of “cycloalkyl” include dihydro-indene and tetrahydronaphthalene wherein the point of attachment for each group is on the cycloalkyl ring.

For example,

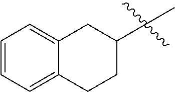 [SMILES: *C1CCc2ccccc2C1]

is an “cycloalkyl” group.

However,

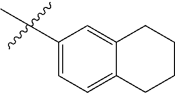 [SMILES: *c1ccc2c(c1)CCCC2]

is an “aryl” group.

### Embodiments of “Heterocycle”

In certain embodiments “heterocycle” refers to a cyclic ring with one nitrogen and 3, 4, or 5, carbon atoms.

In certain embodiments “heterocycle” refers to a cyclic ring with one nitrogen and 4 or 5 carbon atoms and a double bond.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

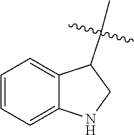 [SMILES: *C1CNc2ccccc21]

is a “heterocycle” group.

However,

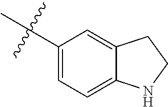 [SMILES: *c1ccc2c(c1)CCN2]

is an “aryl” group.

Non-limiting examples of “heterocycle” also include:

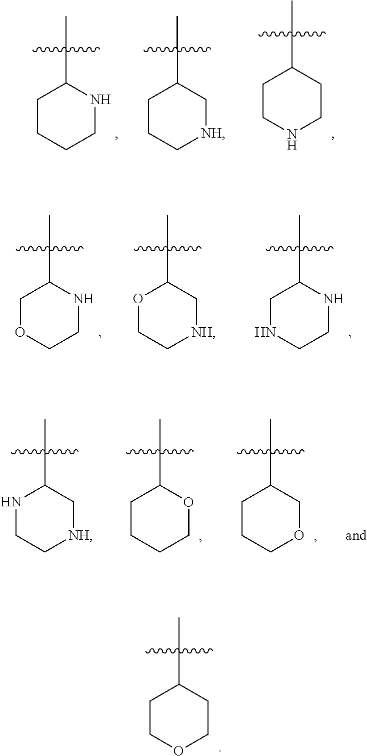

Additional non-limiting examples of “heterocycle” include:

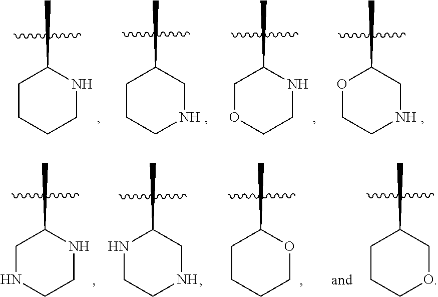

Additional non-limiting examples of “heterocycle” include:

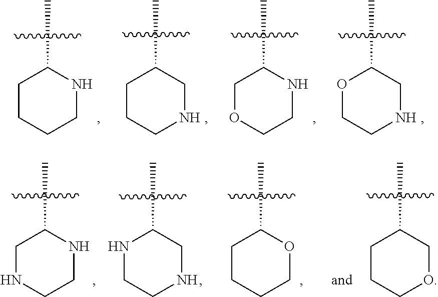

Non-limiting examples of “heterocycle” also include:

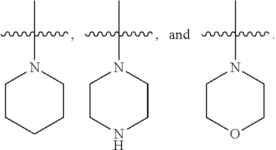

Non-limiting examples of “heterocycle” also include:

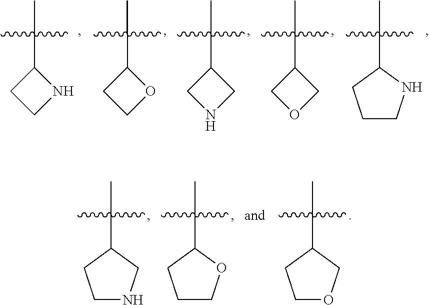

Additional non-limiting examples of “heterocycle” include:

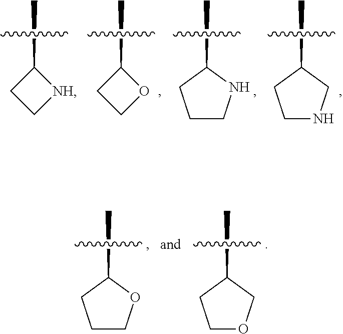

Additional non-limiting examples of “heterocycle” include:

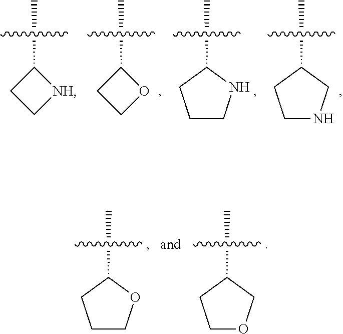

### Embodiments of R¹

In certain embodiments R¹ is hydrogen.

In certain embodiments R¹ is halogen.

In certain embodiments R¹ is —F.

In certain embodiments R¹ is —Cl.

In certain embodiments R¹ is —Br.

In certain embodiments R¹ is —I.

In certain embodiments R¹ is alkyl.

In certain embodiments R¹ is methyl.

In certain embodiments R¹ is ethyl.

In certain embodiments R¹ is n-propyl.

In certain embodiments R¹ is isopropyl.

In certain embodiments R¹ is haloalkyl.

In certain embodiments R¹ is —CF₃.

In certain embodiments R¹ is —OP(O)(OR⁹)₂.

In certain embodiments R¹ is —OP(O)(OH)₂.

In certain embodiments R¹ is —SR⁹.

In certain embodiments R¹ is —SH.

In certain embodiments R¹ is —SCF₃.

In certain embodiments R¹ is —SMe.

In certain embodiments R¹ is —NR⁹R¹⁰.

In certain embodiments R¹ is —NHR¹⁰.

In certain embodiments R¹ is —NH₂.

In certain embodiments R¹ is —NHMe.

In certain embodiments R¹ is —NMe₂.

In certain embodiments R¹ is —OR⁹.

In certain embodiments R¹ is —OH.

In certain embodiments R¹ is —OCF₃.

In certain embodiments R¹ is —OCH₃.

In certain embodiments R¹ is selected from hydrogen, F, CH₃, and —OMe.

### Embodiments of R²

In certain embodiments R² is hydrogen.

In certain embodiments R² is halogen.

In certain embodiments R² is —F.

In certain embodiments R² is —Cl.

In certain embodiments R² is —Br.

In certain embodiments R² is —I.

In certain embodiments R² is alkyl.

In certain embodiments R² is methyl.

In certain embodiments R² is ethyl.

In certain embodiments R² is n-propyl.

In certain embodiments R² is isopropyl.

In certain embodiments R² is haloalkyl.

In certain embodiments R² is —CF₃.

In certain embodiments R² is —OP(O)(OR⁹)₂.

In certain embodiments R² is —OP(O)(OH)₂.

In certain embodiments R² is —SR⁹.

In certain embodiments R² is —SH.

In certain embodiments R² is —SCF₃.

In certain embodiments R² is —SMe.

In certain embodiments R² is —NR⁹R¹⁰.

In certain embodiments R² is —NHR¹⁰.

In certain embodiments R² is —NH₂.

In certain embodiments R² is —NHMe.

In certain embodiments R² is —NMe₂.

In certain embodiments R² is —OR⁹.

In certain embodiments R² is —OH.

In certain embodiments R² is —OCF₃.

In certain embodiments R² is —OCH₃.

In certain embodiments R² is selected from hydrogen, F, CH₃, and —OMe.

### Embodiments of R⁴

In certain embodiments R⁴ is hydrogen.

In certain embodiments R⁴ is halogen.

In certain embodiments R⁴ is —F.

In certain embodiments R⁴ is —Cl.

In certain embodiments R⁴ is —Br.

In certain embodiments R⁴ is —I.

In certain embodiments R⁴ is alkyl.

In certain embodiments R⁴ is methyl.

In certain embodiments R⁴ is ethyl.

In certain embodiments R⁴ is n-propyl.

In certain embodiments R⁴ is isopropyl.

In certain embodiments R⁴ is haloalkyl.

In certain embodiments R⁴ is —CF₃.

In certain embodiments R⁴ is —OP(O)(OR⁹)₂.

In certain embodiments R⁴ is —OP(O)(OH)₂.

In certain embodiments R⁴ is —SR⁹.

In certain embodiments R⁴ is —SH.

In certain embodiments R⁴ is —SCF₃.

In certain embodiments R⁴ is —SMe.

In certain embodiments R⁴ is —NR⁹R¹⁰.

In certain embodiments R⁴ is —NHR¹⁰.

In certain embodiments R⁴ is —NH₂.

In certain embodiments R⁴ is —NHMe.

In certain embodiments R⁴ is —NMe₂.

In certain embodiments R⁴ is —OR⁹.

In certain embodiments R⁴ is —OH.

In certain embodiments R⁴ is —OCF₃.

In certain embodiments R⁴ is —OCH₃.

In certain embodiments R⁴ is selected from hydrogen, F, CH₃, and —OMe.

### Embodiments of R⁵

In certain embodiments R⁵ is hydrogen.

In certain embodiments R⁵ is halogen.

In certain embodiments R⁵ is —F.

In certain embodiments R⁵ is —Cl.

In certain embodiments R⁵ is —Br.

In certain embodiments R⁵ is —I.

In certain embodiments R⁵ is alkyl.

In certain embodiments R⁵ is methyl.

In certain embodiments R⁵ is ethyl.

In certain embodiments R⁵ is n-propyl.

In certain embodiments R⁵ is isopropyl.

In certain embodiments R⁵ is haloalkyl.

In certain embodiments R⁵ is —CF₃.

In certain embodiments R⁵ is —OP(O)(OR⁹)₂.

In certain embodiments R⁵ is —OP(O)(OH)₂.

In certain embodiments R⁵ is —SR⁹.

In certain embodiments R⁵ is —SH.

In certain embodiments R⁵ is —SCF₃.

In certain embodiments R⁵ is —SMe.

In certain embodiments R⁵ is —NR⁹R¹⁰.

In certain embodiments R⁵ is —NHR¹⁰.

In certain embodiments R⁵ is —NH₂.

In certain embodiments R⁵ is —NHMe.

In certain embodiments R⁵ is —NMe₂.

In certain embodiments R⁵ is —OR⁹.

In certain embodiments R⁵ is —OH.

In certain embodiments R⁵ is —OCF₃.

In certain embodiments R⁵ is —OCH₃.

In certain embodiments R⁵ is selected from hydrogen, F, CH₃, and —OMe.

### Embodiments of R⁶

In certain embodiments R⁶ is hydrogen.

In certain embodiments R⁶ is halogen.

In certain embodiments R⁶ is —F.

In certain embodiments R⁶ is —Cl.

In certain embodiments R⁶ is —Br.

In certain embodiments R⁶ is —I.

In certain embodiments R⁶ is alkyl.

In certain embodiments R⁶ is methyl.

In certain embodiments R⁶ is ethyl.

In certain embodiments R⁶ is n-propyl.

In certain embodiments R⁶ is isopropyl.

In certain embodiments R⁶ is haloalkyl.

In certain embodiments R⁶ is —CF₃.

In certain embodiments R⁶ is —OP(O)(OR⁹)₂.

In certain embodiments R⁶ is —OP(O)(OH)₂.

In certain embodiments R⁶ is —SR⁹.

In certain embodiments R⁶ is —SH.

In certain embodiments R⁶ is —SCF₃.

In certain embodiments R⁶ is —SMe.

In certain embodiments R⁶ is —NR⁹R¹⁰.

In certain embodiments R⁶ is —NHR¹⁰.

In certain embodiments R⁶ is —NH₂.

In certain embodiments R⁶ is —NHMe.

In certain embodiments R⁶ is —NMe₂.

In certain embodiments R⁶ is —OR⁹.

In certain embodiments R⁶ is —OH.

In certain embodiments R⁶ is —OCF₃.

In certain embodiments R⁶ is —OCH₃.

In certain embodiments R⁶ is selected from hydrogen, F, CH₃, and —OMe.

### Embodiments of R⁷

In certain embodiments R⁷ is hydrogen.

In certain embodiments R⁷ is halogen.

In certain embodiments R⁷ is —F.

In certain embodiments R⁷ is —Cl.

In certain embodiments R⁷ is —Br.

In certain embodiments R⁷ is —I.

In certain embodiments R⁷ is alkyl.

In certain embodiments R⁷ is methyl.

In certain embodiments R⁷ is ethyl.

In certain embodiments R⁷ is n-propyl.

In certain embodiments R⁷ is isopropyl.

In certain embodiments R⁷ is haloalkyl.

In certain embodiments R⁷ is —CF₃.

In certain embodiments R⁷ is —OP(O)(OR⁹)₂.

In certain embodiments R⁷ is —OP(O)(OH)₂.

In certain embodiments R⁷ is —SR⁹.

In certain embodiments R⁸ is —SH.

In certain embodiments R⁷ is —SCF₃.

In certain embodiments R⁷ is —SMe.

In certain embodiments R⁷ is —NR⁹R¹⁰.

In certain embodiments R⁷ is —NHR¹⁰.

In certain embodiments R⁷ is —NH₂.

In certain embodiments R⁷ is —NHMe.

In certain embodiments R⁷ is —NMe₂.

In certain embodiments R⁷ is —OR⁹.

In certain embodiments R⁷ is —OH.

In certain embodiments R⁷ is —OCF₃.

In certain embodiments R⁷ is —OCH₃.

In certain embodiments R⁷ is selected from hydrogen, F, CH₃, and —OMe.

### Embodiments of R⁸

In certain embodiments R⁸ is hydrogen.

In certain embodiments R⁸ is halogen.

In certain embodiments R⁸ is —F.

In certain embodiments R⁸ is —Cl.

In certain embodiments R⁸ is —Br.

In certain embodiments R⁸ is —I.

In certain embodiments R⁸ is alkyl.

In certain embodiments R⁸ is methyl.

In certain embodiments R⁸ is ethyl.

In certain embodiments R⁸ is n-propyl.

In certain embodiments R⁸ is isopropyl.

In certain embodiments R⁸ is haloalkyl.

In certain embodiments R⁸ is —CF₃.

In certain embodiments R⁸ is —OP(O)(OR⁹)₂.

In certain embodiments R⁸ is —OP(O)(OH)₂.

In certain embodiments R⁸ is —SR⁹.

In certain embodiments R⁸ is —SH.

In certain embodiments R⁸ is —SCF₃.

In certain embodiments R⁸ is —SMe.

In certain embodiments R⁸ is —NR⁹R¹⁰.

In certain embodiments R⁸ is —NHR¹⁰.

In certain embodiments R⁸ is —NH₂.

In certain embodiments R⁸ is —NHMe.

In certain embodiments R⁸ is —NMe₂.

In certain embodiments R⁸ is —OR⁹.

In certain embodiments R⁸ is —OH.

In certain embodiments R⁸ is —OCF₃.

In certain embodiments R⁸ is —OCH₃.

In certain embodiments R⁸ is selected from hydrogen, F, CH₃, and —OMe.

### Embodiments of R⁹

In certain embodiments R⁹ is hydrogen.

In certain embodiments R⁹ is —CH₃.

In certain embodiments R⁹ is —CH₂X.

In certain embodiments R⁹ is —CHX₂.

In certain embodiments R⁹ is —CX₃.

In certain embodiments R⁹ is —CF₃.

In certain embodiments R⁹ is —CH₂CH₃.

In certain embodiments R⁹ is —CH₂CH₂X.

In certain embodiments R⁹ is —CH₂ CHX₂.

In certain embodiments R⁹ is —CH₂CX₃.

### Embodiments of R¹⁰

In certain embodiments R¹⁰ is hydrogen.

In certain embodiments R¹⁰ is —CH₃.

In certain embodiments R¹⁰ is —CH₂X.

In certain embodiments R¹⁰ is —CHX₂.

In certain embodiments R¹⁰ is —CX₃.

In certain embodiments R¹⁰ is —CF₃.

In certain embodiments R¹⁰ is —CH₂CH₃.

In certain embodiments R¹⁰ is —CH₂CH₂X.

In certain embodiments R¹⁰ is —CH₂ CHX₂.

In certain embodiments R¹⁰ is —CH₂CX₃.

### Embodiments of R¹¹

In certain embodiments R¹¹ is hydrogen.

In certain embodiments R¹¹ is methyl.

In certain embodiments R¹¹ is ethyl.

In certain embodiments R¹¹ is propyl.

In certain embodiments R¹¹ is —CH₂CH₂X.

In certain embodiments R¹¹ is —CH₂ CHX₂.

In certain embodiments R¹¹ is —CH₂CX₃.

In certain embodiments R¹¹ is —CH₂OH.

In certain embodiments R¹¹ is —CH₂CH₂OH.

### Embodiments of R²

In certain embodiments R¹² is hydrogen.

In certain embodiments R¹² is —OH.

In certain embodiments R¹² is methyl.

In certain embodiments R¹² is ethyl.

In certain embodiments R¹² is propyl.

In certain embodiments R¹² is —CH₂CH₂X.

In certain embodiments R¹² is —CH₂ CHX₂.

In certain embodiments R¹² is —CH₂CX₃.

In certain embodiments R¹² is —CH₂OH.

In certain embodiments R¹² is —CH₂CH₂OH.

### Embodiments of R¹³

In certain embodiments R¹³ is —OH.

In certain embodiments R¹³ is methyl.

In certain embodiments R¹³ is ethyl.

In certain embodiments R¹³ is propyl.

In certain embodiments R¹³ is —CH₂CH₂X.

In certain embodiments R¹³ is —CH₂ CHX₂.

In certain embodiments R¹³ is —CH₂CX₃.

In certain embodiments R¹³ is —CH₂OH.

In certain embodiments R¹³ is —CH₂CH₂OH.

### Embodiments of R^{13A}

In certain embodiments R^{13A} is methyl.

In certain embodiments R^{13A} is ethyl.

In certain embodiments R^{13A} is propyl.

In certain embodiments R^{13A} is —CH₂CH₂X.

In certain embodiments R^{13A} is —CH₂ CHX₂.

In certain embodiments R^{13A} is —CH₂CX₃.

In certain embodiments R^{13A} is —CH₂OH.

In certain embodiments R^{13A} is —CH₂CH₂OH.

### Embodiments of R^{13B}

In certain embodiments R^{13B} is —OH.

In certain embodiments R^{13B} is methyl.

In certain embodiments R^{13B} is ethyl.

In certain embodiments R^{13B} is propyl.

In certain embodiments R^{13B} is —CH₂CH₂X.

In certain embodiments R^{13B} is —CH₂ CHX₂.

In certain embodiments R^{13B} is —CH₂CX₃.

In certain embodiments R^{13B} is —CH₂OH.

In certain embodiments R^{13B} is —CH₂CH₂OH.

### Embodiments of R^{A1}

In certain embodiments R^{A1} is hydrogen.

In certain embodiments R^{A1} is —CH₃.

In certain embodiments R^{A1} is —CH₂X.

In certain embodiments R^{A1} is —CHX₂.

In certain embodiments R^{A1} is —CX₃.

In certain embodiments R^{A1} is —CF₃.

In certain embodiments R^{A1} is —CH₂CH₃.

In certain embodiments R^{A1} is —CH₂CH₂X.

In certain embodiments R^{A1} is —CH₂ CHX₂.

In certain embodiments R^{A1} is —CH₂CX₃.

In certain embodiments R^{A1} is —CH₂OH.

In certain embodiments R^{A1} is —CH₂CH₂OH.

### Embodiments of R^{A2}

In certain embodiments R^{A2} is —CH₃.

In certain embodiments R^{A2} is —CH₂X.

In certain embodiments R^{A2} is —CHX₂.

In certain embodiments R^{A2} is —CX₃.

In certain embodiments R^{A2} is —CH₂CH₃.

In certain embodiments R^{A2} is —CH₂CH₂X.

In certain embodiments R^{A2} is —CH₂ CHX₂.

In certain embodiments R^{A2} is —CH₂CX₃.

In certain embodiments R^{A2} is —CH₂OH.

In certain embodiments R^{A2} is —CH₂CH₂OH.

### Embodiments of R^{A3}

In certain embodiments R^{A3} is —CH₂X.

In certain embodiments R^{A3} is —CHX₂.

In certain embodiments R^{A3} is —CX₃.

In certain embodiments R^{A3} is —CH₂CH₂X.

In certain embodiments R^{A3} is —CH₂ CHX₂.

In certain embodiments R^{A3} is —CH₂CX₃.

In certain embodiments R^{A3} is —CH₂OH.

In certain embodiments R^{A3} is —CH₂CH₂OH.

### Embodiments of R^{B}1

In certain embodiments R^{B1} is

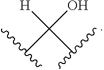 [SMILES: *C(*)O ; "."]

In certain embodiments R^{B1} is

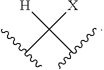

In certain embodiments R^{B1} is

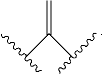 [SMILES: *C(*)=C ; "."]

In certain embodiments R^{B1} is

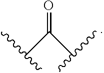 [SMILES: *C(*)=O ; "."]

### Embodiments of R^{B2}

In certain embodiments R^{B2} is

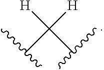 [SMILES: *C* ; "."]

In certain embodiments R^{B2} is

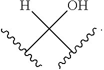 [SMILES: *C(*)O ; "."]

In certain embodiments R^{B2} is

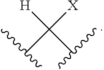

In certain embodiments R^{B2} is

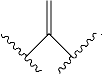 [SMILES: *C(*)=C ; "."]

In certain embodiments R^{B2} is

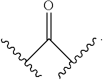 [SMILES: *C(*)=O ; "."]

### Embodiments of X

In certain embodiments X is —F.

In certain embodiments X is —Cl.

In certain embodiments X is —Br.

### Embodiments of R^{P}

In certain embodiments R^{P1} is —C(O)R^{13C};

In certain embodiments R^{P1} is -alkyl-C(O)R^{13C};

In certain embodiments R^{P1} is -alkyl-OC(O)R^{13C};

In certain embodiments R^{P1} is —C(O)CH₃.

In certain embodiments R^{P1} is —CH₂C(O)CH₃.

In certain embodiments R^{P1} is —C(O)CH₂CH₃.

In certain embodiments R^{P1} is —CH₂OC(O)CH₃.

In certain embodiments R^{P1} is —CH₂CH₂OC(O)CH₃.

### Embodiments of R^{13C}

In certain embodiments R^{13C} is methyl.

In certain embodiments R^{13C} is ethyl.

In certain embodiments R^{13C} is propyl.

In certain embodiments R^{13C} is —CH₂X.

In certain embodiments R^{13C} is —CHX₂.

In certain embodiments R^{13C} is —CX₃.

In certain embodiments R^{13C} is —CF₃.

In certain embodiments R^{13C} is —CH₂CH₃.

In certain embodiments R^{13C} is —CH₂CH₂X.

In certain embodiments R^{13C} is —CH₂ CHX₂.

In certain embodiments R^{13C} is —CH₂CX₃.

### Embodiments of R¹²

In certain embodiments R¹² is alanine.

In certain embodiments R¹² is arginine.

In certain embodiments R^{P2} is asparagine.

In certain embodiments R^{P2} is aspartic acid.

In certain embodiments R^{P2} is cysteine.

In certain embodiments R^{P2} is glutamine.

In certain embodiments R^{P2} is glutamic acid.

In certain embodiments R^{P2} is glycine.

In certain embodiments R^{P2} is histidine.

In certain embodiments R^{P2} is isoleucine.

In certain embodiments R^{P2} is leucine.

In certain embodiments R^{P2} is lysine.

In certain embodiments R^{P2} is methionine.

In certain embodiments R^{P2} is phenylalanine.

In certain embodiments R^{P2} is proline.

In certain embodiments R^{P2} is serine.

In certain embodiments R^{P2} is threonine.

In certain embodiments R^{P2} is tryptophan.

In certain embodiments R^{P2} is tyrosine.

In certain embodiments R^{P2} is valine.

### Additional Embodiments

In certain embodiments the compound of the present invention is of Formula:

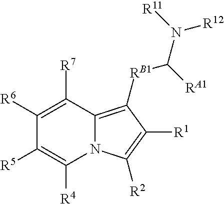

or pharmaceutically acceptable salt or salt mixture thereof.

In certain embodiments the compound of the present invention is of Formula:

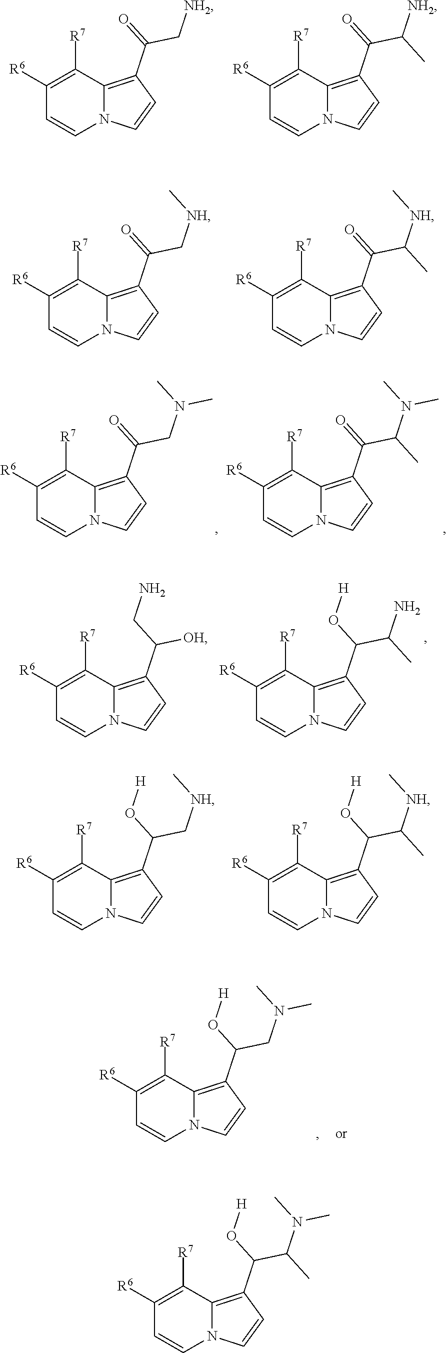

or pharmaceutically acceptable salt or salt mixture thereof.

In certain embodiments the compound of the present invention is of Formula:

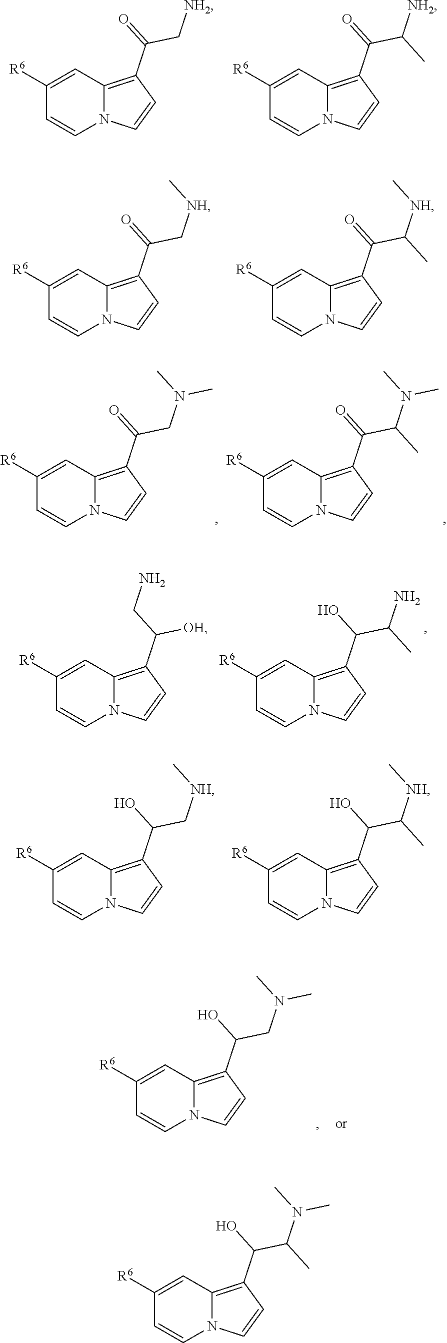

or pharmaceutically acceptable salt or salt mixture thereof.

In certain embodiments the compound of the present invention is of Formula:

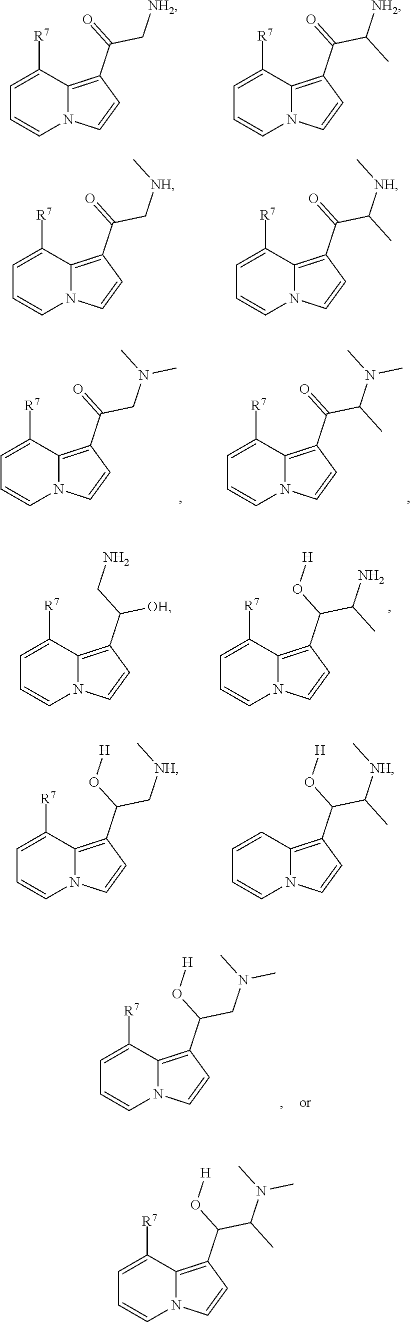

or pharmaceutically acceptable salt or salt mixture thereof.

In certain embodiments the compound of the present invention is of Formula:

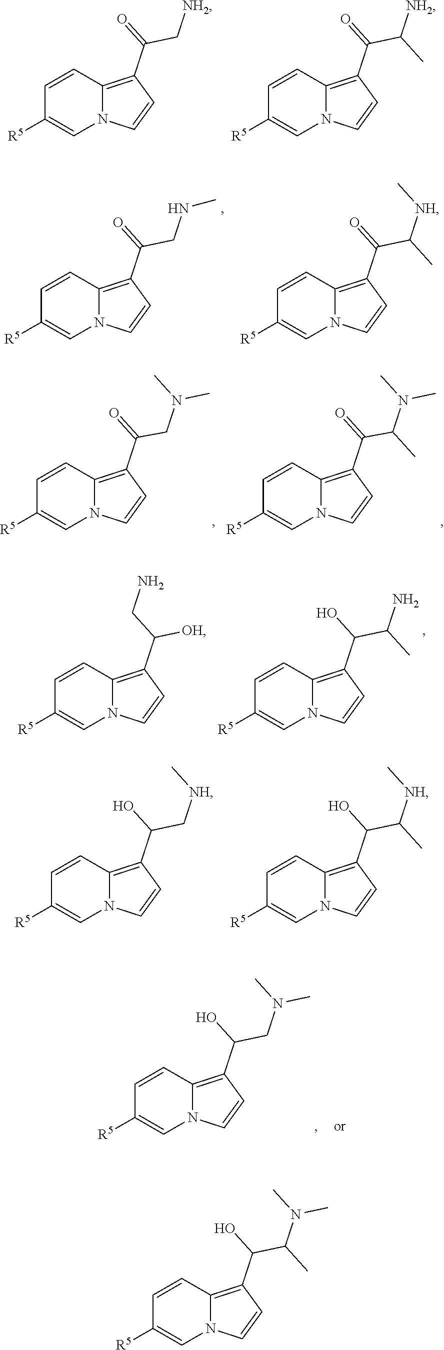

or pharmaceutically acceptable salt or salt mixture thereof.

In certain embodiments the compound of the present invention is of Formula:

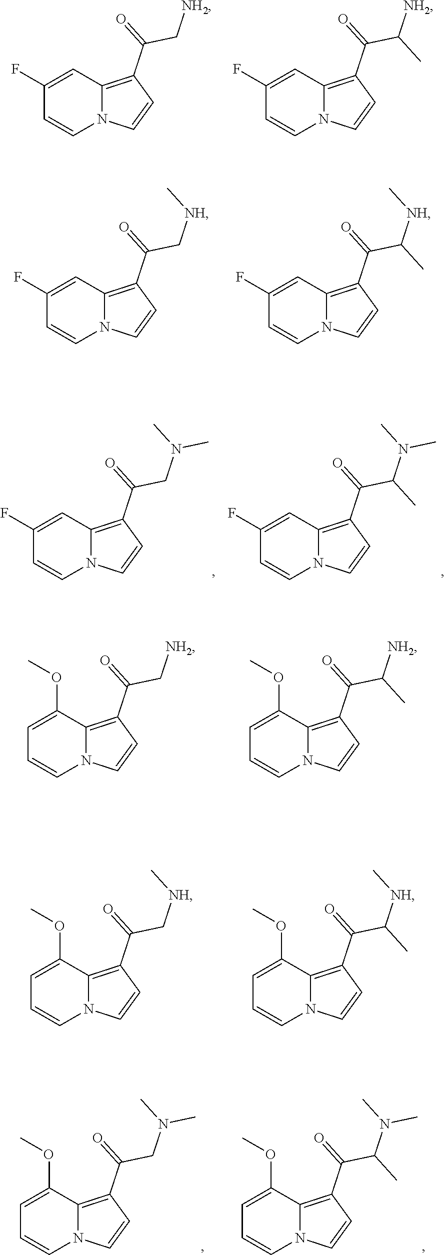

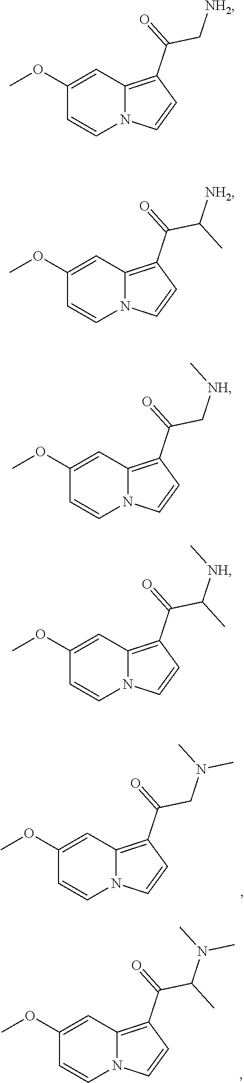

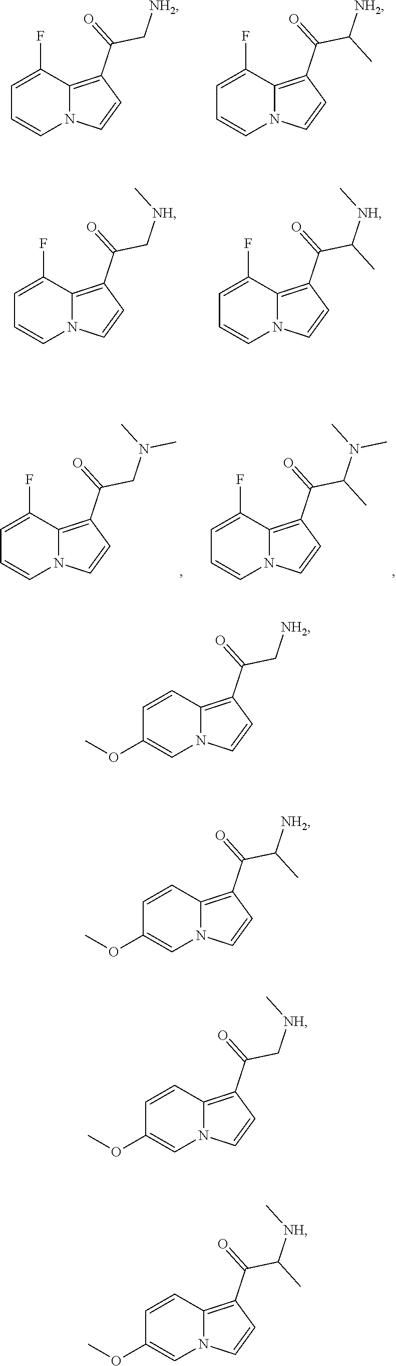

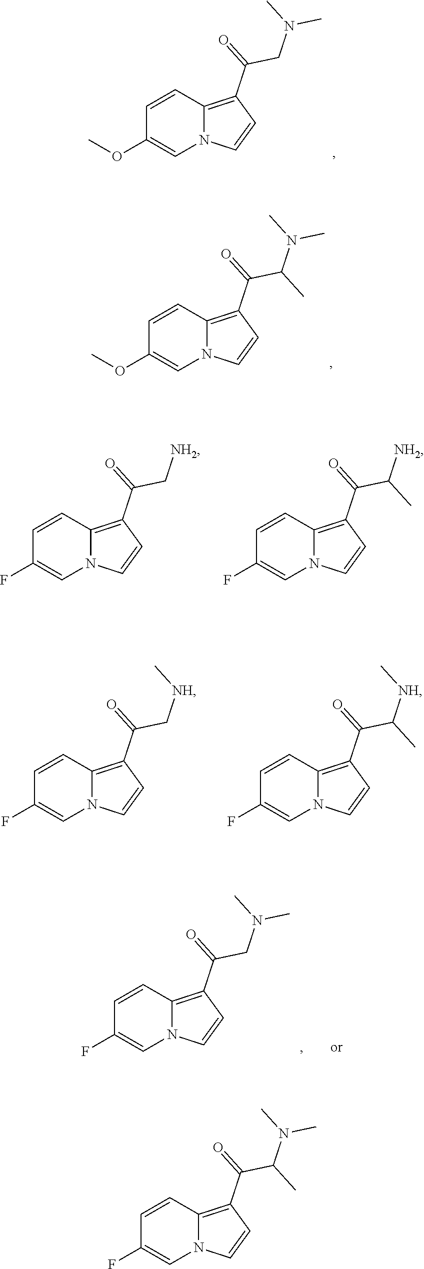

or pharmaceutically acceptable salt or salt mixture thereof.

In certain embodiments the compound of the present invention is of Formula:

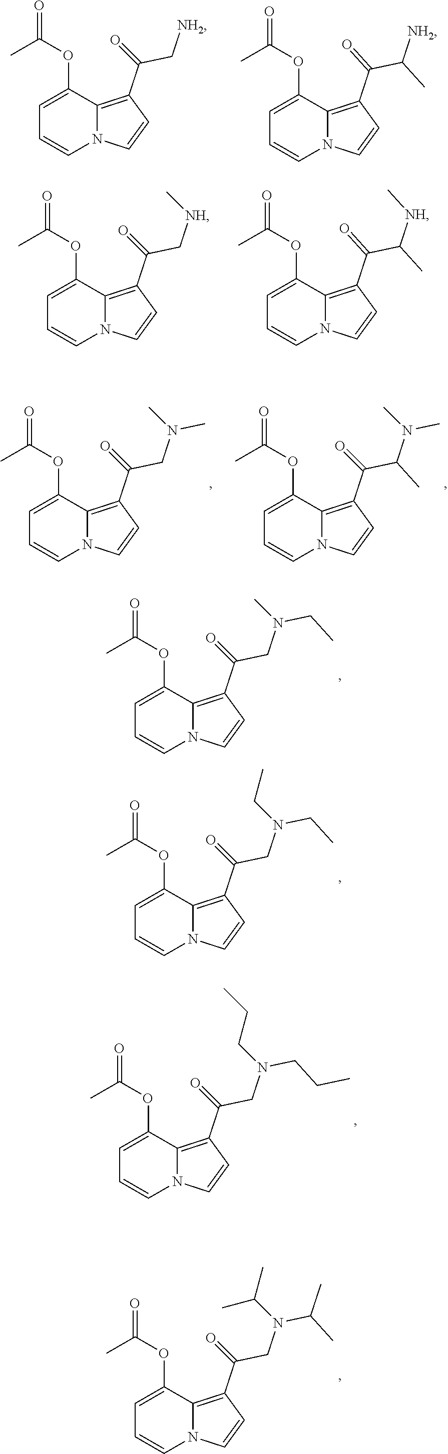

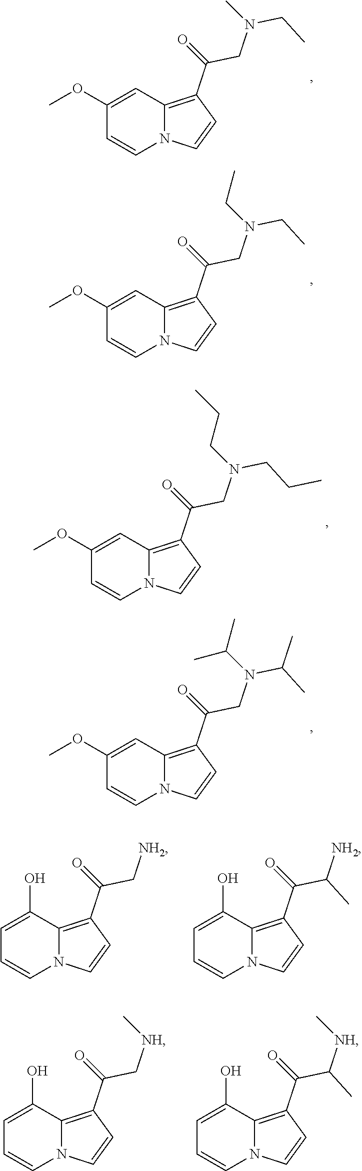

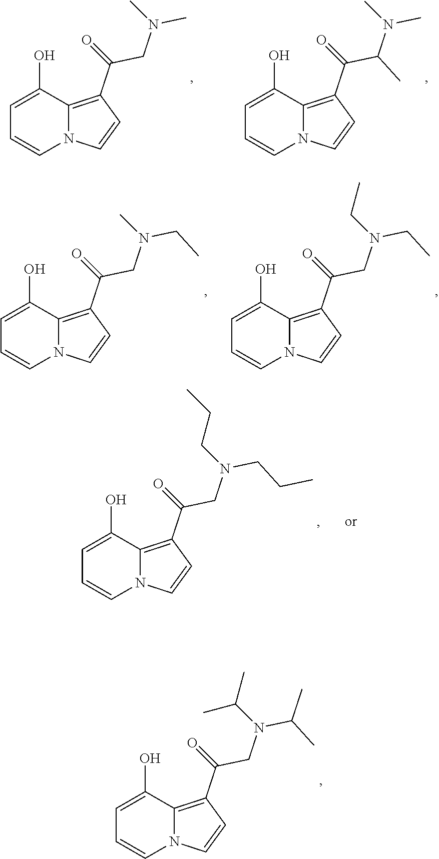

or pharmaceutically acceptable salt or salt mixture thereof.

In certain embodiments the compound of the present invention is of Formula:

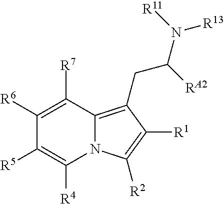

or pharmaceutically acceptable salt or salt mixture thereof.

In certain embodiments the compound of the present invention is of Formula:

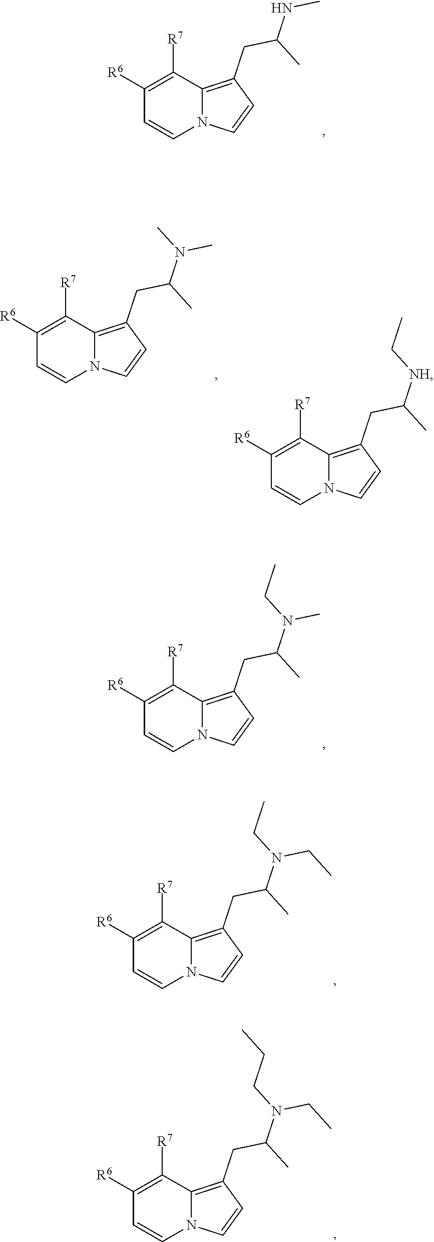

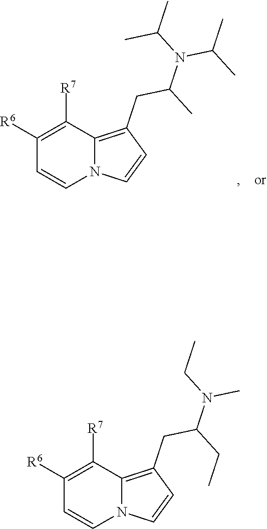

or pharmaceutically acceptable salt or salt mixture thereof.

In certain embodiments the compound of the present invention is of Formula:

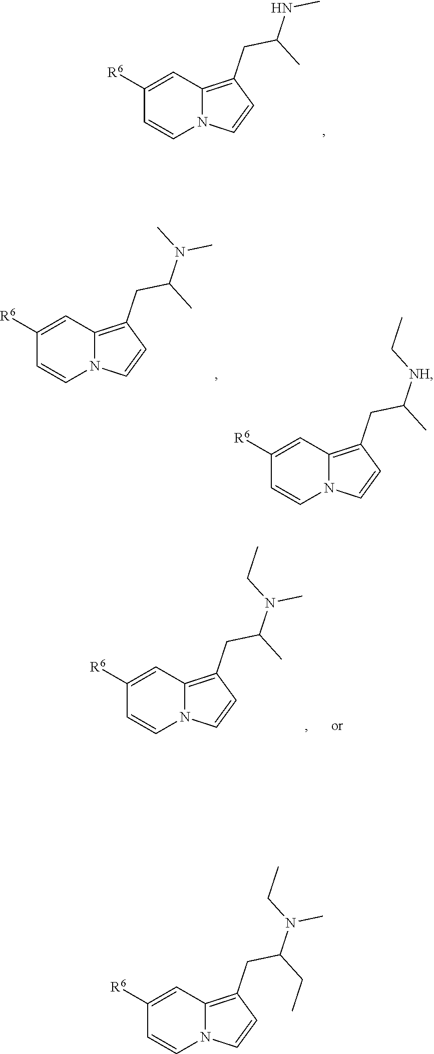

or pharmaceutically acceptable salt or salt mixture thereof.

In certain embodiments the compound of the present invention is of Formula:

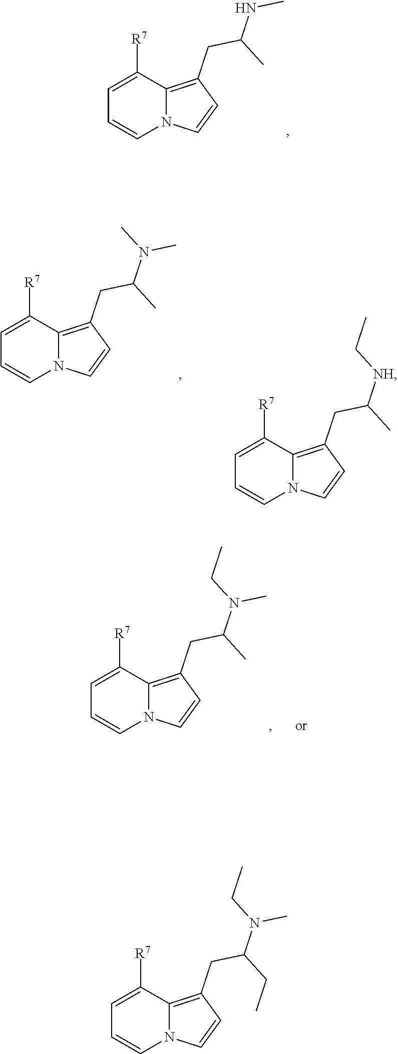

or pharmaceutically acceptable salt or salt mixture thereof.

In certain embodiments the compound of the present invention is of Formula:

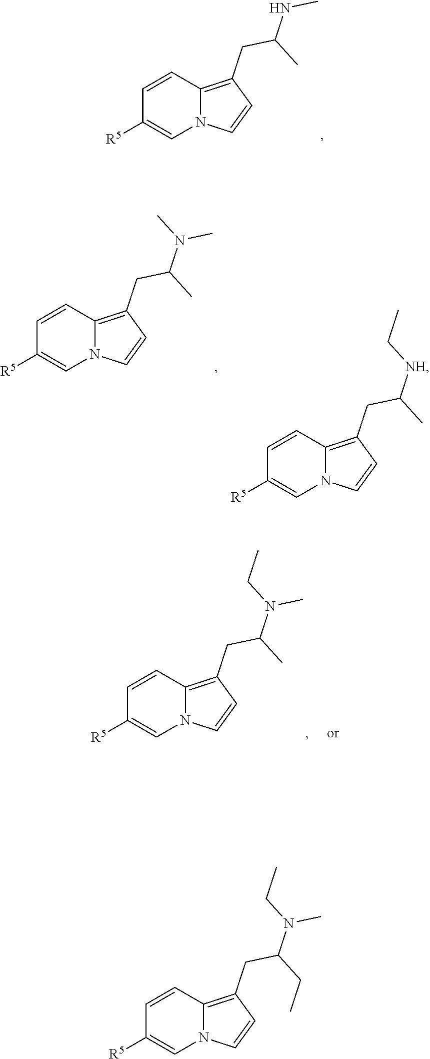

or pharmaceutically acceptable salt or salt mixture thereof.

In certain embodiments the compound of the present invention is of Formula:

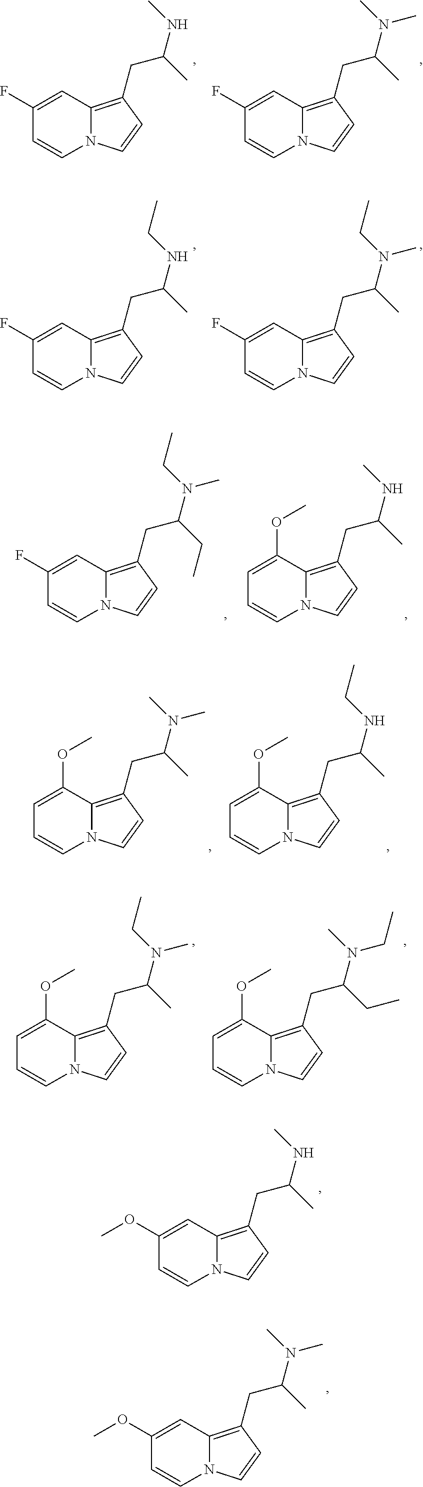

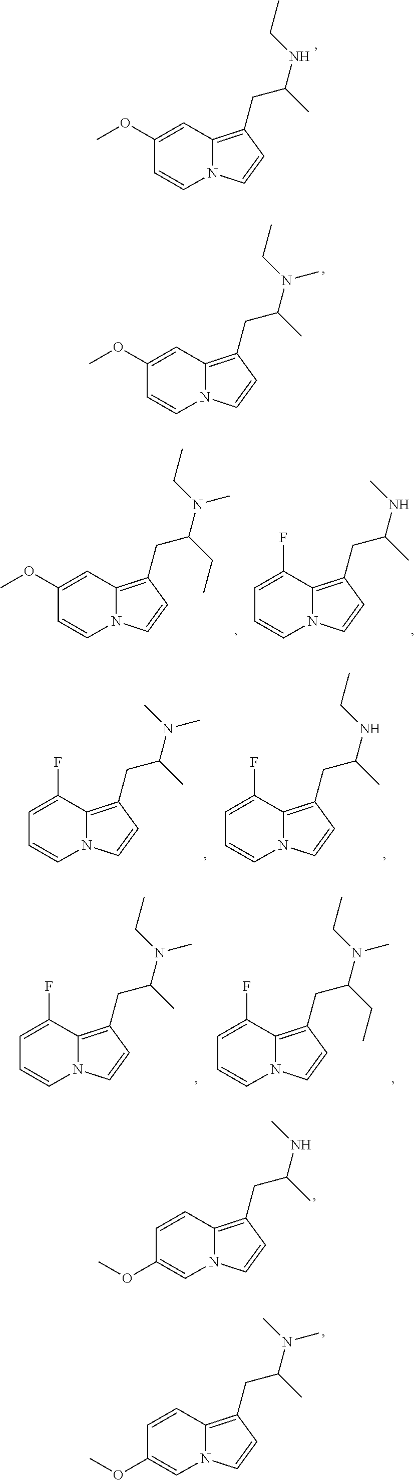

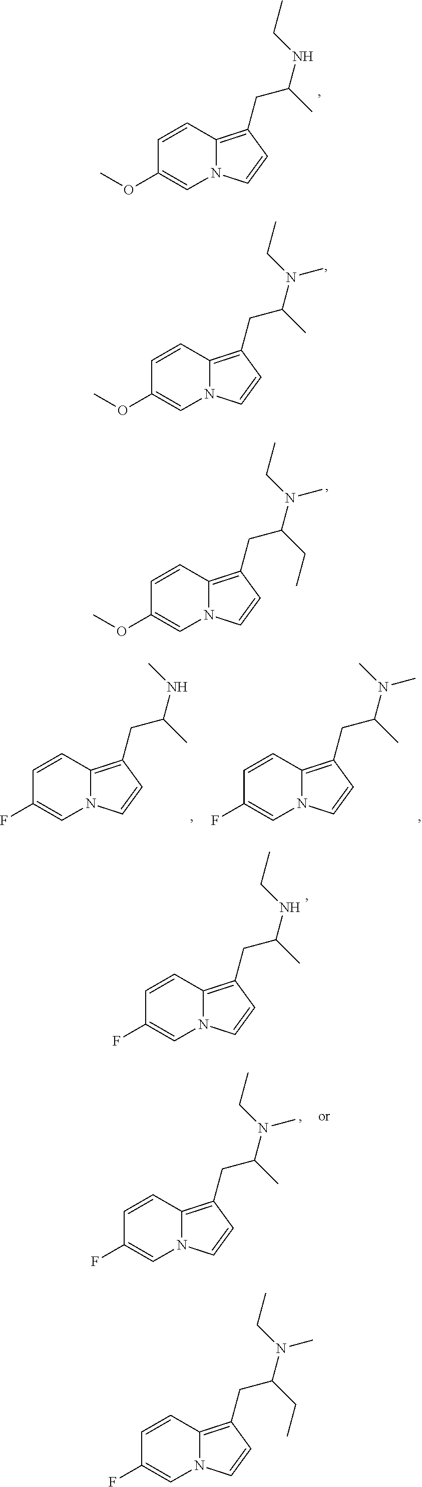

or pharmaceutically acceptable salt or salt mixture thereof.

In certain embodiments the compound of the present invention is of Formula:

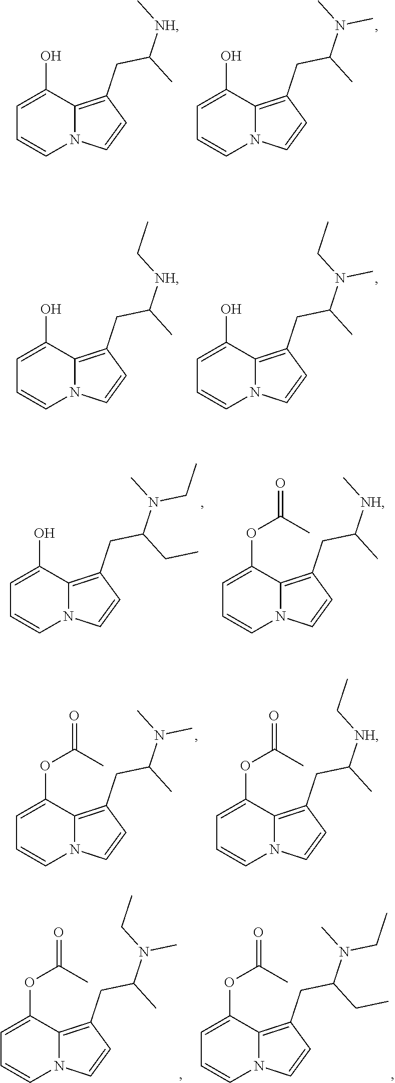

or pharmaceutically acceptable salt or salt mixture thereof.

In certain embodiments the compound of the present invention is of Formula:

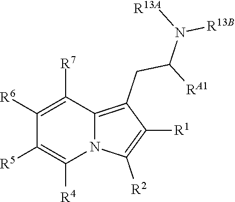

or pharmaceutically acceptable salt or salt mixture thereof.

In other embodiments the compound of the present invention is of Formula:

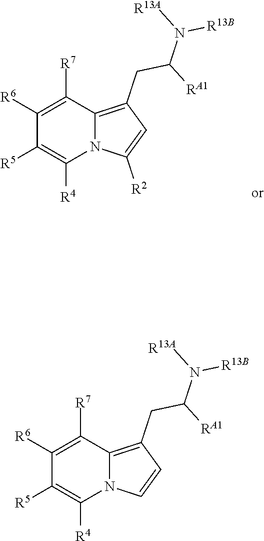

or pharmaceutically acceptable salt or salt mixture thereof.

In certain embodiments the compound of the present invention is of Formula:

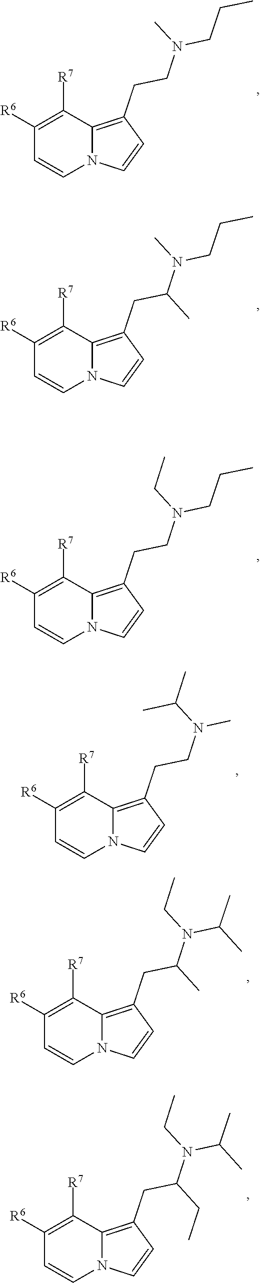

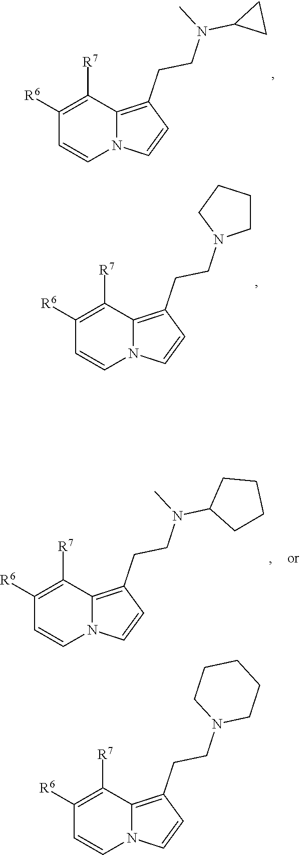

or pharmaceutically acceptable salt or salt mixture thereof.

In certain embodiments the compound of the present invention is of Formula:

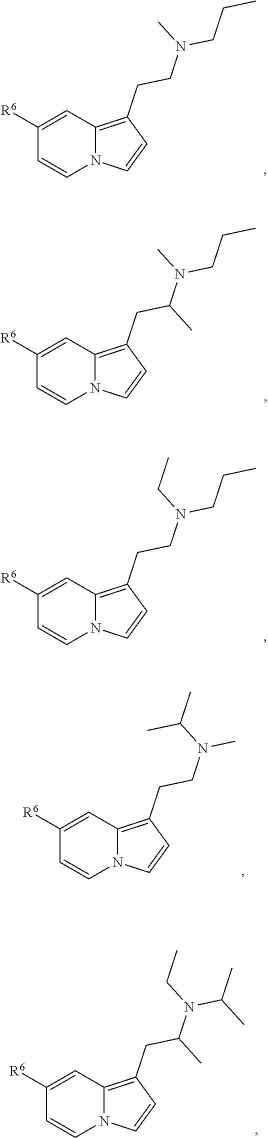

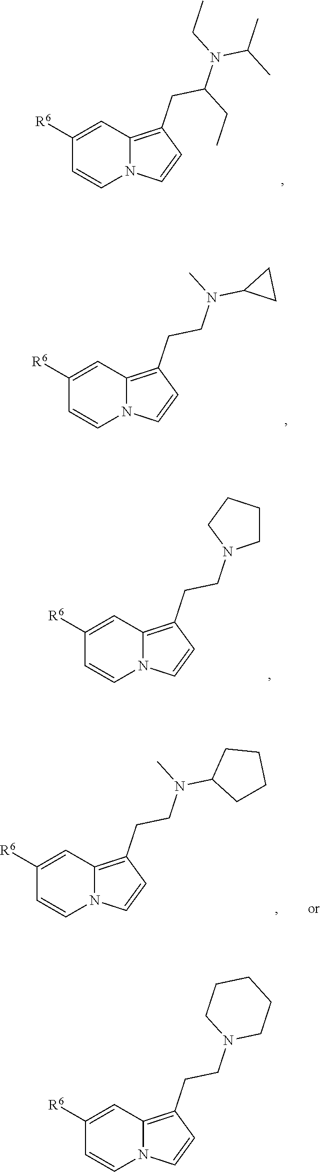

or pharmaceutically acceptable salt or salt mixture thereof.

In certain embodiments the compound of the present invention is of Formula:

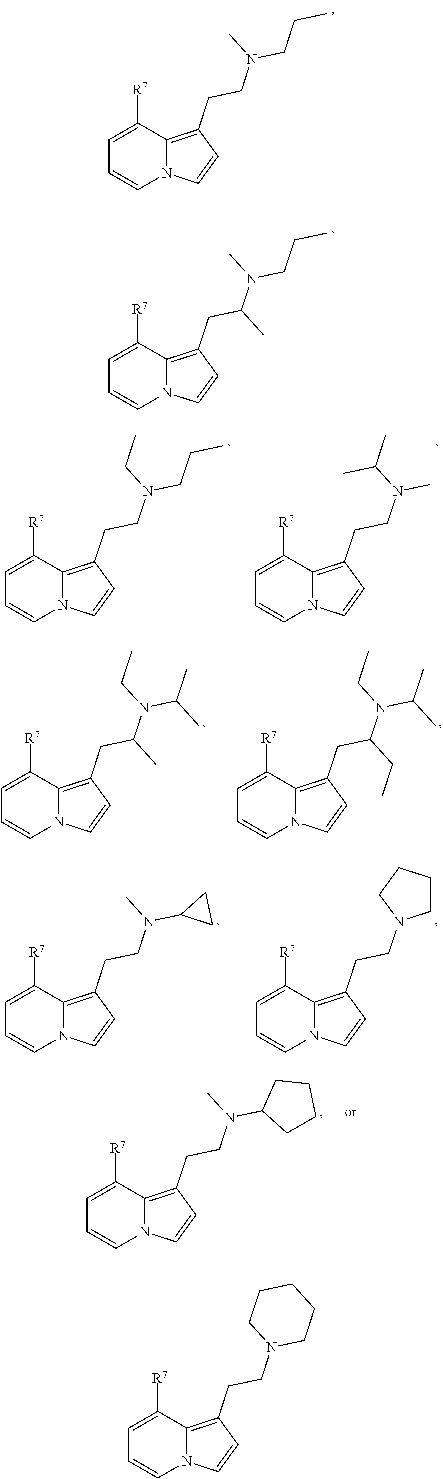

or pharmaceutically acceptable salt or salt mixture thereof.

In certain embodiments the compound of the present invention is of Formula:

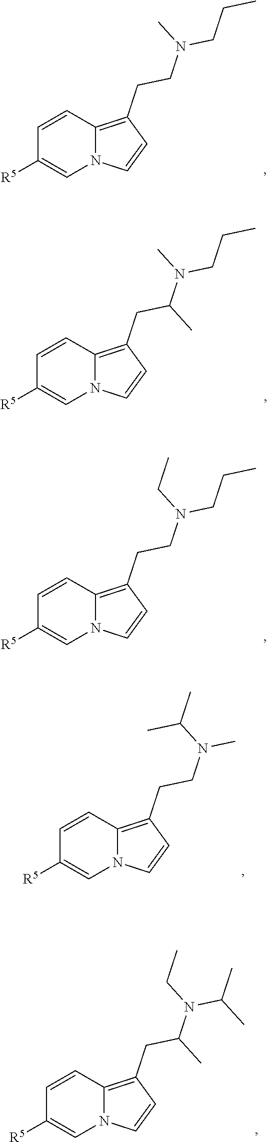

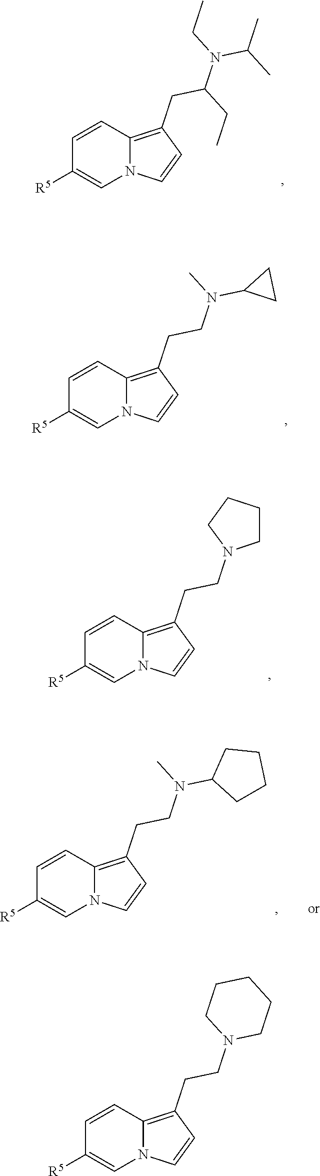

or pharmaceutically acceptable salt or salt mixture thereof.

In certain embodiments the compound of the present invention is selected from:

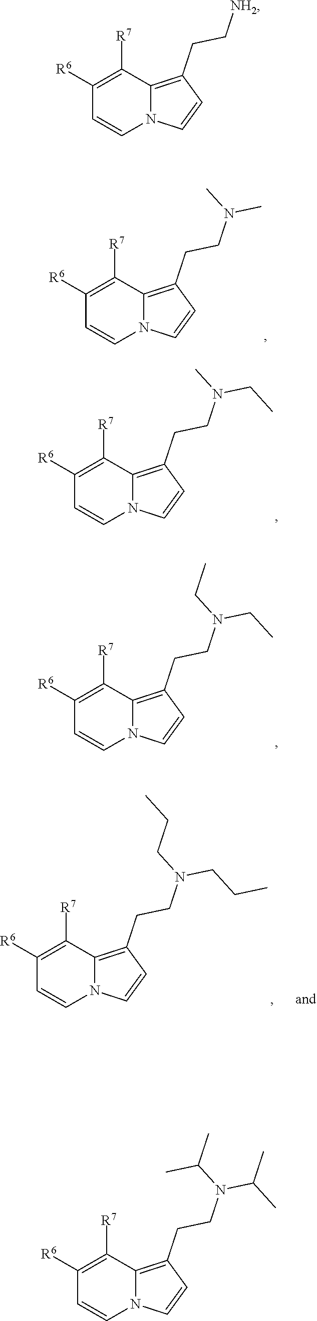

or a pharmaceutically acceptable salt or mixture of salts thereof.

In certain embodiments the compound of the present invention is selected from:

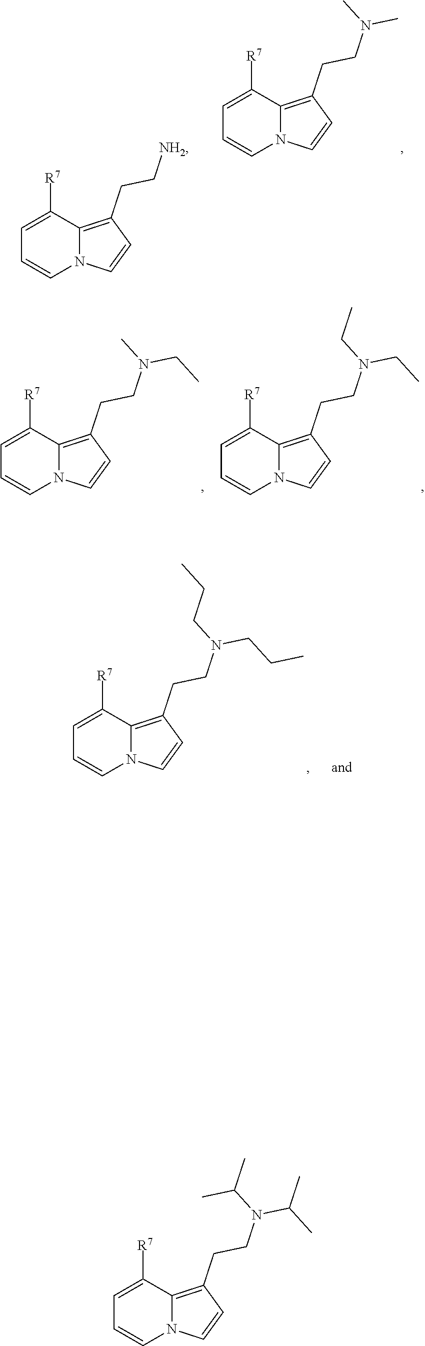

or a pharmaceutically acceptable salt or mixture of salts thereof.

In certain embodiments the compound of the present invention is selected from:

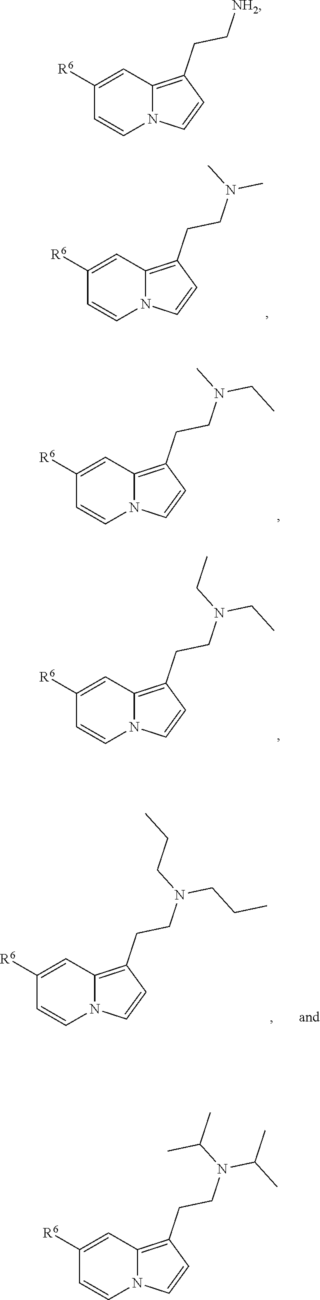

or a pharmaceutically acceptable salt or mixture of salts thereof.

In certain embodiments the compound of the present invention is selected from:

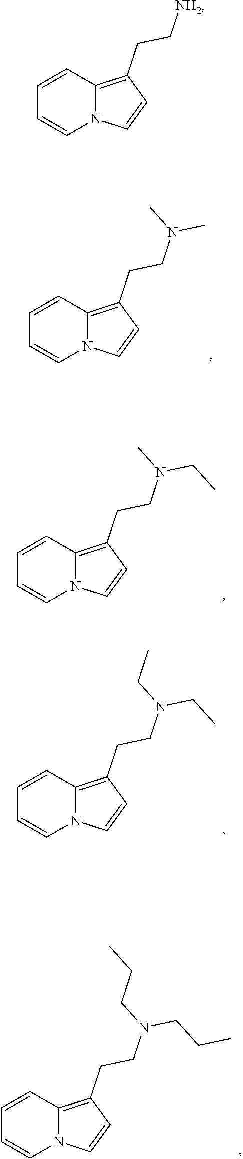 [SMILES: NCCc1ccn2ccccc12 ; CN(C)CCc1ccn2ccccc12 ; CCN(C)CCc1ccn2ccccc12 ; CCN(CC)CCc1ccn2ccccc12 ; CCCN(CCC)CCc1ccn2ccccc12]

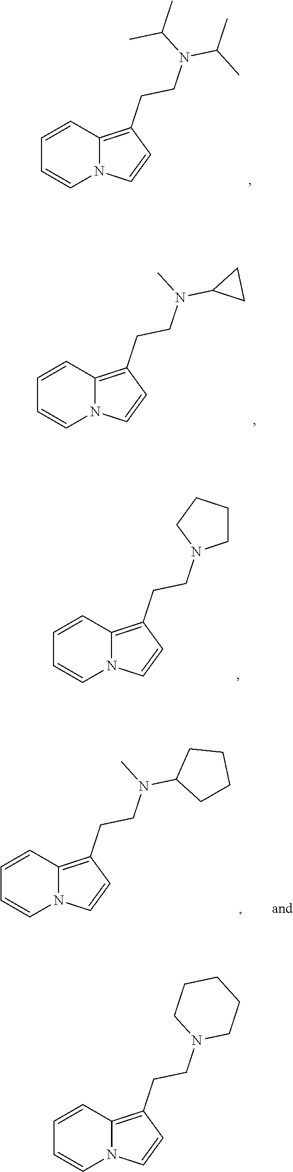 [SMILES: CC(C)N(CCc1ccn2ccccc12)C(C)C ; CN(CCc1ccn2ccccc12)C1CC1 ; c1ccn2ccc(CCN3CCCC3)c2c1 ; CN(CCc1ccn2ccccc12)C1CCCC1 ; c1ccn2ccc(CCN3CCCCC3)c2c1]

or a pharmaceutically acceptable salt or mixture of salts thereof.

In certain embodiments the compound of the present invention is selected from:

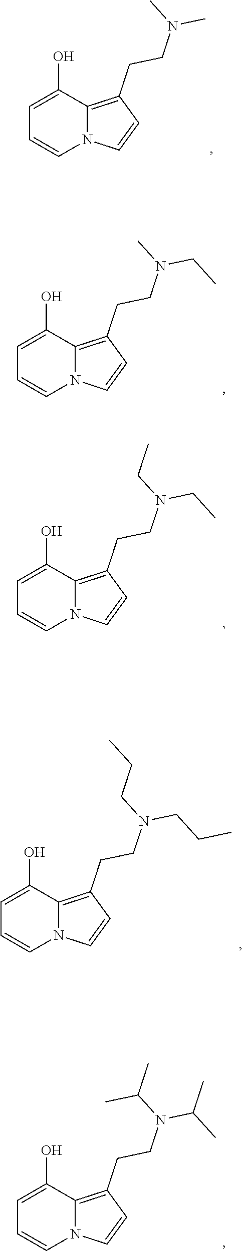 [SMILES: CN(C)CCc1ccn2cccc(O)c12 ; CCN(C)CCc1ccn2cccc(O)c12 ; CCN(CC)CCc1ccn2cccc(O)c12 ; CCCN(CCC)CCc1ccn2cccc(O)c12 ; CC(C)N(CCc1ccn2cccc(O)c12)C(C)C]

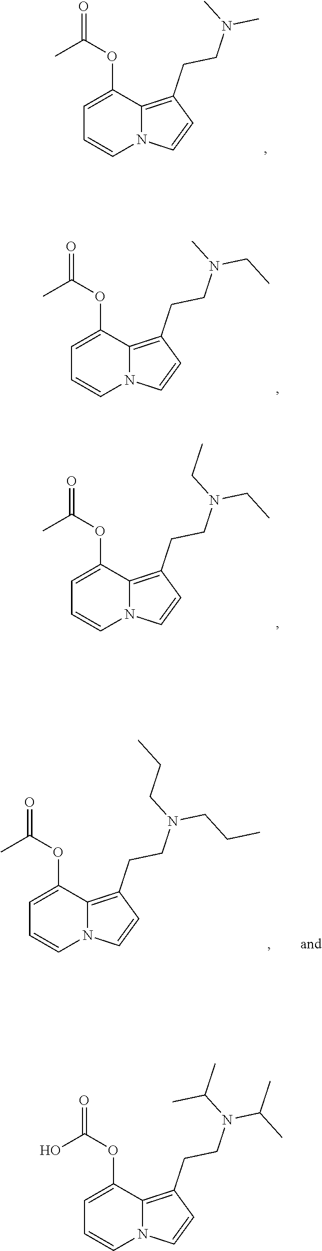

or a pharmaceutically acceptable salt or mixture of salts thereof.

In certain embodiments the compound of the present invention is selected from:

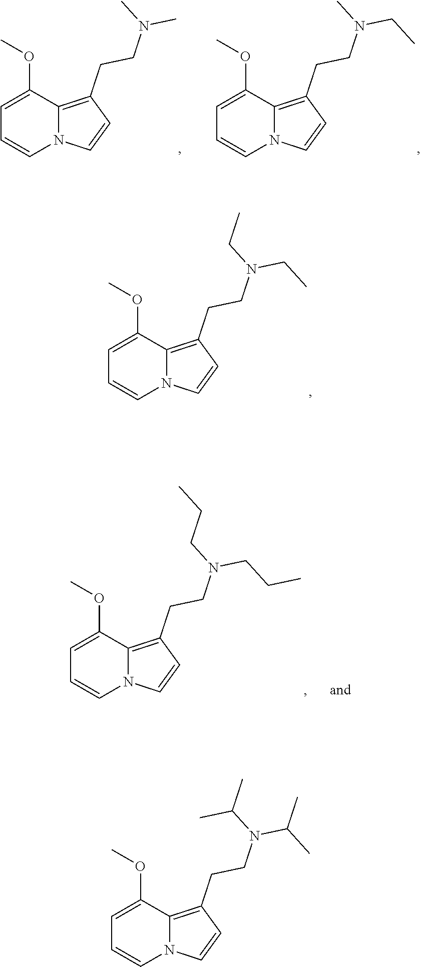

or a pharmaceutically acceptable salt or mixture of salts thereof.

In certain embodiments the compound of the present invention is selected from:

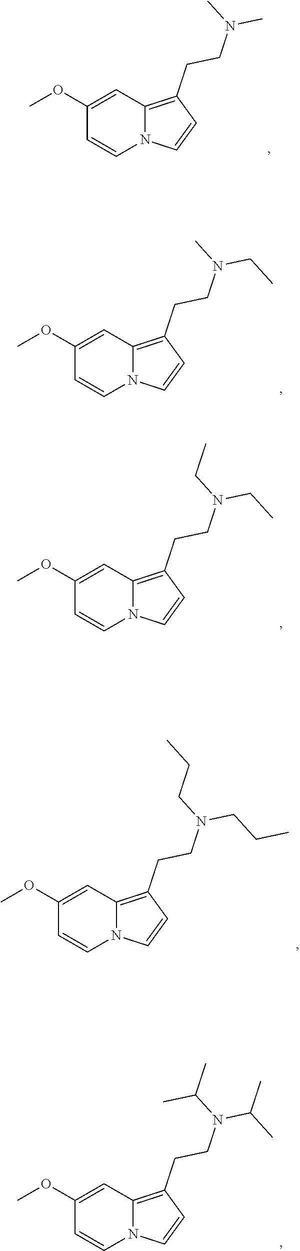

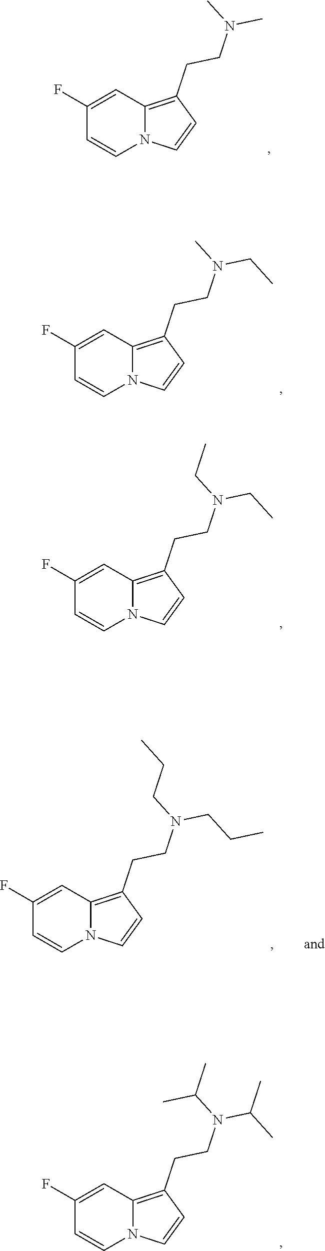 [SMILES: CN(C)CCc1ccn2ccc(F)cc12 ; CCN(C)CCc1ccn2ccc(F)cc12 ; CCN(CC)CCc1ccn2ccc(F)cc12 ; CCCN(CCC)CCc1ccn2ccc(F)cc12 ; CC(C)N(CCc1ccn2ccc(F)cc12)C(C)C]

or a pharmaceutically acceptable salt or mixture of salts thereof.

In certain embodiments the compound of the present invention is selected from:

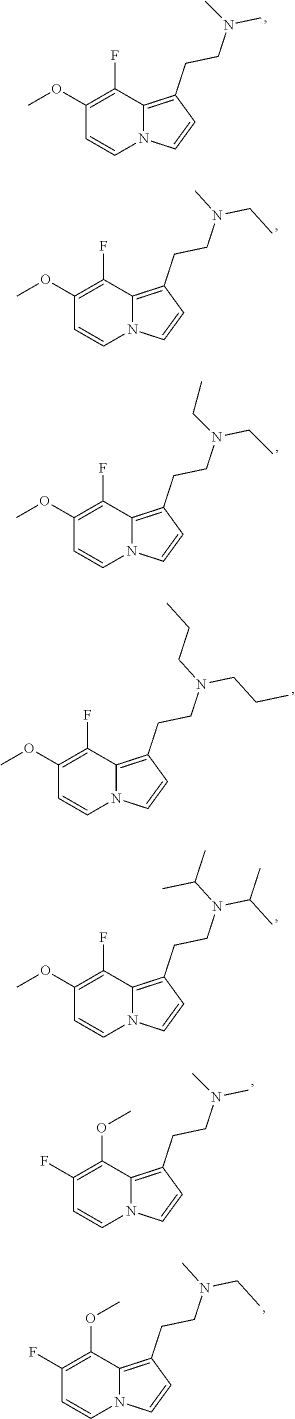

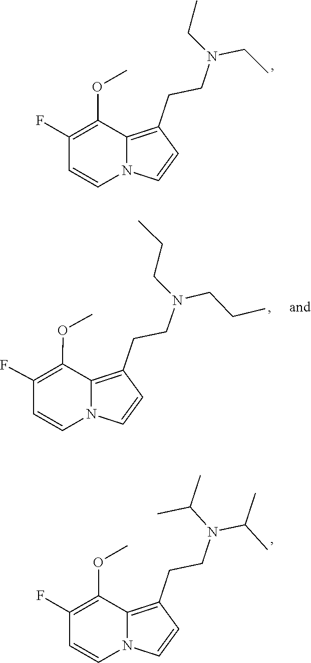

or a pharmaceutically acceptable salt or mixture of salts thereof.

In certain embodiments the compound of the present invention is of Formula:

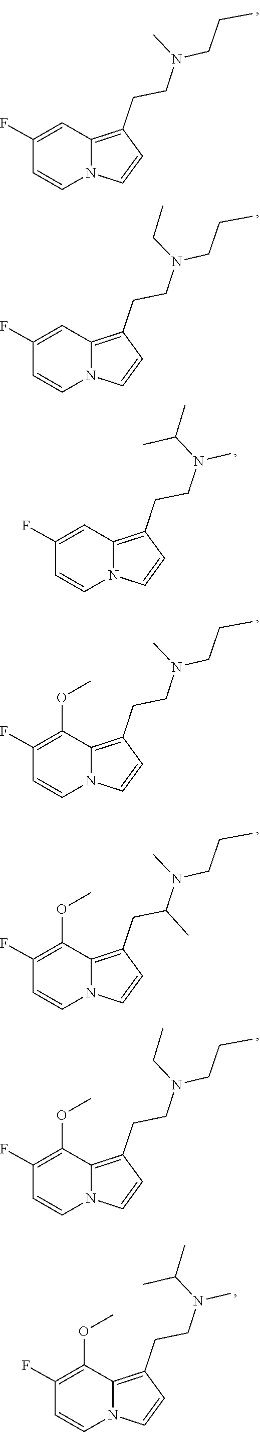

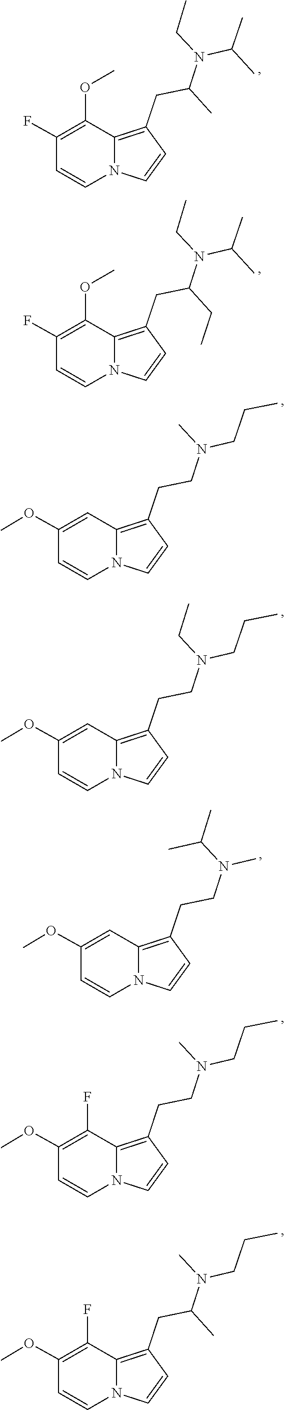

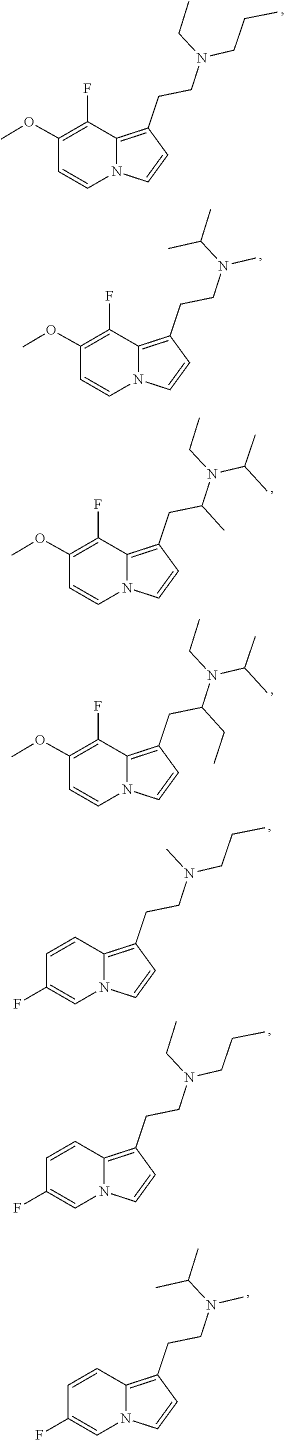

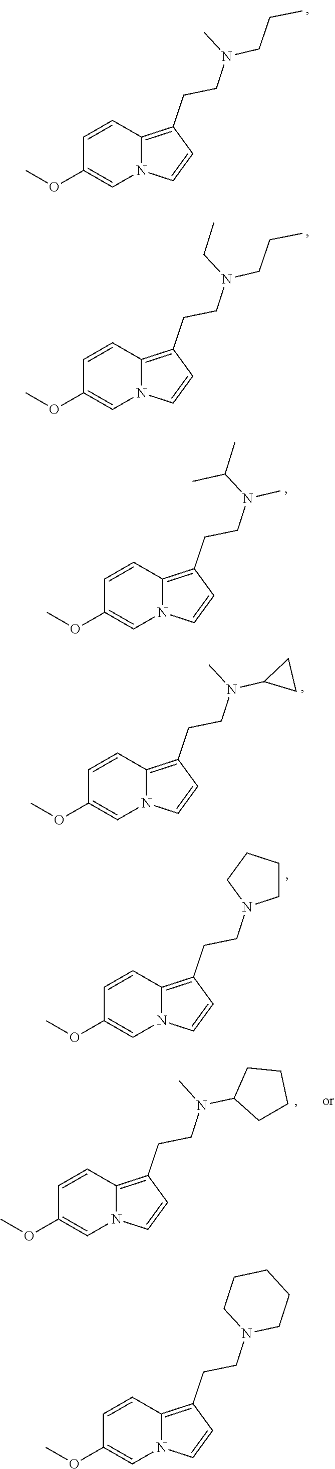

or pharmaceutically acceptable salt or salt mixture thereof.

In certain embodiments the compound of the present invention is of Formula:

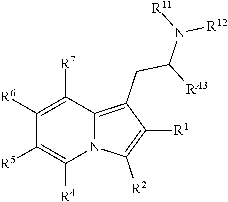

or pharmaceutically acceptable salt or salt mixture thereof.

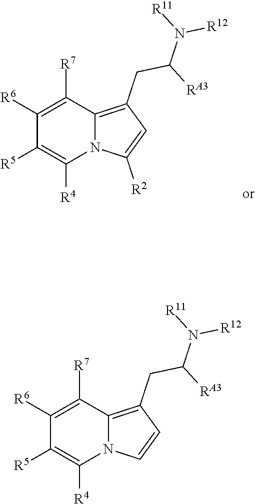

or pharmaceutically acceptable salt or salt mixture thereof.

In certain embodiments the compound of the present invention is of Formula:

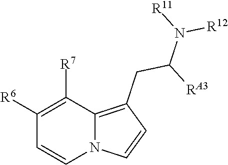

or pharmaceutically acceptable salt or salt mixture thereof.

In certain embodiments the compound of the present invention is of Formula:

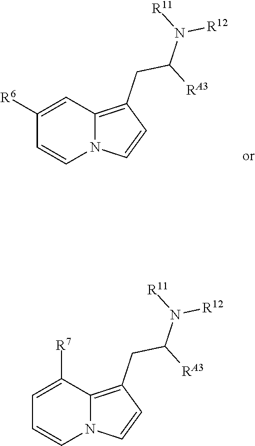

or pharmaceutically acceptable salt or salt mixture thereof.

In certain embodiments the compound of the present invention is of Formula:

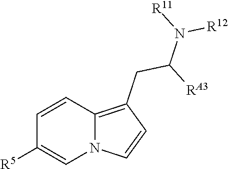

In certain embodiments the compound of the present invention is of Formula:

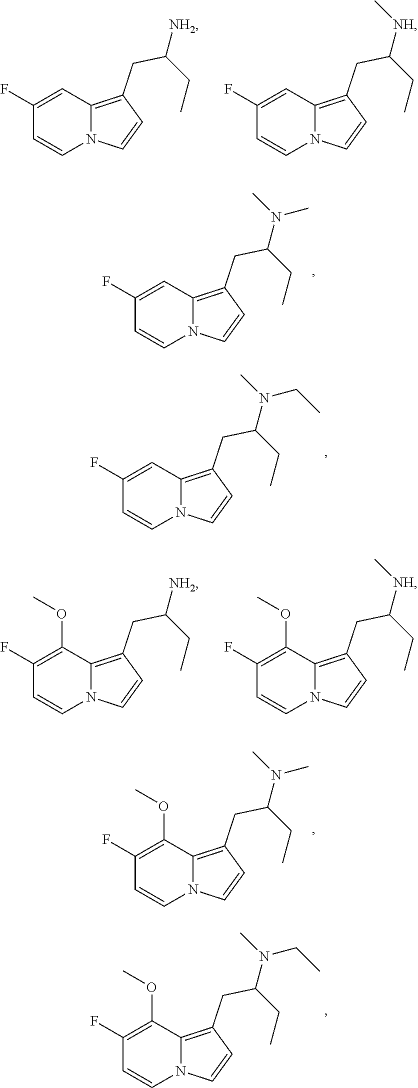

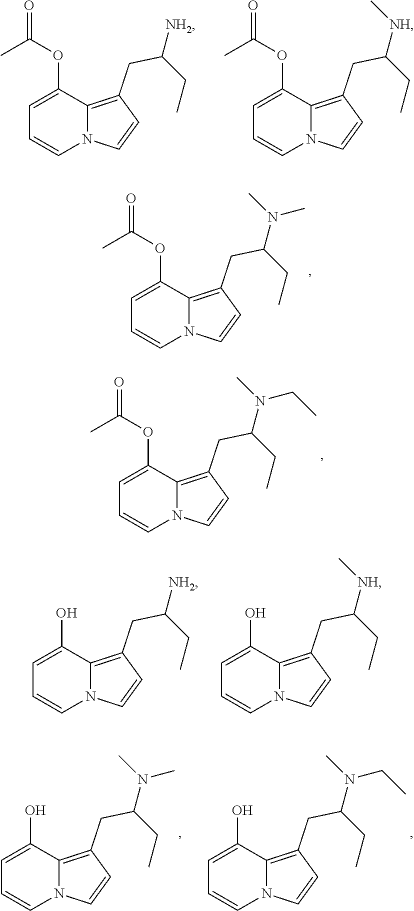

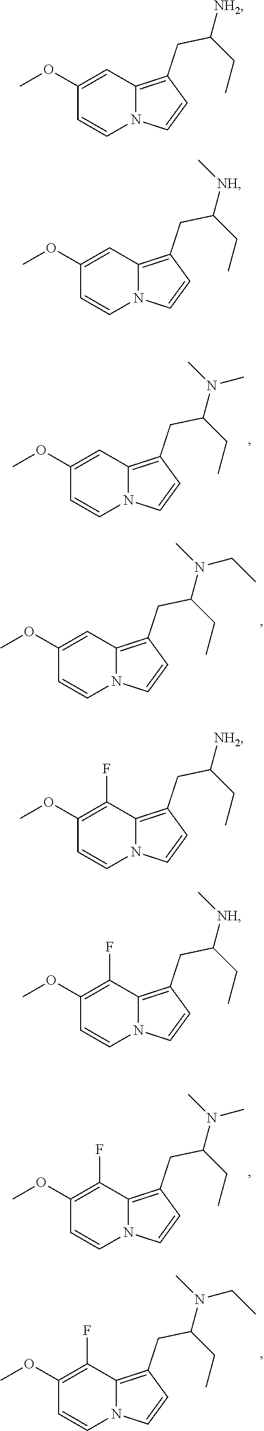

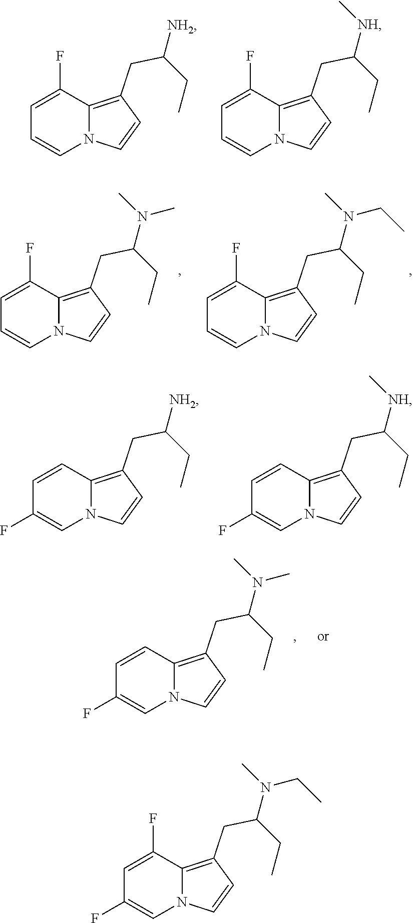

or pharmaceutically acceptable salt or salt mixture thereof.

In certain embodiments the compound of the present invention is of Formula:

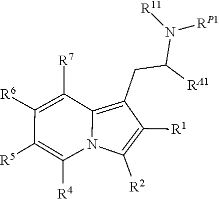

or pharmaceutically acceptable salt or salt mixture thereof.

In other embodiments the compound of the present invention is of Formula:

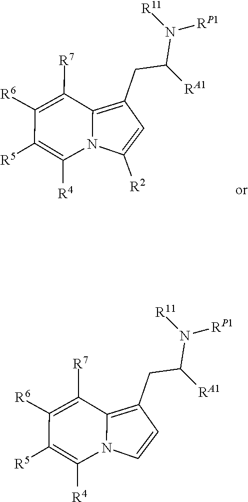

or pharmaceutically acceptable salt or salt mixture thereof.

In certain embodiments the compound of the present invention is of Formula:

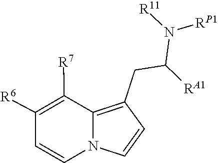

or pharmaceutically acceptable salt or salt mixture thereof.

In certain embodiments the compound of the present invention is of Formula:

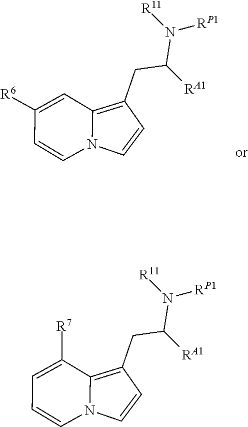

or pharmaceutically acceptable salt or salt mixture thereof.

In certain embodiments the compound of the present invention is of Formula:

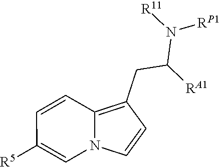

or pharmaceutically acceptable salt or salt mixture thereof.

In certain embodiments the compound of the present invention is of Formula:

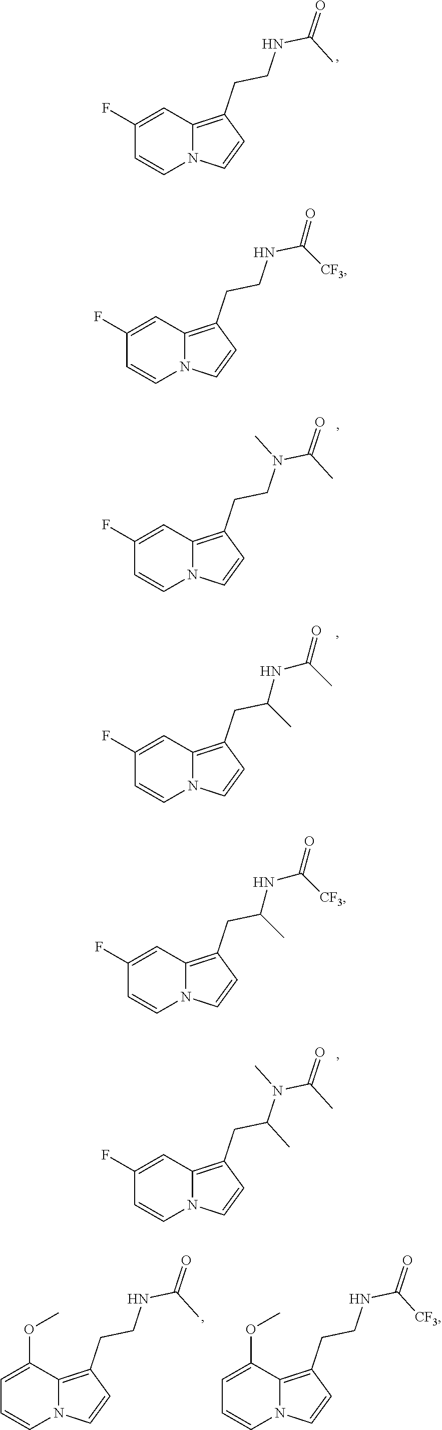

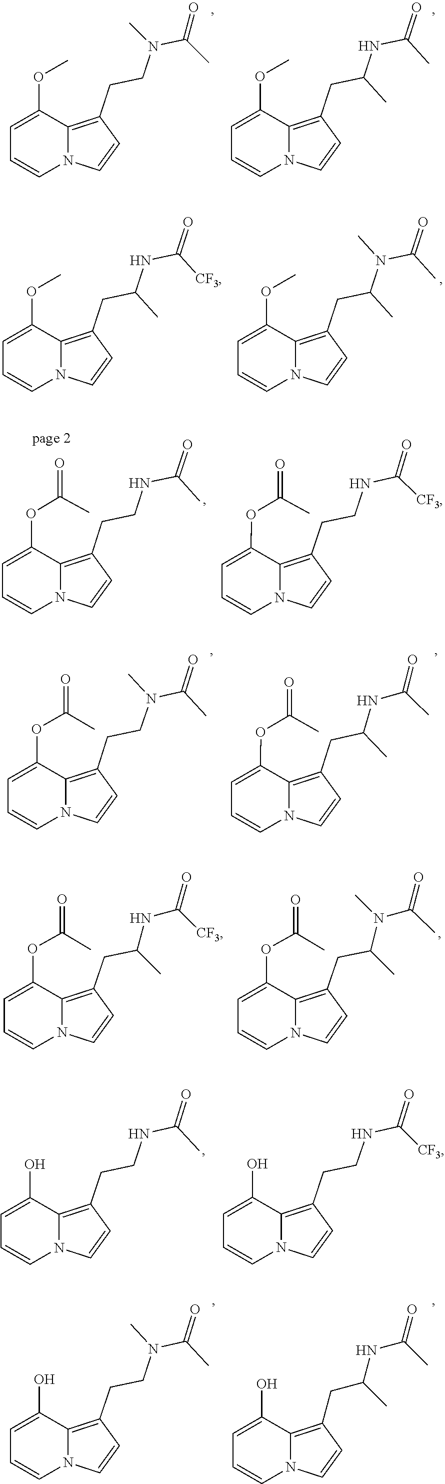

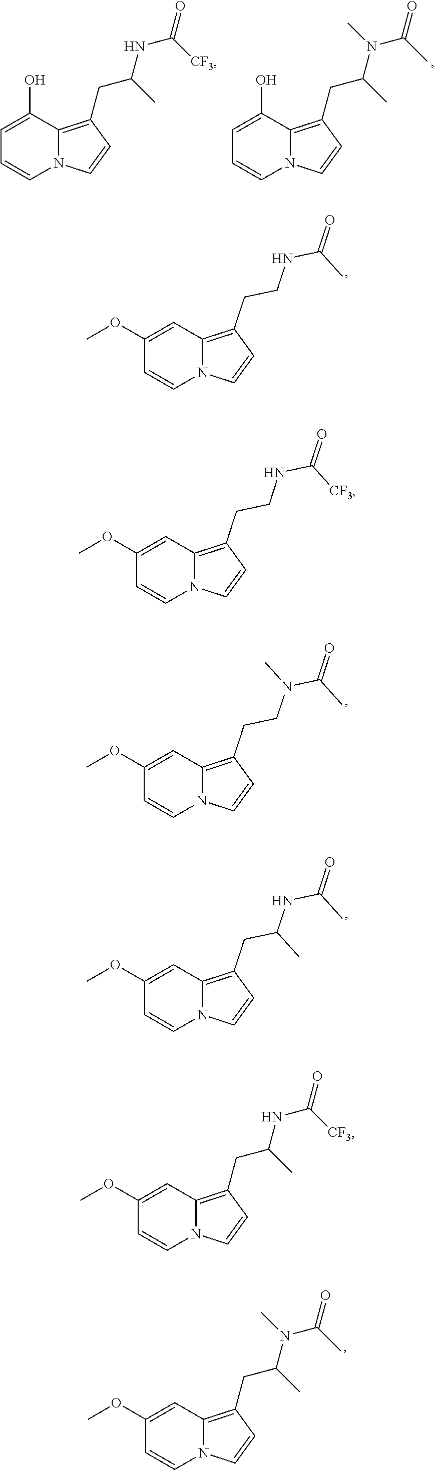

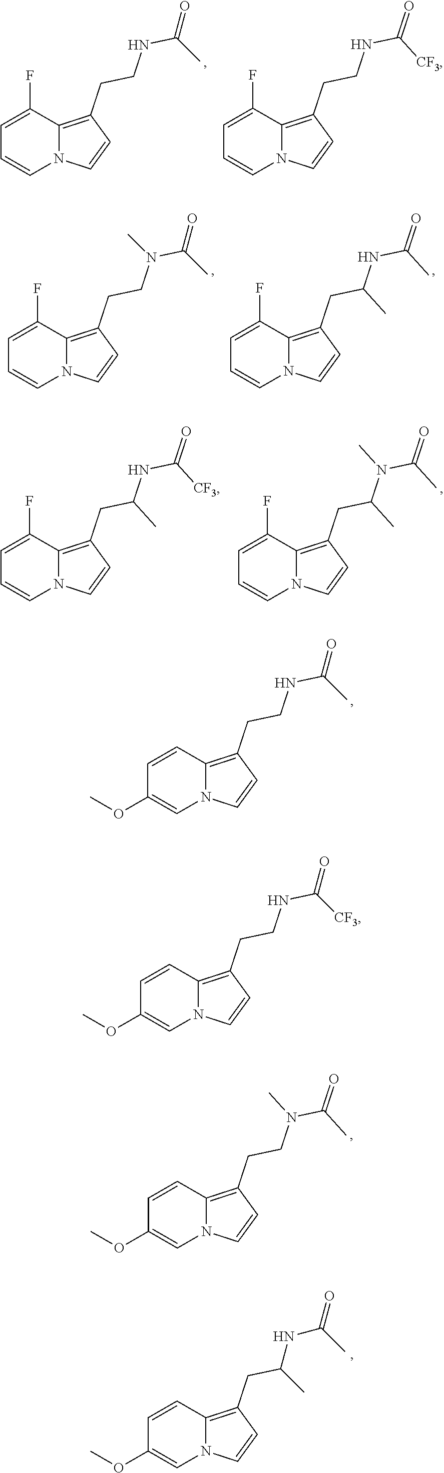

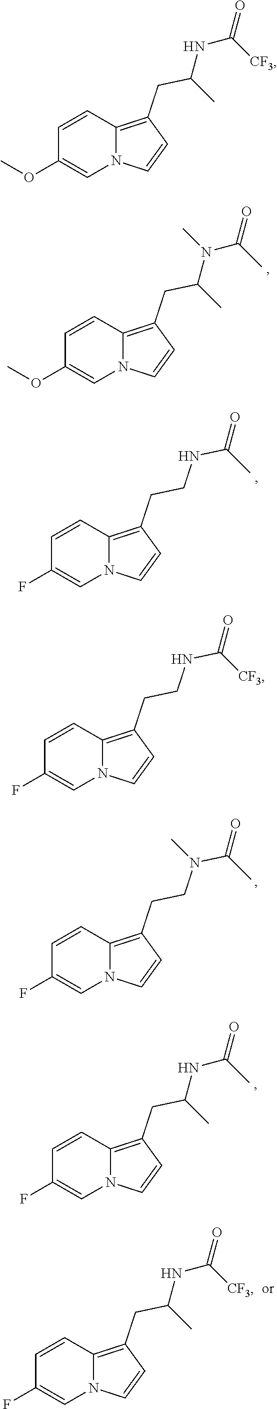

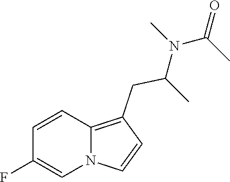 [SMILES: CC(=O)N(C)C(C)Cc1ccn2cc(F)ccc12]

or pharmaceutically acceptable salt or salt mixture thereof.

In certain embodiments the compound of the present invention is of Formula:

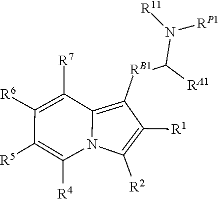

or pharmaceutically acceptable salt or salt mixture thereof.

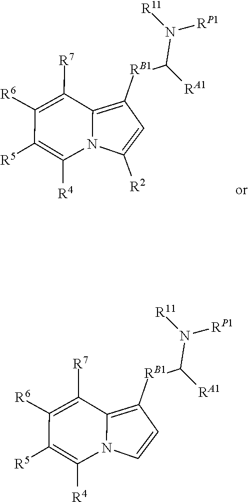

or pharmaceutically acceptable salt or salt mixture thereof.

In certain embodiments the compound of the present invention is of Formula:

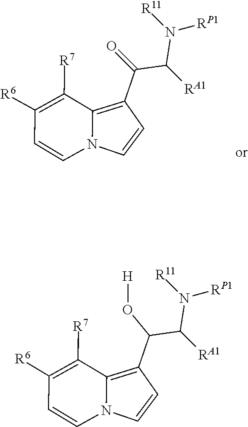

or pharmaceutically acceptable salt or salt mixture thereof.

In certain embodiments the compound of the present invention is of Formula:

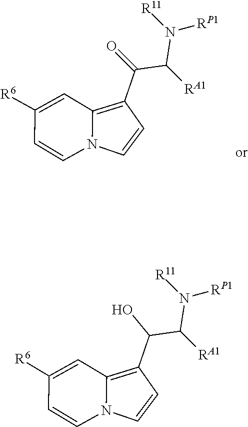

or pharmaceutically acceptable salt or salt mixture thereof.

In certain embodiments the compound of the present invention is of Formula:

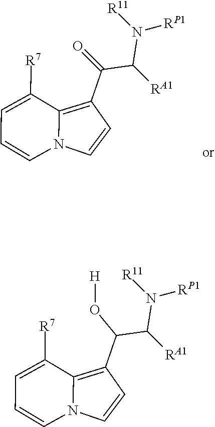

or pharmaceutically acceptable salt or salt mixture thereof.

In certain embodiments the compound of the present invention is of Formula:

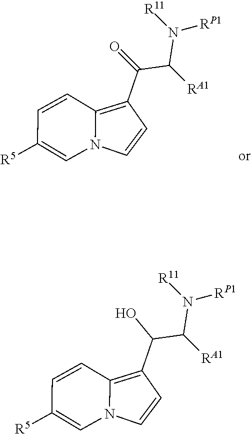

or pharmaceutically acceptable salt or salt mixture thereof.

In certain embodiments the compound of the present invention is of Formula:

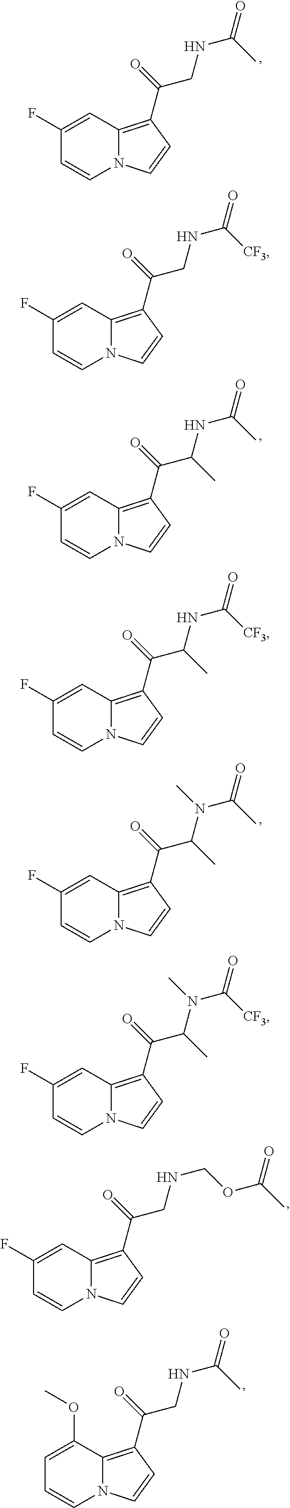

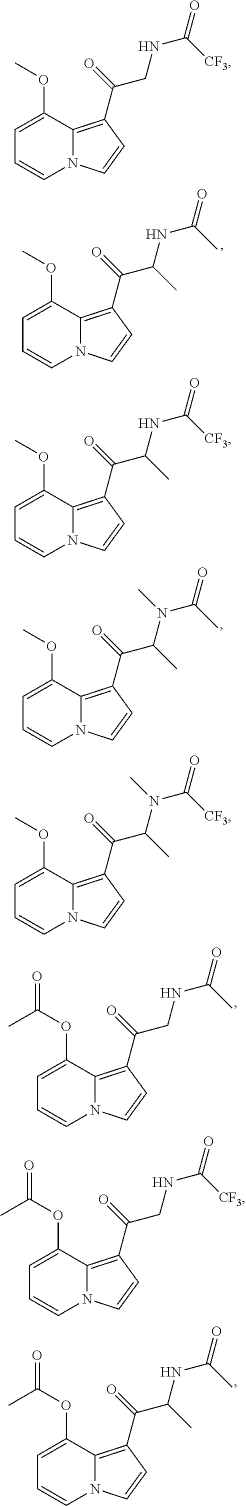

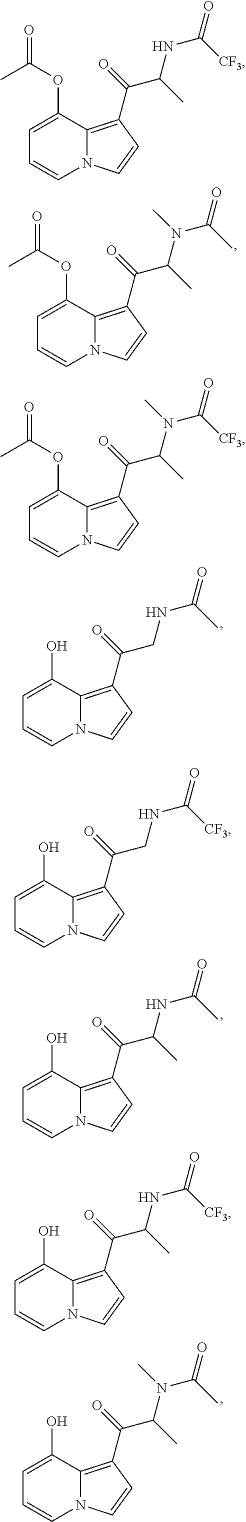

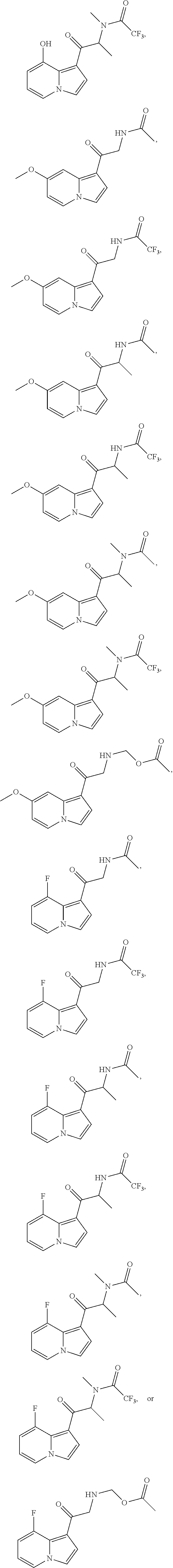

or pharmaceutically acceptable salt or salt mixture thereof.

In certain embodiments the compound of the present invention is of Formula:

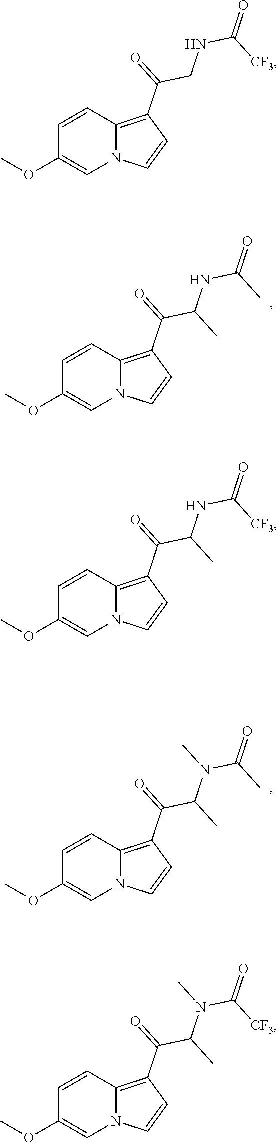

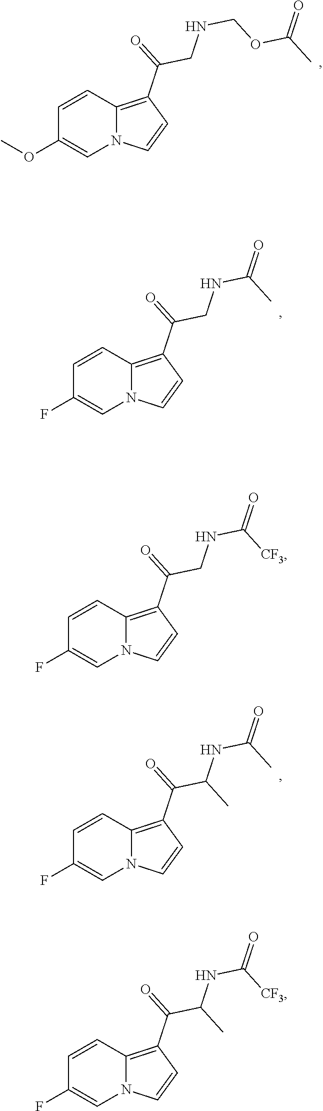 [SMILES: COc1ccc2c(C(=O)CNCOC(C)=O)ccn2c1 ; CC(=O)NCC(=O)c1ccn2cc(F)ccc12 ; O=C(CNC(=O)C(F)(F)F)c1ccn2cc(F)ccc12 ; CC(=O)NC(C)C(=O)c1ccn2cc(F)ccc12 ; CC(NC(=O)C(F)(F)F)C(=O)c1ccn2cc(F)ccc12]

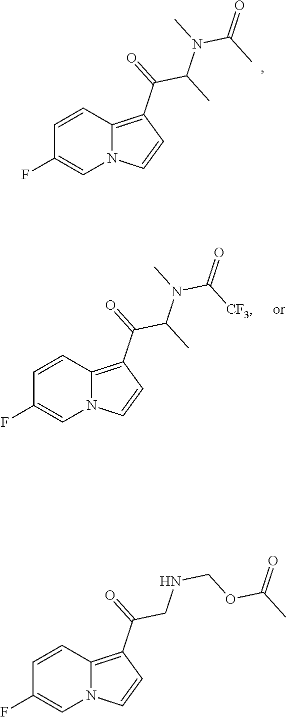 [SMILES: CC(=O)N(C)C(C)C(=O)c1ccn2cc(F)ccc12 ; CC(C(=O)c1ccn2cc(F)ccc12)N(C)C(=O)C(F)(F)F ; CC(=O)OCNCC(=O)c1ccn2cc(F)ccc12]

In certain embodiments the compound of the present invention is of Formula:

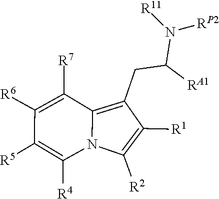

or pharmaceutically acceptable salt or salt mixture thereof.

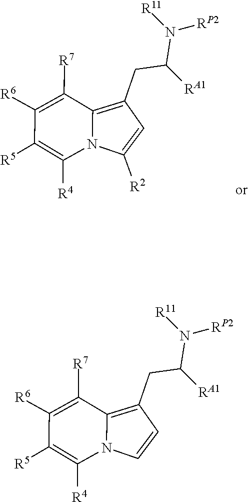

or pharmaceutically acceptable salt or salt mixture thereof.

In certain embodiments the compound of the present invention is of Formula:

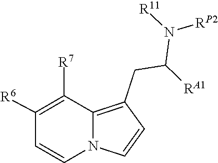

or pharmaceutically acceptable salt or salt mixture thereof.

In certain embodiments the compound of the present invention is of Formula:

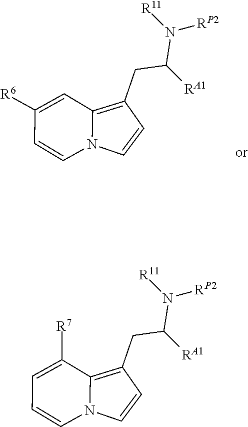

or pharmaceutically acceptable salt or salt mixture thereof.

In certain embodiments the compound of the present invention is of Formula:

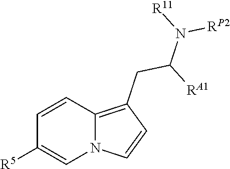

or pharmaceutically acceptable salt or salt mixture thereof.

In certain embodiments the compound of the present invention is of Formula:

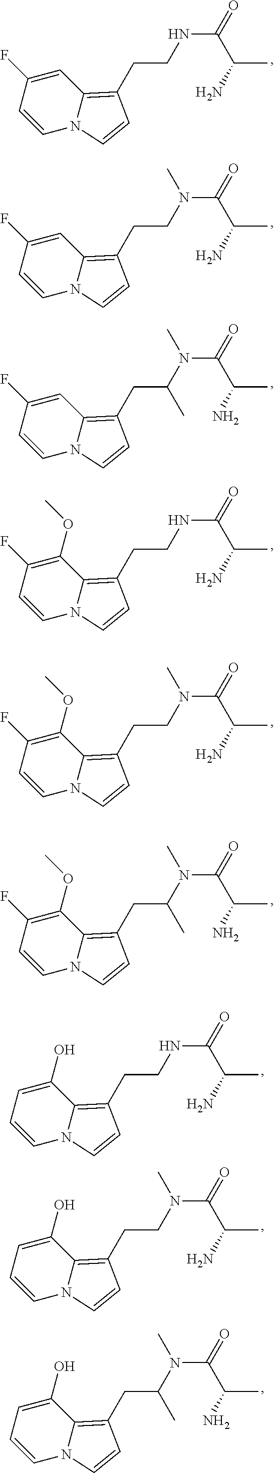

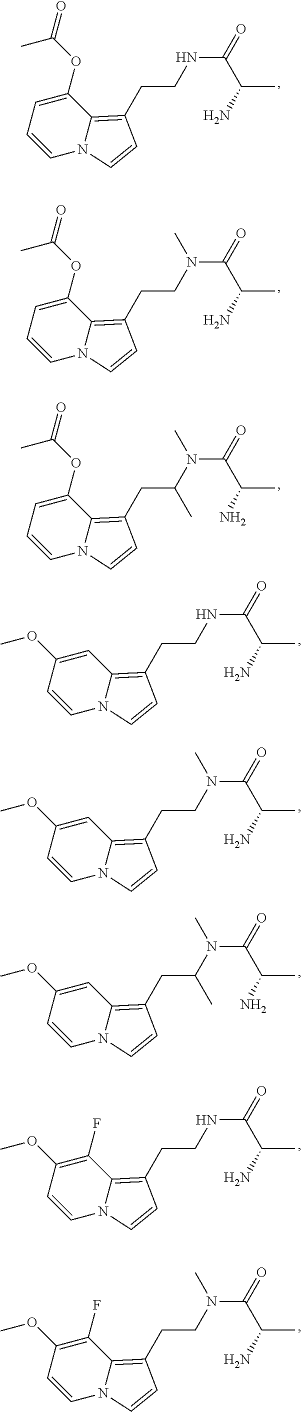

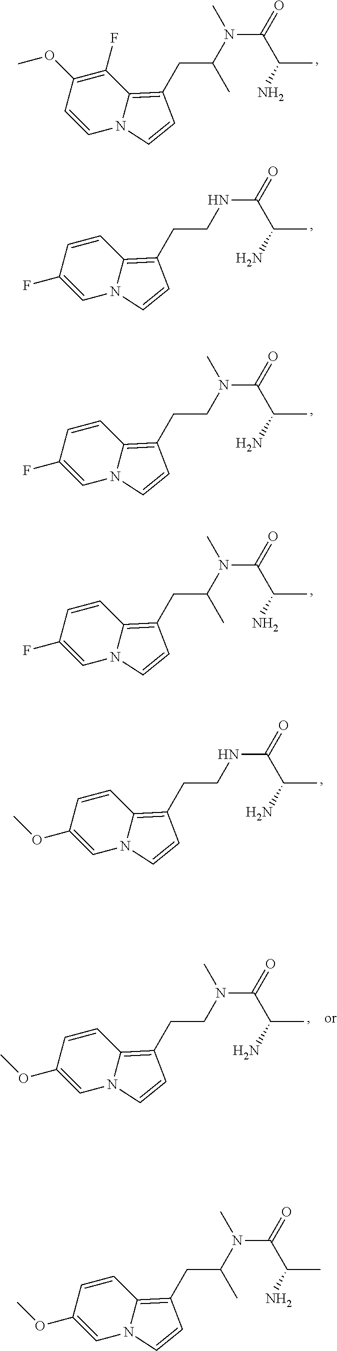

or pharmaceutically acceptable salt or salt mixture thereof.

In certain embodiments the compound of the present invention is of Formula:

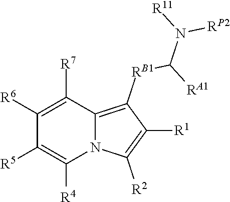

or pharmaceutically acceptable salt or salt mixture thereof.

In other embodiments the compound of the present invention is of Formula:

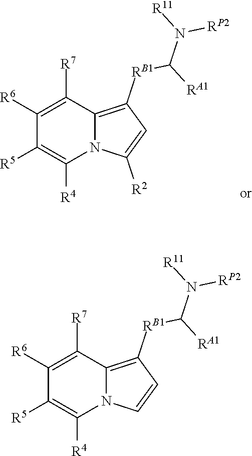

or pharmaceutically acceptable salt or salt mixture thereof.

In certain embodiments the compound of the present invention is of Formula:

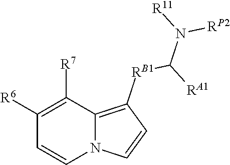

or pharmaceutically acceptable salt or salt mixture thereof.

In certain embodiments the compound of the present invention is of Formula:

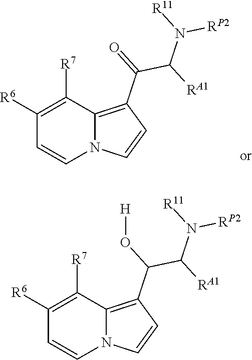

or pharmaceutically acceptable salt or salt mixture thereof.

In certain embodiments the compound of the present invention is of Formula:

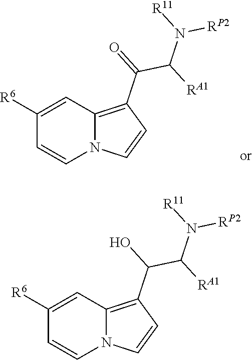

or pharmaceutically acceptable salt or salt mixture thereof.

In certain embodiments the compound of the present invention is of Formula:

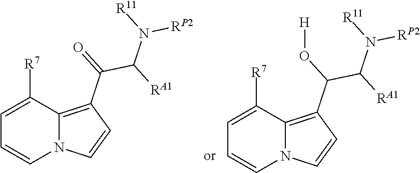

or pharmaceutically acceptable salt or salt mixture thereof.

In certain embodiments the compound of the present invention is of Formula:

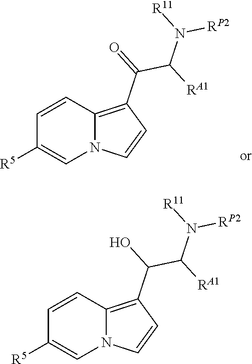

or pharmaceutically acceptable salt or salt mixture thereof.

In certain embodiments the compound of the present invention is of Formula:

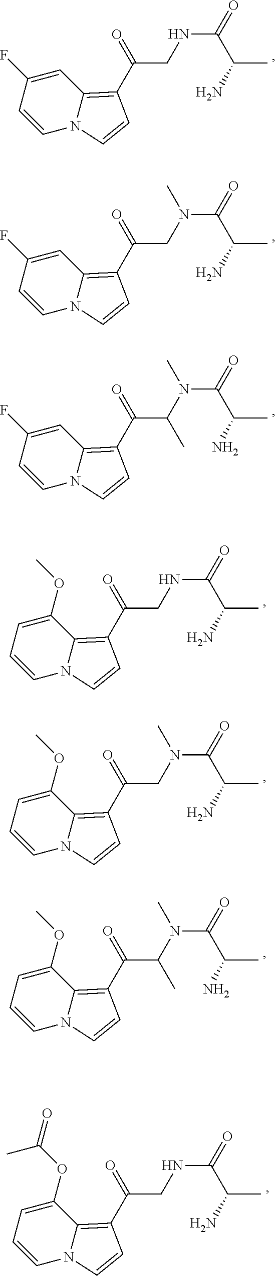

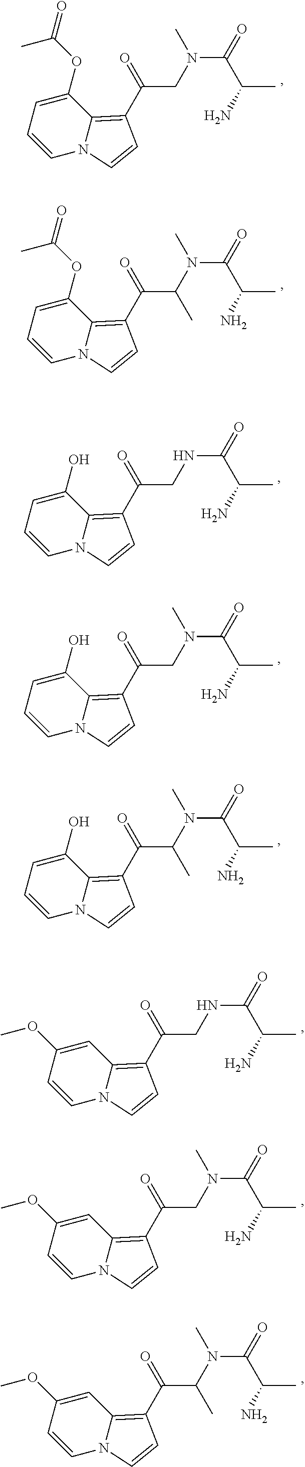

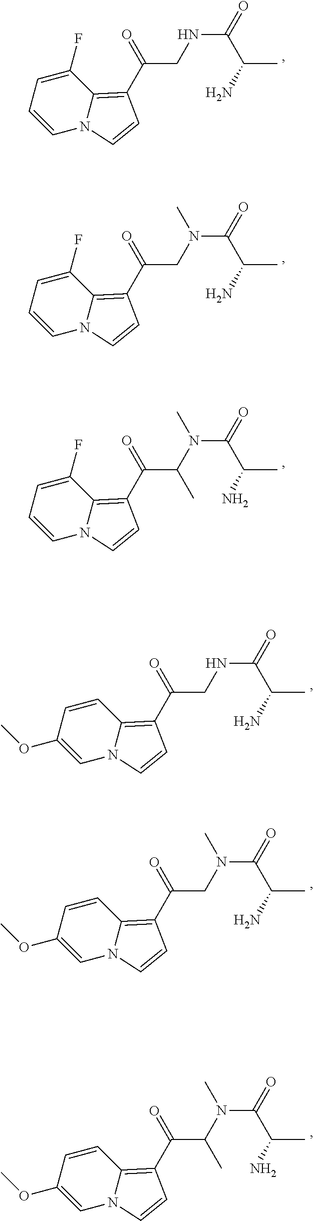

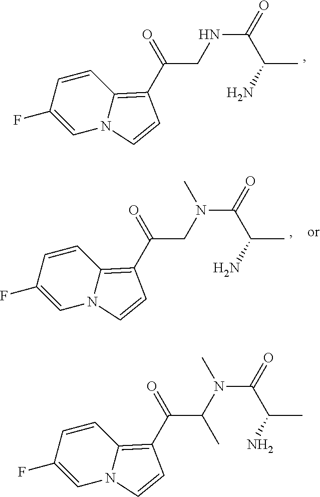 [SMILES: C[C@H](N)C(=O)NCC(=O)c1ccn2cc(F)ccc12 ; C[C@H](N)C(=O)N(C)CC(=O)c1ccn2cc(F)ccc12 ; CC(C(=O)c1ccn2cc(F)ccc12)N(C)C(=O)[C@H](C)N]

or pharmaceutically acceptable salt or salt mixture thereof.

In certain embodiments the compound of the present invention is of Formula:

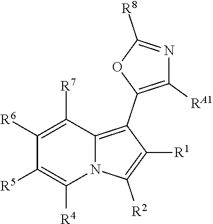

or pharmaceutically acceptable salt or salt mixture thereof.

In other embodiments the compound of the present invention is of Formula:

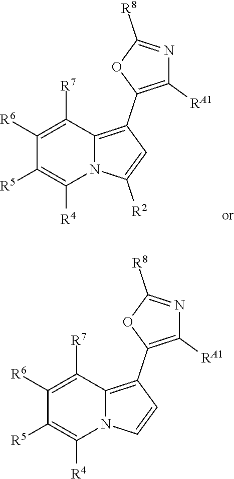

or pharmaceutically acceptable salt or salt mixture thereof.

In certain embodiments the compound of the present invention is of Formula:

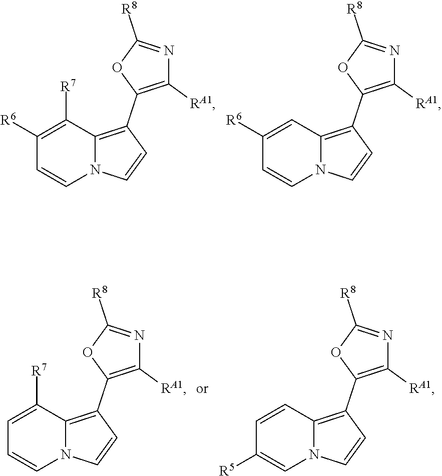

or pharmaceutically acceptable salt or salt mixture thereof.

In certain embodiments the compound of the present invention is of Formula:

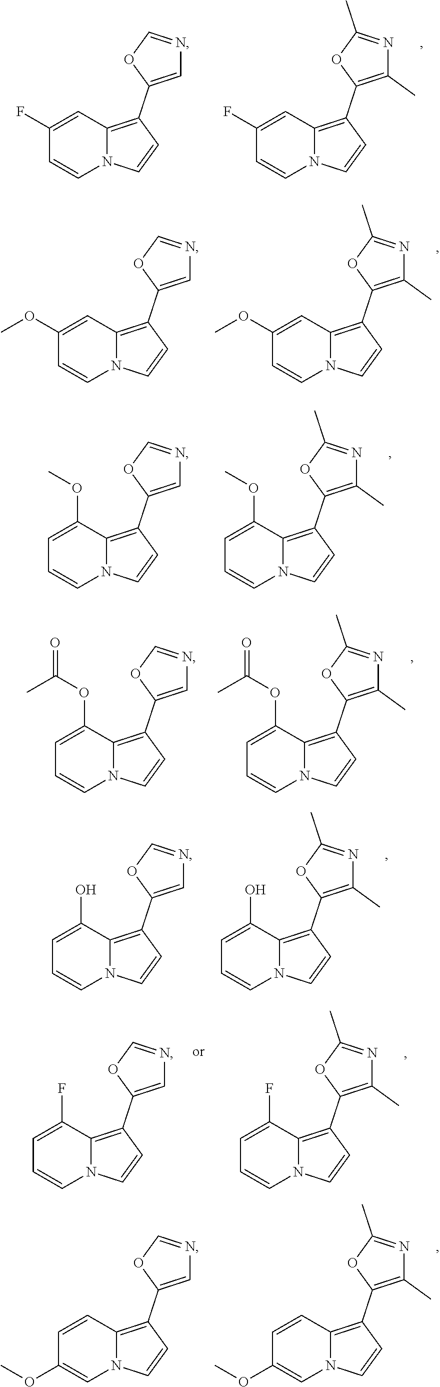

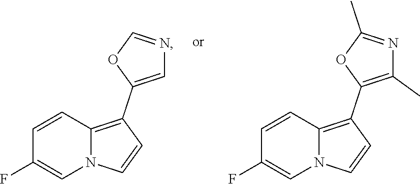 [SMILES: Fc1ccc2c(-c3cnco3)ccn2c1 ; "or" ; Cc1nc(C)c(-c2ccn3cc(F)ccc23)o1]

or pharmaceutically acceptable salt or salt mixture thereof.

In certain embodiments a compound, pharmaceutical composition, or method described below is provided.

1. A compound of Formula:

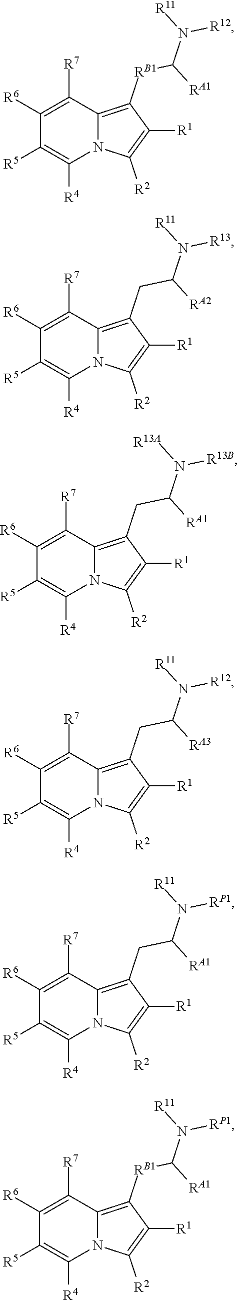

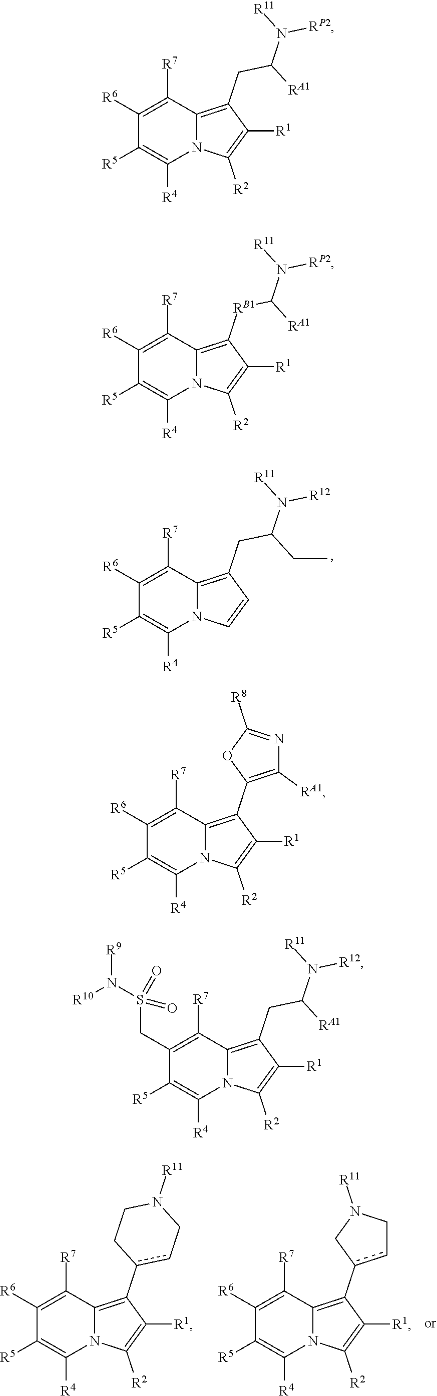

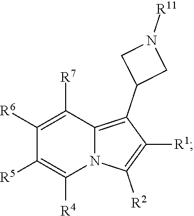

or a pharmaceutically acceptable salt or salt mixture thereof;
   wherein:
[symbol] is a single or double bond;
R^{A1} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R^{A2} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R^{A3} is —CH₂X, —CHX₂, —CX₃, —CH₂CH₂X, —CH₂ CHX₂, —CH₂CX₃, —CH₂OH, or —CH₂CH₂OH;
R^{B1} is

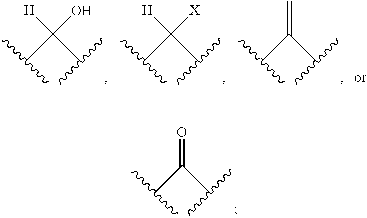

R¹, R², R⁴, R⁵ R⁶, R⁷, and R⁸ are independently selected from the group consisting of hydrogen, halogen, alkyl, haloalkyl, —OP(O)(OR⁹)₂, —SR⁹, —N⁹R¹, —NR^{P1}R¹⁰, —NR^{P2}R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹¹ is hydrogen, —(C₁-C₆)alkyl, —CH₂CH₂X, —CH₂ CHX₂, —CH₂CX₃, —CH₂CH₂OH, or hydroxy;
R¹³ is —(C₁-C₆)alkyl, —CH₂CH₂X, —CH₂ CHX₂, —CH₂CX₃, —CH₂CH₂OH, or hydroxy;
R^{13A} is —(C₁-C₆)alkyl, —CH₂CH₂X, —CH₂ CHX₂, —CH₂CX₃, or —CH₂CH₂OH;
R^{13B} is —(C₃-C₆)alkyl, —CH₂CH₂X, —CH₂ CHX₂, —CH₂CX₃, —CH₂CH₂OH, or hydroxy;
R¹³1 is alkyl, haloalkyl, —OP(O)(OR⁹)₂, —SR⁹, —NR⁹R¹⁰, —OR⁹, -alkyl-OP(O)(OR⁹)₂, -alkyl-SR⁹, -alkyl-NR⁹R¹⁰, or -alkyl-OR⁹;
R¹¹ is alkyl, haloalkyl, —NR⁹R¹⁰, or —OR⁹;
R^{P1} is selected at each instance from the group consisting of —C(O)R^{13C}, -alkyl-OC(O)R^{13C}, and -alkyl-C(O)R^{13C};
R^{P2} is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R¹⁸ and —S(O)₂R¹⁸; and
R¹⁸ is independently selected from the group consisting of hydrogen, alkyl, aryl, arylalkyl, cycloalkyl, heterocycle, heteroaryl, and heteroarylalkyl.
   2. The compound of embodiment 1, wherein the compound is of formula:

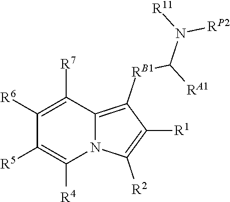

or a pharmaceutically acceptable salt or salt mixture thereof.
   3. The compound of embodiment 1, wherein the compound is of formula:

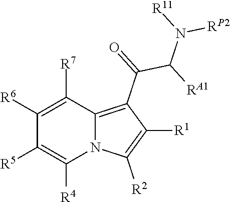

or a pharmaceutically acceptable salt or salt mixture thereof.
   4. The compound of embodiment 1, wherein the compound is selected from:

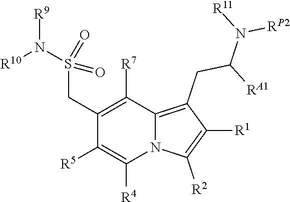

or a pharmaceutically acceptable salt or salt mixture thereof.
   5. The compound of any one of embodiments 1-4, wherein R¹² is hydrogen.
   6. The compound of any one of embodiments 1-4, wherein R¹² is methyl.
   7. The compound of any one of embodiments 1-4, wherein R¹² is ethyl.
   8. The compound of any one of embodiments 1-4, wherein R¹² is isopropyl.
   9. The compound of embodiment 1, wherein the compound is of formula:

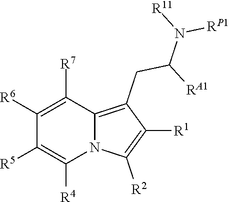

or a pharmaceutically acceptable salt or salt mixture thereof.
   10. The compound of embodiment 1, wherein the compound is of formula:

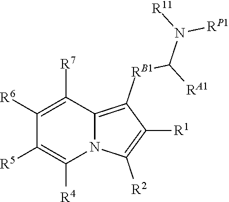

or a pharmaceutically acceptable salt or salt mixture thereof.
   11. The compound of embodiment 9 or 10, wherein R^{P1} is —C(O)R^{13C}.
   12. The compound of embodiment 11, wherein R^{13C} is C₁-C₆alkyl.
   13. The compound of embodiment 1 of formula:

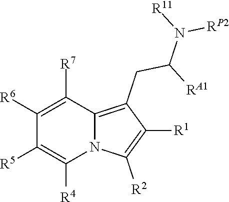

or a pharmaceutically acceptable salt or salt mixture thereof.
   14. The compound of embodiment 1, wherein the compound is of formula:

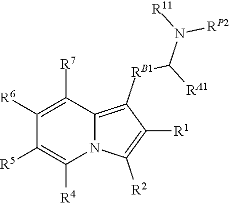

or a pharmaceutically acceptable salt or salt mixture thereof.
   15. The compound of embodiment 13 or 14, wherein R¹¹ is an amino acid connected through the C-terminus to the ethyl amine moiety.
   16. The compound of embodiment 13 or 14, wherein R^{P2} is a peptide comprising 2, 3, or 4 amino acids connected through the C-terminus to the ethyl amine moiety.
   17. The compound of embodiment 1, wherein the compound is of formula:

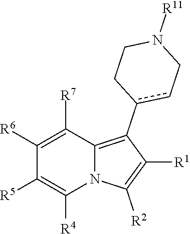

or a pharmaceutically acceptable salt or salt mixture thereof.
   18. The compound of embodiment 1, wherein the compound is of formula:

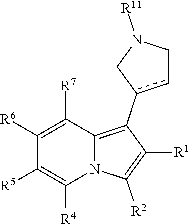

or a pharmaceutically acceptable salt or salt mixture thereof.
   19. The compound of embodiment 1, wherein the compound is of formula:

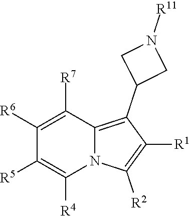

or a pharmaceutically acceptable salt or salt mixture thereof.
   20. The compound of embodiment 1, wherein the compound is of formula:

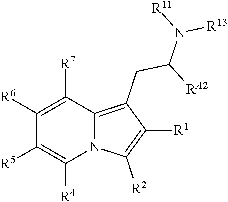

or a pharmaceutically acceptable salt or salt mixture thereof.
   21. The compound of embodiment 20, wherein R^{A2} is methyl.
   22. The compound of embodiment 20, wherein R^{A2} is ethyl.
   23. The compound of any one of embodiments 20-22, wherein R¹³ is methyl.
   24. The compound of any one of embodiments 20-22, wherein R¹³ is ethyl.
   25. The compound of any one of embodiments 20-22, wherein R¹³ is isopropyl.
   26. The compound of embodiment 1, wherein the compound is of formula:

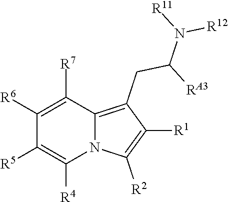

or a pharmaceutically acceptable salt or salt mixture thereof.
   27. The compound of embodiment 26, wherein R^{A3} is trifluoromethyl or fluoromethyl.
   28. The compound of any one of embodiments 26-27, wherein R¹² is methyl.
   29. The compound of any one of embodiments 26-27, wherein R¹² is ethyl.
   30. The compound of any one of embodiments 26-27, wherein R¹² is isopropyl.
   31. The compound of any one of embodiments 26-27, wherein R¹² is hydrogen.
   32. The compound of any one of embodiments 1-31, wherein R¹¹ is hydrogen.
   33. The compound of any one of embodiments 1-31, wherein R¹¹ is methyl.
   34. The compound of any one of embodiments 1-31, wherein R¹¹ is ethyl.
   35. The compound of any one of embodiments 1-31, wherein R¹¹ is isopropyl.
   36. The compound of embodiment 1, wherein the compound is of formula:

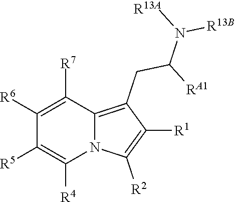

or a pharmaceutically acceptable salt or salt mixture thereof.
   37. The compound of embodiment 1, wherein the compound is of formula:

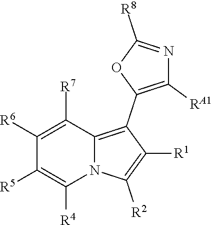

or a pharmaceutically acceptable salt or salt mixture thereof.
   38. The compound of any one of embodiments 1-37, wherein R^{A1} is hydrogen.
   39. The compound of any one of embodiments 1-37, wherein R^{A1} is methyl.
   40. The compound of any one of embodiments 1-37, wherein R^{A1} is ethyl.
   41. The compound of any one of embodiments 1-40, wherein R¹ is hydrogen.
   42. The compound of any one of embodiments 1-41, wherein R² is hydrogen.
   43. The compound of any one of embodiments 1-42, wherein R⁴ is hydrogen.
   44. The compound of any one of embodiments 1-43, wherein R⁵ is hydrogen.
   45. The compound of any one of embodiments 1-44, wherein R⁶ is hydrogen.
   46. The compound of any one of embodiments 1-45, wherein R⁷ is hydrogen.
   47. The compound of embodiment 1, wherein the compound is selected from:

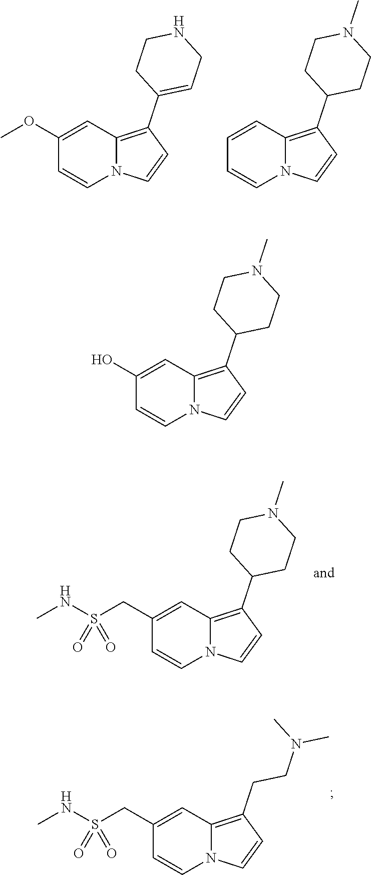

or a pharmaceutically acceptable salt thereof.
   48. The compound of embodiment 1, wherein the compound is selected from:
or a pharmaceutically acceptable salt thereof.

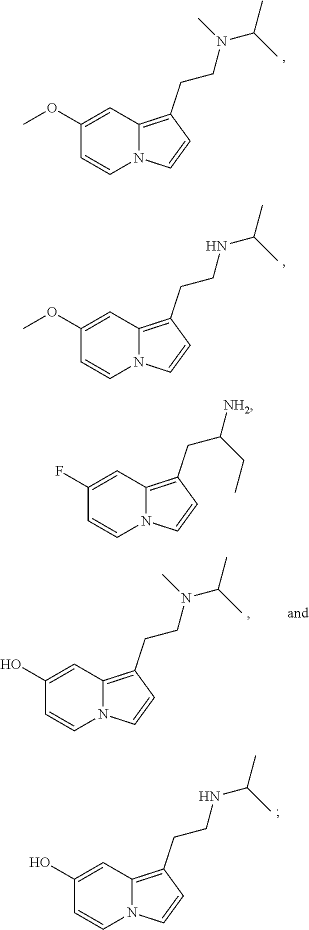

49. The compound of embodiment 1, wherein the compound is selected from:

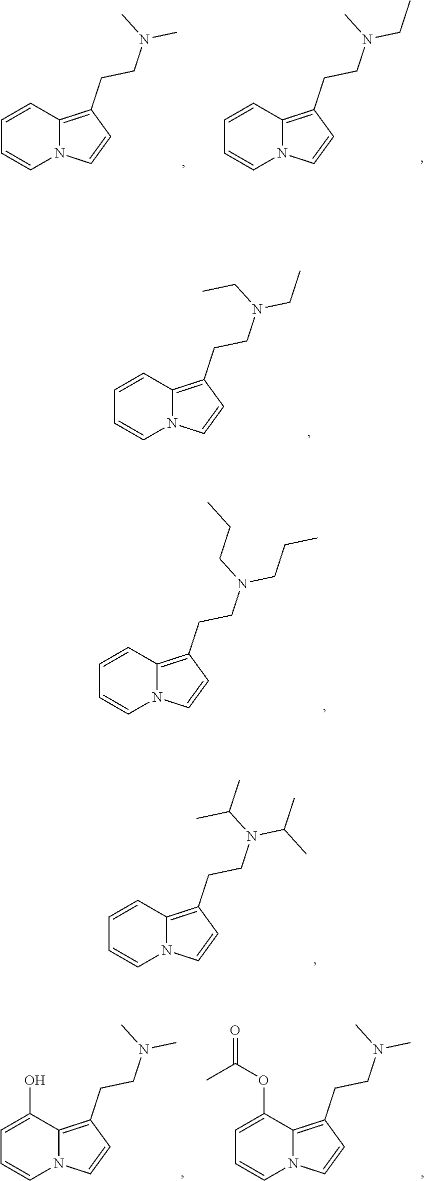

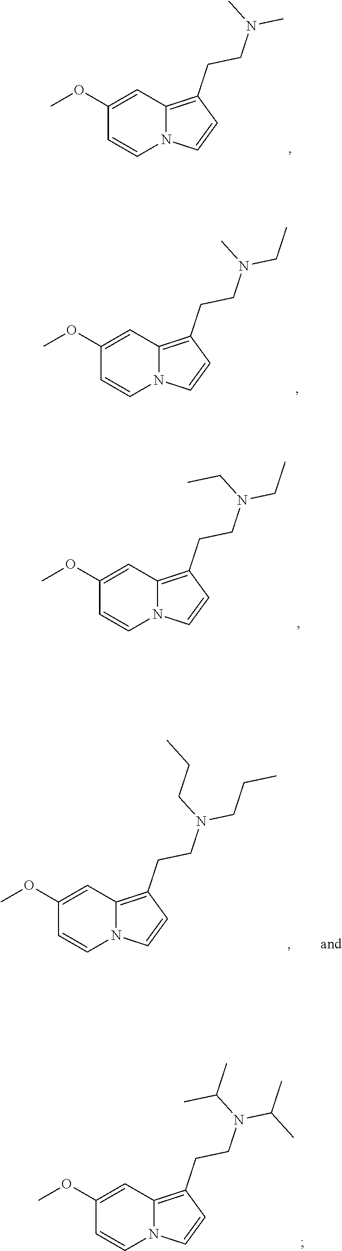

or a pharmaceutically acceptable salt thereof.
   50. The compound of any of embodiments 1-49, wherein the compound has entactogenic properties.
   51. The compound of any of embodiments 1-49, wherein the compound has serotonin-receptor-dependent properties.
   52. The compound of any of embodiments 1-49, with decreased hallucinogenic effects relative to MDMA.
   53. The compound of any of embodiments 1-49, with decreased unwanted psychoactive effects relative to MDMA.
   54. The compound of any of embodiments 1-49, with decreased physiological effects relative to MDMA.
   55. The compound of any of embodiments 1-49, with decreased abuse potential relative to MDMA.
   56. The compound of any of embodiments 1-49, with decreased hallucinogenic effects relative to a clinically used 5-HT_{2A} agonist.
   57. The compound of any of embodiments 1-49, with decreased unwanted psychoactive effects relative to a clinically used 5-HT_{2A} agonist.
   58. The compound of any of embodiments 1-49, with decreased physiological effects relative to a clinically used 5-HT_{2A} agonist.
   59. The compound of any of embodiments 1-58 that shows the therapeutic effect of emotional openness.
   60. The compound of any of embodiments 1-59, wherein the pharmaceutically acceptable salt(s) is selected from HCl, sulfate, aspartate, saccharate, fumarate, succinate, phosphate, oxalate, acetate, amino acid anion, gluconate, maleate, malate, citrate, mesylate, nitrate or tartrate, or a mixture thereof.
   61. The compound of embodiment 60 that is also a serotonin reuptake inhibitor.
   62. The compound of any of embodiments 1-61 that has minimal or no direct agonism of 5-HT_{2A}.
   63. The compound of any of embodiments 1-61 that is a direct 5-HT_{2A} agonist.
   64. The compound of any of embodiments 1-61 that is a serotonin releaser.
   65. The compound of any of embodiments 1-61 that is both a direct 5-HT_{2A} agonist and a serotonin releaser.
   66. The compound of any of embodiments 1-61 that is a psychoplastogen.
   67. The compound of any of embodiments 1-61 that is a direct 5-HT_{1B} agonist and direct 5-HT_{1D} agonist.
   68. The compound of any of embodiments 1-61 that is a direct 5-HT_{1B} agonist.
   69. The compound of any of embodiments 1-61 that is a direct 5-HT_{1B} agonist and partial or full 5-HT_{2A} agonist with higher potency for 5-HT_{1B} compared to 5-HT_{2A}.
   70. The compound of any of embodiments 1-61 that is both a serotonin releaser and 5-HT_{2B} antagonist.
   71. The compound of any of embodiments 1-49, wherein the compound is an enantiomerically enriched mixture or pure enantiomer.
   72. The enantiomerically enriched mixture or pure enantiomer of embodiment 71, wherein the compound has entactogenic properties.
   73. The enantiomerically enriched mixture or pure enantiomer of embodiment 71, wherein the compound has serotonin-receptor-dependent properties.
   74. The enantiomerically enriched mixture or pure enantiomer of embodiment 71, with decreased hallucinogenic effects relative to MDMA.
   75. The enantiomerically enriched mixture or pure enantiomer of embodiment 71, with decreased unwanted psychoactive effects relative to MDMA.
   76. The enantiomerically enriched mixture or pure enantiomer of embodiment 71, with decreased physiological effects relative to MDMA.
   77. The enantiomerically enriched mixture or pure enantiomer of embodiment 71, with decreased abuse potential relative to MDMA.
   78. The enantiomerically enriched mixture or pure enantiomer of embodiment 71, with decreased hallucinogenic effects relative to a clinically used 5-HT_{2A} agonist.
   79. The enantiomerically enriched mixture or pure enantiomer of embodiment 71, with decreased unwanted psychoactive effects relative to a clinically used 5-HT_{2A} agonist.
   80. The enantiomerically enriched mixture or pure enantiomer of embodiment 71, with decreased physiological effects relative to a clinically used 5-HT_{2A} agonist.
   81. The enantiomerically enriched mixture or pure enantiomer of embodiment 71, with decreased side effects relative to a clinically used triptan.
   82. The enantiomerically enriched mixture or pure enantiomer of embodiment 71, with decreased side effects relative to fenfluramine or another clinically used anti-seizure medicine.
   83. The enantiomerically enriched mixture or pure enantiomer of embodiment 71, with decreased side effects relative to fenfluramine, where those decreased side effects include decreased heart valve disorders.
   84. The enantiomerically enriched mixture or pure enantiomer of any of embodiments 71-83 that shows the therapeutic effect of emotional openness.
   85. The enantiomerically enriched mixture or pure enantiomer of any of embodiments 71-84 wherein the pharmaceutically acceptable salt(s) is selected from HCl, sulfate, aspartate, saccharate, fumarate, succinate, phosphate, oxalate, acetate, amino acid anion, gluconate, maleate, malate, citrate, mesylate, nitrate or tartrate, or a mixture thereof.
   86. The enantiomerically enriched mixture or pure enantiomer of any one of embodiments 71-85 that is also a serotonin reuptake inhibitor.
   87. The enantiomerically enriched mixture or pure enantiomer of any one of embodiments 71-86 that has minimal or no direct agonism of 5-HT_{2A}.
   88. The enantiomerically enriched mixture or pure enantiomer of any one of embodiments 71-87 that is a direct 5-HT_{2A} agonist.
   89. The enantiomerically enriched mixture or pure enantiomer of any one of embodiments 71-88 that is a serotonin releaser.
   90. The enantiomerically enriched mixture or pure enantiomer of any one of embodiments 71-89 that is both a direct 5-HT_{2A} agonist and a serotonin releaser.
   91. The enantiomerically enriched mixture or pure enantiomer of any one of embodiments 71-90 that is a psychoplastogen.
   92. The enantiomerically enriched mixture or pure enantiomer of embodiment 71 wherein the enantiomerically enriched mixture or pure enantiomer is an enantiomerically enriched mixture.
   93. The enantiomerically enriched mixture of embodiment 72, wherein the compound has entactogenic properties.
   94. The enantiomerically enriched mixture of embodiment 72, wherein the compound has serotonin-receptor-dependent properties.
   95. The enantiomerically enriched mixture of embodiment 72, with decreased hallucinogenic effects relative to MDMA.
   96. The enantiomerically enriched mixture of embodiment 72, with decreased unwanted psychoactive effects relative to MDMA.
   97. The enantiomerically enriched mixture of embodiment 72, with decreased physiological effect relative to MDMA.
   98. The enantiomerically enriched mixture of embodiment 72, with decreased abuse potential relative to MDMA.
   99. The enantiomerically enriched mixture of embodiment 72, with decreased hallucinogenic effects relative to a clinically used 5-HT_{2A} agonist.
   100. The enantiomerically enriched mixture of embodiment 72, with decreased unwanted psychoactive effects relative to a clinically used 5-HT_{2A} agonist.
   101. The enantiomerically enriched mixture of embodiment 72, with decreased physiological effects relative to a clinically used 5-HT_{2A} agonist.
   102. The enantiomerically enriched mixture of any of embodiments 72-101 that shows the therapeutic effect of emotional openness.
   103. The enantiomerically enriched mixture of any of embodiments 72-102 wherein the pharmaceutically acceptable salt(s) is selected from HCl, sulfate, aspartate, saccharate, fumarate, succinate, phosphate, oxalate, acetate, amino acid anion, gluconate, maleate, malate, citrate, mesylate, nitrate or tartrate, or a mixture thereof.
   104. The enantiomerically enriched mixture of embodiment 72-103 that is also a serotonin reuptake inhibitor.
   105. The enantiomerically enriched mixture of any one of embodiments 72-103 that has minimal or no direct agonism of 5-HT_{2A}.
   106. The enantiomerically enriched mixture of any one of embodiments 72-103 that is a direct 5-HT_{2A} agonist.
   107. The enantiomerically enriched mixture of any one of embodiments 72-103 that is a serotonin releaser.
   108. The enantiomerically enriched mixture of any one of embodiments 72-103 that is both a direct 5-HT_{2A} agonist and a serotonin releaser.
   109. The enantiomerically enriched mixture of any one of embodiments 72-103 that is a psychoplastogen.
   110. A pharmaceutical composition comprising an effective patient-treating amount of a compound, pure enantiomer, or enantiomerically enriched mixture of any one of embodiments 1-109 and a pharmaceutically acceptable carrier or excipient.
   111. The pharmaceutical composition of embodiment 110 wherein the composition is administered systemically.
   112. The pharmaceutical composition of embodiment 110 wherein the composition is administered orally.
   113. The pharmaceutical composition of embodiment 110 wherein the composition is administered to mucosal tissue.
   114. The pharmaceutical composition of embodiment 110 wherein the composition is administered rectally.
   115. The pharmaceutical composition of embodiment 110 wherein the composition is administered topically.
   116. The pharmaceutical composition of embodiment 110 wherein the composition is administered subcutaneously.
   117. The pharmaceutical composition of embodiment 110 wherein the composition is administered intravenously.
   118. The pharmaceutical composition of embodiment 110 wherein the composition is administered intramuscularly.
   119. The pharmaceutical composition of embodiment 110 wherein the composition is administered via inhalation.
   120. The pharmaceutical composition of embodiment 112 wherein the composition is administered as a tablet.
   121. The pharmaceutical composition of embodiment 112 wherein the composition is administered as a gelcap.
   122. The pharmaceutical composition of embodiment 112 wherein the composition is administered as a capsule.
   123. The pharmaceutical composition of embodiment 112 wherein the composition is administered as an aqueous emulsion.
   124. The pharmaceutical composition of embodiment 112 wherein the composition is administered as an aqueous solution.
   125. The pharmaceutical composition of embodiment 112 wherein the composition is administered as a pill.
   126. The pharmaceutical composition of embodiment 113 wherein the composition is administered as a buccal tablet.
   127. The pharmaceutical composition of embodiment 113 wherein the composition is administered as a sublingual tablet.
   128. The pharmaceutical composition of embodiment 113 wherein the composition is administered as a sublingual strip.
   129. The pharmaceutical composition of embodiment 113 wherein the composition is administered as a sublingual liquid.
   130. The pharmaceutical composition of embodiment 113 wherein the composition is administered as a sublingual spray.
   131. The pharmaceutical composition of embodiment 113 wherein the composition is administered as a sublingual gel.
   132. The pharmaceutical composition of embodiment 115 wherein the composition is administered as a cream.
   133. The pharmaceutical composition of embodiment 115 wherein the composition is administered as a topical solution.
   134. The pharmaceutical composition of embodiment 117 wherein the composition is administered as an aqueous solution.
   135. The pharmaceutical composition of embodiment 119 wherein the composition is administered as a powder.
   136. The pharmaceutical composition of embodiment 119 wherein the composition is administered as an aerosol.
   137. A method for treating a central nervous system disorder comprising administering an effective amount of a compound, pure enantiomer, or enantiomerically enriched mixture of any one of embodiments 1-109 or a pharmaceutical composition of any one of embodiments 110-136 to a host in need thereof.
   138. A method for treating a central nervous system disorder comprising administering an effective amount of a compound, pure enantiomer, or enantiomerically enriched mixture Formula II to a host in need thereof.

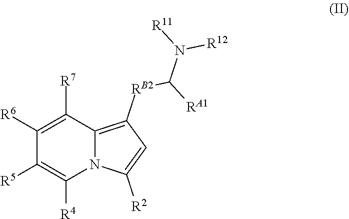

or a pharmaceutically acceptable salt or salt mixture thereof;
wherein
R^{B2} is

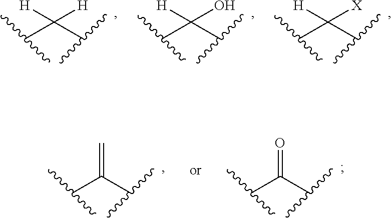

R^{A1} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R², R⁴, R⁵ R⁶, and R⁷ are independently selected from the group consisting of hydrogen, halogen, alkyl, haloalkyl, —OP(O)(OR⁹)₂, —SR⁹, —NR⁹R¹⁰, —NR^{P1}R¹⁰, —NR²R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹² is hydrogen, —(C₁-C₆)alkyl, —CH₂CH₂X, —CH₂ CHX₂, —CH₂CX₃, —CH₂CH₂OH, or hydroxy;
R¹⁷ is alkyl, haloalkyl, —NR⁹R¹⁰, or —OR⁹;
R¹¹ is selected at each instance from the group consisting of —C(O)R^{13C}, -alkyl-OC(O)R^{13C}, and -alkyl-C(O)R^{13C};
R^{P2} is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R¹⁸ and —S(O)₂R¹⁸; and
R⁸ is independently selected from the group consisting of hydrogen, alkyl, aryl, arylalkyl, cycloalkyl, heterocycle, heteroaryl, and heteroarylalkyl.
   139. The method of embodiment 137 or 138 wherein the host is a human.
   140. The method of any one of embodiments 137-139 wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism, dissociative disorders and headache disorders.
   141. The method of any one of embodiments 137-139 wherein the central nervous system disorder is post-traumatic stress disorder.
   142. The method of any one of embodiments 137-139 wherein the central nervous system disorder is adjustment disorder.
   143. The method of any one of embodiments 137-139 wherein the central nervous system disorder is generalized anxiety.
   144. The method of any one of embodiments 137-139 wherein the central nervous system disorder is social anxiety.
   145. The method of any one of embodiments 137-139 wherein the central nervous system disorder is depression.
   146. The method of any one of embodiments 137-139 wherein the central nervous system disorder is a substance use disorder.
   147. The method of any one of embodiments 137-139 wherein the central nervous system disorder is an attachment disorder.
   148. The method of any one of embodiments 137-139 wherein the central nervous system disorder is schizophrenia.
   149. The method of any one of embodiments 137-139 wherein the central nervous system disorder is a headache disorder.
   150. The method of any one of embodiments 137-139 wherein the central nervous system disorder is a migraine disorder.
   151. The method of any one of embodiments 137-139 wherein the central nervous system disorder is a seizure disorder.
   152. The method of any one of embodiments 137-139 wherein the central nervous system disorder is an eating disorder.
   153. The method of embodiment 152 wherein the eating disorder is bulimia.
   154. The method of embodiment 152 wherein the eating disorder is binge eating.
   155. The method of embodiment 152 wherein the eating disorder is anorexia.
   156. The method of any one of embodiments 137-139 wherein the central nervous system disorder is a neurological disorder.
   157. The method of embodiment 156 wherein the neurological disorder is stroke.
   158. The method of embodiment 156 wherein the neurological disorder is brain trauma.
   159. The method of embodiment 156 wherein the neurological disorder is dementia.
   160. The method of embodiment 156 wherein the neurological disorder is a neurodegenerative disease or disorder.
   161. The method of embodiment 160 wherein the neurodegenerative disease or disorder is selected from: Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.
   162. The method of any one of embodiments 137-161 wherein the compound, pure enantiomer, or enantiomerically enriched mixture is administered in a clinical setting.
   163. The method of any one of embodiments 137-161 wherein the compound, pure enantiomer, or enantiomerically enriched mixture is administered in an at-home setting.
   164. The method of any one of embodiments 137-161 wherein the compound, pure enantiomer, or enantiomerically enriched mixture is administered during a psychotherapy session.
   165. The method of any one of embodiments 137-161 wherein the compound, pure enantiomer, or enantiomerically enriched mixture is administered during a counseling session.
   166. A method for treating an inflammatory or metabolic disorder comprising administering an effective amount of a compound, pure enantiomer, or enantiomerically enriched mixture of any one of embodiments 1-109 or a pharmaceutical composition of any one of embodiments 110-136 to a host in need thereof.
   167. A method for treating an inflammatory or metabolic disorder comprising administering an effective amount of a compound, pure enantiomer, or enantiomerically enriched mixture Formula II to a host in need thereof.

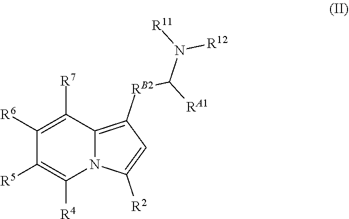

or a pharmaceutically acceptable salt or salt mixture thereof;
wherein
R^{B2} is

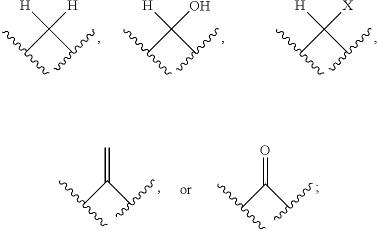

R^{A1} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R², R⁴, R⁵ R⁶, and R⁷ are independently selected from the group consisting of hydrogen, halogen, alkyl, haloalkyl, —OP(O)(OR⁹)₂, —SR⁹, —NR⁹R¹⁰, —NR^{P1}R¹⁰, —NR^{P2}R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹² is hydrogen, —(C₁-C₆)alkyl, —CH₂CH₂X, —CH₂ CHX₂, —CH₂CX₃, —CH₂CH₂OH, or hydroxy;
R¹⁷ is alkyl, haloalkyl, —NR⁹R¹⁰, or —OR⁹;
R^{P1} is selected at each instance from the group consisting of —C(O)R^{13C}, -alkyl-OC(O)R^{13C}, and -alkyl-C(O)R^{13C};
R¹¹ is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R¹⁸ and —S(O)₂R¹⁸; and
R¹⁸ is independently selected from the group consisting of hydrogen, alkyl, aryl, arylalkyl, cycloalkyl, heterocycle, heteroaryl, and heteroarylalkyl.
   168. The method of embodiment 166 or 167, wherein the host is a human.
   169. The method of any one of embodiments 166-168, wherein the disorder is an inflammatory disorder.
   170. The method of embodiment 169, wherein the level of inflammation is reduced.
   171. The method of embodiment 170, wherein the reduction in inflammation is determined by a decrease in TNF-mediated proinflammatory markers.
   172. The method of embodiment 171, wherein the decrease in TNF-mediated proinflammatory markers is a decrease in intracellular adhesion molecule 1 (ICAM-1), vascular adhesion molecule 1 (VCAM-1), interleukin (IL)-6 gene expression, nitric-oxide synthase activity, or nuclear translocation of nuclear factor KB.
   173. The method of any one of embodiments 167-172 wherein the disorder is asthma.
   174. The method of embodiment 170 wherein the reduction in inflammation is determined by a decrease in one or more markers of asthma severity.
   175. The method of embodiment 174 wherein the markers of asthma severity are selected from the group consisting of a decrease in airways hyper-responsiveness, mucus hyperproduction, airways inflammation, and pulmonary eosinophil recruitment.
   176. The method of embodiment 170 wherein the reduction in inflammation is determined by a decrease in expression levels of mRNA for inflammatory markers, by normalized glucose homeostasis, or by reduced circulating cholesterol levels.
   177. The method of embodiment 176 wherein the decrease in expression levels of mRNA for inflammatory markers is a decrease in interleukin (IL)-6 gene expression in vascular tissue.
   178. The method of any one of embodiments 166-168, wherein the disorder is a metabolic disorder.
   179. The method of embodiment 178 wherein the metabolic disorder is selected from the group consisting of: arthritis, rheumatoid arthritis, juvenile idiopathic arthritis, psoriasis, Crohn's disease, inflammatory bowel syndrome, ulcerative colitis, ankylosing spondylitis, non-infectious uveitis, cryopyrin associated periodic syndrome, TNF receptor 1-associated periodic syndrome, diabetes, atherosclerosis, metabolic syndrome, obesity, renal failure, hypertension, and cancer.
   180. The method of embodiment 178 wherein the metabolic disorder is arthritis.
   181. The method of embodiment 178 wherein the metabolic disorder is metabolic syndrome or type II diabetes.
   182. A compound, pure enantiomer, or enantiomerically enriched mixture or pharmaceutical composition thereof according to any one of embodiments 1-136 for use in treating a central nervous system disorder in a host.
   183. A compound, pure enantiomer, or enantiomerically enriched mixture of Formula II for use in treating a central nervous system disorder in a host:

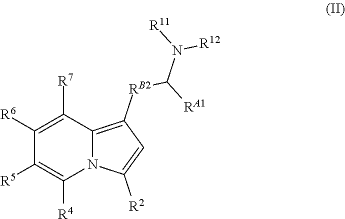

wherein
R^{B2} is

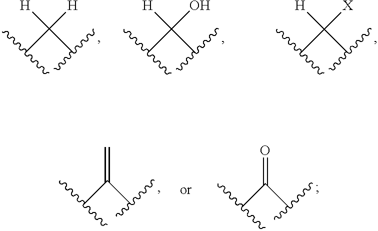

R^{A1} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R², R⁴, R⁵ R⁶, and R⁷ are independently selected from the group consisting of hydrogen, halogen, alkyl, haloalkyl, —OP(O)(OR⁹)₂, —SR⁹, —NR⁹R¹⁰, —NR^{P1}R¹⁰, —NR^{P2}R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹² is hydrogen, —(C₁-C₆)alkyl, —CH₂CH₂X, —CH₂ CHX₂, —CH₂CX₃, —CH₂CH₂OH, or hydroxy;
R¹⁷ is alkyl, haloalkyl, —NR⁹R¹⁰, or —OR⁹;
R^{P1} is selected at each instance from the group consisting of —C(O)R^{13C}, -alkyl-OC(O)R^{13C}, and -alkyl-C(O)R^{13C};
R^{P2} is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R¹⁸ and —S(O)₂R¹⁸; and
R¹⁸ is independently selected from the group consisting of hydrogen, alkyl, aryl, arylalkyl, cycloalkyl, heterocycle, heteroaryl, and heteroarylalkyl.
   184. The compound of embodiment 182 or 183 wherein the host is a human.
   185. The compound of any one of embodiments 182-184 wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism, dissociative disorders and headache disorders.
   186. The compound of any one of embodiments 182-184 wherein the central nervous system disorder is post-traumatic stress disorder.
   187. The compound of any one of embodiments 182-184 wherein the central nervous system disorder is adjustment disorder.
   188. The compound of any one of embodiments 182-184 wherein the central nervous system disorder is generalized anxiety.
   189. The compound of any one of embodiments 182-184 wherein the central nervous system disorder is social anxiety.
   190. The compound of any one of embodiments 182-184 wherein the central nervous system disorder is depression.
   191. The compound of any one of embodiments 182-184 wherein the central nervous system disorder is a substance use disorder.
   192. The compound of any one of embodiments 182-184 wherein the central nervous system disorder is an attachment disorder.
   193. The compound of any one of embodiments 182-184 wherein the central nervous system disorder is schizophrenia.
   194. The compound of any one of embodiments 182-184 wherein the central nervous system disorder is a headache disorder.
   195. The compound of any one of embodiments 182-184 wherein the central nervous system disorder is a migraine disorder.
   196. The compound of any one of embodiments 182-184 wherein the central nervous system disorder is a seizure disorder.
   197. The compound of any one of embodiments 182-184 wherein the central nervous system disorder is an eating disorder.
   198. The compound of embodiment 197 wherein the eating disorder is bulimia.
   199. The compound of embodiment 197 wherein the eating disorder is binge eating.
   200. The compound of embodiment 197 wherein the eating disorder is anorexia.
   201. The compound of any one of embodiments 182-184 wherein the central nervous system disorder is a neurological disorder.
   202. The compound of embodiment 201 wherein the neurological disorder is stroke.
   203. The compound of embodiment 201 wherein the neurological disorder is brain trauma.
   204. The compound of embodiment 201 wherein the neurological disorder is dementia.
   205. The compound of embodiment 201 wherein the neurological disorder is a neurodegenerative disease or disorder.
   206. The compound of embodiment 205 wherein the neurodegenerative disease or disorder is selected from: Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.
   207. The compound of any one of embodiments 182-206 wherein the compound, pure enantiomer, or enantiomerically enriched mixture is administered in a clinical setting.
   208. The compound of any one of embodiments 182-206 wherein the compound, pure enantiomer, or enantiomerically enriched mixture is administered in an at-home setting.
   209. The compound of any one of embodiments 182-206 wherein the compound, pure enantiomer, or enantiomerically enriched mixture is administered during a psychotherapy session.
   210. The compound of any one of embodiments 182-206 wherein the compound, pure enantiomer, or enantiomerically enriched mixture is administered during a counseling session.
   211. A compound, pure enantiomer, or enantiomerically enriched mixture or pharmaceutical composition thereof according to any one of embodiments 1-136 for use in the treatment of an inflammatory or metabolic disorder in a host.
   212. A compound, pure enantiomer, or enantiomerically enriched mixture of Formula II for use in the treatment of an inflammatory or metabolic disorder in a host

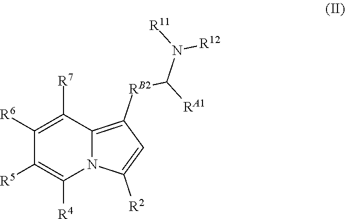

wherein
R^{B2} is

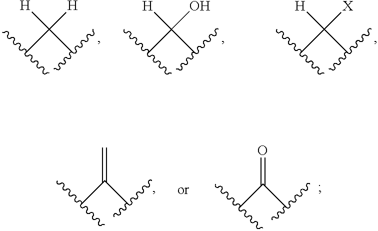

R^{A1} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R², R⁴, R⁵ R⁶, and R⁷ are independently selected from the group consisting of hydrogen, halogen, alkyl, haloalkyl, —OP(O)(OR⁹)₂, —SR⁹, —NR⁹R¹⁰, —NR^{P1}R¹⁰, —NR^{P2}R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹² is hydrogen, —(C₁-C₆)alkyl, —CH₂CH₂X, —CH₂ CHX₂, —CH₂CX₃, —CH₂CH₂OH, or hydroxy;
R¹⁷ is alkyl, haloalkyl, —NR⁹R¹⁰, or —OR⁹;
R^{P1} is selected at each instance from the group consisting of —C(O)R^{13C}, -alkyl-OC(O)R^{13C}, and -alkyl-C(O)R^{13C};
R^{P2} is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R¹⁸ and —S(O)₂R¹⁸; and
R¹⁸ is independently selected from the group consisting of hydrogen, alkyl, aryl, arylalkyl, cycloalkyl, heterocycle, heteroaryl, and heteroarylalkyl.
   213. The compound of embodiment 211 or 212, wherein the host is a human.
   214. The compound of any one of embodiments 211-213, wherein the disorder is an inflammatory disorder.
   215. The compound of embodiment 214, wherein the level of inflammation is reduced.
   216. The compound of embodiment 215, wherein the reduction in inflammation is determined by a decrease in TNF-mediated proinflammatory markers.
   217. The compound of embodiment 216, wherein the decrease in TNF-mediated proinflammatory markers is a decrease in intracellular adhesion molecule 1 (ICAM-1), vascular adhesion molecule 1 (VCAM-1), interleukin (IL)-6 gene expression, nitric-oxide synthase activity, or nuclear translocation of nuclear factor KB.
   218. The compound of any one of embodiments 211-217, wherein the disorder is asthma.
   219. The compound of embodiment 215 wherein the reduction in inflammation is determined by a decrease in one or more markers of asthma severity.
   220. The compound of embodiment 219 wherein the markers of asthma severity are selected from the group consisting of a decrease in airways hyper-responsiveness, mucus hyperproduction, airways inflammation, and pulmonary eosinophil recruitment.
   221. The compound of embodiment 215 wherein the reduction in inflammation is determined by a decrease in expression levels of mRNA for inflammatory markers, by normalized glucose homeostasis, or by reduced circulating cholesterol levels.
   222. The compound of embodiment 221 wherein the decrease in expression levels of mRNA for inflammatory markers is a decrease in interleukin (IL)-6 gene expression in vascular tissue.
   223. The compound of any one of embodiments 211-213, wherein the disorder is a metabolic disorder.
   224. The compound of embodiment 223 wherein the metabolic disorder is selected from the group consisting of: arthritis, rheumatoid arthritis, juvenile idiopathic arthritis, psoriasis, Crohn's disease, inflammatory bowel syndrome, ulcerative colitis, ankylosing spondylitis, non-infectious uveitis, cryopyrin associated periodic syndrome, TNF receptor 1-associated periodic syndrome, diabetes, atherosclerosis, metabolic syndrome, obesity, renal failure, hypertension, and cancer.
   225. The compound of embodiment 223 wherein the metabolic disorder is arthritis.
   226. The compound of embodiment 223 wherein the metabolic disorder is metabolic syndrome or type II diabetes.
   227. Use of a compound, pure enantiomer, or enantiomerically enriched mixture or pharmaceutical composition thereof according to any one of embodiments 1-136 in the manufacture of a medicament for treating a central nervous system disorder in a host.
   228. Use of a compound, pure enantiomer, or enantiomerically enriched mixture of Formula II in the manufacture of a medicament for treating a central nervous system disorder in a host:

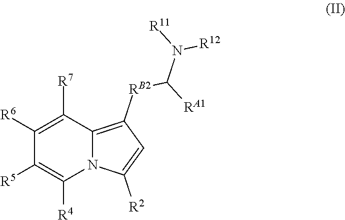

wherein

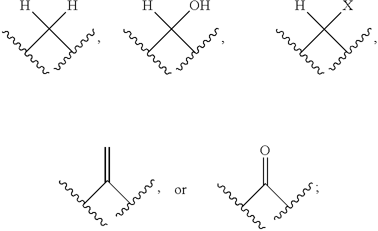

R^{B2} is
R^{A1} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R², R⁴, R⁵ R⁶, and R⁷ are independently selected from the group consisting of hydrogen, halogen, alkyl, haloalkyl, —OP(O)(OR⁹)₂, —SR⁹, —NR⁹R¹⁰, —NR^{P1}R¹⁰, —NR^{P2}R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹² is hydrogen, —(C₁-C₆)alkyl, —CH₂CH₂X, —CH₂ CHX₂, —CH₂CX₃, —CH₂CH₂OH, or hydroxy;
R¹⁷ is alkyl, haloalkyl, —NR⁹R¹⁰, or —OR⁹;
R^{P1} is selected at each instance from the group consisting of —C(O)R^{13C}, -alkyl-OC(O)R^{13C}, and -alkyl-C(O)R^{13C};
R^{P2} is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R¹⁸ and —S(O)₂R¹⁸; and
R¹⁸ is independently selected from the group consisting of hydrogen, alkyl, aryl, arylalkyl, cycloalkyl, heterocycle, heteroaryl, and heteroarylalkyl.
   229. The use of embodiment 227 or 228 wherein the host is a human.
   230. The use of any one of embodiments 227-229 wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism, dissociative disorders and headache disorders.
   231. The use of any one of embodiments 227-229 wherein the central nervous system disorder is post-traumatic stress disorder.
   232. The use of any one of embodiments 227-229 wherein the central nervous system disorder is adjustment disorder.
   233. The use of any one of embodiments 227-229 wherein the central nervous system disorder is generalized anxiety.
   234. The use of any one of embodiments 227-229 wherein the central nervous system disorder is social anxiety.
   235. The use of any one of embodiments 227-229 wherein the central nervous system disorder is depression.
   236. The use of any one of embodiments 227-229 wherein the central nervous system disorder is a substance use disorder.
   237. The use of any one of embodiments 227-229 wherein the central nervous system disorder is an attachment disorder.
   238. The use of any one of embodiments 227-229 wherein the central nervous system disorder is schizophrenia.
   239. The use of any one of embodiments 227-229 wherein the central nervous system disorder is a headache disorder.
   240. The use of any one of embodiments 227-229 wherein the central nervous system disorder is a migraine disorder.
   241. The use of any one of embodiments 227-229 wherein the central nervous system disorder is a seizure disorder.
   242. The use of any one of embodiments 227-229 wherein the central nervous system disorder is an eating disorder.
   243. The use of embodiment 242 wherein the eating disorder is bulimia.
   244. The use of embodiment 242 wherein the eating disorder is binge eating.
   245. The use of embodiment 242 wherein the eating disorder is anorexia.
   246. The use of any one of embodiments 227-229 wherein the central nervous system disorder is a neurological disorder.
   247. The use of embodiment 246 wherein the neurological disorder is stroke.
   248. The use of embodiment 246 wherein the neurological disorder is brain trauma.
   249. The use of embodiment 246 wherein the neurological disorder is dementia.
   250. The use of embodiment 246 wherein the neurological disorder is a neurodegenerative disease or disorder.
   251. The use of embodiment 250 wherein the neurodegenerative disease or disorder is selected from: Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.
   252. The use of any one of embodiments 227-251 wherein the compound, pure enantiomer, or enantiomerically enriched mixture is administered in a clinical setting.
   253. The use of any one of embodiments 227-251 wherein the compound, pure enantiomer, or enantiomerically enriched mixture is administered in an at-home setting.
   254. The use of any one of embodiments 227-251 wherein the compound, pure enantiomer, or enantiomerically enriched mixture is administered during a psychotherapy session.
   255. The use of any one of embodiments 227-251 wherein the compound, pure enantiomer, or enantiomerically enriched mixture is administered during a counseling session.
   256. Use of a compound, pure enantiomer, or enantiomerically enriched mixture or pharmaceutical composition thereof according to any one of embodiments 1-136 in the manufacture of a medicament for treating an inflammatory or metabolic disorder in a host.
   257. Use of a compound, pure enantiomer, or enantiomerically enriched mixture of Formula II in the manufacture of a medicament for treating an inflammatory or metabolic disorder in a host

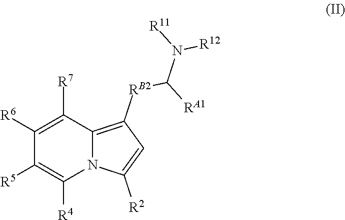

wherein
R^{B2} is

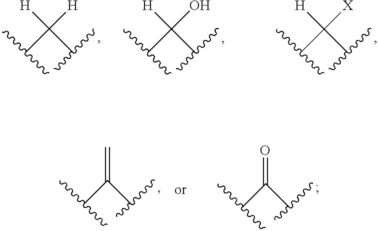

R^{A1} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R², R⁴, R⁵ R⁶, and R⁷ are independently selected from the group consisting of hydrogen, halogen, alkyl, haloalkyl, —OP(O)(OR⁹)₂, —SR⁹, —NR⁹R¹⁰, —NR^{P1}R¹⁰, —NR²R¹⁰, —OR⁹, —OR^{P1}, —OR^{P2}, alkenyl, alkynyl, aminoalkyl, —S(O)₂R¹⁷, -alkyl-S(O)₂R¹⁷, —NR⁹S(O)₂R¹⁷, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹² is hydrogen, —(C₁-C₆)alkyl, —CH₂CH₂X, —CH₂ CHX₂, —CH₂CX₃, —CH₂CH₂OH, or hydroxy;
R¹⁷ is alkyl, haloalkyl, —NR⁹R¹⁰, or —OR⁹; R^{P1} is selected at each instance from the group consisting of —C(O)R^{13C}, -alkyl-OC(O)R^{13C}, and -alkyl-C(O)R^{13C};
R^{P2} is selected at each instance from the group consisting of an amino acid or peptide comprising 2, 3, or 4 amino acids wherein each amino acid is independently selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and each amino acid is further optionally substituted as allowed by valence with 1, 2, 3, or 4 independently selected optional substituents selected from halogen, —R¹⁴, —OR¹⁴, —SR¹⁴, —NR¹⁴R¹⁵, —CH₂X, —CHX₂, —CX₃, —CN, —NO₂, —S(O)₂alkyl, —OS(O)₂alkyl, —P(O)(OR¹⁴)(OR¹⁵), —C(O)alkyl, —C(S)alkyl, —C(O)OR¹⁴, —C(O)NR¹⁴R¹⁵, —C(S)OR¹⁴, —NR¹⁶C(O)NR¹⁴R¹⁵, and —NR¹⁶C(S)NR¹⁴R¹⁵
X at each instance is independently selected from F, Cl, Br, and I;
R¹⁴, R¹⁵, and R¹⁶, are independently selected from the group consisting of hydrogen, alkyl, aryl, arylalkyl, cycloalkyl, heterocycle, heteroaryl, heteroarylalkyl, —C(O)R¹⁸ and —S(O)₂R¹⁸; and
R¹¹ is independently selected from the group consisting of hydrogen, alkyl, aryl, arylalkyl, cycloalkyl, heterocycle, heteroaryl, and heteroarylalkyl.
   258. The use of embodiment 256 or 257, wherein the host is a human.
   259. The use of any one of embodiments 256-258, wherein the disorder is an inflammatory disorder.
   260. The use of embodiment 259, wherein the level of inflammation is reduced.
   261. The use of embodiment 260, wherein the reduction in inflammation is determined by a decrease in TNF-mediated proinflammatory markers.
   262. The use of embodiment 261, wherein the decrease in TNF-mediated proinflammatory markers is a decrease in intracellular adhesion molecule 1 (ICAM-1), vascular adhesion molecule 1 (VCAM-1), interleukin (IL)-6 gene expression, nitric-oxide synthase activity, or nuclear translocation of nuclear factor KB.
   263. The use of any one of embodiments 256-262, wherein the disorder is asthma.
   264. The use of embodiment 260, wherein the reduction in inflammation is determined by a decrease in one or more markers of asthma severity.
   265. The use of embodiment 264, wherein the markers of asthma severity are selected from the group consisting of a decrease in airways hyper-responsiveness, mucus hyperproduction, airways inflammation, and pulmonary eosinophil recruitment.
   266. The use of embodiment 260, wherein the reduction in inflammation is determined by a decrease in expression levels of mRNA for inflammatory markers, by normalized glucose homeostasis, or by reduced circulating cholesterol levels.
   267. The use of embodiment 266, wherein the decrease in expression levels of mRNA for inflammatory markers is a decrease in interleukin (IL)-6 gene expression in vascular tissue.
   268. The use of any one of embodiments 256-258, wherein the disorder is a metabolic disorder.
   269. The use of embodiment 268, wherein the metabolic disorder is selected from the group consisting of: arthritis, rheumatoid arthritis, juvenile idiopathic arthritis, psoriasis, Crohn's disease, inflammatory bowel syndrome, ulcerative colitis, ankylosing spondylitis, non-infectious uveitis, cryopyrin associated periodic syndrome, TNF receptor 1-associated periodic syndrome, diabetes, atherosclerosis, metabolic syndrome, obesity, renal failure, hypertension, and cancer.
   270. The use of embodiment 268, wherein the metabolic disorder is arthritis.
   271. The use of embodiment 268, wherein the metabolic disorder is metabolic syndrome or type II diabetes.

### Optional Substituents

In certain embodiments a moiety described herein that can be substituted with 1, 2, 3, or 4 substituents is substituted with one substituent.

In certain embodiments a moiety described herein that can be substituted with 1, 2, 3, or 4 substituents is substituted with two substituents.

In certain embodiments a moiety described herein that can be substituted with 1, 2, 3, or 4 substituents is substituted with three substituents.

In certain embodiments a moiety described herein that can be substituted with 1, 2, 3, or 4 substituents is substituted with four substituents.

### Chirality

In certain embodiments the S-enantiomer of a compound described above is enantiomerically enriched for example about 55:45, about 60:40, about 65:35, about 70:30, about 75:25, about 80:20, about 85:15, about 90:10, about 95:5, about 96:4, about 97:3, about 98:2, or about 99:1, or greater than about 99% S-enantiomer.

In certain embodiments the R-enantiomer of a compound described above is enantiomerically enriched for example about 55:45, about 60:40, about 65:35, about 70:30, about 75:25, about 80:20, about 85:15, about 90:10, about 95:5, about 96:4, about 97:3, about 98:2, or about 99:1, or greater than about 99% R-enantiomer.

The carbon alpha to the amine is chiral when R^{A} is not hydrogen. The invention includes a compound of either the R- or S-stereochemistry at this carbon. An isolated R- or S-enantiomeric compound of the present invention can be used as a pure enantiomer or combined with the other enantiomer in any ratio that produces the desired effects. This can be an equal ratio (racemic), or in which one enantiomer is present in a greater amount than the other, referred to herein as an enantiomerically enriched mixture. Typically in the present application, the chiral carbon referred to in the term “enantiomerically enriched” is that carbon alpha to the amine in the provided structures.

### Embodiments of Formula IB

In certain aspects a compound of Formula IB is provided:

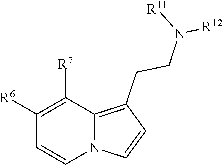

or a pharmaceutically acceptable salt or salt mixture thereof.

In certain embodiments the compound of Formula IB is a 5-HT_{2A} agonist. In certain embodiments the compound of Table II is a 5-HT_{2A} agonist.

TABLE II

| Compound | Position | | | |
|---|---|---|---|---|
| # | R¹¹ | R¹² | R⁶ | R⁷ |
| 1 | CH3 | CH3 | H | H |
| 2 | CH3 | CH2CH3 | H | H |
| 3 | CH3 | (CH2)3CH3 | H | H |
| 4 | CH3 | CH(CH3)2 | H | H |
| 5 | CH3 | CH2CH2CH3 | H | H |
| 6 | CH3 | CH2CH═CH2 | H | H |
| 7 | CH2CH3 | CH2CH3 | H | H |
| 8 | CH2CH3 | (CH2)3CH3 | H | H |
| 9 | CH2CH3 | CH(CH3)2 | H | H |
| 10 | CH2CH3 | CH2CH2CH3 | H | H |
| 11 | CH2CH3 | CH2CH═CH2 | H | H |
| 12 | (CH2)3CH3 | (CH2)3CH3 | H | H |
| 13 | (CH2)3CH3 | CH(CH3)2 | H | H |
| 14 | (CH2)3CH3 | CH2CH2CH3 | H | H |
| 15 | (CH2)3CH3 | CH2CH═CH2 | H | H |
| 16 | CH(CH3)2 | CH(CH3)2 | H | H |
| 17 | CH(CH3)2 | CH2CH2CH3 | H | H |
| 18 | CH(CH3)2 | CH2CH═CH2 | H | H |
| 19 | CH2CH2CH3 | CH2CH2CH3 | H | H |
| 20 | CH2CH2CH3 | CH2CH═CH2 | H | H |
| 21 | CH2CH═CH2 | CH2CH═CH2 | H | H |
| 22 | CH3 | CH3 | OH | H |
| 23 | CH3 | CH2CH3 | OH | H |
| 24 | CH3 | (CH2)3CH3 | OH | H |
| 25 | CH3 | CH(CH3)2 | OH | H |
| 26 | CH3 | CH2CH2CH3 | OH | H |
| 27 | CH3 | CH2CH═CH2 | OH | H |
| 28 | CH2CH3 | CH2CH3 | OH | H |
| 29 | CH2CH3 | (CH2)3CH3 | OH | H |
| 30 | CH2CH3 | CH(CH3)2 | OH | H |
| 31 | CH2CH3 | CH2CH2CH3 | OH | H |
| 32 | CH2CH3 | CH2CH═CH2 | OH | H |
| 33 | (CH2)3CH3 | (CH2)3CH3 | OH | H |
| 34 | (CH2)3CH3 | CH(CH3)2 | OH | H |
| 35 | (CH2)3CH3 | CH2CH2CH3 | OH | H |
| 36 | (CH2)3CH3 | CH2CH═CH2 | OH | H |
| 37 | CH(CH3)2 | CH(CH3)2 | OH | H |
| 38 | CH(CH3)2 | CH2CH2CH3 | OH | H |
| 39 | CH(CH3)2 | CH2CH═CH2 | OH | H |
| 40 | CH2CH2CH3 | CH2CH2CH3 | OH | H |
| 41 | CH2CH2CH3 | CH2CH═CH2 | OH | H |
| 42 | CH2CH═CH2 | CH2CH═CH2 | OH | H |
| 43 | CH3 | CH3 | OC(═O)CH3 | H |
| 44 | CH3 | CH2CH3 | OC(═O)CH3 | H |
| 45 | CH3 | (CH2)3CH3 | OC(═O)CH3 | H |
| 46 | CH3 | CH(CH3)2 | OC(═O)CH3 | H |
| 47 | CH3 | CH2CH2CH3 | OC(═O)CH3 | H |
| 48 | CH3 | CH2CH═CH2 | OC(═O)CH3 | H |
| 49 | CH2CH3 | CH2CH3 | OC(═O)CH3 | H |
| 50 | CH2CH3 | (CH2)3CH3 | OC(═O)CH3 | H |
| 51 | CH2CH3 | CH(CH3)2 | OC(═O)CH3 | H |
| 52 | CH2CH3 | CH2CH2CH3 | OC(═O)CH3 | H |
| 53 | CH2CH3 | CH2CH═CH2 | OC(═O)CH3 | H |
| 54 | (CH2)3CH3 | (CH2)3CH3 | OC(═O)CH3 | H |
| 55 | (CH2)3CH3 | CH(CH3)2 | OC(═O)CH3 | H |
| 56 | (CH2)3CH3 | CH2CH2CH3 | OC(═O)CH3 | H |
| 57 | (CH2)3CH3 | CH2CH═CH2 | OC(═O)CH3 | H |
| 58 | CH(CH3)2 | CH(CH3)2 | OC(═O)CH3 | H |
| 59 | CH(CH3)2 | CH2CH2CH3 | OC(═O)CH3 | H |
| 60 | CH(CH3)2 | CH2CH═CH2 | OC(═O)CH3 | H |
| 61 | CH2CH2CH3 | CH2CH2CH3 | OC(═O)CH3 | H |
| 62 | CH2CH2CH3 | CH2CH═CH2 | OC(═O)CH3 | H |
| 63 | CH2CH═CH2 | CH2CH═CH2 | OC(═O)CH3 | H |
| 64 | CH3 | CH3 | OP(═O)(OH2) | H |
| 65 | CH3 | CH2CH3 | OP(═O)(OH2) | H |
| 66 | CH3 | (CH2)3CH3 | OP(═O)(OH2) | H |
| 67 | CH3 | CH(CH3)2 | OP(═O)(OH2) | H |
| 68 | CH3 | CH2CH2CH3 | OP(═O)(OH2) | H |
| 69 | CH3 | CH2CH═CH2 | OP(═O)(OH2) | H |
| 70 | CH2CH3 | CH2CH3 | OP(═O)(OH2) | H |
| 71 | CH2CH3 | (CH2)3CH3 | OP(═O)(OH2) | H |
| 72 | CH2CH3 | CH(CH3)2 | OP(═O)(OH2) | H |
| 73 | CH2CH3 | CH2CH2CH3 | OP(═O)(OH2) | H |
| 74 | CH2CH3 | CH2CH═CH2 | OP(═O)(OH2) | H |
| 75 | (CH2)3CH3 | (CH2)3CH3 | OP(═O)(OH2) | H |
| 76 | (CH2)3CH3 | CH(CH3)2 | OP(═O)(OH2) | H |
| 77 | (CH2)3CH3 | CH2CH2CH3 | OP(═O)(OH2) | H |
| 78 | (CH2)3CH3 | CH2CH═CH2 | OP(═O)(OH2) | H |
| 79 | CH(CH3)2 | CH(CH3)2 | OP(═O)(OH2) | H |
| 80 | CH(CH3)2 | CH2CH2CH3 | OP(═O)(OH2) | H |
| 81 | CH(CH3)2 | CH2CH═CH2 | OP(═O)(OH2) | H |
| 82 | CH2CH2CH3 | CH2CH2CH3 | OP(═O)(OH2) | H |
| 83 | CH2CH2CH3 | CH2CH═CH2 | OP(═O)(OH2) | H |
| 84 | CH2CH═CH2 | CH2CH═CH2 | OP(═O)(OH2) | H |
| 85 | CH3 | CH3 | CH3 | H |
| 86 | CH3 | CH2CH3 | CH3 | H |
| 87 | CH3 | (CH2)3CH3 | CH3 | H |
| 88 | CH3 | CH(CH3)2 | CH3 | H |
| 89 | CH3 | CH2CH2CH3 | CH3 | H |
| 90 | CH3 | CH2CH═CH2 | CH3 | H |
| 91 | CH2CH3 | CH2CH3 | CH3 | H |
| 92 | CH2CH3 | (CH2)3CH3 | CH3 | H |
| 93 | CH2CH3 | CH(CH3)2 | CH3 | H |
| 94 | CH2CH3 | CH2CH2CH3 | CH3 | H |
| 95 | CH2CH3 | CH2CH═CH2 | CH3 | H |
| 96 | (CH2)3CH3 | (CH2)3CH3 | CH3 | H |
| 97 | (CH2)3CH3 | CH(CH3)2 | CH3 | H |
| 98 | (CH2)3CH3 | CH2CH2CH3 | CH3 | H |
| 99 | (CH2)3CH3 | CH2CH═CH2 | CH3 | H |
| 100 | CH(CH3)2 | CH(CH3)2 | CH3 | H |
| 101 | CH(CH3)2 | CH2CH2CH3 | CH3 | H |
| 102 | CH(CH3)2 | CH2CH═CH2 | CH3 | H |
| 103 | CH2CH2CH3 | CH2CH2CH3 | CH3 | H |
| 104 | CH2CH2CH3 | CH2CH═CH2 | CH3 | H |
| 105 | CH2CH═CH2 | CH2CH═CH2 | CH3 | H |
| 106 | CH3 | CH3 | Br | H |
| 107 | CH3 | CH2CH3 | Br | H |
| 108 | CH3 | (CH2)3CH3 | Br | H |
| 109 | CH3 | CH(CH3)2 | Br | H |
| 110 | CH3 | CH2CH2CH3 | Br | H |
| 111 | CH3 | CH2CH═CH2 | Br | H |
| 112 | CH2CH3 | CH2CH3 | Br | H |
| 113 | CH2CH3 | (CH2)3CH3 | Br | H |
| 114 | CH2CH3 | CH(CH3)2 | Br | H |
| 115 | CH2CH3 | CH2CH2CH3 | Br | H |
| 116 | CH2CH3 | CH2CH═CH2 | Br | H |
| 117 | (CH2)3CH3 | (CH2)3CH3 | Br | H |
| 118 | (CH2)3CH3 | CH(CH3)2 | Br | H |
| 119 | (CH2)3CH3 | CH2CH2CH3 | Br | H |
| 120 | (CH2)3CH3 | CH2CH═CH2 | Br | H |
| 121 | CH(CH3)2 | CH(CH3)2 | Br | H |
| 122 | CH(CH3)2 | CH2CH2CH3 | Br | H |
| 123 | CH(CH3)2 | CH2CH═CH2 | Br | H |
| 124 | CH2CH2CH3 | CH2CH2CH3 | Br | H |
| 125 | CH2CH2CH3 | CH2CH═CH2 | Br | H |
| 126 | CH2CH═CH2 | CH2CH═CH2 | Br | H |
| 127 | CH3 | CH3 | Cl | H |
| 128 | CH3 | CH2CH3 | Cl | H |
| 129 | CH3 | (CH2)3CH3 | Cl | H |
| 130 | CH3 | CH(CH3)2 | Cl | H |
| 131 | CH3 | CH2CH2CH3 | Cl | H |
| 132 | CH3 | CH2CH═CH2 | Cl | H |
| 133 | CH2CH3 | CH2CH3 | Cl | H |
| 134 | CH2CH3 | (CH2)3CH3 | Cl | H |
| 135 | CH2CH3 | CH(CH3)2 | Cl | H |
| 136 | CH2CH3 | CH2CH2CH3 | Cl | H |
| 137 | CH2CH3 | CH2CH═CH2 | Cl | H |
| 138 | (CH2)3CH3 | (CH2)3CH3 | Cl | H |
| 139 | (CH2)3CH3 | CH(CH3)2 | Cl | H |
| 140 | (CH2)3CH3 | CH2CH2CH3 | Cl | H |
| 141 | (CH2)3CH3 | CH2CH═CH2 | Cl | H |
| 142 | CH(CH3)2 | CH(CH3)2 | Cl | H |
| 143 | CH(CH3)2 | CH2CH2CH3 | Cl | H |
| 144 | CH(CH3)2 | CH2CH═CH2 | Cl | H |
| 145 | CH2CH2CH3 | CH2CH2CH3 | Cl | H |
| 146 | CH2CH2CH3 | CH2CH═CH2 | Cl | H |
| 147 | CH2CH═CH2 | CH2CH═CH2 | Cl | H |
| 148 | CH3 | CH3 | F | H |
| 149 | CH3 | CH2CH3 | F | H |
| 150 | CH3 | (CH2)3CH3 | F | H |
| 151 | CH3 | CH(CH3)2 | F | H |
| 152 | CH3 | CH2CH2CH3 | F | H |
| 153 | CH3 | CH2CH═CH2 | F | H |
| 154 | CH2CH3 | CH2CH3 | F | H |
| 155 | CH2CH3 | (CH2)3CH3 | F | H |
| 156 | CH2CH3 | CH(CH3)2 | F | H |
| 157 | CH2CH3 | CH2CH2CH3 | F | H |
| 158 | CH2CH3 | CH2CH═CH2 | F | H |
| 159 | (CH2)3CH3 | (CH2)3CH3 | F | H |
| 160 | (CH2)3CH3 | CH(CH3)2 | F | H |
| 161 | (CH2)3CH3 | CH2CH2CH3 | F | H |
| 162 | (CH2)3CH3 | CH2CH═CH2 | F | H |
| 163 | CH(CH3)2 | CH(CH3)2 | F | H |
| 164 | CH(CH3)2 | CH2CH2CH3 | F | H |
| 165 | CH(CH3)2 | CH2CH═CH2 | F | H |
| 166 | CH2CH2CH3 | CH2CH2CH3 | F | H |
| 167 | CH2CH2CH3 | CH2CH═CH2 | F | H |
| 168 | CH2CH═CH2 | CH2CH═CH2 | F | H |
| 169 | CH3 | CH3 | I | H |
| 170 | CH3 | CH2CH3 | I | H |
| 171 | CH3 | (CH2)3CH3 | I | H |
| 172 | CH3 | CH(CH3)2 | I | H |
| 173 | CH3 | CH2CH2CH3 | I | H |
| 174 | CH3 | CH2CH═CH2 | I | H |
| 175 | CH2CH3 | CH2CH3 | I | H |
| 176 | CH2CH3 | (CH2)3CH3 | I | H |
| 177 | CH2CH3 | CH(CH3)2 | I | H |
| 178 | CH2CH3 | CH2CH2CH3 | I | H |
| 179 | CH2CH3 | CH2CH═CH2 | I | H |
| 180 | (CH2)3CH3 | (CH2)3CH3 | I | H |
| 181 | (CH2)3CH3 | CH(CH3)2 | I | H |
| 182 | (CH2)3CH3 | CH2CH2CH3 | I | H |
| 183 | (CH2)3CH3 | CH2CH═CH2 | I | H |
| 184 | CH(CH3)2 | CH(CH3)2 | I | H |
| 185 | CH(CH3)2 | CH2CH2CH3 | I | H |
| 186 | CH(CH3)2 | CH2CH═CH2 | I | H |
| 187 | CH2CH2CH3 | CH2CH2CH3 | I | H |
| 188 | CH2CH2CH3 | CH2CH═CH2 | I | H |
| 189 | CH2CH═CH2 | CH2CH═CH2 | I | H |
| 190 | CH3 | CH3 | H | OH |
| 191 | CH3 | CH2CH3 | H | OH |
| 192 | CH3 | (CH2)3CH3 | H | OH |
| 193 | CH3 | CH(CH3)2 | H | OH |
| 194 | CH3 | CH2CH2CH3 | H | OH |
| 195 | CH3 | CH2CH═CH2 | H | OH |
| 196 | CH2CH3 | CH2CH3 | H | OH |
| 197 | CH2CH3 | (CH2)3CH3 | H | OH |
| 198 | CH2CH3 | CH(CH3)2 | H | OH |
| 199 | CH2CH3 | CH2CH2CH3 | H | OH |
| 200 | CH2CH3 | CH2CH═CH2 | H | OH |
| 201 | (CH2)3CH3 | (CH2)3CH3 | H | OH |
| 202 | (CH2)3CH3 | CH(CH3)2 | H | OH |
| 203 | (CH2)3CH3 | CH2CH2CH3 | H | OH |
| 204 | (CH2)3CH3 | CH2CH═CH2 | H | OH |
| 205 | CH(CH3)2 | CH(CH3)2 | H | OH |
| 206 | CH(CH3)2 | CH2CH2CH3 | H | OH |
| 207 | CH(CH3)2 | CH2CH═CH2 | H | OH |
| 208 | CH2CH2CH3 | CH2CH2CH3 | H | OH |
| 209 | CH2CH2CH3 | CH2CH═CH2 | H | OH |
| 210 | CH2CH═CH2 | CH2CH═CH2 | H | OH |
| 211 | CH3 | CH3 | H | OC(═O)CH3 |
| 212 | CH3 | CH2CH3 | H | OC(═O)CH3 |
| 213 | CH3 | (CH2)3CH3 | H | OC(═O)CH3 |
| 214 | CH3 | CH(CH3)2 | H | OC(═O)CH3 |
| 215 | CH3 | CH2CH2CH3 | H | OC(═O)CH3 |
| 216 | CH3 | CH2CH═CH2 | H | OC(═O)CH3 |
| 217 | CH2CH3 | CH2CH3 | H | OC(═O)CH3 |
| 218 | CH2CH3 | (CH2)3CH3 | H | OC(═O)CH3 |
| 219 | CH2CH3 | CH(CH3)2 | H | OC(═O)CH3 |
| 220 | CH2CH3 | CH2CH2CH3 | H | OC(═O)CH3 |
| 221 | CH2CH3 | CH2CH═CH2 | H | OC(═O)CH3 |
| 222 | (CH2)3CH3 | (CH2)3CH3 | H | OC(═O)CH3 |
| 223 | (CH2)3CH3 | CH(CH3)2 | H | OC(═O)CH3 |
| 224 | (CH2)3CH3 | CH2CH2CH3 | H | OC(═O)CH3 |
| 225 | (CH2)3CH3 | CH2CH═CH2 | H | OC(═O)CH3 |
| 226 | CH(CH3)2 | CH(CH3)2 | H | OC(═O)CH3 |
| 227 | CH(CH3)2 | CH2CH2CH3 | H | OC(═O)CH3 |
| 228 | CH(CH3)2 | CH2CH═CH2 | H | OC(═O)CH3 |
| 229 | CH2CH2CH3 | CH2CH2CH3 | H | OC(═O)CH3 |
| 230 | CH2CH2CH3 | CH2CH═CH2 | H | OC(═O)CH3 |
| 231 | CH2CH═CH2 | CH2CH═CH2 | H | OC(═O)CH3 |
| 232 | CH3 | CH3 | H | OP(═O)(OH2) |
| 233 | CH3 | CH2CH3 | H | OP(═O)(OH2) |
| 234 | CH3 | (CH2)3CH3 | H | OP(═O)(OH2) |
| 235 | CH3 | CH(CH3)2 | H | OP(═O)(OH2) |
| 236 | CH3 | CH2CH2CH3 | H | OP(═O)(OH2) |
| 237 | CH3 | CH2CH═CH2 | H | OP(═O)(OH2) |
| 238 | CH2CH3 | CH2CH3 | H | OP(═O)(OH2) |
| 239 | CH2CH3 | (CH2)3CH3 | H | OP(═O)(OH2) |
| 240 | CH2CH3 | CH(CH3)2 | H | OP(═O)(OH2) |
| 241 | CH2CH3 | CH2CH2CH3 | H | OP(═O)(OH2) |
| 242 | CH2CH3 | CH2CH═CH2 | H | OP(═O)(OH2) |
| 243 | (CH2)3CH3 | (CH2)3CH3 | H | OP(═O)(OH2) |
| 244 | (CH2)3CH3 | CH(CH3)2 | H | OP(═O)(OH2) |
| 245 | (CH2)3CH3 | CH2CH2CH3 | H | OP(═O)(OH2) |
| 246 | (CH2)3CH3 | CH2CH═CH2 | H | OP(═O)(OH2) |
| 247 | CH(CH3)2 | CH(CH3)2 | H | OP(═O)(OH2) |
| 248 | CH(CH3)2 | CH2CH2CH3 | H | OP(═O)(OH2) |
| 249 | CH(CH3)2 | CH2CH═CH2 | H | OP(═O)(OH2) |
| 250 | CH2CH2CH3 | CH2CH2CH3 | H | OP(═O)(OH2) |
| 251 | CH2CH2CH3 | CH2CH═CH2 | H | OP(═O)(OH2) |
| 252 | CH2CH═CH2 | CH2CH═CH2 | H | OP(═O)(OH2) |
| 253 | CH3 | CH3 | H | CH3 |
| 254 | CH3 | CH2CH3 | H | CH3 |
| 255 | CH3 | (CH2)3CH3 | H | CH3 |
| 256 | CH3 | CH(CH3)2 | H | CH3 |
| 257 | CH3 | CH2CH2CH3 | H | CH3 |
| 258 | CH3 | CH2CH═CH2 | H | CH3 |
| 259 | CH2CH3 | CH2CH3 | H | CH3 |
| 260 | CH2CH3 | (CH2)3CH3 | H | CH3 |
| 261 | CH2CH3 | CH(CH3)2 | H | CH3 |
| 262 | CH2CH3 | CH2CH2CH3 | H | CH3 |
| 263 | CH2CH3 | CH2CH═CH2 | H | CH3 |
| 264 | (CH2)3CH3 | (CH2)3CH3 | H | CH3 |
| 265 | (CH2)3CH3 | CH(CH3)2 | H | CH3 |
| 266 | (CH2)3CH3 | CH2CH2CH3 | H | CH3 |
| 267 | (CH2)3CH3 | CH2CH═CH2 | H | CH3 |
| 268 | CH(CH3)2 | CH(CH3)2 | H | CH3 |
| 269 | CH(CH3)2 | CH2CH2CH3 | H | CH3 |
| 270 | CH(CH3)2 | CH2CH═CH2 | H | CH3 |
| 271 | CH2CH2CH3 | CH2CH2CH3 | H | CH3 |
| 272 | CH2CH2CH3 | CH2CH═CH2 | H | CH3 |
| 273 | CH2CH═CH2 | CH2CH═CH2 | H | CH3 |
| 274 | CH3 | CH3 | H | Br |
| 275 | CH3 | CH2CH3 | H | Br |
| 276 | CH3 | (CH2)3CH3 | H | Br |
| 277 | CH3 | CH(CH3)2 | H | Br |
| 278 | CH3 | CH2CH2CH3 | H | Br |
| 279 | CH3 | CH2CH═CH2 | H | Br |
| 280 | CH2CH3 | CH2CH3 | H | Br |
| 281 | CH2CH3 | (CH2)3CH3 | H | Br |
| 282 | CH2CH3 | CH(CH3)2 | H | Br |
| 283 | CH2CH3 | CH2CH2CH3 | H | Br |
| 284 | CH2CH3 | CH2CH═CH2 | H | Br |
| 285 | (CH2)3CH3 | (CH2)3CH3 | H | Br |
| 286 | (CH2)3CH3 | CH(CH3)2 | H | Br |
| 287 | (CH2)3CH3 | CH2CH2CH3 | H | Br |
| 288 | (CH2)3CH3 | CH2CH═CH2 | H | Br |
| 289 | CH(CH3)2 | CH(CH3)2 | H | Br |
| 290 | CH(CH3)2 | CH2CH2CH3 | H | Br |
| 291 | CH(CH3)2 | CH2CH═CH2 | H | Br |
| 292 | CH2CH2CH3 | CH2CH2CH3 | H | Br |
| 293 | CH2CH2CH3 | CH2CH═CH2 | H | Br |
| 294 | CH2CH═CH2 | CH2CH═CH2 | H | Br |
| 295 | CH3 | CH3 | H | Cl |
| 296 | CH3 | CH2CH3 | H | Cl |
| 297 | CH3 | (CH2)3CH3 | H | Cl |
| 298 | CH3 | CH(CH3)2 | H | Cl |
| 299 | CH3 | CH2CH2CH3 | H | Cl |
| 300 | CH3 | CH2CH═CH2 | H | Cl |
| 301 | CH2CH3 | CH2CH3 | H | Cl |
| 302 | CH2CH3 | (CH2)3CH3 | H | Cl |
| 303 | CH2CH3 | CH(CH3)2 | H | Cl |
| 304 | CH2CH3 | CH2CH2CH3 | H | Cl |
| 305 | CH2CH3 | CH2CH═CH2 | H | Cl |
| 306 | (CH2)3CH3 | (CH2)3CH3 | H | Cl |
| 307 | (CH2)3CH3 | CH(CH3)2 | H | Cl |
| 308 | (CH2)3CH3 | CH2CH2CH3 | H | Cl |
| 309 | (CH2)3CH3 | CH2CH═CH2 | H | Cl |
| 310 | CH(CH3)2 | CH(CH3)2 | H | Cl |
| 311 | CH(CH3)2 | CH2CH2CH3 | H | Cl |
| 312 | CH(CH3)2 | CH2CH═CH2 | H | Cl |
| 313 | CH2CH2CH3 | CH2CH2CH3 | H | Cl |
| 314 | CH2CH2CH3 | CH2CH═CH2 | H | Cl |
| 315 | CH2CH═CH2 | CH2CH═CH2 | H | Cl |
| 316 | CH3 | CH3 | H | F |
| 317 | CH3 | CH2CH3 | H | F |
| 318 | CH3 | (CH2)3CH3 | H | F |
| 319 | CH3 | CH(CH3)2 | H | F |
| 320 | CH3 | CH2CH2CH3 | H | F |
| 321 | CH3 | CH2CH═CH2 | H | F |
| 322 | CH2CH3 | CH2CH3 | H | F |
| 323 | CH2CH3 | (CH2)3CH3 | H | F |
| 324 | CH2CH3 | CH(CH3)2 | H | F |
| 325 | CH2CH3 | CH2CH2CH3 | H | F |
| 326 | CH2CH3 | CH2CH═CH2 | H | F |
| 327 | (CH2)3CH3 | (CH2)3CH3 | H | F |
| 328 | (CH2)3CH3 | CH(CH3)2 | H | F |
| 329 | (CH2)3CH3 | CH2CH2CH3 | H | F |
| 330 | (CH2)3CH3 | CH2CH═CH2 | H | F |
| 331 | CH(CH3)2 | CH(CH3)2 | H | F |
| 332 | CH(CH3)2 | CH2CH2CH3 | H | F |
| 333 | CH(CH3)2 | CH2CH═CH2 | H | F |
| 334 | CH2CH2CH3 | CH2CH2CH3 | H | F |
| 335 | CH2CH2CH3 | CH2CH═CH2 | H | F |
| 336 | CH2CH═CH2 | CH2CH═CH2 | H | F |
| 337 | CH3 | CH3 | H | I |
| 338 | CH3 | CH2CH3 | H | I |
| 339 | CH3 | (CH2)3CH3 | H | I |
| 340 | CH3 | CH(CH3)2 | H | I |
| 341 | CH3 | CH2CH2CH3 | H | I |
| 342 | CH3 | CH2CH═CH2 | H | I |
| 343 | CH2CH3 | CH2CH3 | H | I |
| 344 | CH2CH3 | (CH2)3CH3 | H | I |
| 345 | CH2CH3 | CH(CH3)2 | H | I |
| 346 | CH2CH3 | CH2CH2CH3 | H | I |
| 347 | CH2CH3v | CH2CH═CH2 | H | I |
| 348 | (CH2)3CH3 | (CH2)3CH3 | H | I |
| 349 | (CH2)3CH3 | CH(CH3)2 | H | I |
| 350 | (CH2)3CH3 | CH2CH2CH3 | H | I |
| 351 | (CH2)3CH3 | CH2CH═CH2 | H | I |
| 352 | CH(CH3)2 | CH(CH3)2 | H | I |
| 353 | CH(CH3)2 | CH2CH2CH3 | H | I |
| 354 | CH(CH3)2 | CH2CH═CH2 | H | I |
| 355 | CH2CH2CH3 | CH2CH2CH3 | H | I |
| 356 | CH2CH2CH3 | CH2CH═CH2 | H | I |
| 357 | CH2CH═CH2 | CH2CH═CH2 | H | I |

### PRODRUGS OF THE PRESENT INVENTION

In certain embodiments, an entactogen prodrug is provided. In some embodiments, the entactogen prodrug comprises at least one amino acid directly bonded to the entactogen. In some embodiments, the at least one amino acid is selected from Table IIIA, Table IIIB, or Table IIIC. In some embodiments, the at least one amino acid comprises at least two amino acids as a peptide.

In certain embodiments the compound of Formula I is selected from:

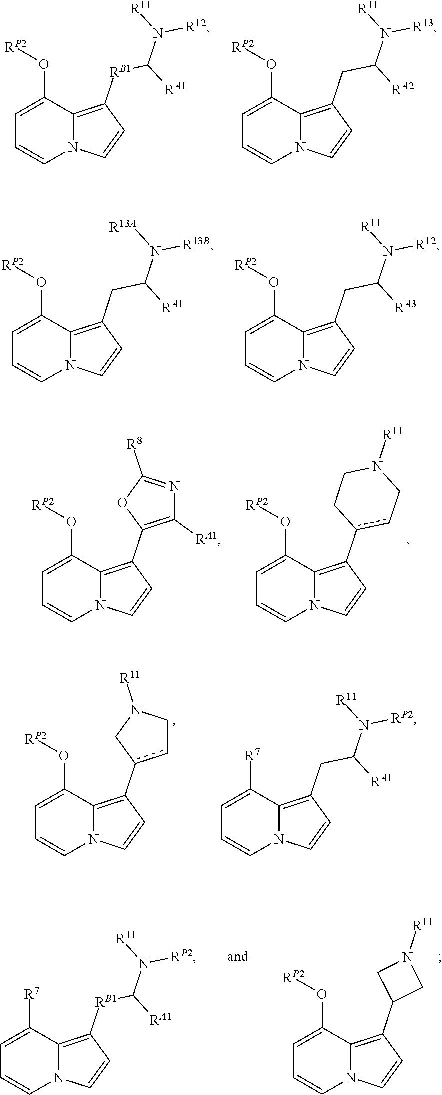

or a pharmaceutically acceptable salt or salt mixture thereof.

In certain embodiments the compound of Formula I is selected from:

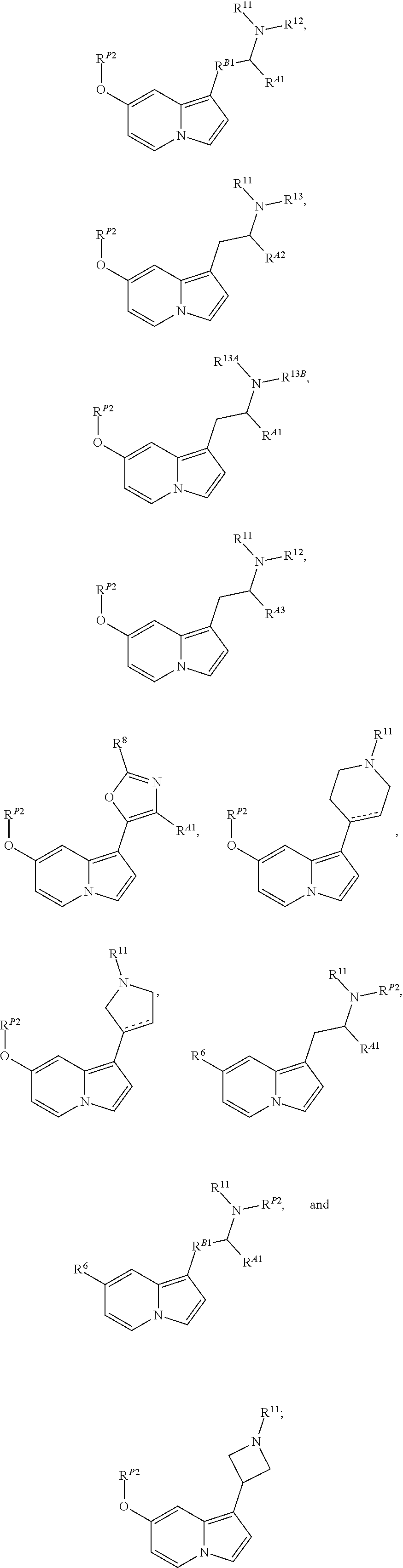

or a pharmaceutically acceptable salt or salt mixture thereof.

The following examples provide nonexhaustive illustrations of amino acids contemplated in some embodiments. However, this molecule is used for illustrative purposes and other possibilities inherent in the definition of R^{P2} are contemplated. Similarly, geometric and other isomers are also contemplated.

### Natural Amino Acids

In certain embodiments, the amino acid used in the present invention is a natural amino acid. Natural amino acids include those which are incorporated into proteins, known as proteinogenic amino acids. The present invention also includes the use of non-proteinogenic natural amino acids. These are amino acids which can be found in nature but are not typically incorporated into proteins. Non-proteinogenic amino acids include α-, β-, and γ-amino acids.

TABLE IIIA
Non-Limiting Examples of Natural Amino Acids Useful as Prodrug Moieties

| | |
|---|---|
| Amino Acid Name | Structure (L-Configuration shown) |

Proteinogenic Amino Acids with Charged Side Chains (at neutral pH)

| | |
|---|---|
| Arginine | 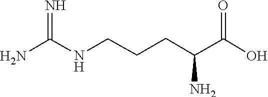 [SMILES: N=C(N)NCCC[C@H](N)C(=O)O] |
| Histidine | 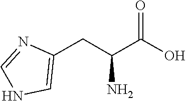 [SMILES: N[C@@H](Cc1c[nH]cn1)C(=O)O] |
| Lysine | 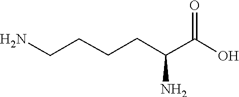 |
| Aspartic Acid | 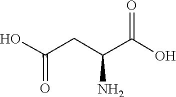 |
| Glutamic Acid | 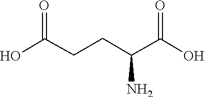 |

Proteinogenic Amino Acids with Polar Uncharged Side Chains

| | |
|---|---|
| Serine | 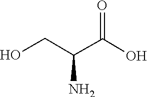 |
| Threonine | 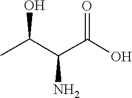 |
| Asparagine | 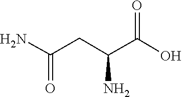 |
| Glutamine | 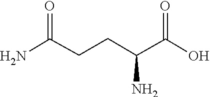 |

Proteinogenic Amino Acids with Hydrophobic Side Chains

| | |
|---|---|
| Alanine | 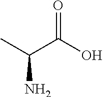 |
| Valine | 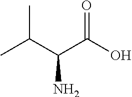 |
| Isoleucine | 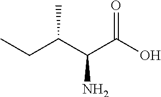 |
| Leucine | 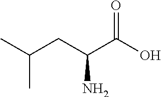 |
| Methionine | 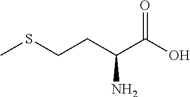 |
| Phenylalanine | 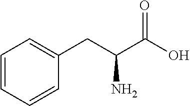 [SMILES: N[C@@H](Cc1ccccc1)C(=O)O] |
| Tyrosine | 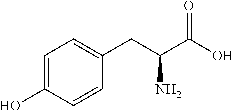 [SMILES: N[C@@H](Cc1ccc(O)cc1)C(=O)O] |
| Tryptophan | 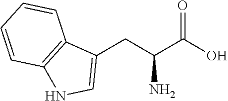 [SMILES: N[C@@H](Cc1c[nH]c2ccccc12)C(=O)O] |

Proteinogenic Amino Acids with Other Side Chains

| | |
|---|---|
| Cysteine | 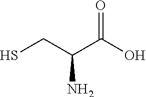 |
| Glycine | 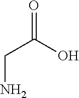 |
| Proline | 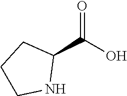 |

Non-Proteinogenic Amino Acids

| | |
|---|---|
| Aminoisobutyric acid | 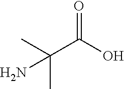 |
| D-alanine | 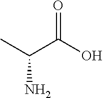 |
| β-alanine | 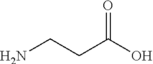 |
| γ-Aminobutyric acid | 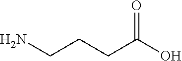 |
| Norvaline | 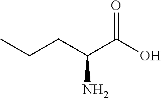 |
| Norleucine | 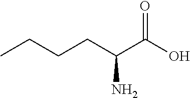 |
| Pyrrolysine (non-proteinogenic in eukaryotes) | 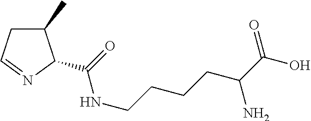 [SMILES: C[C@@H]1CC=N[C@H]1C(=O)NCCCCC(N)C(=O)O] |

### Non-Natural Amino Acids

In certain embodiments, the amino acid used in the present invention is non-naturally occurring amino acid. Non-natural amino acids do not occur in nature and are instead made synthetically.

TABLE IIIB
Non-Limiting Examples of Non-Natural Amino Acids Useful as Prodrug Moieties

| | |
|---|---|
| | Structure (L-Configuration |
| Amino Acid Name | shown) |

Non-naturally Occurring Amino Acids

| | |
|---|---|
| p-propargyloxyphenylalanine | 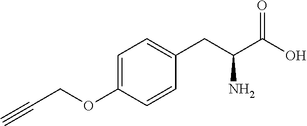 [SMILES: C#CCOc1ccc(C[C@H](N)C(=O)O)cc1] |
| p-benzoylphenylalanine | 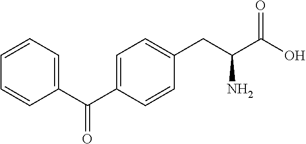 [SMILES: N[C@@H](Cc1ccc(C(=O)c2ccccc2)cc1)C(=O)O] |
| p-acetylphenylalanine | 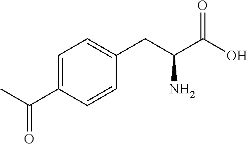 |
| pentafluorophenylalanine | 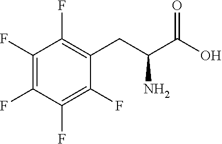 [SMILES: N[C@@H](Cc1c(F)c(F)c(F)c(F)c1F)C(=O)O] |
| (1S,2R,3S,4R)-3-aminobicyclo[2.2.1]heptane-2- carboxylic acid | 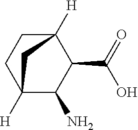 [SMILES: N[C@H]1[C@@H]2CC[C@@H](C2)[C@H]1C(=O)O] |
| 2-aminooctanedioic acid | 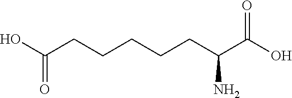 [SMILES: N[C@@H](CCCCCC(=O)O)C(=O)O] |
| 3-(4-thiazolyl)alanine | 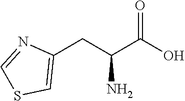 [SMILES: N[C@@H](Cc1cscn1)C(=O)O] |

### Charged Amino Acids

At physiological pH, the side chain of some amino acids bears an ionic charge. The charge depends on the acidity or basicity of the side chain. In certain embodiments, use of a charged amino acid changes the rate at which the compound of the present invention is transported by amino acid transporters. For example, use of a negatively charged amino acid such as glutamate may alter the rate of transport by an anionic amino acid-selective transporter. A charged amino acid may also be used in the present invention to increase the solubility of the compound. Ionic compounds are highly soluble in aqueous solutions such as those found in biological systems, and an appended ionic charge can provide additional solubility to an otherwise insoluble or sparingly soluble active benzofuran compound. A charged compound of the present invention will contain a pharmaceutically acceptable counter ion in the formulation. Pharmaceutically acceptable counter ions include any of the pharmaceutically acceptable salts described herein.

### Polar Uncharged Amino Acids

The side chains of other amino acids remain electronically neutral at physiological pH, yet still contain hydrogen bond donor and/or acceptor sites. In certain embodiments, use of a polar uncharged amino acid with the present invention alters the aqueous solubility of the compound. This is because additional hydrogen bonding sites can provide opportunities for improved solvation by polar protic water molecules. In some embodiments, a polar uncharged amino acid is used to alter the solubility of the compound of the present invention without introduction of a pharmaceutically acceptable salt as a counterion. Avoiding the use of a counterion can change the handling properties, consistency, morphic form, and shelf stability considerations in preparing formulations of the compound of the present invention.

### Hydrophobic Amino Acids

Some amino acids contain side chains with aliphatic groups (for example valine and leucine) or aromatic hydrocarbon rings (for example phenylalanine and tryptophan). Because these amino acids have fewer polar groups, or none at all, relative to their number of carbons, they are generally hydrophobic. Amino acids with large, hydrophobic side chains are also suitable for use in the present invention. In some embodiments, a compound of the present invention bonded to an amino acid containing a hydrophobic side chain is a substrate to a hydrophobic side chain selective amino acid or peptide transporter. Additionally, compounds of the present invention containing hydrophobic amino acids may dramatically alter the pharmacokinetic and pharmacodynamic properties of the active species.

In certain embodiments R^{P2} is at least two amino acids. In some embodiments, the at least two amino acids is a valine bonded to a valine via a peptide bond. In some embodiments, the at least two amino acids is three glycines bonded via peptide bonds.

In certain embodiments, R^{P2} is a single amino acid.

In certain embodiments, R^{P2} is a peptide

In certain embodiments, R^{P2} is a dipeptide.

In certain embodiments, R^{P2} is a tripeptide.

In certain embodiments, R^{P2} is a tetrapeptide.

In certain embodiments, R^{P2} includes one or more proteinogenic amino acids.

In certain embodiments, R^{P2} includes one or more natural amino acids.

In certain embodiments, R^{P2} includes one or more non-naturally occurring amino acids.

In certain embodiments, R^{P2} includes one or more amino acids in the D-configuration.

In certain embodiments, R^{P2} includes one or more amino acids in the L-configuration.

In certain embodiments, R^{P2} is not stereochemically enriched.

In certain embodiments, R^{P2} is aliphatic.

In certain embodiments, R^{P2} is polar uncharged.

In certain embodiments, R^{P2} is hydrophobic.

In certain embodiments, R^{P2} is electrically charged.

In certain embodiments, R^{P2} is amide-containing.

In certain embodiments, R^{P2} is sulfur-containing.

In certain embodiments, R^{P2} is aromatic.

In certain embodiments, R^{P2} is cationic.

In certain embodiments, R^{P2} is anionic.

In certain embodiments, R^{P2} is alanine.

In certain embodiments, R^{P2} is cysteine.

In certain embodiments, R^{P2} is aspartic acid.

In certain embodiments, R^{P2} is glutamic acid.

In certain embodiments, R^{P2} is phenylalanine.

In certain embodiments, R^{P2} is glycine.

In certain embodiments, R^{P2} is histidine.

In certain embodiments, R^{P2} is isoleucine.

In certain embodiments, R^{P2} is lysine.

In certain embodiments, R^{P2} is leucine.

In certain embodiments, R^{P2} is methionine.

In certain embodiments, R^{P2} is asparagine.

In certain embodiments, R^{P2} is proline.

In certain embodiments, R^{P2} is glutamine.

In certain embodiments, R^{P2} is arginine

In certain embodiments, R^{P2} is serine.

In certain embodiments, R^{P2} is threonine.

In certain embodiments, R^{P2} is valine.

In certain embodiments, R^{P2} is tryptophan.

In certain embodiments, R^{P2} is tyrosine.

In certain embodiments, R^{P2} is selenocysteine.

In certain embodiments, R^{P2} is pyrrolysine.

In certain embodiments, R^{P2} is α,α-dimethylglycine.

In certain embodiments, R^{P2} is p-propargyloxyphenylalanine.

In certain embodiments, R^{P2} is p-benzoylphenylalanine.

In certain embodiments, R^{P2} is p-acetylphenylalanine.

In certain embodiments, R^{P2} is pentafluorophenylalanine.

In certain embodiments, R^{P2} is (1S,2R,3S,4R)-3-aminobicyclo[2.2.1]heptane-2-carboxylic acid.

In certain embodiments, R^{P2} is 2-aminooctanedioic acid.

In certain embodiments, R^{P2} is 3-(4-thiazolyl)alanine.

TABLE IIIC
Exemplary Amino Acids and Peptides

| Example Number | Example R^{P2} |
|---|---|
| A-1 | Gly |
| A-2 | Gly-Gly |
| A-3 | Gly-Gly-Gly |
| A-4 | Phe |
| A-5 | Trp |
| A-6 | Trp-Trp |
| A-7 | Met |
| A-8 | Met-Met |
| A-9 | Met-Met-Met |
| A-10 | N-acetyl-Met |
| A-11 | Cys |
| A-12 | Cys-Cys |
| A-13 | N-acetyl-Cys |
| A-14 | D-ribose-Cys |
| A-15 | His |
| A-16 | His-His |
| A-17 | Ala-His |
| A-18 | Arg |
| A-19 | Arg-Arg |
| A-20 | Tyr |
| A-21 | Tyr-Tyr |
| A-22 | Val |
| A-23 | Val-Val |

In some embodiments, an indolizine has one or more protecting group moieties for example bound to the ethyl amine or to a hydroxyl substituent. Table IV below provides nonexhaustive illustrations of protecting groups contemplated in some embodiments. However, these are intended for illustrative purposes and other possibilities inherent in the definitions are contemplated. Similarly, geometric and other isomers are also contemplated. In alternative embodiments R^{P1} is selected from the groups listed in Table IV.

In certain embodiments the compound of Formula I is selected from:

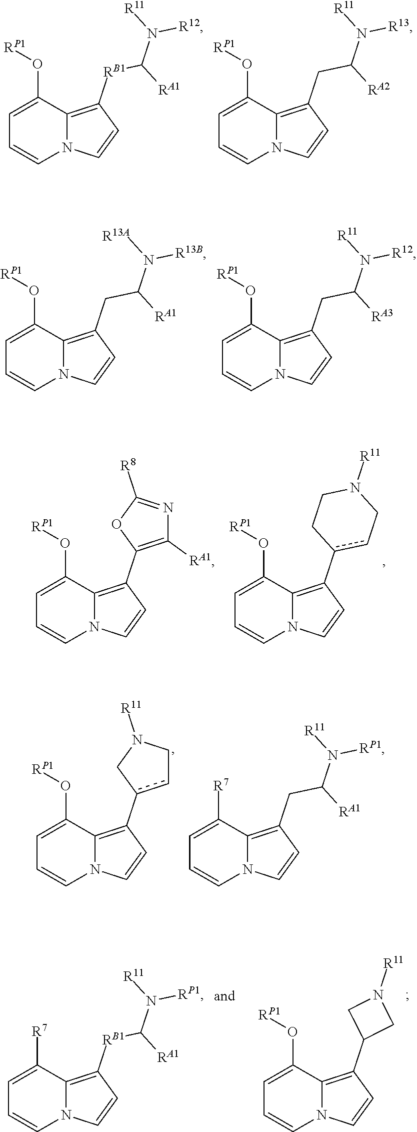

or a pharmaceutically acceptable salt or salt mixture thereof.

In certain embodiments the compound of Formula I is selected from:

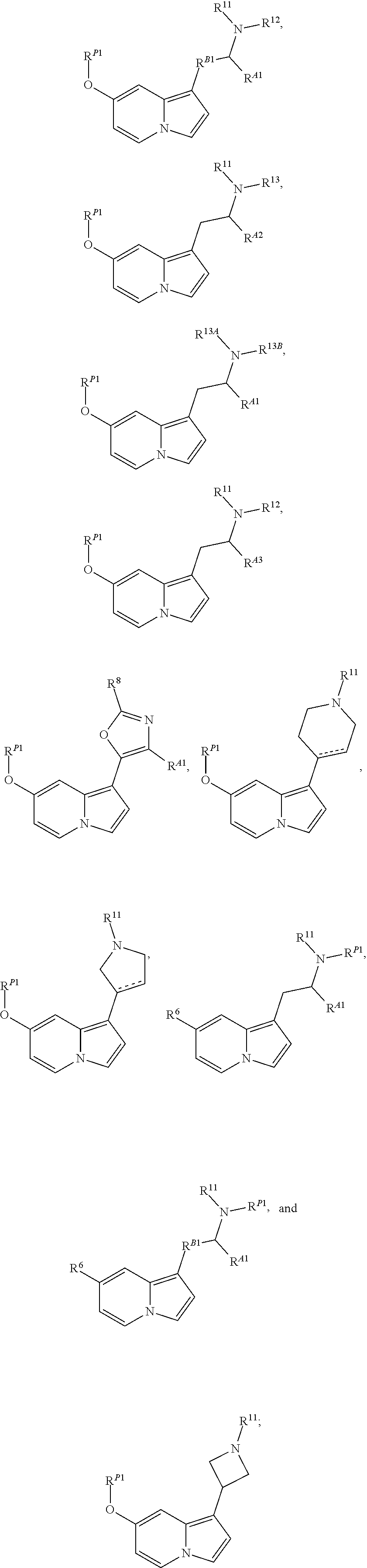

or a pharmaceutically acceptable salt or salt mixture thereof.

In certain embodiments R^{P1} is —C(O)CH₃.

In certain embodiments R^{P1} is —C(O)CH₂CH₃.

In certain embodiments R^{P1} is —C(O)OCH₃.

In certain embodiments R^{P1} is —C(O)OCH₂CH₃.

In certain embodiments R^{P1} is —C(O)CF₃.

In certain embodiments R^{P1} is —C(O)OCF₃.

In certain embodiments R^{P} is —C(O)CH₂CF₃.

In certain embodiments R^{F} is —C(O)OCH₂CF₃.

TABLE IV
Exemplary Prodrug Groups

| Example Number | Prodrug Group |
|---|---|
| F-1 | 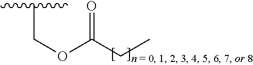 |
| F-2 | 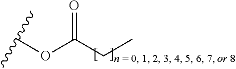 |
| F-3 | 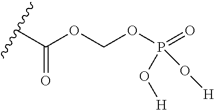 [SMILES: *C(=O)OCOP(=O)(O)O] |
| F-4 | 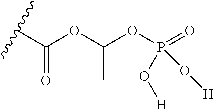 [SMILES: *C(=O)OC(C)OP(=O)(O)O] |
| F-5 | 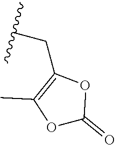 [SMILES: *Cc1oc(=O)oc1C] |
| F-6 | 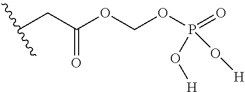 [SMILES: *CC(=O)OCOP(=O)(O)O] |
| F-7 | 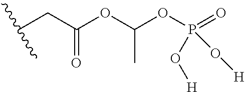 [SMILES: *CC(=O)OC(C)OP(=O)(O)O] |
| F-8 | 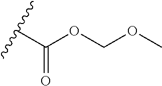 [SMILES: *C(=O)OCOC] |
| F-9 | 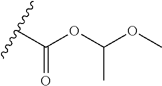 [SMILES: *C(=O)OC(C)OC] |
| F-10 | 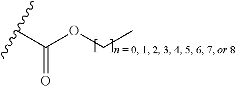 |
| F-11 | 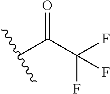 [SMILES: *C(=O)C(F)(F)F] |
| F-12 | 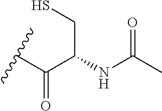 [SMILES: *C(=O)[C@H](CS)NC(C)=O] |
| F-13 | 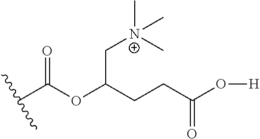 [SMILES: *C(=O)OC(CCC(=O)O)C[N+](C)(C)C] |
| F-14 | 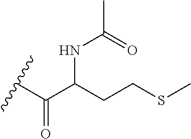 [SMILES: *C(=O)C(CCSC)NC(C)=O] |
| F-15 | 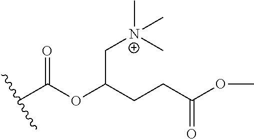 [SMILES: *C(=O)OC(CCC(=O)OC)C[N+](C)(C)C] |
| F-16 | 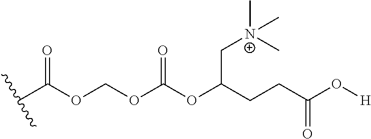 [SMILES: *C(=O)OCOC(=O)OC(CCC(=O)O)C[N+](C)(C)C] |
| F-17 | 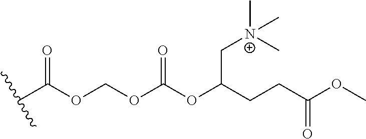 [SMILES: *C(=O)OCOC(=O)OC(CCC(=O)OC)C[N+](C)(C)C] |
| F-18 | 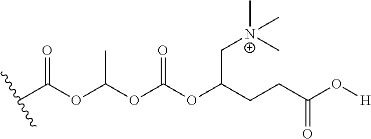 [SMILES: *C(=O)OC(C)OC(=O)OC(CCC(=O)O)C[N+](C)(C)C] |
| F-19 | 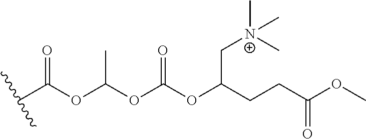 [SMILES: *C(=O)OC(C)OC(=O)OC(CCC(=O)OC)C[N+](C)(C)C] |
| F-20 | 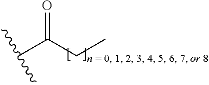 |
| F-21 | 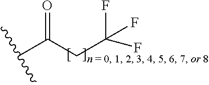 |
| F-22 | 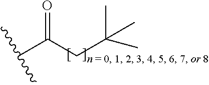 |

Wherein in the structures above compounds with a positive charge are balanced by an appropriate anion such as for example chloride or an anionic group within the molecule to form a zwitterion. In some embodiments, the entactogen prodrug is one of the exemplary and nonexhaustive structures that can be formed by taking preferred compounds from Table II and adding structures from Tables III and IV to the amine of the preferred compound, in accordance with the description provided herein.

In preferred embodiments, pharmaceutical compositions are disclosed which comprise a compound of Formula I, either racemic, as pure enantiomers, or in some combination of enantiomers, and which may be in association with another active agent, as well as with a pharmaceutically acceptable carrier, diluent, or excipient.

Where structures of Formula I have unfilled valencies, then the valencies are to be filled with hydrogens or isotopes thereof, e.g., hydrogen-1 (protium) and hydrogen-2 (deuterium).

It will be understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The structures of Formula I are amines and are typically basic in nature. They accordingly react with inorganic and organic acids to form pharmaceutically acceptable acid addition salts. In certain embodiments the free amines of this invention are oily and have decreased stability at room temperature, in this embodiment it is preferable to convert the free amines to their pharmaceutically acceptable acid addition salts for ease of handling and administration, since the latter are often solid at room temperature.

Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like.

Exemplary salts include 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, isothionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like. (See Berge et al. (1977) “Pharmaceutical Salts,” J. Pharm. Sci. 66:1-19.) Preferred pharmaceutically acceptable salts are those employing a hydrochloride anion.

One of ordinary skill will understand that the compounds of the invention shall also include the prodrugs thereof. Prodrugs are compounds that are metabolized or otherwise transformed inside the body to the active pharmacologic agent(s) of interest. Examples include addition of hydroxy groups (Tsujikawa et al. 2011. Xenobiotica, 41(7), 578-584; Yamamoto et al. 1984. Xenobiotica, 14(11), 867-875), acyloxyalkoxycarbonyl derivatives, amino acids, or peptides (Vig et al. 2013. Advanced Drug Delivery Reviews, 65(10), 1370-1385) to the amine, which can be removed within the body by chemical reactions or enzymes, but other prodrugs and precursors should be understood to be within the scope of the invention (Simplicio, Clancy, & Gilmer. 2008. Molecules, 13(3), 519-547; Shah, Chauhan, Chauhan, & Mishra (Eds.). 2020. Recent Advancement in Prodrugs. CRC Press).

### Additional Compounds

In alternative embodiments the compound of Formula I or Formula II is of formula:

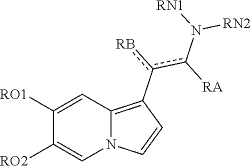

In some embodiments, each of RN1 and RN2 is independently selected from the group consisting of hydrogen, (C₁-C₆)alkyl, substituted (C₁-C₆)alkyl, hydroxy, Y, CH₂Y, and =CHY.

In some embodiments, R^{A1} is selected from the group consisting of hydrogen, CH₃, CH₂X, CHX₂, CX₃, CH₂CH₃, CH₂CH₂X, CH₂ CHX₂, CH₂CX₃, CH₂OH, and CH₂CH₂OH, where X is halogen.

In some embodiments, RB is selected from the group consisting of hydrogen, ═CH₂, hydroxy, oxo, halogen, and OY. It will be appreciated that RB may be attached with a double bond (e.g., where RB is an oxo, ═O) or with a single bond (e.g., where RB is a hydroxy, OH; or where RB is initially an oxo, but enolization or keto-enol tautomerization creates a hydroxy, and vice versa). Accordingly, the dashed line in Formula I represents an optional double bond (as it also does in any other chemical structure depicted herein; however, portrayal of an optional double bond using a dashed line is for purposes of clarity, and even where not so portrayed, it will be appreciated that chemical structures will obey chemical bonding rules, as would be understood by those of ordinary skill in the art).

In some embodiments, RB and RN1, taken together, form a five-membered ring as OCH(Y) with the oxygen bound to the β-carbon (i.e., at RB) and the carbon bound to the nitrogen (i.e., at RN1).

In some embodiments, each of RO1 and R⁰² is independently selected from the group consisting of hydrogen, halogen, (C₁—C)alkyl, (C₁-C₄)alkenyl, (C₂-C₈)alkynyl, (C₁-C₄)alkoxy, O(C₁—C)alkyl, hydroxy, CH₂X, CHX₂, CX₃, amino, alkylamino, alkylsulfinyl, alkylsulfonyl, OS(═O)₂CH₃, OS(═O)₂CH₂X, OS(═O)₂ CHX₂, OS(═O)₂CX₃, OP(═O)(OH)₂, OP(═O)(OR³)₂, and —SR³; wherein R³ is independently selected from the group consisting of hydrogen, hydroxy, CH₂X, CHX₂, CX₃, optionally substituted (C₁—C)alkyl, (C₂-C₅)alkenyl, (C₃-C₈)cycloalkyl, aryl, ═O(OH), and (═O)₂—OH, where X is halogen.

In some embodiments, Y is selected from the group consisting of (A)ₙ, R^{Z}Y¹(A)ₙ, and R^{Z}Y¹, wherein (A)ₙ is a peptide unit formed with optionally substituted amino acid units, and n is independently 1, 2, 3, or 4.

In some embodiments, R^{Z}Y¹ or at least one occurrence of R^{Z}Y¹ (without preference to attachment site) is a prodrug or prodrug fragment listed in Table TIV, or its derivative, while any additional occurrence of R^{Z}Y¹ (at any other attachment site) also may be selected from hydrogen, hydroxy, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, or a C₂₃ bile acid moiety.

“C₂₃ bile acid moiety” refers to a fragment derived from naturally occurring bile acids by removal of the carboxyl group. Preferred C₂₃ bile acid moieties are derived from the following structures:

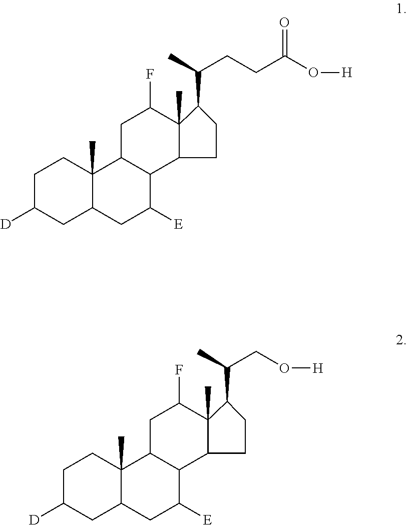

where D, E, and F are independently hydrogen, oxo, or OH.

In certain aspects “derived from a drug” refers to a fragment that is structurally related to such a drug. The structure of the fragment is identical to the drug except where a hydrogen atom attached to a heteroatom (N or O) has been replaced with a covalent bond to another group (typically, a promoiety). Note that when a drug is a salt form of a carboxylic, phosphonic or phosphoric acid, the corresponding structural fragment derived from such a drug is considered to be derived from the protonated acid form.

In certain aspects “drug” refers to a compound that exhibits therapeutic and/or prophylactic and/or diagnostic utility when administered in effective amounts to a mammal.

“Hydroxy” means the radical OH.

“Oxo” means the divalent radical ═O.

In some embodiments, (A)ₙ is selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, (A)ₙ comprises at least two amino acids as a peptide. In some embodiments, the at least two amino acids is a valine bonded to a valine via a peptide bond. In some embodiments, the at least two amino acids is three glycines bonded via peptide bonds.

In preferred embodiments, pharmaceutical compositions are disclosed which comprise a compound of Formula I, either racemic, as pure enantiomers, or in some combination of enantiomers, and which may be in association with another active agent, as well as with a pharmaceutically acceptable carrier, diluent, or excipient.

TABLE TII
Embodiments of Formula I

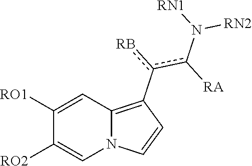

| Com- | | | | | | |
|---|---|---|---|---|---|---|
| pound | RN1 | RN2 | RA | RB | RO1 | RO2 |
| T-1 | CH2CH3 | CH2CH3 | CH2CH3 | Oxo | H | Halo |
| T-2 | CH2CH3 | CH2CH3 | CH2CH3 | Oxo | Halo | H |
| T-3 | CH2CH3 | CH2CH3 | CH2CH3 | Oxo | H | Halo |
| T-4 | CH2CH3 | CH2CH3 | CH2CH3 | Oxo | Halo | H |
| T-5 | CH2CH3 | CH2CH3 | CH2CH3 | Oxo | H | Halo |
| T-6 | CH2CH3 | CH2CH3 | CH2CH3 | Oxo | Halo | H |
| T-7 | CH2CH3 | CH2CH3 | CH2CH3 | H | H | H |
| T-8 | CH2CH3 | CH2CH3 | CH2CH3 | H | H | Halo |
| T-9 | CH2CH3 | CH2CH3 | CH2CH3 | H | H | XCH2CH3 |
| T-10 | CH2CH3 | CH2CH3 | CH2CH3 | H | H | XCH3 |
| T-11 | CH2CH3 | CH2CH3 | CH2CH3 | H | Halo | H |
| T-12 | CH2CH3 | CH2CH3 | CH2CH3 | H | XCH2CH3 | H |
| T-13 | CH2CH3 | CH2CH3 | CH2CH3 | H | XCH3 | H |
| T-14 | CH2CH3 | CH2CH3 | CH3 | Oxo | H | Halo |
| T-15 | CH2CH3 | CH2CH3 | CH3 | Oxo | Halo | H |
| T-16 | CH2CH3 | CH2CH3 | CH3 | Oxo | H | Halo |
| T-17 | CH2CH3 | CH2CH3 | CH3 | Oxo | Halo | H |
| T-18 | CH2CH3 | CH2CH3 | CH3 | Oxo | H | Halo |
| T-19 | CH2CH3 | CH2CH3 | CH3 | Oxo | Halo | H |
| T-20 | CH2CH3 | CH2CH3 | CH3 | H | H | H |
| T-21 | CH2CH3 | CH2CH3 | CH3 | H | H | Halo |
| T-22 | CH2CH3 | CH2CH3 | CH3 | H | H | XCH2CH3 |
| T-23 | CH2CH3 | CH2CH3 | CH3 | H | H | XCH3 |
| T-24 | CH2CH3 | CH2CH3 | CH3 | H | Halo | H |
| T-25 | CH2CH3 | CH2CH3 | CH3 | H | XCH2CH3 | H |
| T-26 | CH2CH3 | CH2CH3 | CH3 | H | XCH3 | H |
| T-27 | CH2CH3 | CH2CH3 | H | Oxo | H | Halo |
| T-28 | CH2CH3 | CH2CH3 | H | Oxo | Halo | H |
| T-29 | CH2CH3 | CH2CH3 | H | Oxo | H | Halo |
| T-30 | CH2CH3 | CH2CH3 | H | Oxo | Halo | H |
| T-31 | CH2CH3 | CH2CH3 | H | Oxo | H | Halo |
| T-32 | CH2CH3 | CH2CH3 | H | Oxo | Halo | H |
| T-33 | CH2CH3 | CH2CH3 | H | H | H | H |
| T-34 | CH2CH3 | CH2CH3 | H | H | H | Halo |
| T-35 | CH2CH3 | CH2CH3 | H | H | H | XCH2CH3 |
| T-36 | CH2CH3 | CH2CH3 | H | H | H | XCH3 |
| T-37 | CH2CH3 | CH2CH3 | H | H | Halo | H |
| T-38 | CH2CH3 | CH2CH3 | H | H | XCH2CH3 | H |
| T-39 | CH2CH3 | CH2CH3 | H | H | XCH3 | H |
| T-40 | CH3 | CH3 | CH2CH3 | Oxo | H | Halo |
| T-41 | CH3 | CH3 | CH2CH3 | Oxo | Halo | H |
| T-42 | CH3 | CH3 | CH2CH3 | Oxo | H | Halo |
| T-43 | CH3 | CH3 | CH2CH3 | Oxo | Halo | H |
| T-44 | CH3 | CH3 | CH2CH3 | Oxo | H | Halo |
| T-45 | CH3 | CH3 | CH2CH3 | Oxo | Halo | H |
| T-46 | CH3 | CH3 | CH2CH3 | H | H | H |
| T-47 | CH3 | CH3 | CH2CH3 | H | H | Halo |
| T-48 | CH3 | CH3 | CH2CH3 | H | H | XCH2CH3 |
| T-49 | CH3 | CH3 | CH2CH3 | H | H | XCH3 |
| T-50 | CH3 | CH3 | CH2CH3 | H | Halo | H |
| T-51 | CH3 | CH3 | CH2CH3 | H | XCH2CH3 | H |
| T-52 | CH3 | CH3 | CH2CH3 | H | XCH3 | H |
| T-53 | CH3 | CH3 | CH3 | Oxo | H | Halo |
| T-54 | CH3 | CH3 | CH3 | Oxo | Halo | H |
| T-55 | CH3 | CH3 | CH3 | Oxo | H | Halo |
| T-56 | CH3 | CH3 | CH3 | Oxo | Halo | H |
| T-57 | CH3 | CH3 | CH3 | Oxo | H | Halo |
| T-58 | CH3 | CH3 | CH3 | Oxo | Halo | H |
| T-59 | CH3 | CH3 | CH3 | H | H | H |
| T-60 | CH3 | CH3 | CH3 | H | H | Halo |
| T-61 | CH3 | CH3 | CH3 | H | H | XCH2CH3 |
| T-62 | CH3 | CH3 | CH3 | H | H | XCH3 |
| T-63 | CH3 | CH3 | CH3 | H | Halo | H |
| T-64 | CH3 | CH3 | CH3 | H | XCH2CH3 | H |
| T-65 | CH3 | CH3 | CH3 | H | XCH3 | H |
| T-66 | CH3 | CH3 | H | Oxo | H | Halo |
| T-67 | CH3 | CH3 | H | Oxo | Halo | H |
| T-68 | CH3 | CH3 | H | Oxo | H | Halo |
| T-69 | CH3 | CH3 | H | Oxo | Halo | H |
| T-70 | CH3 | CH3 | H | Oxo | H | Halo |
| T-71 | CH3 | CH3 | H | Oxo | Halo | H |
| T-72 | H | CH2CH3 | CH2CH3 | Oxo | H | Halo |
| T-73 | H | CH2CH3 | CH2CH3 | Oxo | Halo | H |
| T-74 | H | CH2CH3 | CH2CH3 | Oxo | H | Halo |
| T-75 | H | CH2CH3 | CH2CH3 | Oxo | Halo | H |
| T-76 | H | CH2CH3 | CH2CH3 | Oxo | H | H |
| T-77 | H | CH2CH3 | CH2CH3 | Oxo | H | Halo |
| T-78 | H | CH2CH3 | CH2CH3 | Oxo | H | XCH2CH3 |
| T-79 | H | CH2CH3 | CH2CH3 | Oxo | H | XCH3 |
| T-80 | H | CH2CH3 | CH2CH3 | Oxo | Halo | H |
| T-81 | H | CH2CH3 | CH2CH3 | Oxo | XCH2CH3 | H |
| T-82 | H | CH2CH3 | CH2CH3 | Oxo | XCH3 | H |
| T-83 | H | CH2CH3 | CH2CH3 | H | H | H |
| T-84 | H | CH2CH3 | CH2CH3 | H | H | Halo |
| T-85 | H | CH2CH3 | CH2CH3 | H | H | XCH2CH3 |
| T-86 | H | CH2CH3 | CH2CH3 | H | H | XCH3 |
| T-87 | H | CH2CH3 | CH2CH3 | H | Halo | H |
| T-88 | H | CH2CH3 | CH2CH3 | H | XCH2CH3 | H |
| T-89 | H | CH2CH3 | CH2CH3 | H | XCH3 | H |
| T-90 | H | CH2CH3 | CH2CH3 | H | H | H |
| T-91 | H | CH2CH3 | CH2CH3 | H | H | Halo |
| T-92 | H | CH2CH3 | CH2CH3 | H | H | XCH2CH3 |
| T-93 | H | CH2CH3 | CH2CH3 | H | H | XCH3 |
| T-94 | H | CH2CH3 | CH2CH3 | H | Halo | H |
| T-95 | H | CH2CH3 | CH2CH3 | H | XCH2CH3 | H |
| T-96 | H | CH2CH3 | CH2CH3 | H | XCH3 | H |
| T-97 | H | CH2CH3 | CH3 | Oxo | H | Halo |
| T-98 | H | CH2CH3 | CH3 | Oxo | Halo | H |
| T-99 | H | CH2CH3 | CH3 | Oxo | H | Halo |
| T-100 | H | CH2CH3 | CH3 | Oxo | Halo | H |
| T-101 | H | CH2CH3 | CH3 | Oxo | H | H |
| T-102 | H | CH2CH3 | CH3 | Oxo | H | Halo |
| T-103 | H | CH2CH3 | CH3 | Oxo | H | XCH2CH3 |
| T-104 | H | CH2CH3 | CH3 | Oxo | H | XCH3 |
| T-105 | H | CH2CH3 | CH3 | Oxo | Halo | H |
| T-106 | H | CH2CH3 | CH3 | Oxo | XCH2CH3 | H |
| T-107 | H | CH2CH3 | CH3 | Oxo | XCH3 | H |
| T-108 | H | CH2CH3 | CH3 | H | H | H |
| T-109 | H | CH2CH3 | CH3 | H | H | Halo |
| T-110 | H | CH2CH3 | CH3 | H | H | XCH2CH3 |
| T-111 | H | CH2CH3 | CH3 | H | H | XCH3 |
| T-112 | H | CH2CH3 | CH3 | H | Halo | H |
| T-113 | H | CH2CH3 | CH3 | H | XCH2CH3 | H |
| T-114 | H | CH2CH3 | CH3 | H | XCH3 | H |
| T-115 | H | CH2CH3 | CH3 | H | H | H |
| T-116 | H | CH2CH3 | CH3 | H | H | Halo |
| T-117 | H | CH2CH3 | CH3 | H | H | XCH2CH3 |
| T-118 | H | CH2CH3 | CH3 | H | H | XCH3 |
| T-119 | H | CH2CH3 | CH3 | H | Halo | H |
| T-120 | H | CH2CH3 | CH3 | H | XCH2CH3 | H |
| T-121 | H | CH2CH3 | CH3 | H | XCH3 | H |
| T-122 | H | CH2CH3 | H | Oxo | H | Halo |
| T-123 | H | CH2CH3 | H | Oxo | Halo | H |
| T-124 | H | CH2CH3 | H | Oxo | H | Halo |
| T-125 | H | CH2CH3 | H | Oxo | Halo | H |
| T-126 | H | CH2CH3 | H | Oxo | H | H |
| T-127 | H | CH2CH3 | H | Oxo | H | Halo |
| T-128 | H | CH2CH3 | H | Oxo | H | XCH2CH3 |
| T-129 | H | CH2CH3 | H | Oxo | H | XCH3 |
| T-130 | H | CH2CH3 | H | Oxo | Halo | H |
| T-131 | H | CH2CH3 | H | Oxo | XCH2CH3 | H |
| T-132 | H | CH2CH3 | H | Oxo | XCH3 | H |
| T-133 | H | CH2CH3 | H | H | H | H |
| T-134 | H | CH2CH3 | H | H | H | Halo |
| T-135 | H | CH2CH3 | H | H | H | XCH2CH3 |
| T-136 | H | CH2CH3 | H | H | H | XCH3 |
| T-137 | H | CH2CH3 | H | H | Halo | H |
| T-138 | H | CH2CH3 | H | H | XCH2CH3 | H |
| T-139 | H | CH2CH3 | H | H | XCH3 | H |
| T-140 | H | CH2CH3 | H | H | H | H |
| T-141 | H | CH2CH3 | H | H | H | XCH2CH3 |
| T-142 | H | CH2CH3 | H | H | H | XCH3 |
| T-143 | H | CH2CH3 | H | H | XCH2CH3 | H |
| T-144 | H | CH2CH3 | H | H | XCH3 | H |
| T-145 | H | CH3 | CH2CH3 | Oxo | H | Halo |
| T-146 | H | CH3 | CH2CH3 | Oxo | Halo | H |
| T-147 | H | CH3 | CH2CH3 | Oxo | H | Halo |
| T-148 | H | CH3 | CH2CH3 | Oxo | Halo | H |
| T-149 | H | CH3 | CH2CH3 | Oxo | H | H |
| T-150 | H | CH3 | CH2CH3 | Oxo | H | Halo |
| T-151 | H | CH3 | CH2CH3 | Oxo | H | XCH2CH3 |
| T-152 | H | CH3 | CH2CH3 | Oxo | H | XCH3 |
| T-153 | H | CH3 | CH2CH3 | Oxo | Halo | H |
| T-154 | H | CH3 | CH2CH3 | Oxo | XCH2CH3 | H |
| T-155 | H | CH3 | CH2CH3 | Oxo | XCH3 | H |
| T-156 | H | CH3 | CH2CH3 | H | H | H |
| T-157 | H | CH3 | CH2CH3 | H | H | Halo |
| T-158 | H | CH3 | CH2CH3 | H | H | XCH2CH3 |
| T-159 | H | CH3 | CH2CH3 | H | H | XCH3 |
| T-160 | H | CH3 | CH2CH3 | H | Halo | H |
| T-161 | H | CH3 | CH2CH3 | H | XCH2CH3 | H |
| T-162 | H | CH3 | CH2CH3 | H | XCH3 | H |
| T-163 | H | CH3 | CH2CH3 | H | H | H |
| T-164 | H | CH3 | CH2CH3 | H | H | Halo |
| T-165 | H | CH3 | CH2CH3 | H | H | XCH2CH3 |
| T-166 | H | CH3 | CH2CH3 | H | H | XCH3 |
| T-167 | H | CH3 | CH2CH3 | H | Halo | H |
| T-168 | H | CH3 | CH2CH3 | H | XCH2CH3 | H |
| T-169 | H | CH3 | CH2CH3 | H | XCH3 | H |
| T-170 | H | CH3 | CH3 | Oxo | H | Halo |
| T-171 | H | CH3 | CH3 | Oxo | Halo | H |
| T-172 | H | CH3 | CH3 | Oxo | H | Halo |
| T-173 | H | CH3 | CH3 | Oxo | Halo | H |
| T-174 | H | CH3 | CH3 | Oxo | H | H |
| T-175 | H | CH3 | CH3 | Oxo | H | Halo |
| T-176 | H | CH3 | CH3 | Oxo | H | XCH2CH3 |
| T-177 | H | CH3 | CH3 | Oxo | H | XCH3 |
| T-178 | H | CH3 | CH3 | Oxo | Halo | H |
| T-179 | H | CH3 | CH3 | Oxo | XCH2CH3 | H |
| T-180 | H | CH3 | CH3 | Oxo | XCH3 | H |
| T-181 | H | CH3 | CH3 | H | H | H |
| T-182 | H | CH3 | CH3 | H | H | Halo |
| T-183 | H | CH3 | CH3 | H | H | XCH2CH3 |
| T-184 | H | CH3 | CH3 | H | H | XCH3 |
| T-185 | H | CH3 | CH3 | H | Halo | H |
| T-186 | H | CH3 | CH3 | H | XCH2CH3 | H |
| T-187 | H | CH3 | CH3 | H | XCH3 | H |
| T-188 | H | CH3 | CH3 | H | H | H |
| T-189 | H | CH3 | CH3 | H | H | Halo |
| T-190 | H | CH3 | CH3 | H | H | XCH2CH3 |
| T-191 | H | CH3 | CH3 | H | H | XCH3 |
| T-192 | H | CH3 | CH3 | H | Halo | H |
| T-193 | H | CH3 | CH3 | H | XCH2CH3 | H |
| T-194 | H | CH3 | CH3 | H | XCH3 | H |
| T-195 | H | CH3 | H | Oxo | H | Halo |
| T-196 | H | CH3 | H | Oxo | Halo | H |
| T-197 | H | CH3 | H | Oxo | H | Halo |
| T-198 | H | CH3 | H | Oxo | Halo | H |
| T-199 | H | CH3 | H | Oxo | H | H |
| T-200 | H | CH3 | H | Oxo | H | Halo |
| T-201 | H | CH3 | H | Oxo | H | XCH2CH3 |
| T-202 | H | CH3 | H | Oxo | H | XCH3 |
| T-203 | H | CH3 | H | Oxo | Halo | H |
| T-204 | H | CH3 | H | Oxo | XCH2CH3 | H |
| T-205 | H | CH3 | H | Oxo | XCH3 | H |
| T-206 | H | CH3 | H | H | H | H |
| T-207 | H | CH3 | H | H | H | Halo |
| T-208 | H | CH3 | H | H | H | XCH2CH3 |
| T-209 | H | CH3 | H | H | H | XCH3 |
| T-210 | H | CH3 | H | H | Halo | H |
| T-211 | H | CH3 | H | H | XCH2CH3 | H |
| T-212 | H | CH3 | H | H | XCH3 | H |
| T-213 | H | CH3 | H | H | H | H |
| T-214 | H | CH3 | H | H | H | XCH2CH3 |
| T-215 | H | CH3 | H | H | H | XCH3 |
| T-216 | H | CH3 | H | H | XCH2CH3 | H |
| T-217 | H | CH3 | H | H | XCH3 | H |
| T-218 | H | H | CH2CH3 | Oxo | H | Halo |
| T-219 | H | H | CH2CH3 | Oxo | H | XCH3 |
| T-220 | H | H | CH2CH3 | Oxo | Halo | H |
| T-221 | H | H | CH2CH3 | Oxo | XCH3 | H |
| T-222 | H | H | CH2CH3 | Oxo | H | Halo |
| T-223 | H | H | CH2CH3 | Oxo | Halo | H |
| T-224 | H | H | CH2CH3 | Oxo | H | H |
| T-225 | H | H | CH2CH3 | Oxo | H | Halo |
| T-226 | H | H | CH2CH3 | Oxo | H | XCH2CH3 |
| T-227 | H | H | CH2CH3 | Oxo | H | XCH3 |
| T-228 | H | H | CH2CH3 | Oxo | Halo | H |
| T-229 | H | H | CH2CH3 | Oxo | XCH2CH3 | H |
| T-230 | H | H | CH2CH3 | Oxo | XCH3 | H |
| T-231 | H | H | CH2CH3 | H | H | H |
| T-232 | H | H | CH2CH3 | H | H | Halo |
| T-233 | H | H | CH2CH3 | H | H | XCH2CH3 |
| T-234 | H | H | CH2CH3 | H | H | XCH3 |
| T-235 | H | H | CH2CH3 | H | Halo | H |
| T-236 | H | H | CH2CH3 | H | XCH2CH3 | H |
| T-237 | H | H | CH2CH3 | H | XCH3 | H |
| T-238 | H | H | CH2CH3 | H | H | XCH2CH3 |
| T-239 | H | H | CH2CH3 | H | H | XCH3 |
| T-240 | H | H | CH2CH3 | H | XCH2CH3 | H |
| T-241 | H | H | CH2CH3 | H | XCH3 | H |
| T-242 | H | H | CH2CH3 | H | H | H |
| T-243 | H | H | CH2CH3 | H | H | XCH2CH3 |
| T-244 | H | H | CH2CH3 | H | H | XCH3 |
| T-245 | H | H | CH2CH3 | H | XCH2CH3 | H |
| T-246 | H | H | CH2CH3 | H | XCH3 | H |
| T-247 | H | H | CH3 | Oxo | H | Halo |
| T-248 | H | H | CH3 | Oxo | H | XCH3 |
| T-249 | H | H | CH3 | Oxo | Halo | H |
| T-250 | H | H | CH3 | Oxo | XCH3 | H |
| T-251 | H | H | CH3 | Oxo | H | Halo |
| T-252 | H | H | CH3 | Oxo | Halo | H |
| T-253 | H | H | CH3 | Oxo | H | H |
| T-254 | H | H | CH3 | Oxo | H | Halo |
| T-255 | H | H | CH3 | Oxo | H | XCH2CH3 |
| T-256 | H | H | CH3 | Oxo | H | XCH3 |
| T-257 | H | H | CH3 | Oxo | Halo | H |
| T-258 | H | H | CH3 | Oxo | XCH2CH3 | H |
| T-259 | H | H | CH3 | Oxo | XCH3 | H |
| T-260 | H | H | CH3 | H | H | H |
| T-261 | H | H | CH3 | H | H | Halo |
| T-262 | H | H | CH3 | H | H | XCH2CH3 |
| T-263 | H | H | CH3 | H | H | XCH3 |
| T-264 | H | H | CH3 | H | Halo | H |
| T-265 | H | H | CH3 | H | XCH2CH3 | H |
| T-266 | H | H | CH3 | H | XCH3 | H |
| T-267 | H | H | CH3 | H | H | XCH2CH3 |
| T-268 | H | H | CH3 | H | XCH2CH3 | H |
| T-269 | H | H | CH3 | H | H | H |
| T-270 | H | H | CH3 | H | H | XCH2CH3 |
| T-271 | H | H | CH3 | H | H | XCH3 |
| T-272 | H | H | CH3 | H | XCH2CH3 | H |
| T-273 | H | H | CH3 | H | XCH3 | H |
| T-274 | H | H | H | Oxo | H | Halo |
| T-275 | H | H | H | Oxo | H | XCH3 |
| T-276 | H | H | H | Oxo | Halo | H |
| T-277 | H | H | H | Oxo | XCH3 | H |
| T-278 | H | H | H | Oxo | H | Halo |
| T-279 | H | H | H | Oxo | Halo | H |
| T-280 | H | H | H | Oxo | H | H |
| T-281 | H | H | H | Oxo | H | Halo |
| T-282 | H | H | H | Oxo | H | XCH2CH3 |
| T-283 | H | H | H | Oxo | H | XCH3 |
| T-284 | H | H | H | Oxo | Halo | H |
| T-285 | H | H | H | Oxo | XCH2CH3 | H |
| T-286 | H | H | H | Oxo | XCH3 | H |
| T-287 | H | H | H | H | H | Halo |
| T-288 | H | H | H | H | Halo | H |
| T-289 | H | H | H | H | H | XCH2CH3 |
| T-290 | H | H | H | H | XCH2CH3 | H |
| T-291 | H | H | H | H | H | H |
| T-292 | H | H | H | H | H | XCH2CH3 |
| T-293 | H | H | H | H | H | XCH3 |
| T-294 | H | H | H | H | XCH2CH3 | H |
| T-295 | H | H | H | H | XCH3 | H |
| T-296 | Y | CH2CH3 | CH2CH3 | Oxo | H | H |
| T-297 | Y | CH2CH3 | CH2CH3 | Oxo | H | Halo |
| T-298 | Y | CH2CH3 | CH2CH3 | Oxo | H | XCH2CH3 |
| T-299 | Y | CH2CH3 | CH2CH3 | Oxo | H | XCH3 |
| T-300 | Y | CH2CH3 | CH2CH3 | Oxo | Halo | H |
| T-301 | Y | CH2CH3 | CH2CH3 | Oxo | XCH2CH3 | H |
| T-302 | Y | CH2CH3 | CH2CH3 | Oxo | XCH3 | H |
| T-303 | Y | CH2CH3 | CH2CH3 | Oxo | H | H |
| T-304 | Y | CH2CH3 | CH2CH3 | Oxo | H | Halo |
| T-305 | Y | CH2CH3 | CH2CH3 | Oxo | H | XCH2CH3 |
| T-306 | Y | CH2CH3 | CH2CH3 | Oxo | H | XCH3 |
| T-307 | Y | CH2CH3 | CH2CH3 | Oxo | Halo | H |
| T-308 | Y | CH2CH3 | CH2CH3 | Oxo | XCH2CH3 | H |
| T-309 | Y | CH2CH3 | CH2CH3 | Oxo | XCH3 | H |
| T-310 | Y | CH2CH3 | CH2CH3 | Oxo | H | H |
| T-311 | Y | CH2CH3 | CH2CH3 | Oxo | H | Halo |
| T-312 | Y | CH2CH3 | CH2CH3 | Oxo | H | XCH2CH3 |
| T-313 | Y | CH2CH3 | CH2CH3 | Oxo | H | XCH3 |
| T-314 | Y | CH2CH3 | CH2CH3 | Oxo | Halo | H |
| T-315 | Y | CH2CH3 | CH2CH3 | Oxo | XCH2CH3 | H |
| T-316 | Y | CH2CH3 | CH2CH3 | Oxo | XCH3 | H |
| T-317 | Y | CH2CH3 | CH2CH3 | H | H | H |
| T-318 | Y | CH2CH3 | CH2CH3 | H | H | Halo |
| T-319 | Y | CH2CH3 | CH2CH3 | H | H | XCH2CH3 |
| T-320 | Y | CH2CH3 | CH2CH3 | H | H | XCH3 |
| T-321 | Y | CH2CH3 | CH2CH3 | H | Halo | H |
| T-322 | Y | CH2CH3 | CH2CH3 | H | XCH2CH3 | H |
| T-323 | Y | CH2CH3 | CH2CH3 | H | XCH3 | H |
| T-324 | Y | CH2CH3 | CH2CH3 | H | H | H |
| T-325 | Y | CH2CH3 | CH2CH3 | H | H | Halo |
| T-326 | Y | CH2CH3 | CH2CH3 | H | H | XCH2CH3 |
| T-327 | Y | CH2CH3 | CH2CH3 | H | H | XCH3 |
| T-328 | Y | CH2CH3 | CH2CH3 | H | Halo | H |
| T-329 | Y | CH2CH3 | CH2CH3 | H | XCH2CH3 | H |
| T-330 | Y | CH2CH3 | CH2CH3 | H | XCH3 | H |
| T-331 | Y | CH2CH3 | CH2CH3 | H | H | H |
| T-332 | Y | CH2CH3 | CH2CH3 | H | H | Halo |
| T-333 | Y | CH2CH3 | CH2CH3 | H | H | XCH2CH3 |
| T-334 | Y | CH2CH3 | CH2CH3 | H | H | XCH3 |
| T-335 | Y | CH2CH3 | CH2CH3 | H | Halo | H |
| T-336 | Y | CH2CH3 | CH2CH3 | H | XCH2CH3 | H |
| T-337 | Y | CH2CH3 | CH2CH3 | H | XCH3 | H |
| T-338 | Y | CH2CH3 | CH3 | Oxo | H | H |
| T-339 | Y | CH2CH3 | CH3 | Oxo | H | Halo |
| T-340 | Y | CH2CH3 | CH3 | Oxo | H | XCH2CH3 |
| T-341 | Y | CH2CH3 | CH3 | Oxo | H | XCH3 |
| T-342 | Y | CH2CH3 | CH3 | Oxo | Halo | H |
| T-343 | Y | CH2CH3 | CH3 | Oxo | XCH2CH3 | H |
| T-344 | Y | CH2CH3 | CH3 | Oxo | XCH3 | H |
| T-345 | Y | CH2CH3 | CH3 | Oxo | H | H |
| T-346 | Y | CH2CH3 | CH3 | Oxo | H | Halo |
| T-347 | Y | CH2CH3 | CH3 | Oxo | H | XCH2CH3 |
| T-348 | Y | CH2CH3 | CH3 | Oxo | H | XCH3 |
| T-349 | Y | CH2CH3 | CH3 | Oxo | Halo | H |
| T-350 | Y | CH2CH3 | CH3 | Oxo | XCH2CH3 | H |
| T-351 | Y | CH2CH3 | CH3 | Oxo | XCH3 | H |
| T-352 | Y | CH2CH3 | CH3 | Oxo | H | H |
| T-353 | Y | CH2CH3 | CH3 | Oxo | H | Halo |
| T-354 | Y | CH2CH3 | CH3 | Oxo | H | XCH2CH3 |
| T-355 | Y | CH2CH3 | CH3 | Oxo | H | XCH3 |
| T-356 | Y | CH2CH3 | CH3 | Oxo | Halo | H |
| T-357 | Y | CH2CH3 | CH3 | Oxo | XCH2CH3 | H |
| T-358 | Y | CH2CH3 | CH3 | Oxo | XCH3 | H |
| T-359 | Y | CH2CH3 | CH3 | H | H | H |
| T-360 | Y | CH2CH3 | CH3 | H | H | Halo |
| T-361 | Y | CH2CH3 | CH3 | H | H | XCH2CH3 |
| T-362 | Y | CH2CH3 | CH3 | H | H | XCH3 |
| T-363 | Y | CH2CH3 | CH3 | H | Halo | H |
| T-364 | Y | CH2CH3 | CH3 | H | XCH2CH3 | H |
| T-365 | Y | CH2CH3 | CH3 | H | XCH3 | H |
| T-366 | Y | CH2CH3 | CH3 | H | H | H |
| T-367 | Y | CH2CH3 | CH3 | H | H | Halo |
| T-368 | Y | CH2CH3 | CH3 | H | H | XCH2CH3 |
| T-369 | Y | CH2CH3 | CH3 | H | H | XCH3 |
| T-370 | Y | CH2CH3 | CH3 | H | Halo | H |
| T-371 | Y | CH2CH3 | CH3 | H | XCH2CH3 | H |
| T-372 | Y | CH2CH3 | CH3 | H | XCH3 | H |
| T-373 | Y | CH2CH3 | CH3 | H | H | H |
| T-374 | Y | CH2CH3 | CH3 | H | H | Halo |
| T-375 | Y | CH2CH3 | CH3 | H | H | XCH2CH3 |
| T-376 | Y | CH2CH3 | CH3 | H | H | XCH3 |
| T-377 | Y | CH2CH3 | CH3 | H | Halo | H |
| T-378 | Y | CH2CH3 | CH3 | H | XCH2CH3 | H |
| T-379 | Y | CH2CH3 | CH3 | H | XCH3 | H |
| T-380 | Y | CH2CH3 | H | Oxo | H | H |
| T-381 | Y | CH2CH3 | H | Oxo | H | Halo |
| T-382 | Y | CH2CH3 | H | Oxo | H | XCH2CH3 |
| T-383 | Y | CH2CH3 | H | Oxo | H | XCH3 |
| T-384 | Y | CH2CH3 | H | Oxo | Halo | H |
| T-385 | Y | CH2CH3 | H | Oxo | XCH2CH3 | H |
| T-386 | Y | CH2CH3 | H | Oxo | XCH3 | H |
| T-387 | Y | CH2CH3 | H | Oxo | H | H |
| T-388 | Y | CH2CH3 | H | Oxo | H | Halo |
| T-389 | Y | CH2CH3 | H | Oxo | H | XCH2CH3 |
| T-390 | Y | CH2CH3 | H | Oxo | H | XCH3 |
| T-391 | Y | CH2CH3 | H | Oxo | Halo | H |
| T-392 | Y | CH2CH3 | H | Oxo | XCH2CH3 | H |
| T-393 | Y | CH2CH3 | H | Oxo | XCH3 | H |
| T-394 | Y | CH2CH3 | H | Oxo | H | H |
| T-395 | Y | CH2CH3 | H | Oxo | H | Halo |
| T-396 | Y | CH2CH3 | H | Oxo | H | XCH2CH3 |
| T-397 | Y | CH2CH3 | H | Oxo | H | XCH3 |
| T-398 | Y | CH2CH3 | H | Oxo | Halo | H |
| T-399 | Y | CH2CH3 | H | Oxo | XCH2CH3 | H |
| T-400 | Y | CH2CH3 | H | Oxo | XCH3 | H |
| T-401 | Y | CH2CH3 | H | H | H | H |
| T-402 | Y | CH2CH3 | H | H | H | Halo |
| T-403 | Y | CH2CH3 | H | H | H | XCH2CH3 |
| T-404 | Y | CH2CH3 | H | H | H | XCH3 |
| T-405 | Y | CH2CH3 | H | H | Halo | H |
| T-406 | Y | CH2CH3 | H | H | XCH2CH3 | H |
| T-407 | Y | CH2CH3 | H | H | XCH3 | H |
| T-408 | Y | CH2CH3 | H | H | H | H |
| T-409 | Y | CH2CH3 | H | H | H | XCH2CH3 |
| T-410 | Y | CH2CH3 | H | H | H | XCH3 |
| T-411 | Y | CH2CH3 | H | H | XCH2CH3 | H |
| T-412 | Y | CH2CH3 | H | H | XCH3 | H |
| T-413 | Y | CH2CH3 | H | H | H | H |
| T-414 | Y | CH2CH3 | H | H | H | XCH2CH3 |
| T-415 | Y | CH2CH3 | H | H | H | XCH3 |
| T-416 | Y | CH2CH3 | H | H | XCH2CH3 | H |
| T-417 | Y | CH2CH3 | H | H | XCH3 | H |
| T-418 | Y | CH3 | CH2CH3 | Oxo | H | H |
| T-419 | Y | CH3 | CH2CH3 | Oxo | H | Halo |
| T-420 | Y | CH3 | CH2CH3 | Oxo | H | XCH2CH3 |
| T-421 | Y | CH3 | CH2CH3 | Oxo | H | XCH3 |
| T-422 | Y | CH3 | CH2CH3 | Oxo | Halo | H |
| T-423 | Y | CH3 | CH2CH3 | Oxo | XCH2CH3 | H |
| T-424 | Y | CH3 | CH2CH3 | Oxo | XCH3 | H |
| T-425 | Y | CH3 | CH2CH3 | Oxo | H | H |
| T-426 | Y | CH3 | CH2CH3 | Oxo | H | Halo |
| T-427 | Y | CH3 | CH2CH3 | Oxo | H | XCH2CH3 |
| T-428 | Y | CH3 | CH2CH3 | Oxo | H | XCH3 |
| T-429 | Y | CH3 | CH2CH3 | Oxo | Halo | H |
| T-430 | Y | CH3 | CH2CH3 | Oxo | XCH2CH3 | H |
| T-431 | Y | CH3 | CH2CH3 | Oxo | XCH3 | H |
| T-432 | Y | CH3 | CH2CH3 | Oxo | H | H |
| T-433 | Y | CH3 | CH2CH3 | Oxo | H | Halo |
| T-434 | Y | CH3 | CH2CH3 | Oxo | H | XCH2CH3 |
| T-435 | Y | CH3 | CH2CH3 | Oxo | H | XCH3 |
| T-436 | Y | CH3 | CH2CH3 | Oxo | Halo | H |
| T-437 | Y | CH3 | CH2CH3 | Oxo | XCH2CH3 | H |
| T-438 | Y | CH3 | CH2CH3 | Oxo | XCH3 | H |
| T-439 | Y | CH3 | CH2CH3 | H | H | H |
| T-440 | Y | CH3 | CH2CH3 | H | H | Halo |
| T-441 | Y | CH3 | CH2CH3 | H | H | XCH2CH3 |
| T-442 | Y | CH3 | CH2CH3 | H | H | XCH3 |
| T-443 | Y | CH3 | CH2CH3 | H | Halo | H |
| T-444 | Y | CH3 | CH2CH3 | H | XCH2CH3 | H |
| T-445 | Y | CH3 | CH2CH3 | H | XCH3 | H |
| T-446 | Y | CH3 | CH2CH3 | H | H | H |
| T-447 | Y | CH3 | CH2CH3 | H | H | Halo |
| T-448 | Y | CH3 | CH2CH3 | H | H | XCH2CH3 |
| T-449 | Y | CH3 | CH2CH3 | H | H | XCH3 |
| T-450 | Y | CH3 | CH2CH3 | H | Halo | H |
| T-451 | Y | CH3 | CH2CH3 | H | XCH2CH3 | H |
| T-452 | Y | CH3 | CH2CH3 | H | XCH3 | H |
| T-453 | Y | CH3 | CH2CH3 | H | H | H |
| T-454 | Y | CH3 | CH2CH3 | H | H | Halo |
| T-455 | Y | CH3 | CH2CH3 | H | H | XCH2CH3 |
| T-456 | Y | CH3 | CH2CH3 | H | H | XCH3 |
| T-457 | Y | CH3 | CH2CH3 | H | Halo | H |
| T-458 | Y | CH3 | CH2CH3 | H | XCH2CH3 | H |
| T-459 | Y | CH3 | CH2CH3 | H | XCH3 | H |
| T-460 | Y | CH3 | CH3 | Oxo | H | H |
| T-461 | Y | CH3 | CH3 | Oxo | H | Halo |
| T-462 | Y | CH3 | CH3 | Oxo | H | XCH2CH3 |
| T-463 | Y | CH3 | CH3 | Oxo | H | XCH3 |
| T-464 | Y | CH3 | CH3 | Oxo | Halo | H |
| T-465 | Y | CH3 | CH3 | Oxo | XCH2CH3 | H |
| T-466 | Y | CH3 | CH3 | Oxo | XCH3 | H |
| T-467 | Y | CH3 | CH3 | Oxo | H | H |
| T-468 | Y | CH3 | CH3 | Oxo | H | Halo |
| T-469 | Y | CH3 | CH3 | Oxo | H | XCH2CH3 |
| T-470 | Y | CH3 | CH3 | Oxo | H | XCH3 |
| T-471 | Y | CH3 | CH3 | Oxo | Halo | H |
| T-472 | Y | CH3 | CH3 | Oxo | XCH2CH3 | H |
| T-473 | Y | CH3 | CH3 | Oxo | XCH3 | H |
| T-474 | Y | CH3 | CH3 | Oxo | H | H |
| T-475 | Y | CH3 | CH3 | Oxo | H | Halo |
| T-476 | Y | CH3 | CH3 | Oxo | H | XCH2CH3 |
| T-477 | Y | CH3 | CH3 | Oxo | H | XCH3 |
| T-478 | Y | CH3 | CH3 | Oxo | Halo | H |
| T-479 | Y | CH3 | CH3 | Oxo | XCH2CH3 | H |
| T-480 | Y | CH3 | CH3 | Oxo | XCH3 | H |
| T-481 | Y | CH3 | CH3 | H | H | H |
| T-482 | Y | CH3 | CH3 | H | H | Halo |
| T-483 | Y | CH3 | CH3 | H | H | XCH2CH3 |
| T-484 | Y | CH3 | CH3 | H | H | XCH3 |
| T-485 | Y | CH3 | CH3 | H | Halo | H |
| T-486 | Y | CH3 | CH3 | H | XCH2CH3 | H |
| T-487 | Y | CH3 | CH3 | H | XCH3 | H |
| T-488 | Y | CH3 | CH3 | H | H | H |
| T-489 | Y | CH3 | CH3 | H | H | Halo |
| T-490 | Y | CH3 | CH3 | H | H | XCH2CH3 |
| T-491 | Y | CH3 | CH3 | H | H | XCH3 |
| T-492 | Y | CH3 | CH3 | H | Halo | H |
| T-493 | Y | CH3 | CH3 | H | XCH2CH3 | H |
| T-494 | Y | CH3 | CH3 | H | XCH3 | H |
| T-495 | Y | CH3 | CH3 | H | H | H |
| T-496 | Y | CH3 | CH3 | H | H | Halo |
| T-497 | Y | CH3 | CH3 | H | H | XCH2CH3 |
| T-498 | Y | CH3 | CH3 | H | H | XCH3 |
| T-499 | Y | CH3 | CH3 | H | Halo | H |
| T-500 | Y | CH3 | CH3 | H | XCH2CH3 | H |
| T-501 | Y | CH3 | CH3 | H | XCH3 | H |
| T-502 | Y | CH3 | H | Oxo | H | H |
| T-503 | Y | CH3 | H | Oxo | H | Halo |
| T-504 | Y | CH3 | H | Oxo | H | XCH2CH3 |
| T-505 | Y | CH3 | H | Oxo | H | XCH3 |
| T-506 | Y | CH3 | H | Oxo | Halo | H |
| T-507 | Y | CH3 | H | Oxo | XCH2CH3 | H |
| T-508 | Y | CH3 | H | Oxo | XCH3 | H |
| T-509 | Y | CH3 | H | Oxo | H | H |
| T-510 | Y | CH3 | H | Oxo | H | Halo |
| T-511 | Y | CH3 | H | Oxo | H | XCH2CH3 |
| T-512 | Y | CH3 | H | Oxo | H | XCH3 |
| T-513 | Y | CH3 | H | Oxo | Halo | H |
| T-514 | Y | CH3 | H | Oxo | XCH2CH3 | H |
| T-515 | Y | CH3 | H | Oxo | XCH3 | H |
| T-516 | Y | CH3 | H | Oxo | H | H |
| T-517 | Y | CH3 | H | Oxo | H | Halo |
| T-518 | Y | CH3 | H | Oxo | H | XCH2CH3 |
| T-519 | Y | CH3 | H | Oxo | H | XCH3 |
| T-520 | Y | CH3 | H | Oxo | Halo | H |
| T-521 | Y | CH3 | H | Oxo | XCH2CH3 | H |
| T-522 | Y | CH3 | H | Oxo | XCH3 | H |
| T-523 | Y | CH3 | H | H | H | H |
| T-524 | Y | CH3 | H | H | H | Halo |
| T-525 | Y | CH3 | H | H | H | XCH2CH3 |
| T-526 | Y | CH3 | H | H | H | XCH3 |
| T-527 | Y | CH3 | H | H | Halo | H |
| T-528 | Y | CH3 | H | H | XCH2CH3 | H |
| T-529 | Y | CH3 | H | H | XCH3 | H |
| T-530 | Y | CH3 | H | H | H | H |
| T-531 | Y | CH3 | H | H | H | XCH2CH3 |
| T-532 | Y | CH3 | H | H | H | XCH3 |
| T-533 | Y | CH3 | H | H | XCH2CH3 | H |
| T-534 | Y | CH3 | H | H | XCH3 | H |
| T-535 | Y | CH3 | H | H | H | H |
| T-536 | Y | CH3 | H | H | H | XCH2CH3 |
| T-537 | Y | CH3 | H | H | H | XCH3 |
| T-538 | Y | CH3 | H | H | XCH2CH3 | H |
| T-539 | Y | CH3 | H | H | XCH3 | H |
| T-540 | Y | H | CH2CH3 | Oxo | H | H |
| T-541 | Y | H | CH2CH3 | Oxo | H | Halo |
| T-542 | Y | H | CH2CH3 | Oxo | H | XCH2CH3 |
| T-543 | Y | H | CH2CH3 | Oxo | H | XCH3 |
| T-544 | Y | H | CH2CH3 | Oxo | Halo | H |
| T-545 | Y | H | CH2CH3 | Oxo | XCH2CH3 | H |
| T-546 | Y | H | CH2CH3 | Oxo | XCH3 | H |
| T-547 | Y | H | CH2CH3 | Oxo | H | H |
| T-548 | Y | H | CH2CH3 | Oxo | H | Halo |
| T-549 | Y | H | CH2CH3 | Oxo | H | XCH2CH3 |
| T-550 | Y | H | CH2CH3 | Oxo | H | XCH3 |
| T-551 | Y | H | CH2CH3 | Oxo | Halo | H |
| T-552 | Y | H | CH2CH3 | Oxo | XCH2CH3 | H |
| T-553 | Y | H | CH2CH3 | Oxo | XCH3 | H |
| T-554 | Y | H | CH2CH3 | Oxo | H | H |
| T-555 | Y | H | CH2CH3 | Oxo | H | Halo |
| T-556 | Y | H | CH2CH3 | Oxo | H | XCH2CH3 |
| T-557 | Y | H | CH2CH3 | Oxo | H | XCH3 |
| T-558 | Y | H | CH2CH3 | Oxo | Halo | H |
| T-559 | Y | H | CH2CH3 | Oxo | XCH2CH3 | H |
| T-560 | Y | H | CH2CH3 | Oxo | XCH3 | H |
| T-561 | Y | H | CH2CH3 | H | H | H |
| T-562 | Y | H | CH2CH3 | H | H | Halo |
| T-563 | Y | H | CH2CH3 | H | H | XCH2CH3 |
| T-564 | Y | H | CH2CH3 | H | H | XCH3 |
| T-565 | Y | H | CH2CH3 | H | Halo | H |
| T-566 | Y | H | CH2CH3 | H | XCH2CH3 | H |
| T-567 | Y | H | CH2CH3 | H | XCH3 | H |
| T-568 | Y | H | CH2CH3 | H | H | H |
| T-569 | Y | H | CH2CH3 | H | H | XCH2CH3 |
| T-570 | Y | H | CH2CH3 | H | H | XCH3 |
| T-571 | Y | H | CH2CH3 | H | XCH2CH3 | H |
| T-572 | Y | H | CH2CH3 | H | XCH3 | H |
| T-573 | Y | H | CH2CH3 | H | H | H |
| T-574 | Y | H | CH2CH3 | H | H | XCH2CH3 |
| T-575 | Y | H | CH2CH3 | H | H | XCH3 |
| T-576 | Y | H | CH2CH3 | H | XCH2CH3 | H |
| T-577 | Y | H | CH2CH3 | H | XCH3 | H |
| T-578 | Y | H | CH3 | Oxo | H | H |
| T-579 | Y | H | CH3 | Oxo | H | Halo |
| T-580 | Y | H | CH3 | Oxo | H | XCH2CH3 |
| T-581 | Y | H | CH3 | Oxo | H | XCH3 |
| T-582 | Y | H | CH3 | Oxo | Halo | H |
| T-583 | Y | H | CH3 | Oxo | XCH2CH3 | H |
| T-584 | Y | H | CH3 | Oxo | XCH3 | H |
| T-585 | Y | H | CH3 | Oxo | H | H |
| T-586 | Y | H | CH3 | Oxo | H | Halo |
| T-587 | Y | H | CH3 | Oxo | H | XCH2CH3 |
| T-588 | Y | H | CH3 | Oxo | H | XCH3 |
| T-589 | Y | H | CH3 | Oxo | Halo | H |
| T-590 | Y | H | CH3 | Oxo | XCH2CH3 | H |
| T-591 | Y | H | CH3 | Oxo | XCH3 | H |
| T-592 | Y | H | CH3 | Oxo | H | H |
| T-593 | Y | H | CH3 | Oxo | H | Halo |
| T-594 | Y | H | CH3 | Oxo | H | XCH2CH3 |
| T-595 | Y | H | CH3 | Oxo | H | XCH3 |
| T-596 | Y | H | CH3 | Oxo | Halo | H |
| T-597 | Y | H | CH3 | Oxo | XCH2CH3 | H |
| T-598 | Y | H | CH3 | Oxo | XCH3 | H |
| T-599 | Y | H | CH3 | H | H | H |
| T-600 | Y | H | CH3 | H | H | Halo |
| T-601 | Y | H | CH3 | H | H | XCH2CH3 |
| T-602 | Y | H | CH3 | H | H | XCH3 |
| T-603 | Y | H | CH3 | H | Halo | H |
| T-604 | Y | H | CH3 | H | XCH2CH3 | H |
| T-605 | Y | H | CH3 | H | XCH3 | H |
| T-606 | Y | H | CH3 | H | H | H |
| T-607 | Y | H | CH3 | H | H | XCH2CH3 |
| T-608 | Y | H | CH3 | H | H | XCH3 |
| T-609 | Y | H | CH3 | H | XCH2CH3 | H |
| T-610 | Y | H | CH3 | H | XCH3 | H |
| T-611 | Y | H | CH3 | H | H | H |
| T-612 | Y | H | CH3 | H | H | XCH2CH3 |
| T-613 | Y | H | CH3 | H | H | XCH3 |
| T-614 | Y | H | CH3 | H | XCH2CH3 | H |
| T-615 | Y | H | CH3 | H | XCH3 | H |
| T-616 | Y | H | H | Oxo | H | H |
| T-617 | Y | H | H | Oxo | H | Halo |
| T-618 | Y | H | H | Oxo | H | XCH2CH3 |
| T-619 | Y | H | H | Oxo | H | XCH3 |
| T-620 | Y | H | H | Oxo | Halo | H |
| T-621 | Y | H | H | Oxo | XCH2CH3 | H |
| T-622 | Y | H | H | Oxo | XCH3 | H |
| T-623 | Y | H | H | Oxo | H | H |
| T-624 | Y | H | H | Oxo | H | Halo |
| T-625 | Y | H | H | Oxo | H | XCH2CH3 |
| T-626 | Y | H | H | Oxo | H | XCH3 |
| T-627 | Y | H | H | Oxo | Halo | H |
| T-628 | Y | H | H | Oxo | XCH2CH3 | H |
| T-629 | Y | H | H | Oxo | XCH3 | H |
| T-630 | Y | H | H | Oxo | H | H |
| T-631 | Y | H | H | Oxo | H | Halo |
| T-632 | Y | H | H | Oxo | H | XCH2CH3 |
| T-633 | Y | H | H | Oxo | H | XCH3 |
| T-634 | Y | H | H | Oxo | Halo | H |
| T-635 | Y | H | H | Oxo | XCH2CH3 | H |
| T-636 | Y | H | H | Oxo | XCH3 | H |
| T-637 | Y | H | H | H | H | H |
| T-638 | Y | H | H | H | H | Halo |
| T-639 | Y | H | H | H | H | XCH2CH3 |
| T-640 | Y | H | H | H | H | XCH3 |
| T-641 | Y | H | H | H | Halo | H |
| T-642 | Y | H | H | H | XCH2CH3 | H |
| T-643 | Y | H | H | H | XCH3 | H |
| T-644 | Y | H | H | H | H | H |
| T-645 | Y | H | H | H | H | XCH2CH3 |
| T-646 | Y | H | H | H | H | XCH3 |
| T-647 | Y | H | H | H | XCH2CH3 | H |
| T-648 | Y | H | H | H | XCH3 | H |
| T-649 | Y | H | H | H | H | H |
| T-650 | Y | H | H | H | H | XCH2CH3 |
| T-651 | Y | H | H | H | H | XCH3 |
| T-652 | Y | H | H | H | XCH2CH3 | H |
| T-653 | Y | H | H | H | XCH3 | H |
| T-654 | Y | Y | CH2CH3 | Oxo | H | H |
| T-655 | Y | Y | CH2CH3 | Oxo | H | Halo |
| T-656 | Y | Y | CH2CH3 | Oxo | H | XCH2CH3 |
| T-657 | Y | Y | CH2CH3 | Oxo | H | XCH3 |
| T-658 | Y | Y | CH2CH3 | Oxo | Halo | H |
| T-659 | Y | Y | CH2CH3 | Oxo | XCH2CH3 | H |
| T-660 | Y | Y | CH2CH3 | Oxo | XCH3 | H |
| T-661 | Y | Y | CH2CH3 | Oxo | H | H |
| T-662 | Y | Y | CH2CH3 | Oxo | H | Halo |
| T-663 | Y | Y | CH2CH3 | Oxo | H | XCH2CH3 |
| T-664 | Y | Y | CH2CH3 | Oxo | H | XCH3 |
| T-665 | Y | Y | CH2CH3 | Oxo | Halo | H |
| T-666 | Y | Y | CH2CH3 | Oxo | XCH2CH3 | H |
| T-667 | Y | Y | CH2CH3 | Oxo | XCH3 | H |
| T-668 | Y | Y | CH2CH3 | Oxo | H | H |
| T-669 | Y | Y | CH2CH3 | Oxo | H | Halo |
| T-670 | Y | Y | CH2CH3 | Oxo | H | XCH2CH3 |
| T-671 | Y | Y | CH2CH3 | Oxo | H | XCH3 |
| T-672 | Y | Y | CH2CH3 | Oxo | Halo | H |
| T-673 | Y | Y | CH2CH3 | Oxo | XCH2CH3 | H |
| T-674 | Y | Y | CH2CH3 | Oxo | XCH3 | H |
| T-675 | Y | Y | CH2CH3 | H | H | H |
| T-676 | Y | Y | CH2CH3 | H | H | Halo |
| T-677 | Y | Y | CH2CH3 | H | H | XCH2CH3 |
| T-678 | Y | Y | CH2CH3 | H | H | XCH3 |
| T-679 | Y | Y | CH2CH3 | H | Halo | H |
| T-680 | Y | Y | CH2CH3 | H | XCH2CH3 | H |
| T-681 | Y | Y | CH2CH3 | H | XCH3 | H |
| T-682 | Y | Y | CH2CH3 | H | H | H |
| T-683 | Y | Y | CH2CH3 | H | H | XCH2CH3 |
| T-684 | Y | Y | CH2CH3 | H | H | XCH3 |
| T-685 | Y | Y | CH2CH3 | H | XCH2CH3 | H |
| T-686 | Y | Y | CH2CH3 | H | XCH3 | H |
| T-687 | Y | Y | CH2CH3 | H | H | H |
| T-688 | Y | Y | CH2CH3 | H | H | XCH2CH3 |
| T-689 | Y | Y | CH2CH3 | H | H | XCH3 |
| T-690 | Y | Y | CH2CH3 | H | XCH2CH3 | H |
| T-691 | Y | Y | CH2CH3 | H | XCH3 | H |
| T-692 | Y | Y | CH3 | Oxo | H | H |
| T-693 | Y | Y | CH3 | Oxo | H | Halo |
| T-694 | Y | Y | CH3 | Oxo | H | XCH2CH3 |
| T-695 | Y | Y | CH3 | Oxo | H | XCH3 |
| T-696 | Y | Y | CH3 | Oxo | Halo | H |
| T-697 | Y | Y | CH3 | Oxo | XCH2CH3 | H |
| T-698 | Y | Y | CH3 | Oxo | XCH3 | H |
| T-699 | Y | Y | CH3 | Oxo | H | H |
| T-700 | Y | Y | CH3 | Oxo | H | Halo |
| T-701 | Y | Y | CH3 | Oxo | H | XCH2CH3 |
| T-702 | Y | Y | CH3 | Oxo | H | XCH3 |
| T-703 | Y | Y | CH3 | Oxo | Halo | H |
| T-704 | Y | Y | CH3 | Oxo | XCH2CH3 | H |
| T-705 | Y | Y | CH3 | Oxo | XCH3 | H |
| T-706 | Y | Y | CH3 | Oxo | H | H |
| T-707 | Y | Y | CH3 | Oxo | H | Halo |
| T-708 | Y | Y | CH3 | Oxo | H | OCH2CH3 |
| T-709 | Y | Y | CH3 | Oxo | H | OCH3 |
| T-710 | Y | Y | CH3 | Oxo | Halo | H |
| T-711 | Y | Y | CH3 | Oxo | XCH2CH3 | H |
| T-712 | Y | Y | CH3 | Oxo | XCH3 | H |
| T-713 | Y | Y | CH3 | H | H | H |
| T-714 | Y | Y | CH3 | H | H | Halo |
| T-715 | Y | Y | CH3 | H | H | XCH2CH3 |
| T-716 | Y | Y | CH3 | H | H | XCH3 |
| T-717 | Y | Y | CH3 | H | Halo | H |
| T-718 | Y | Y | CH3 | H | XCH2CH3 | H |
| T-719 | Y | Y | CH3 | H | XCH3 | H |
| T-720 | Y | Y | CH3 | H | H | H |
| T-721 | Y | Y | CH3 | H | H | XCH2CH3 |
| T-722 | Y | Y | CH3 | H | H | XCH3 |
| T-723 | Y | Y | CH3 | H | XCH2CH3 | H |
| T-724 | Y | Y | CH3 | H | XCH3 | H |
| T-725 | Y | Y | CH3 | H | H | H |
| T-726 | Y | Y | CH3 | H | H | XCH2CH3 |
| T-727 | Y | Y | CH3 | H | H | XCH3 |
| T-728 | Y | Y | CH3 | H | XCH2CH3 | H |
| T-729 | Y | Y | CH3 | H | XCH3 | H |
| T-730 | Y | Y | H | Oxo | H | H |
| T-731 | Y | Y | H | Oxo | H | Halo |
| T-732 | Y | Y | H | Oxo | H | XCH2CH3 |
| T-733 | Y | Y | H | Oxo | H | XCH3 |
| T-734 | Y | Y | H | Oxo | Halo | H |
| T-735 | Y | Y | H | Oxo | XCH2CH3 | H |
| T-736 | Y | Y | H | Oxo | XCH3 | H |
| T-737 | Y | Y | H | Oxo | H | H |
| T-738 | Y | Y | H | Oxo | H | Halo |
| T-739 | Y | Y | H | Oxo | H | XCH2CH3 |
| T-740 | Y | Y | H | Oxo | H | XCH3 |
| T-741 | Y | Y | H | Oxo | Halo | H |
| T-742 | Y | Y | H | Oxo | XCH2CH3 | H |
| T-743 | Y | Y | H | Oxo | XCH3 | H |
| T-744 | Y | Y | H | Oxo | H | H |
| T-745 | Y | Y | H | Oxo | H | Halo |
| T-746 | Y | Y | H | Oxo | H | XCH2CH3 |
| T-747 | Y | Y | H | Oxo | H | XCH3 |
| T-748 | Y | Y | H | Oxo | Halo | H |
| T-749 | Y | Y | H | Oxo | XCH2CH3 | H |
| T-750 | Y | Y | H | Oxo | XCH3 | H |
| T-751 | Y | Y | H | H | H | H |
| T-752 | Y | Y | H | H | H | Halo |
| T-753 | Y | Y | H | H | H | XCH2CH3 |
| T-754 | Y | Y | H | H | H | XCH3 |
| T-755 | Y | Y | H | H | Halo | H |
| T-756 | Y | Y | H | H | XCH2CH3 | H |
| T-757 | Y | Y | H | H | XCH3 | H |
| T-758 | Y | Y | H | H | H | H |
| T-759 | Y | Y | H | H | H | XCH2CH3 |
| T-760 | Y | Y | H | H | H | XCH3 |
| T-761 | Y | Y | H | H | XCH2CH3 | H |
| T-762 | Y | Y | H | H | XCH3 | H |
| T-763 | Y | Y | H | H | H | H |
| T-764 | Y | Y | H | H | H | XCH2CH3 |
| T-765 | Y | Y | H | H | H | XCH3 |
| T-766 | Y | Y | H | H | XCH2CH3 | H |
| T-767 | Y | Y | H | H | XCH3 | H |

Structures of Formula I also exhibit ring-chain tautomerism (see e.g., Fulop et al. 1987. The Journal of Organic Chemistry, 52(17), 3821-3825; Johansen & Bundgaard. 1983. Journal of pharmaceutical sciences, 72(11), 1294-1298), for instance because of the reactivity of the enol form and of the enolate oxygen. Thus, preferred embodiments of Formula I include rings containing the nitrogen and oxygen, as in the following example, and in the structures of Table TV. Such ring structures act as prodrugs for the enol form of a beta-ketone indolizine and will readily form from the enol, for example when Y is joined to the nitrogen via an additional linker carbon that is in turn joined to the nitrogen with a double bond (e.g., where RN1 is =CHY, a five-membered ring such as below can be formed, such ring structures also falling within the scope of the invention).

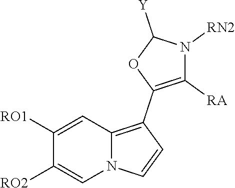

In some embodiments, an entactogen prodrug is provided. In some embodiments, the entactogen prodrug comprises at least one amino acid directly bonded to the entactogen. In some embodiments, the at least one amino acid is selected from Table I. In some embodiments, the at least one amino acid comprises at least two amino acids as a peptide. In some embodiments, the at least two amino acids is a valine bonded to a valine via a peptide bond. In some embodiments, the at least two amino acids is three glycines bonded via peptide bonds.

The following examples provide nonexhaustive illustrations of (A)ₙ contemplated in some embodiments. However, this molecule is used for illustrative purposes and other possibilities inherent in the definition of (A)ₙ are contemplated. Similarly, geometric and other isomers are also contemplated.

TABLE TIII
Exemplary Amino Acids and Peptides

| Example Number | Example (A)ₙ |
|---|---|
| A-1 | Gly |
| A-2 | Gly-Gly |
| A-3 | Gly-Gly-Gly |
| A-4 | Phe |
| A-5 | Trp |
| A-6 | Trp-Trp |
| A-7 | Met |
| A-8 | Met-Met |
| A-9 | Met-Met-Met |
| A-10 | N-acetyl-Met |
| A-11 | Cys |
| A-12 | Cys-Cys |
| A-13 | N-acetyl-Cys |
| A-14 | D-ribose-Cys |
| A-15 | His |
| A-16 | His-His |
| A-17 | Ala-His |
| A-18 | Arg |
| A-19 | Arg-Arg |
| A-20 | Tyr |
| A-21 | Tyr-Tyr |
| A-22 | Val |
| A-23 | Val-Val |

In some embodiments, an indolizine has one or more Y moieties comprising either R^{Z}Y¹ directly conjugated to the indolizine or R^{Z}Y¹ bonded to (A)ₙ which is in turn conjugated to the indolizine. Table TIV below provides nonexhaustive illustrations of R^{Z}Y¹ contemplated in some embodiments. However, these are intended for illustrative purposes and other possibilities inherent in the definition of R^{Z}Y¹ are contemplated. Similarly, geometric and other isomers are also contemplated.

TABLE TIV
Exemplary R^{Z}Y¹

| R^{Z}Y¹ | |
|---|---|
| Example | |
| Number | R^{Z}Y¹ |
| F-1 | 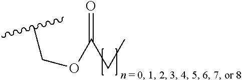 |
| F-2 | 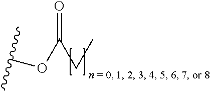 |
| F-3 | 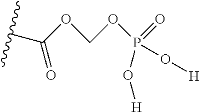 [SMILES: *C(=O)OCOP(=O)(O)O] |
| F-4 | 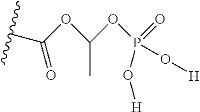 [SMILES: *C(=O)OC(C)OP(=O)(O)O] |
| F-5 | 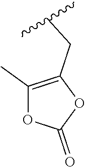 [SMILES: *Cc1oc(=O)oc1C] |
| F-6 | 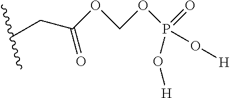 [SMILES: *CC(=O)OCOP(=O)(O)O] |
| F-7 | 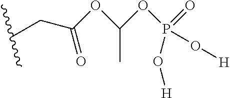 [SMILES: *CC(=O)OC(C)OP(=O)(O)O] |
| F-8 | 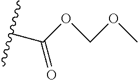 [SMILES: *C(=O)OCOC] |
| F-9 | 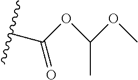 [SMILES: *C(=O)OC(C)OC] |
| F-10 | 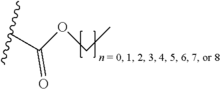 |
| F-11 | 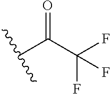 [SMILES: *C(=O)C(F)(F)F] |
| F-12 | 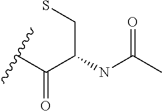 [SMILES: *C(=O)[C@H](CS)NC(C)=O] |
| F-13 | 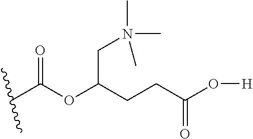 |
| F-14 | 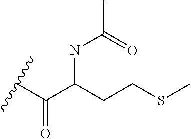 [SMILES: *C(=O)C(CCSC)NC(C)=O] |
| F-15 | 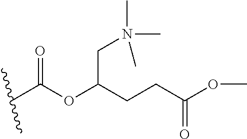 |
| F-16 | 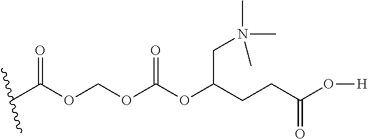 |
| F-17 | 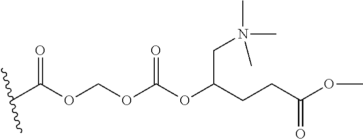 |
| F-18 | 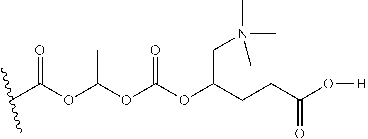 |
| F-19 | 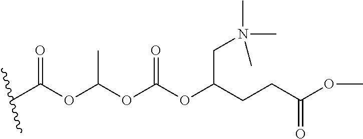 |
| F-20 | 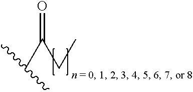 |
| F-21 | 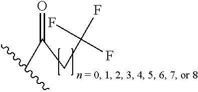 |
| F-22 | 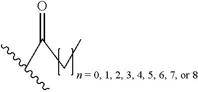 |
| F-23 | 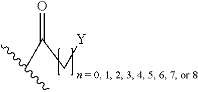 |
| F-24 | 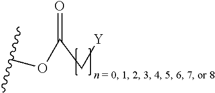 |

In some embodiments, the entactogen prodrug is one of the exemplary and nonexhaustive structures that can be formed by taking preferred compounds from Table TII and adding structures from Tables TIII and TIV to the amine of the preferred compound, in accordance with the description provided herein.

Thus, in some preferred embodiments, the entactogen prodrug is a preferred compound from Table TII, wherein one or both of RN1 and RN2 is Y (i.e., T-296 through T-767), and each Y is any of: (A)ₙ directly conjugated to the indolizine, R^{Z}Y¹ directly conjugated to the indolizine, or R^{Z}Y¹ bonded to (A)ₙ which is in turn conjugated to the indolizine, where such conjugation is at the amine nitrogen, and where any (A)ₙ is independently selected from the structures of Table TIII (i.e., A-1 through A-23), and any R^{Z}Y¹ is independently selected from the structures of Table TIV (i.e., F-1 through F-24).

In some such preferred embodiments, if more than one R^{Z}Y¹ is present, a first R^{Z}Y¹ is selected from the group consisting of Table TIV members and their derivatives, and any additional RY¹ is selected from the group consisting of Table TIV members and their derivatives, or is selected from the group consisting of hydrogen, hydroxy, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, and a C₂₃ bile acid moiety.

In preferred embodiments, pharmaceutical compositions are disclosed which comprise a compound of Formula I, either racemic, as pure enantiomers, or in some combination of enantiomers, and which may be in association with another active agent, as well as with a pharmaceutically acceptable carrier, diluent, or excipient.

In alternative embodiments the compound of Formula I or Formula II is of formula:

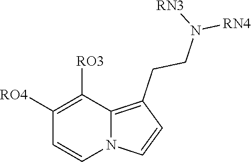

In some embodiments, each of R⁰³ and R⁰⁴ is independently selected from the group consisting of hydrogen, halogen, (C₁-C₆)alkyl, (C₁-C₄)alkenyl, (C₂-C₈)alkynyl, (C₁-C₄)alkoxy, O(C₁-C₆)alkyl, OC(═O)CH₃, hydroxy, CH₂X, CHX₂, CX₃, amino, alkylamino, alkylsulfinyl, alkylsulfonyl, OS(═O)₂CH₃, OS(═O)₂CH₂X, OS(═O)₂ CHX₂, OS(═O)₂CX₃, OP(═O)(OH)₂, OP(═O)(OR³)₂, and —SR³; wherein R³ is independently selected from the group consisting of hydrogen, hydroxy, CH₂X, CHX₂, CX₃, optionally substituted (C₁-C₆)alkyl, (C₂-C₈)alkenyl, (C₃-C₈)cycloalkyl, aryl, ═O(OH), and (═O)₂—OH; where X is halogen.

In some embodiments, each of RN3 and RN4 is independently selected from the group consisting of hydrogen, (C₁-C₆)alkyl, substituted (C₁-C₆)alkyl, CH₂CH═CH₂, hydroxy, and halogen.

These structures are effective for stimulating 5-HT_{2A} receptors, thereby altering CNS functioning, improving CNS disorders, decreasing inflammation, and improving inflammatory and metabolic disorders.

In certain embodiments, pharmaceutical compositions are disclosed that comprise a compound of Formula I, either racemic, as pure enantiomers, or in some combination of enantiomers, and which may be in association with another active agent, as well as with a pharmaceutically acceptable carrier, diluent, or excipient.

Exemplary, but non-exhaustive, certain embodiments of Formula I are given in Table Si below. Abbreviations are as previously defined, with the addition that X indicates either oxygen or sulfur.

TABLE S1
Additional Indolizine Compounds

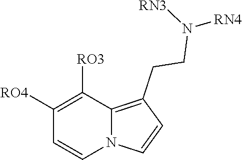

| | Position | | | |
|---|---|---|---|---|
| Compound # | RN3 | RN4 | RO3 | RO4 |
| S-1 | CH3 | CH3 | H | H |
| S-2 | CH3 | CH2CH3 | H | H |
| S-3 | CH3 | (CH2)3CH3 | H | H |
| S-4 | CH3 | CH(CH3)2 | H | H |
| S-5 | CH3 | CH2CH2CH3 | H | H |
| S-6 | CH3 | CH2CH═CH2 | H | H |
| S-7 | CH2CH3 | CH2CH3 | H | H |
| S-8 | CH2CH3 | (CH2)3CH3 | H | H |
| S-9 | CH2CH3 | CH(CH3)2 | H | H |
| S-10 | CH2CH3 | CH2CH2CH3 | H | H |
| S-11 | CH2CH3 | CH2CH═CH2 | H | H |
| S-12 | (CH2)3CH3 | (CH2)3CH3 | H | H |
| S-13 | (CH2)3CH3 | CH(CH3)2 | H | H |
| S-14 | (CH2)3CH3 | CH2CH2CH3 | H | H |
| S-15 | (CH2)3CH3 | CH2CH═CH2 | H | H |
| S-16 | CH(CH3)2 | CH(CH3)2 | H | H |
| S-17 | CH(CH3)2 | CH2CH2CH3 | H | H |
| S-18 | CH(CH3)2 | CH2CH═CH2 | H | H |
| S-19 | CH2CH2CH3 | CH2CH2CH3 | H | H |
| S-20 | CH2CH2CH3 | CH2CH═CH2 | H | H |
| S-21 | CH2CH═CH2 | CH2CH═CH2 | H | H |
| S-22 | CH3 | CH3 | OH | H |
| S-23 | CH3 | CH2CH3 | OH | H |
| S-24 | CH3 | (CH2)3CH3 | OH | H |
| S-25 | CH3 | CH(CH3)2 | OH | H |
| S-26 | CH3 | CH2CH2CH3 | OH | H |
| S-27 | CH3 | CH2CH═CH2 | OH | H |
| S-28 | CH2CH3 | CH2CH3 | OH | H |
| S-29 | CH2CH3 | (CH2)3CH3 | OH | H |
| S-30 | CH2CH3 | CH(CH3)2 | OH | H |
| S-31 | CH2CH3 | CH2CH2CH3 | OH | H |
| S-32 | CH2CH3 | CH2CH═CH2 | OH | H |
| S-33 | (CH2)3CH3 | (CH2)3CH3 | OH | H |
| S-34 | (CH2)3CH3 | CH(CH3)2 | OH | H |
| S-35 | (CH2)3CH3 | CH2CH2CH3 | OH | H |
| S-36 | (CH2)3CH3 | CH2CH═CH2 | OH | H |
| S-37 | CH(CH3)2 | CH(CH3)2 | OH | H |
| S-38 | CH(CH3)2 | CH2CH2CH3 | OH | H |
| S-39 | CH(CH3)2 | CH2CH═CH2 | OH | H |
| S-40 | CH2CH2CH3 | CH2CH2CH3 | OH | H |
| S-41 | CH2CH2CH3 | CH2CH═CH2 | OH | H |
| S-42 | CH2CH═CH2 | CH2CH═CH2 | OH | H |
| S-43 | CH3 | CH3 | OC(═O)CH3 | H |
| S-44 | CH3 | CH2CH3 | OC(═O)CH3 | H |
| S-45 | CH3 | (CH2)3CH3 | OC(═O)CH3 | H |
| S-46 | CH3 | CH(CH3)2 | OC(═O)CH3 | H |
| S-47 | CH3 | CH2CH2CH3 | OC(═O)CH3 | H |
| S-48 | CH3 | CH2CH═CH2 | OC(═O)CH3 | H |
| S-49 | CH2CH3 | CH2CH3 | OC(═O)CH3 | H |
| S-50 | CH2CH3 | (CH2)3CH3 | OC(═O)CH3 | H |
| S-51 | CH2CH3 | CH(CH3)2 | OC(═O)CH3 | H |
| S-52 | CH2CH3 | CH2CH2CH3 | OC(═O)CH3 | H |
| S-53 | CH2CH3 | CH2CH═CH2 | OC(═O)CH3 | H |
| S-54 | (CH2)3CH3 | (CH2)3CH3 | OC(═O)CH3 | H |
| S-55 | (CH2)3CH3 | CH(CH3)2 | OC(═O)CH3 | H |
| S-56 | (CH2)3CH3 | CH2CH2CH3 | OC(═O)CH3 | H |
| S-57 | (CH2)3CH3 | CH2CH═CH2 | OC(═O)CH3 | H |
| S-58 | CH(CH3)2 | CH(CH3)2 | OC(═O)CH3 | H |
| S-59 | CH(CH3)2 | CH2CH2CH3 | OC(═O)CH3 | H |
| S-60 | CH(CH3)2 | CH2CH═CH2 | OC(═O)CH3 | H |
| S-61 | CH2CH2CH3 | CH2CH2CH3 | OC(═O)CH3 | H |
| S-62 | CH2CH2CH3 | CH2CH═CH2 | OC(═O)CH3 | H |
| S-63 | CH2CH═CH2 | CH2CH═CH2 | OC(═O)CH3 | H |
| S-64 | CH3 | CH3 | OP(═O)(OH2) | H |
| S-65 | CH3 | CH2CH3 | OP(═O)(OH2) | H |
| S-66 | CH3 | (CH2)3CH3 | OP(═O)(OH2) | H |
| S-67 | CH3 | CH(CH3)2 | OP(═O)(OH2) | H |
| S-68 | CH3 | CH2CH2CH3 | OP(═O)(OH2) | H |
| S-69 | CH3 | CH2CH═CH2 | OP(═O)(OH2) | H |
| S-70 | CH2CH3 | CH2CH3 | OP(═O)(OH2) | H |
| S-71 | CH2CH3 | (CH2)3CH3 | OP(═O)(OH2) | H |
| S-72 | CH2CH3 | CH(CH3)2 | OP(═O)(OH2) | H |
| S-73 | CH2CH3 | CH2CH2CH3 | OP(═O)(OH2) | H |
| S-74 | CH2CH3 | CH2CH═CH2 | OP(═O)(OH2) | H |
| S-75 | (CH2)3CH3 | (CH2)3CH3 | OP(═O)(OH2) | H |
| S-76 | (CH2)3CH3 | CH(CH3)2 | OP(═O)(OH2) | H |
| S-77 | (CH2)3CH3 | CH2CH2CH3 | OP(═O)(OH2) | H |
| S-78 | (CH2)3CH3 | CH2CH═CH2 | OP(═O)(OH2) | H |
| S-79 | CH(CH3)2 | CH(CH3)2 | OP(═O)(OH2) | H |
| S-80 | CH(CH3)2 | CH2CH2CH3 | OP(═O)(OH2) | H |
| S-81 | CH(CH3)2 | CH2CH═CH2 | OP(═O)(OH2) | H |
| S-82 | CH2CH2CH3 | CH2CH2CH3 | OP(═O)(OH2) | H |
| S-83 | CH2CH2CH3 | CH2CH═CH2 | OP(═O)(OH2) | H |
| S-84 | CH2CH═CH2 | CH2CH═CH2 | OP(═O)(OH2) | H |
| S-85 | CH3 | CH3 | CH3 | H |
| S-86 | CH3 | CH2CH3 | CH3 | H |
| S-87 | CH3 | (CH2)3CH3 | CH3 | H |
| S-88 | CH3 | CH(CH3)2 | CH3 | H |
| S-89 | CH3 | CH2CH2CH3 | CH3 | H |
| S-90 | CH3 | CH2CH═CH2 | CH3 | H |
| S-91 | CH2CH3 | CH2CH3 | CH3 | H |
| S-92 | CH2CH3 | (CH2)3CH3 | CH3 | H |
| S-93 | CH2CH3 | CH(CH3)2 | CH3 | H |
| S-94 | CH2CH3 | CH2CH2CH3 | CH3 | H |
| S-95 | CH2CH3 | CH2CH═CH2 | CH3 | H |
| S-96 | (CH2)3CH3 | (CH2)3CH3 | CH3 | H |
| S-97 | (CH2)3CH3 | CH(CH3)2 | CH3 | H |
| S-98 | (CH2)3CH3 | CH2CH2CH3 | CH3 | H |
| S-99 | (CH2)3CH3 | CH2CH═CH2 | CH3 | H |
| S-100 | CH(CH3)2 | CH(CH3)2 | CH3 | H |
| S-101 | CH(CH3)2 | CH2CH2CH3 | CH3 | H |
| S-102 | CH(CH3)2 | CH2CH═CH2 | CH3 | H |
| S-103 | CH2CH2CH3 | CH2CH2CH3 | CH3 | H |
| S-104 | CH2CH2CH3 | CH2CH═CH2 | CH3 | H |
| S-105 | CH2CH═CH2 | CH2CH═CH2 | CH3 | H |
| S-106 | CH3 | CH3 | Br | H |
| S-107 | CH3 | CH2CH3 | Br | H |
| S-108 | CH3 | (CH2)3CH3 | Br | H |
| S-109 | CH3 | CH(CH3)2 | Br | H |
| S-110 | CH3 | CH2CH2CH3 | Br | H |
| S-111 | CH3 | CH2CH═CH2 | Br | H |
| S-112 | CH2CH3 | CH2CH3 | Br | H |
| S-113 | CH2CH3 | (CH2)3CH3 | Br | H |
| S-114 | CH2CH3 | CH(CH3)2 | Br | H |
| S-115 | CH2CH3 | CH2CH2CH3 | Br | H |
| S-116 | CH2CH3 | CH2CH═CH2 | Br | H |
| S-117 | (CH2)3CH3 | (CH2)3CH3 | Br | H |
| S-118 | (CH2)3CH3 | CH(CH3)2 | Br | H |
| S-119 | (CH2)3CH3 | CH2CH2CH3 | Br | H |
| S-120 | (CH2)3CH3 | CH2CH═CH2 | Br | H |
| S-121 | CH(CH3)2 | CH(CH3)2 | Br | H |
| S-122 | CH(CH3)2 | CH2CH2CH3 | Br | H |
| S-123 | CH(CH3)2 | CH2CH═CH2 | Br | H |
| S-124 | CH2CH2CH3 | CH2CH2CH3 | Br | H |
| S-125 | CH2CH2CH3 | CH2CH═CH2 | Br | H |
| S-126 | CH2CH═CH2 | CH2CH═CH2 | Br | H |
| S-127 | CH3 | CH3 | Cl | H |
| S-128 | CH3 | CH2CH3 | Cl | H |
| S-129 | CH3 | (CH2)3CH3 | Cl | H |
| S-130 | CH3 | CH(CH3)2 | Cl | H |
| S-131 | CH3 | CH2CH2CH3 | Cl | H |
| S-132 | CH3 | CH2CH═CH2 | Cl | H |
| S-133 | CH2CH3 | CH2CH3 | Cl | H |
| S-134 | CH2CH3 | (CH2)3CH3 | Cl | H |
| S-135 | CH2CH3 | CH(CH3)2 | Cl | H |
| S-136 | CH2CH3 | CH2CH2CH3 | Cl | H |
| S-137 | CH2CH3 | CH2CH═CH2 | Cl | H |
| S-138 | (CH2)3CH3 | (CH2)3CH3 | Cl | H |
| S-139 | (CH2)3CH3 | CH(CH3)2 | Cl | H |
| S-140 | (CH2)3CH3 | CH2CH2CH3 | Cl | H |
| S-141 | (CH2)3CH3 | CH2CH═CH2 | Cl | H |
| S-142 | CH(CH3)2 | CH(CH3)2 | Cl | H |
| S-143 | CH(CH3)2 | CH2CH2CH3 | Cl | H |
| S-144 | CH(CH3)2 | CH2CH═CH2 | Cl | H |
| S-145 | CH2CH2CH3 | CH2CH2CH3 | Cl | H |
| S-146 | CH2CH2CH3 | CH2CH═CH2 | Cl | H |
| S-147 | CH2CH═CH2 | CH2CH═CH2 | Cl | H |
| S-148 | CH3 | CH3 | F | H |
| S-149 | CH3 | CH2CH3 | F | H |
| S-150 | CH3 | (CH2)3CH3 | F | H |
| S-151 | CH3 | CH(CH3)2 | F | H |
| S-152 | CH3 | CH2CH2CH3 | F | H |
| S-153 | CH3 | CH2CH═CH2 | F | H |
| S-154 | CH2CH3 | CH2CH3 | F | H |
| S-155 | CH2CH3 | (CH2)3CH3 | F | H |
| S-156 | CH2CH3 | CH(CH3)2 | F | H |
| S-157 | CH2CH3 | CH2CH2CH3 | F | H |
| S-158 | CH2CH3 | CH2CH═CH2 | F | H |
| S-159 | (CH2)3CH3 | (CH2)3CH3 | F | H |
| S-160 | (CH2)3CH3 | CH(CH3)2 | F | H |
| S-161 | (CH2)3CH3 | CH2CH2CH3 | F | H |
| S-162 | (CH2)3CH3 | CH2CH═CH2 | F | H |
| S-163 | CH(CH3)2 | CH(CH3)2 | F | H |
| S-164 | CH(CH3)2 | CH2CH2CH3 | F | H |
| S-165 | CH(CH3)2 | CH2CH═CH2 | F | H |
| S-166 | CH2CH2CH3 | CH2CH2CH3 | F | H |
| S-167 | CH2CH2CH3 | CH2CH═CH2 | F | H |
| S-168 | CH2CH═CH2 | CH2CH═CH2 | F | H |
| S-169 | CH3 | CH3 | I | H |
| S-170 | CH3 | CH2CH3 | I | H |
| S-171 | CH3 | (CH2)3CH3 | I | H |
| S-172 | CH3 | CH(CH3)2 | I | H |
| S-173 | CH3 | CH2CH2CH3 | I | H |
| S-174 | CH3 | CH2CH═CH2 | I | H |
| S-175 | CH2CH3 | CH2CH3 | I | H |
| S-176 | CH2CH3 | (CH2)3CH3 | I | H |
| S-177 | CH2CH3 | CH(CH3)2 | I | H |
| S-178 | CH2CH3 | CH2CH2CH3 | I | H |
| S-179 | CH2CH3 | CH2CH═CH2 | I | H |
| S-180 | (CH2)3CH3 | (CH2)3CH3 | I | H |
| S-181 | (CH2)3CH3 | CH(CH3)2 | I | H |
| S-182 | (CH2)3CH3 | CH2CH2CH3 | I | H |
| S-183 | (CH2)3CH3 | CH2CH═CH2 | I | H |
| S-184 | CH(CH3)2 | CH(CH3)2 | I | H |
| S-185 | CH(CH3)2 | CH2CH2CH3 | I | H |
| S-186 | CH(CH3)2 | CH2CH═CH2 | I | H |
| S-187 | CH2CH2CH3 | CH2CH2CH3 | I | H |
| S-188 | CH2CH2CH3 | CH2CH═CH2 | I | H |
| S-189 | CH2CH═CH2 | CH2CH═CH2 | I | H |
| S-190 | CH3 | CH3 | H | OH |
| S-191 | CH3 | CH2CH3 | H | OH |
| S-192 | CH3 | (CH2)3CH3 | H | OH |
| S-193 | CH3 | CH(CH3)2 | H | OH |
| S-194 | CH3 | CH2CH2CH3 | H | OH |
| S-195 | CH3 | CH2CH═CH2 | H | OH |
| S-196 | CH2CH3 | CH2CH3 | H | OH |
| S-197 | CH2CH3 | (CH2)3CH3 | H | OH |
| S-198 | CH2CH3 | CH(CH3)2 | H | OH |
| S-199 | CH2CH3 | CH2CH2CH3 | H | OH |
| S-200 | CH2CH3 | CH2CH═CH2 | H | OH |
| S-201 | (CH2)3CH3 | (CH2)3CH3 | H | OH |
| S-202 | (CH2)3CH3 | CH(CH3)2 | H | OH |
| S-203 | (CH2)3CH3 | CH2CH2CH3 | H | OH |
| S-204 | (CH2)3CH3 | CH2CH═CH2 | H | OH |
| S-205 | CH(CH3)2 | CH(CH3)2 | H | OH |
| S-206 | CH(CH3)2 | CH2CH2CH3 | H | OH |
| S-207 | CH(CH3)2 | CH2CH═CH2 | H | OH |
| S-208 | CH2CH2CH3 | CH2CH2CH3 | H | OH |
| S-209 | CH2CH2CH3 | CH2CH═CH2 | H | OH |
| S-210 | CH2CH═CH2 | CH2CH═CH2 | H | OH |
| S-211 | CH3 | CH3 | H | OC(═O)CH3 |
| S-212 | CH3 | CH2CH3 | H | OC(═O)CH3 |
| S-213 | CH3 | (CH2)3CH3 | H | OC(═O)CH3 |
| S-214 | CH3 | CH(CH3)2 | H | OC(═O)CH3 |
| S-215 | CH3 | CH2CH2CH3 | H | OC(═O)CH3 |
| S-216 | CH3 | CH2CH═CH2 | H | OC(═O)CH3 |
| S-217 | CH2CH3 | CH2CH3 | H | OC(═O)CH3 |
| S-218 | CH2CH3 | (CH2)3CH3 | H | OC(═O)CH3 |
| S-219 | CH2CH3 | CH(CH3)2 | H | OC(═O)CH3 |
| S-220 | CH2CH3 | CH2CH2CH3 | H | OC(═O)CH3 |
| S-221 | CH2CH3 | CH2CH═CH2 | H | OC(═O)CH3 |
| S-222 | (CH2)3CH3 | (CH2)3CH3 | H | OC(═O)CH3 |
| S-223 | (CH2)3CH3 | CH(CH3)2 | H | OC(═O)CH3 |
| S-224 | (CH2)3CH3 | CH2CH2CH3 | H | OC(═O)CH3 |
| S-225 | (CH2)3CH3 | CH2CH═CH2 | H | OC(═O)CH3 |
| S-226 | CH(CH3)2 | CH(CH3)2 | H | OC(═O)CH3 |
| S-227 | CH(CH3)2 | CH2CH2CH3 | H | OC(═O)CH3 |
| S-228 | CH(CH3)2 | CH2CH═CH2 | H | OC(═O)CH3 |
| S-229 | CH2CH2CH3 | CH2CH2CH3 | H | OC(═O)CH3 |
| S-230 | CH2CH2CH3 | CH2CH═CH2 | H | OC(═O)CH3 |
| S-231 | CH2CH═CH2 | CH2CH═CH2 | H | OC(═O)CH3 |
| S-232 | CH3 | CH3 | H | OP(═O)(OH2) |
| S-233 | CH3 | CH2CH3 | H | OP(═O)(OH2) |
| S-234 | CH3 | (CH2)3CH3 | H | OP(═O)(OH2) |
| S-235 | CH3 | CH(CH3)2 | H | OP(═O)(OH2) |
| S-236 | CH3 | CH2CH2CH3 | H | OP(═O)(OH2) |
| S-237 | CH3 | CH2CH═CH2 | H | OP(═O)(OH2) |
| S-238 | CH2CH3 | CH2CH3 | H | OP(═O)(OH2) |
| S-239 | CH2CH3 | (CH2)3CH3 | H | OP(═O)(OH2) |
| S-240 | CH2CH3 | CH(CH3)2 | H | OP(═O)(OH2) |
| S-241 | CH2CH3 | CH2CH2CH3 | H | OP(═O)(OH2) |
| S-242 | CH2CH3 | CH2CH═CH2 | H | OP(═O)(OH2) |
| S-243 | (CH2)3CH3 | (CH2)3CH3 | H | OP(═O)(OH2) |
| S-244 | (CH2)3CH3 | CH(CH3)2 | H | OP(═O)(OH2) |
| S-245 | (CH2)3CH3 | CH2CH2CH3 | H | OP(═O)(OH2) |
| S-246 | (CH2)3CH3 | CH2CH═CH2 | H | OP(═O)(OH2) |
| S-247 | CH(CH3)2 | CH(CH3)2 | H | OP(═O)(OH2) |
| S-248 | CH(CH3)2 | CH2CH2CH3 | H | OP(═O)(OH2) |
| S-249 | CH(CH3)2 | CH2CH═CH2 | H | OP(═O)(OH2) |
| S-250 | CH2CH2CH3 | CH2CH2CH3 | H | OP(═O)(OH2) |
| S-251 | CH2CH2CH3 | CH2CH═CH2 | H | OP(═O)(OH2) |
| S-252 | CH2CH═CH2 | CH2CH═CH2 | H | OP(═O)(OH2) |
| S-253 | CH3 | CH3 | H | CH3 |
| S-254 | CH3 | CH2CH3 | H | CH3 |
| S-255 | CH3 | (CH2)3CH3 | H | CH3 |
| S-256 | CH3 | CH(CH3)2 | H | CH3 |
| S-257 | CH3 | CH2CH2CH3 | H | CH3 |
| S-258 | CH3 | CH2CH═CH2 | H | CH3 |
| S-259 | CH2CH3 | CH2CH3 | H | CH3 |
| S-260 | CH2CH3 | (CH2)3CH3 | H | CH3 |
| S-261 | CH2CH3 | CH(CH3)2 | H | CH3 |
| S-262 | CH2CH3 | CH2CH2CH3 | H | CH3 |
| S-263 | CH2CH3 | CH2CH═CH2 | H | CH3 |
| S-264 | (CH2)3CH3 | (CH2)3CH3 | H | CH3 |
| S-265 | (CH2)3CH3 | CH(CH3)2 | H | CH3 |
| S-266 | (CH2)3CH3 | CH2CH2CH3 | H | CH3 |
| S-267 | (CH2)3CH3 | CH2CH═CH2 | H | CH3 |
| S-268 | CH(CH3)2 | CH(CH3)2 | H | CH3 |
| S-269 | CH(CH3)2 | CH2CH2CH3 | H | CH3 |
| S-270 | CH(CH3)2 | CH2CH═CH2 | H | CH3 |
| S-271 | CH2CH2CH3 | CH2CH2CH3 | H | CH3 |
| S-272 | CH2CH2CH3 | CH2CH═CH2 | H | CH3 |
| S-273 | CH2CH═CH2 | CH2CH═CH2 | H | CH3 |
| S-274 | CH3 | CH3 | H | Br |
| S-275 | CH3 | CH2CH3 | H | Br |
| S-276 | CH3 | (CH2)3CH3 | H | Br |
| S-277 | CH3 | CH(CH3)2 | H | Br |
| S-278 | CH3 | CH2CH2CH3 | H | Br |
| S-279 | CH3 | CH2CH═CH2 | H | Br |
| S-280 | CH2CH3 | CH2CH3 | H | Br |
| S-281 | CH2CH3 | (CH2)3CH3 | H | Br |
| S-282 | CH2CH3 | CH(CH3)2 | H | Br |
| S-283 | CH2CH3 | CH2CH2CH3 | H | Br |
| S-284 | CH2CH3 | CH2CH═CH2 | H | Br |
| S-285 | (CH2)3CH3 | (CH2)3CH3 | H | Br |
| S-286 | (CH2)3CH3 | CH(CH3)2 | H | Br |
| S-287 | (CH2)3CH3 | CH2CH2CH3 | H | Br |
| S-288 | (CH2)3CH3 | CH2CH═CH2 | H | Br |
| S-289 | CH(CH3)2 | CH(CH3)2 | H | Br |
| S-290 | CH(CH3)2 | CH2CH2CH3 | H | Br |
| S-291 | CH(CH3)2 | CH2CH═CH2 | H | Br |
| S-292 | CH2CH2CH3 | CH2CH2CH3 | H | Br |
| S-293 | CH2CH2CH3 | CH2CH═CH2 | H | Br |
| S-294 | CH2CH═CH2 | CH2CH═CH2 | H | Br |
| S-295 | CH3 | CH3 | H | Cl |
| S-296 | CH3 | CH2CH3 | H | Cl |
| S-297 | CH3 | (CH2)3CH3 | H | Cl |
| S-298 | CH3 | CH(CH3)2 | H | Cl |
| S-299 | CH3 | CH2CH2CH3 | H | Cl |
| S-300 | CH3 | CH2CH═CH2 | H | Cl |
| S-301 | CH2CH3 | CH2CH3 | H | Cl |
| S-302 | CH2CH3 | (CH2)3CH3 | H | Cl |
| S-303 | CH2CH3 | CH(CH3)2 | H | Cl |
| S-304 | CH2CH3 | CH2CH2CH3 | H | Cl |
| S-305 | CH2CH3 | CH2CH═CH2 | H | Cl |
| S-306 | (CH2)3CH3 | (CH2)3CH3 | H | Cl |
| S-307 | (CH2)3CH3 | CH(CH3)2 | H | Cl |
| S-308 | (CH2)3CH3 | CH2CH2CH3 | H | Cl |
| S-309 | (CH2)3CH3 | CH2CH═CH2 | H | Cl |
| S-310 | CH(CH3)2 | CH(CH3)2 | H | Cl |
| S-311 | CH(CH3)2 | CH2CH2CH3 | H | Cl |
| S-312 | CH(CH3)2 | CH2CH═CH2 | H | Cl |
| S-313 | CH2CH2CH3 | CH2CH2CH3 | H | Cl |
| S-314 | CH2CH2CH3 | CH2CH═CH2 | H | Cl |
| S-315 | CH2CH═CH2 | CH2CH═CH2 | H | Cl |
| S-316 | CH3 | CH3 | H | F |
| S-317 | CH3 | CH2CH3 | H | F |
| S-318 | CH3 | (CH2)3CH3 | H | F |
| S-319 | CH3 | CH(CH3)2 | H | F |
| S-320 | CH3 | CH2CH2CH3 | H | F |
| S-321 | CH3 | CH2CH═CH2 | H | F |
| S-322 | CH2CH3 | CH2CH3 | H | F |
| S-323 | CH2CH3 | (CH2)3CH3 | H | F |
| S-324 | CH2CH3 | CH(CH3)2 | H | F |
| S-325 | CH2CH3 | CH2CH2CH3 | H | F |
| S-326 | CH2CH3 | CH2CH═CH2 | H | F |
| S-327 | (CH2)3CH3 | (CH2)3CH3 | H | F |
| S-328 | (CH2)3CH3 | CH(CH3)2 | H | F |
| S-329 | (CH2)3CH3 | CH2CH2CH3 | H | F |
| S-330 | (CH2)3CH3 | CH2CH═CH2 | H | F |
| S-331 | CH(CH3)2 | CH(CH3)2 | H | F |
| S-332 | CH(CH3)2 | CH2CH2CH3 | H | F |
| S-333 | CH(CH3)2 | CH2CH═CH2 | H | F |
| S-334 | CH2CH2CH3 | CH2CH2CH3 | H | F |
| S-335 | CH2CH2CH3 | CH2CH═CH2 | H | F |
| S-336 | CH2CH═CH2 | CH2CH═CH2 | H | F |
| S-337 | CH3 | CH3 | H | I |
| S-338 | CH3 | CH2CH3 | H | I |
| S-339 | CH3 | (CH2)3CH3 | H | I |
| S-340 | CH3 | CH(CH3)2 | H | I |
| S-341 | CH3 | CH2CH2CH3 | H | I |
| S-342 | CH3 | CH2CH═CH2 | H | I |
| S-343 | CH2CH3 | CH2CH3 | H | I |
| S-344 | CH2CH3 | (CH2)3CH3 | H | I |
| S-345 | CH2CH3 | CH(CH3)2 | H | I |
| S-346 | CH2CH3 | CH2CH2CH3 | H | I |
| S-347 | CH2CH3v | CH2CH═CH2 | H | I |
| S-348 | (CH2)3CH3 | (CH2)3CH3 | H | I |
| S-349 | (CH2)3CH3 | CH(CH3)2 | H | I |
| S-350 | (CH2)3CH3 | CH2CH2CH3 | H | I |
| S-351 | (CH2)3CH3 | CH2CH═CH2 | H | I |
| S-352 | CH(CH3)2 | CH(CH3)2 | H | I |
| S-353 | CH(CH3)2 | CH2CH2CH3 | H | I |
| S-354 | CH(CH3)2 | CH2CH═CH2 | H | I |
| S-355 | CH2CH2CH3 | CH2CH2CH3 | H | I |
| S-356 | CH2CH2CH3 | CH2CH═CH2 | H | I |
| S-357 | CH2CH═CH2 | CH2CH═CH2 | H | I |

### METHODS OF TREATMENT

The present invention also provides methods for modulating the CNS in mammals or treating an inflammatory or metabolic disease by administering a pharmaceutically effective amount of a compound of the present invention to a host in need thereof, for example a human.

A compound of the present invention can be used in methods for treating a variety of diseases or disorders linked to inadequate functioning of neurotransmission in the CNS of mammals. Included among such disorders are depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders. Also included among such disorders are primary or secondary headaches. Also included among such disorders are seizure disorders, such as epilepsy disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making. Any of these methods can employ a compound of the present invention, either as a racemate, an individual enantiomer, an enantiomerically enriched mixture, or with deuterium-substitution, or more than one of these in combination. When referring to compounds herein, the terms accordingly should be understood to refer not only to the racemates of those structures, but also to single enantiomers, enantiomerically enriched mixtures, and structures with deuterium-substitution(s) or other modifications, as the context indicates and supports.

This invention also provides the use of a compound, pure enantiomer or enantiomerically enriched mixture of Formula I for the manufacture of a medicament for the treatment of maladaptive response to perceived psychological threats. Additionally, this invention provides a pharmaceutical formulation adapted for the treatment of maladaptive response to perceived psychological threats containing a compound, pure enantiomer or enantiomerically enriched mixture of Formula I. Furthermore, this invention includes a method for the treatment of maladaptive response to perceived psychological threats that comprises administering an effective amount of a compound, pure enantiomer or enantiomerically enriched mixture of Formula I, given either in the context of psychotherapy or as a stand-alone treatment.

In some embodiments, the compounds and compositions of the invention are used to promote neuronal survival, neurogenesis, and neuroplasticity, and to treat neuropsychiatric and neurodegenerative conditions or disorders that benefit by such promotion. For example, in some embodiments, a compound or composition of the invention is used in a method of treating stroke, traumatic brain injury (TBI), or other acute injury or condition. In some embodiments, a compound or composition of the invention is used in a method to treat a chronic pathology or condition such as a myasthenia syndrome, multiple sclerosis, epilepsy, schizophrenia, dementia, memory loss, Alzheimer's disease, Parkinson's disease, and Huntington's disease. In other embodiments are methods of administering a compound or composition of the invention to accelerate learning, including in a healthy subject.

In general, it will be readily appreciated that methods of using the compounds and compositions to promote neural plasticity will be useful in treating diseases, disorders, and conditions where the etiology or pathology is correlated with, relates to, or is caused by alterations, changes, or reductions in, or other abnormalities involving, neuroplasticity, including where those abnormalities involving neuroplasticity are associated with inflammation. It will be understood that such treatment may involve any abatement, remission, or diminishment of symptoms; any rendering of the injury, pathology, or condition more tolerable to a patient; any slowing of the rate of degeneration or decline; any rendering of the final point of degeneration less debilitating; and any other improvements to a patient's physical or mental well-being.

In addition to treating various diseases and disorders, the employed methods of stimulating 5-HT_{2A} receptors can be used to improve CNS functioning in non-disease states, such as increasing creativity and aiding decision-making.

Any of these methods can employ a compound of Formula I, either as a racemate, an individual enantiomer, an enantiomerically enriched mixture, or a free base or salt thereof, or a derivative, analogue, or prodrug thereof, or more than one of these in combination. When referring to “Formula I” herein, or when otherwise referring to a “structure” or “compound” of the invention, the term accordingly should be understood to refer not only to the racemates of the compound(s) having that structure, but also to single enantiomers, enantiomerically enriched mixtures, and compounds with substitution(s) or other modifications, as well as other free bases, salts, derivatives, analogues, prodrugs, and the like, as context indicates and supports.

### Methods to Treat Headache Disorders

In certain embodiments, a method of treating a patient with primary or secondary headaches is provided, comprising administering an effective amount of a compound, pure enantiomer, or enantiomerically enriched mixture of a compound of the present invention, or a pharmaceutically acceptable salt thereof. While administration of such a compound or preparation usually occurs as needed (i.e., at the onset of headache or prodromal syndrome), in some cases, administration may be monthly, weekly, daily, twice daily, or a similar interval to achieve adequate symptom relief. Because some headache disorders have cyclical or other patterns to their occurrence, it is contemplated that medication could be taken using a personalized schedule that is based on use of an algorithm to predict the onset of headache. Administration may be oral, but other routes including buccal, sublingual, inhaled, and other parenteral routes are contemplated. In some embodiments, routes with fast onset of therapeutic effects are considered advantageous.

As used herein, primary headaches include, but are not limited to migraine, migraine signs and symptoms without cephalgia, tension-type headaches, cluster headaches and other trigeminal autonomic cephalalgias, new daily persistent headache, hypnic headaches, stabbing headaches, and other primary headache disorders. Secondary headaches referred to herein can refer to those due to trauma or injury, cranial or cervical vascular disorder, non-vascular intracranial disorder, headaches due to substance use or substance withdrawal, and other secondary headaches.

In certain embodiments a compound of the present invention is used to treat a migraine, headache, or cluster headache. Non-limiting examples of migraines include migraine without aura, migraine with aura, chronic migraine, abdominal migraine, acephalgic migraine, silent migraine, migraine with brainstem aura, hemiplegic migraine, retinal migraine, and status migrainosus.

Improvement in headache disorders is typically indicated by improvements (lessening of severity or frequency) in pain, nausea, photophobia, and phonophobia and the accompanying disruption of normal activities (Loder and Burch 2012. Cephalalgia, 32(3), pp. 179-182; Vingen et al. 1998. Cephalalgia, 18(5), pp. 250-256; Sauro et al. 2010. Headache: The Journal of Head and Face Pain, 50(3), pp. 383-395). As such, it is anticipated that the methods of treatment disclosed within will result in improvements of one or more of these signs and symptoms.

### Methods to Treat Seizure Disorders

In certain embodiments, a method of treating a patient with a seizure disorder is provided, comprising administering an effective amount of a compound, pure enantiomer, or enantiomerically enriched mixture of a compound of the present invention, or a pharmaceutically acceptable salt thereof. While administration of such a compound or preparation usually occurs daily or twice daily, in some cases, administration may be as needed (i.e., at the onset of a prodromal syndrome). Because some seizure disorders have cyclical or other patterns to their occurrence, it is contemplated that medication could be taken using a personalized schedule that is based on use of an algorithm to predict the onset of seizures. Administration may be oral, but other routes including buccal, sublingual, inhaled, and other parenteral routes are contemplated. In some embodiments, routes with fast onset of therapeutic effects are considered advantageous.

As used herein, seizure disorders include, but are not limited to focal aware seizures, focal impaired awareness seizures, bilateral tonic-clonic seizures, absence seizures, atyptical absence seizures, tonic-clonic seizures, atonic seizures, clonic seizures, tonic seizures, myoclonic seizures, gelastic seizures, and dacrystic seizures. In certain embodiments a compound of the present invention is used to treat epilepsy. In certain embodiments, the form of epilepsy is severe myoclonic epilepsy of infancy.

Improvement in seizure disorders is understood to mean a decrease in frequency or severity (or both frequency and severity), which can be assessed with both self-report and objective measures (e.g., EEG, including wearable devices) (Cramer and French 2001. Epilepsia, 42(1), pp. 119-129; Karoly, Goldenholz, and Cook 2018. Current opinion in neurology, 31(2), pp. 162-168). As such, it is anticipated that the methods of treatment disclosed within will result in improvements of one or more of these signs and symptoms.

### Non-Limiting Examples of Pharmacotherapeutic Counseling Use

Psychotherapy, cognitive enhancement, or life coaching conducted with a compound or pharmaceutically acceptable salt as described herein employed as an adjunct (hereafter, “pharmacotherapy” or “pharmacotherapy counseling”) is typically conducted in widely spaced sessions with one, two, or rarely three or more administrations of an entactogen per session. These sessions can be as frequent as weekly but are more often approximately monthly or even less frequently. In most cases, a small number of pharmacotherapy counseling sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with an effective amount of a compound of the present invention or an effective amount of enantiomerically enriched compound of the present invention or a pharmaceutically acceptable salt thereof.

The following sections provide detailed examples of pharmacotherapy. While common procedures are described, these are intended as illustrative, non-restrictive examples. It is anticipated that the prescribing physician and therapy team may wish to specify different procedures than those described here based on their clinical judgment concerning the needs of the patient.

The example methods of treatment can also be modified with very minor changes to treat multiple patients at once, including couples or families. Hence, “patient” should be understood to mean one or more individuals.

Use of a Compound or Composition of the Present Invention in Conjunction with Conventional Psychotherapy or Coaching

In certain embodiments, the use of a described indolizine compound or composition of the present invention as pharmacotherapy is integrated into the patient's ongoing psychotherapy or coaching (hereafter abbreviated as “psychotherapy”). If a patient in need of the pharmacotherapy is not in ongoing psychotherapy, then psychotherapy may be initiated and the pharmacotherapy counseling added later, after the prescribing physician and treating psychotherapist, physician, coach, member of the clergy, or other similar professional or someone acting under the supervision of such a professional (hereafter, “therapist”) agree that the pharmacotherapy counseling is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contra-indicated medicines or drugs for an adequate period before and after the pharmacotherapy).

A compound or composition of the invention (or alternately herein for convenience, the “medicine”) is administered shortly before or during a scheduled psychotherapy session, with timing optionally selected so that therapeutic effects begin by the time the psychotherapy session begins. It is to be understood that references to administering the medicine “during” a psychotherapeutic or other session are intended to refer to timing the administration of the medicine such that the therapeutic effects of the medicine at least partly temporally overlap with the therapeutic effects of the session. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy session and the therapist will often aid the patient in recalling the therapeutic effects and help them to incorporate the experiences into their everyday lives.

Pharmacotherapy sessions may be repeated as needed, based on the judgment of the treating physician and therapy team regarding the needs of the patient.

### Use of a Compound or Composition of the Present Invention Outside of Conventional Psychotherapy

In certain embodiments, a compound or composition of the present invention is administered outside of a conventional psychotherapy. This method is a broader, more flexible approach to pharmacotherapy that is not centered on supervision by a therapist. These pharmacotherapy sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy at home.

Before the pharmacotherapy session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (for example, without the involvement of the therapist) or does not employ any stimuli. Optionally, stimuli are selected in real time by the therapist or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted session. The therapist describes the kinds of experience that can be expected during the pharmacotherapy-assisted session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

Selected session goals and any commitments or other agreements regarding conduct between the patient and therapy team are reviewed immediately before administration of the medicine. Depending on the pharmaceutical preparation and route of administration, the therapeutic effects of the medicine usually begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person, or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to the patient aspects, including by an algorithm or artificial intelligence, and “computer” broadly meaning any electronic tool suitable for such purposes, whether worn or attached to a patient (for example, watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (for example, cloud-based systems).

For example, measurable aspects of a patient (for example, facial expression, eye movements, respiration rate, pulse rate, skin color change, patient voice quality or content, patient responses to questions) from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy counseling session has ended, often the next day but sometimes several days later. In this session, the patient discusses their experiences from the pharmacotherapy counseling session with the therapist, who can aid them in recalling the therapeutic effects and help them to incorporate the experiences into their everyday lives.

Pharmacotherapy counseling sessions may be repeated as needed, based on the judgment of the treating physician and therapy team regarding the needs of the patient.

### Methods of Treating Inflammatory and Metabolic Disorders

Compounds disclosed herein are useful in methods for treating a variety of diseases or disorders linked to inflammation in mammals. Included among such disorders are forms of arthritis (including rheumatoid arthritis and juvenile idiopathic arthritis), psoriasis, Crohn's disease, inflammatory bowel syndrome, ulcerative colitis, non-infectious uveitis, cryopyrin associated periodic syndrome, TNF receptor 1-associated periodic syndrome, and ankylosing spondylitis. Also included are metabolic disorders such as diabetes, atherosclerosis, metabolic syndrome, obesity, renal failure, hypertension and cancer. Also included are health consequences of obesity.

Treatment of inflammatory and metabolic disorders will typically (but not always) involve lower doses than those used to treat CNS disorders. Depending on the condition being treated and needs of the patient, medicine will be typically administered to maintain therapeutic concentrations of the medicine at the sites potentially or actually affected by inflammation. This can involve oral, topical, parental, or other routes of administration, potentially involving prodrugs and/or controlled release of the active molecule(s) to maintain therapeutic concentrations over longer durations than can otherwise be achieved. For example, extended-release tablets may be taken once every day with food in order to treat an inflammatory condition.

The anti-inflammatory effects of 5-HT_{2A} agonists can be assessed in a large number of ways because inflammation has many physiological and metabolic effects. One method is to measure in vivo or in vitro changes in response to tumor necrosis factor (TNF, TNF-alpha), which can be exogenously administered or stimulated in response to an inflammatory stimulus such as lipopolysaccharides (LPS). TNF is a cell signaling protein (cytokine) involved in systemic inflammation that is produced primarily by activated macrophages and secondarily by cell types including T helper cells, natural killer cells, neutrophils, mast cells, eosinophils, and neurons. Typically, either intact animals or appropriate cells that express 5-HT_{2A} receptors are exposed to TNF or LPS and well-established methods are used to measure recognized TNF-mediated proinflammatory markers.

For example, Yu and colleagues have shown that 5-HT_{2A} agonists inhibit TNF-stimulated increases in a variety of TNF-mediated proinflammatory markers, including intracellular adhesion molecule 1 (ICAM-1), vascular adhesion molecule 1 (VCAM-1), interleukin (IL)-6 gene expression, nitric-oxide synthase activity, and nuclear translocation of nuclear factor KB in primary aortic smooth muscle cells (Yu et al. 2008. J. Pharm. and Experimental Therapeutics, 327(2), 316-323) as well as in intact animals (Nau et al. 2013. PLoS ONE 8(10): e75426).

Anti-inflammatory effects of 5-HT_{2A} agonists can also be demonstrated in animal models of disease. Murine models of allergic asthma can be used to measure reductions in airways hyper-responsiveness, mucus hyperproduction, airways inflammation, and pulmonary eosinophil recruitment (Nau et al. 2015. Am. J. Physiology-Lung Cellular and Molecular Physiology, 308(2), L191-L198). Murine models of cardiovascular disease, such as High-Fat Diet-Fed Apolipoprotein E Knockout Mice, can be used to measure reduced expression levels of mRNA for inflammatory markers like interleukin (IL)-6 in vascular tissue, normalized glucose homeostasis, and reduced circulating cholesterol levels in mice fed a high-fat Western-style diet (Flanagan et al. 2019. Scientific reports, 9(1), 1-10).

### Additional Combination Therapies

In certain aspects an indolizine described herein or a pharmaceutically acceptable salt or salt mixture thereof is used in combination with an additional active agent to treat a disorder described herein. For example in certain embodiments an indolizine or a pharmaceutically acceptable salt or salt mixture thereof of the present invention is used in combination with a psychedelic compound pharmaceutically acceptable salt or salt mixture thereof to treat a CNS disorder. In certain embodiments this combination of an indolizine and a psychedelic compound decreases the hallucinogenic effect of the psychedelic while retaining the therapeutic effects and provides a therapy that is more effective than the indolizine alone. For example, the indolizine may have 5-HT_{2A} agonist or partial agonist activity and the psychedelic may produce less hallucinogenic effect if administered before, after, or concurrently with the indolizine. In certain embodiments the indolizine or a pharmaceutically acceptable salt or salt mixture thereof is administered in combination with a tryptamine or a pharmaceutically acceptable salt or salt mixture thereof.

Non-limiting examples of tryptamines include DMT, N-methyl,N-ethyl-tryptarine, N,N-diethyltryptamine, N-methyl,N-propyl-tryptamine, N,N-dipropyltryptamine, N-methyl,N-isopropyl-tryptamine, and N,N-diisopropyltryptarnine, or a 4-hydroxy or 4-rethoxy derivative thereof. For example the 4-hydroxy derivative of DMT is

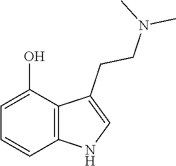

which is also known as psilocin.

Additional examples of tryptamines include:

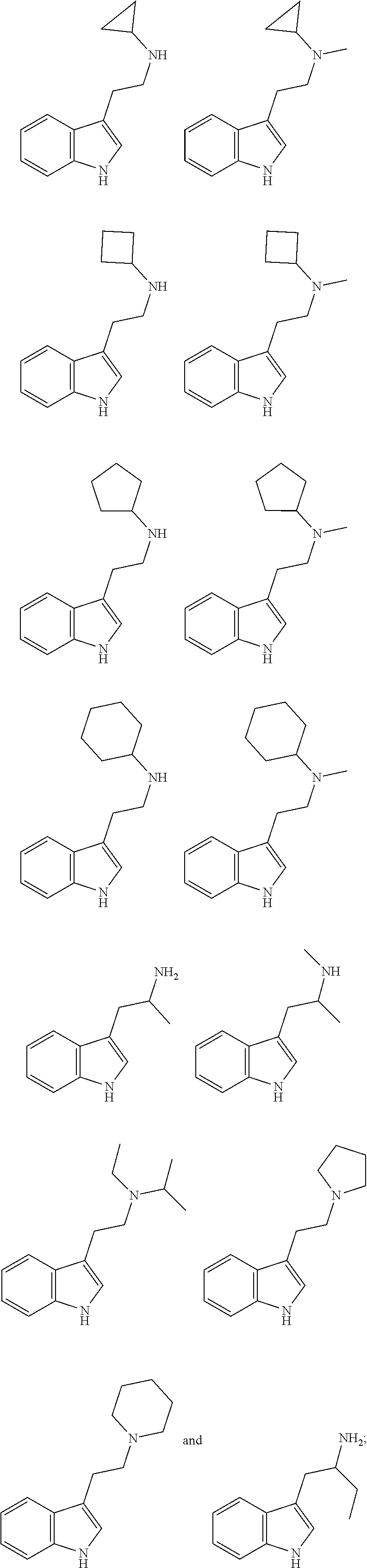

or a 4-hydroxy or 4-methoxy derivative thereof or a pharmaceutically acceptable salt or salt mixture thereof.

Additional examples of psychedelics include AL-LAD, DBT, a,O-DMS, 2,a-DMT, ETH-LAD, harmaline, 4-HO-DBT, 4-HO-DET, 4-H₂PO4-DET, 4-HO-DIPT, 4-HO-DMT, 4-H₂PO4-DMT, 5-HO-DMT, 4-HO-DPT, 4-HO-MET, 4-HO-MIPT, 4-HO-MPT, 4-HO-pyr-T, ibogaine, LSD, MBT, 4,5-MDO-DIPT, 5,6-MDO-DIPT, 4,5-MDO-DMT, 5,6-MDO-DMT, 5,6-MDO-MIPT, 2-Me-DET, 5-MeO-DET, 5-MeO-DPT, 5-MeO-DMt, 4-MeO-MIPT, 5-MeO-MIPT, 5,6-MeO-MIPT, 5-MeO-NMT, 5-MeO-pyr-T, 6-MeO-THH, 5-MeO-TMT, 5-MeS-DMT, MIPT, a-MT, NET, NIPT, NMT, PRO-LAD, tetrahydroharmine, a,N,O-TMS, 5-MeO-DALT, and DALT. Non-limiting examples of synthetic methods that can be used to prepare these materials are provided on https://isomerdesign.com/P¹HKAL/browse.php?domain=tk, and Epke et at 1981, doi:10 1002/jhet 5570180131.

In certain embodiments the psychedelic is DMT or a 4-hydroxy derivative thereof. In certain embodiments the psychedelic is N-rnethyl,N-eihyl-trypta.mine or a 4-hydroxy derivative thereof. In certain embodiments the psychedelic is N.N-diethyltryptamine or a 4-hydroxy derivative thereof. In certain embodiments the psychedelic is N-methyl,N-propyl-tryptamine or a 4-hydroxy derivative thereof. In certain embodiments the psychedelic is N,N-dipropyltryptamine or a 4-hydroxy derivative thereof. In certain embodiments the psychedelic is N-methyL,N-isopropyl-tryptamine or a 4-hydroxy derivative thereof. In certain embodiments the psychedelic is N,N-diisopropyltryptamine or a 4-hydroxy derivative thereof. In certain embodiments the psychedelic is N-cyclopentyl-tryptamine or a 4-hydroxy derivative thereof. In certain embodiments the psychedelic is N-cyclopropyl-tryptamine or a 4-hydroxy derivative thereof.

In other embodiments the psychedelic is a lysergamide for example lysergic acid diethylamide.

In other embodiments the psychedelic is a phenethylamine for example mescaline.

In certain aspects a prodrug of the additional active agent, for example a prodrug of a tryptamine, is administered in combination with an indolizine described herein or a pharmaceutically acceptable salt or salt mixture thereof.

### Pharmaceutical Compositions and Formulations

While it is possible to administer a compound employed in the methods of this invention directly without any formulation, the compounds are usually administered in the form of pharmaceutical compositions comprising a pharmaceutically acceptable carrier, diluent, or excipient, and at least one active ingredient. “Pharmaceutically acceptable” as used in connection with an excipient, carrier, or diluent means an excipient, carrier, or diluent that is useful in preparing a pharmaceutical composition that is generally safe, non-toxic, and neither biologically nor otherwise undesirable for veterinary use and/or human pharmaceutical use.

These compositions can be administered by a variety of routes including oral, mucosal (e.g., buccal, sublingual), rectal, transdermal, subcutaneous, intravenous, intramuscular, inhaled, and intranasal. The compounds employed in the methods of this invention are effective as oral, mucosal, rectal, subcutaneous, intravenous, intramuscular, inhaled, and intranasal compositions. Such compositions are prepared in a manner well known in the pharmaceutical art and comprise at least one active compound. (See, e.g., Remington, 2005, Remington: The science and practice of pharmacy, 21st ed., Lippincott Williams & Wilkins.)

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions, and sterile packaged powders.

Different embodiments of the invention include the following examples: Pharmaceutically acceptable complex derivatives of each drug in each group, including solvates, salts, esters, enantiomers, isomers (stereoisomers and/or constitutional, including ones based on substituting deuterium for hydrogen), derivatives or prodrugs of Formula I. Another embodiment of the invention includes multiple variations in the pharmaceutical dosages of each drug in the combination as further outlined below. Another embodiment of the invention includes various forms of preparations including using solids, liquids, immediate or delayed or extended-release forms. Many types of variations are possible as known to those skilled in the art.

Another embodiment of the invention includes multiple routes of administration, which may differ in different patients according to their preference, co-morbidities, side effect profile, and other factors (IV, PO, transdermal, etc.). Another embodiment of the invention includes the presence of other substances with the active drugs, known to those skilled in the art, such as fillers, carriers, gels, skin patches, lozenges, or other modifications in the preparation to facilitate absorption through various routes (such as gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 0.05 to about 350 mg, more preferably about 0.1 to about 280 mg, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

The active compounds are effective over a wide dosage range. For example, as-needed dosages normally fall within the range of about 0.0007 to about 5 mg/kg. In the treatment of adult humans, the range of about 0.001 to about 4 mg/kg, in single dose, is especially preferred.

However, it will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way. In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

It should be apparent that the compositions of the invention are not limited to combinations of a single compound (i.e., one structure of Formula I), and a single carrier, diluent, or excipient alone, but also include combinations of multiple such structures, and/or multiple carriers, diluents, and excipients. Pharmaceutical compositions of this invention thus may comprise one or more structures of Formula I (or their derivatives and analogs) in combination, together with one or more pharmaceutically-acceptable carriers, diluents, and/or excipients, and additionally with one or more other active compounds.

It is contemplated that the active compounds of the invention may be formulated in a pharmaceutical preparation with other active compounds to increase therapeutic efficacy, decrease unwanted effects, increase stability/shelf-life, and/or alter pharmacokinetics. Such other active compounds include: antioxidants (such alpha-lipoate in acid or salt form, ascorbate in acid or salt form, selenium, or N-acetylcysteine), H2-receptor agonists or antagonists (such as famotidine), stimulants (such as dextroamphetamine, lisdextroamphetamine, or methamphetamine), entactogens (such as MDMA), antiinflammatories (such as ibuprofen or ketoprofen), matrix met alloproteinase inhibitors (such as doxycycline), NOS inhibitors (such as S-methyl-L-thiocitrulline), proton pump inhibitors (such as omeprazole), phosphodiesterase 5 inhibitors (such as sildenafil), drugs with cardiovascular effects (beta antagonists such as propranolol, mixed alpha and beta antagonists such as carvedilol, alpha antagonists such as prazosin, imidazoline receptor agonists such as rilmenidine or moxonidine, serotonin antagonists such as ketanserin or lisuride), norepinephrine transporter blockers (such as reboxetine), acetylcholine nicotinic receptor modulators (such as bupropion, hydroxybupropion, methyllycaconitine, memantine, or mecamylamine), gastrointestinal acidifying agents (such as ascorbic acid or glutamic acid hydrochloride) or alkalinizing agents (such as sodium bicarbonate), NMDA receptor antagonists (such as ketamine), TrkB agonists (such as 7,8-dihydroxyflavone, 7,8,3′-trihydroxyflavone, or N-acetylserotonin), and serotonin receptor agonists (such as 5-methoxy-N-methyl-N-isopropyltryptamine, N,N-Dimethyl-2-(2-methyl-1H-indol-1-yl)ethan-1-amine, psilocin, or psilocybin). These ingredients may be in ion, freebase, or salt form and may be isomers or prodrugs.

In one preferred embodiment, a compound of Formula I, either racemic, an enantiomer, or a mixture of enantiomers, and with zero to five hydrogens replaced with deuterium, are formulated in a pharmaceutical composition that contains a pharmaceutically acceptable salt of dextroamphetamine in the amount of 2 mg, 4 mg, 5 mg, 7 mg, 10 mg, 15 mg, 20 mg, or 25 mg. The required amount of dextroamphetamine will vary depending on the needs of the patient.

In another preferred embodiment, a compound of Formula I, either racemic, an enantiomer, or a mixture of enantiomers, and with zero to five hydrogens replaced with deuterium, are formulated in a pharmaceutical composition that contains a pharmaceutically acceptable salt of dextroamphetamine with dextroamphetamine in a ratio by weight of 1:2, 1:3, 1:4, 1:5 to the compound of Formula I. The required amount of dextroamphetamine will vary depending on the needs of the patient.

In one preferred embodiment, a compound of Formula I, either racemic, an enantiomer, or a mixture of enantiomers, and with zero to five hydrogens replaced with deuterium, are formulated in a pharmaceutical composition that contains a pharmaceutically acceptable salt of MDMA, in an amount between 5 and 180 mg, preferably 15-60 mg. The required amount of MDMA will vary depending on the needs of the patient.

In another preferred embodiment, a compound of Formula I, either racemic, an enantiomer, or a mixture of enantiomers, and with zero to five hydrogens replaced with deuterium, are formulated in a pharmaceutical composition that contains a pharmaceutically acceptable salt of MDMA with MDMA in a ratio by weight of 1:2, 1:3, 1:4, 1:5 to the compound of Formula I. The required amount of MDMA will vary depending on the needs of the patient.

In certain embodiments, a compound of Formula I may be formulated in a pharmaceutically acceptable oral dosage form. Oral dosage forms may include but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived IPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agent of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the active agent of the present invention formulation. In one embodiment, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles are not microencapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (e.g., Methocel®), hydroxypropylmethylcellulose (e.g., Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g., Avicel, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in the solid dosage forms described here include polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, e.g., gum tragacanth and gum acacia, guar gum, xanthans, including xanthan gum, sugars, cellulosic, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining superdisintegrant such as Croscarmellose sodium and different grades of microcrystalline cellulose in different ratios. To aid disintegration, sodium starch glycolate will be added.

In cases where the two (or more) drugs included in the fixed-dose combinations of the present invention are incompatible, cross-contamination can be avoided, e.g., by incorporation of the drugs in different drug layers in the oral dosage form with the inclusion of a barrier layer(s) between the different drug layers, wherein the barrier layer(s) comprise one or more inert/non-functional materials.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol, glycerol, and propylene glycol. Ethers, such as poly(ethylene glycol), petroleum hydrocarbons such as mineral oil and petrolatum, and water may also be used in suspension formulations.

In some embodiments, formulations are provided comprising particles of Formula I and at least one dispersing agent or suspending agent for oral administration to a subject. The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that the drug is absorbed in a controlled manner over time. In certain embodiments, the aqueous dispersion or suspension is an immediate-release formulation. In another embodiment, an aqueous dispersion comprising amorphous particles is formulated such that a portion of the particles of the present invention are absorbed within, e.g., about 0.75 hours after administration and the remaining particles are absorbed 2 to 4 hours after absorption of the earlier particles.

In other embodiments, addition of a complexing agent to the aqueous dispersion results in a larger span of the particles to extend the drug absorption phase of the active agent such that 50-80% of the particles are absorbed in the first hour and about 90% are absorbed by about 4 hours. Dosage forms for oral administration can be aqueous suspensions selected from the group including pharmaceutically acceptable aqueous oral dispersions, emulsions, solutions, and syrups. See, e.g., Singh et al., Encyclopedia of Pharm. Tech., 2nd Ed., 754-757 (2002). In addition to the active agents of the present invention particles, the liquid dosage forms may comprise additives, such as (a) disintegrating agents; (b) dispersing agents; (c) wetting agents; (d) at least one preservative, (e) viscosity enhancing agents, (f) at least one sweetening agent, and (g) at least one flavoring agent.

Examples of disintegrating agents for use in the aqueous suspensions and dispersions include a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g., HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corp., Parsippany, N.J.)).

In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (e.g., HPC, HPC-SL, and HPC-L); hydroxypropyl methylcellulose and hydroxypropyl methylcellulose ethers (e.g., HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3- tetramethyl butyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908® or Poloxamine 908®).

Wetting agents (including surfactants) suitable for the aqueous suspensions and dispersions described herein are known in the art and include acetyl alcohol, glycerol monostearate, polyoxyethylene sorbitan fatty acid esters (e.g., the commercially available Tweens® such as e.g., Tween 20® and Tween 80© (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from about 0.01% to about 0.3% methylparaben by weight to the weight of the aqueous dispersion and about 0.005% to about 0.03% propylparaben by weight to the total aqueous dispersion weight. In yet another embodiment, the aqueous liquid dispersion can comprise methylparaben from about 0.05 to about 0.1 weight % and propylparaben from about 0.01 to about 0.02 weight % of the aqueous dispersion.

Suitable viscosity enhancing agents for the aqueous suspensions or dispersions described herein include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid active agents of the present invention formulations can also comprise inert diluents commonly used in the art, such as water or other solvents, solubilizing agents, emulsifiers, and/or sweeteners.

The formulations of the present invention suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of >1 mg/ml using water-soluble beta cyclodextrins (e.g., beta-sulfobutyl-cyclodextrin and 2-hydroxypropylbeta-cyclodextrin). Proper fluidity can be maintained, for example, by the use of a coating such as a lecithin, by the maintenance of the required particle size in the case of dispersions, and by the use of surfactants.

The formulations of the present invention suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The suspension formulations of the present invention designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention particles and the range of the particle sizes of the active agents of the present invention particles can be used to control the release of the drug by controlling the rate of dissolution in fat or muscle.

In still other embodiments, effervescent powders containing structures of Formula I may be prepared. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, a powder comprising the active agents of the present invention described herein may be formulated to comprise one or more pharmaceutical excipients and flavors. Such a powder may be prepared, for example, by mixing the active agents of the present invention and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

In certain embodiments of the present invention, pharmaceutical compositions containing structures of Formula I may be formulated into a dosage form suitable for parenteral use. For example, the dosage form may be a lyophilized powder, a solution, suspension (e.g., depot suspension).

In other embodiments, pharmaceutical compositions containing structures of Formula I may be formulated into a topical dosage form including a patch, gel, paste, cream, emulsion, liniment, balm, lotion, and ointment.

Tablets of the invention described here can be prepared by methods well known in the art. Various methods for the preparation of the immediate release, modified release, controlled release, and extended-release dosage forms (e.g., as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, PA; Sheth et al. (1980), Compressed tablets, in Pharmaceutical dosage forms, Vol. 1, edited by Lieberman and Lachtman, Dekker, NY.

In certain embodiments, solid dosage forms, e.g., tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include one or a combination of methods: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include spray drying, pan coating, melt granulation, granulation, fluidized bed spray drying or coating (e.g., Wurster coating), tangential coating, top spraying, tableting, extruding and the like.

Compressed tablets are solid dosage forms prepared by compacting the bulk blend compositions and formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the active agents of the present invention. In other embodiments, the film coating aids in patient compliance (e.g., Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, e.g., by placing the bulk blend the active agents of the present invention formulation, described above, inside of a capsule. In some embodiments, the active agents of the present invention (in non-aqueous suspensions and solutions) are placed in a soft gelatin capsule. In other embodiments, the active agents of the present invention formulations are placed in standard gelatin capsules or non-gelatin capsules such as capsules comprising HPMC. In other embodiments, the active agents of the present invention formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the active agents of the present invention formulation is delivered in a capsule form.

In certain preferred embodiments, the formulations of the present invention are fixed-dose combinations of structures of Formula I and at least one other pharmacological agent. Fixed-dose combination formulations may contain the following combinations in the form of single-layer monolithic tablet or multi-layered monolithic tablet or in the form of a core tablet-in-tablet or multi-layered multi-disk tablet or beads inside a capsule or tablets inside a capsule but not limited to: (a) therapeutically efficacious fixed-dose combinations of immediate-release formulations of structures of Formula I and other pharmacological agents; (b) therapeutically efficacious fixed-dose combinations of extended release structures of Formula I and delayed and/or extended-release other pharmacological agents contained in a single dosage form.

The pharmaceutical compositions described herein can be formulated into any suitable dosage form, including aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. Generally speaking, one will desire to administer an amount of the active agents of the present invention that is effective to achieve a plasma level commensurate with the concentrations found to be effective in vivo for a period of time effective to elicit a desired therapeutic effect without abuse liability.

Depending on the desired release profile, the oral solid dosage forms of the present invention may contain a suitable amount of controlled-release agents, extended-release agents, and/or modified-release agents (e.g., delayed-release agents). The pharmaceutical solid oral dosage forms comprising the active agents of the present invention described herein can be further formulated to provide a modified or controlled release of the active agents of the present invention. In some embodiments, the solid dosage forms described herein can be formulated as a delayed release dosage form such as an enteric-coated delayed release oral dosage forms, i.e., as an oral dosage form of a pharmaceutical composition as described herein which utilizes an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention formulations described herein may be administered using a variety of formulations known in the art. For example, such formulations include those described in U.S. Pat. Nos. 5,011,692, 5,017,381, 5,229,135, and 5,840,329. Other dosage forms suitable for use with the active agents of the present invention formulations are described in, for example, U.S. Pat. Nos. 4,871,549, 5,260,068, 5,260,069, 5,508,040, 5,567,441 and 5,837,284.

In one embodiment, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from about 2% to about 75%, preferably from about 2.5% to about 70%, or from about 40% to about 70%, by weight of the total dose of the active agents of the present invention in said formulation, in admixture with one or more binders.

Coatings for providing a controlled, delayed, or extended-release may be applied to structures of Formula I or to a core containing structures of Formula I. The coating may comprise a pharmaceutically acceptable ingredient in an amount sufficient, e.g., to provide an extended release from e.g., about 1 hours to about 7 hours following ingestion before release of structures of Formula I. Suitable coatings include one or more differentially degradable coatings such as, by way of example only, pH-sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (e.g., ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric.

The controlled/delayed/extended-release systems may utilize a hydrophilic polymer, including a water-swellable polymer (e.g., a natural or synthetic gum). The hydrophilic polymer may be any pharmaceutically acceptable polymer which swells and expands in the presence of water to slowly release the active agents of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous-based system and is a spray-dried CAP psuedolatex with particles <1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Extended-release multi-layered matrix tablets may be prepared by using fixed-dose combinations of structures of Formula I with another pharmacological agent. Such formulations may comprise one or more of the drugs within a hydrophilic or hydrophobic polymer matrix. For example, a hydrophilic polymer may comprise guar gum, hydroxypropylmethylcellulose, and xanthan gum as matrix formers. Lubricated formulations may be compressed by a wet granulation method.

Multilayer tablet delivery (e.g., such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (e.g., such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate towards constant release. This technology enables customized levels of controlled release of specific drugs and/or simultaneous release of two different drugs at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different drugs or dual release rate of the same drug in a single dosage form may be prepared by using hydrophilic and hydrophobic polymer matrices. Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In one embodiment, the dosage form is a solid oral dosage form which is an immediate release dosage form whereby >80% of the active agents of the present invention are released within 2 hours after administration. In other embodiments, the invention provides an (e.g., solid oral) dosage form that is a controlled release or pulsatile release dosage form. In such instances, the release may be, e.g., 30 to 60% of the active agents of the present invention particles by weight are released from the dosage form within about 2 hours after administration and about 90% by weight of the active agents of the present invention released from the dosage form, e.g., within about 4 hours after administration. In yet other embodiments, the dosage form includes at least one active agent in an immediate-release form and at least one active agent in the delayed-release form or sustained-release form. In yet other embodiments, the dosage form includes at least two active agents that are released at different rates as determined by in-vitro dissolution testing or via oral administration.

The various release dosage formulations discussed above, and others known to those skilled in the art can be characterized by their disintegration profile. A profile is characterized by the test conditions selected. Thus, the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cₘₐₓ, Tₘₐₓ, and AUC (0-τ).

In certain embodiments, the controlled, delayed or extended-release of one or more of the drugs of the fixed-dose combinations of the invention may be in the form of a capsule having a shell comprising the material of the rate-limiting membrane, including any of the coating materials previously discussed, and filled with the active agents of the present invention particles. A particular advantage of this configuration is that the capsule may be prepared independently of the active agent of the present invention particles; thus, process conditions that would adversely affect the drug can be used to prepare the capsule.

Alternatively, the formulation may comprise a capsule having a shell made of a porous or a pH-sensitive polymer made by a thermal forming process. Another alternative is a capsule shell in the form of an asymmetric membrane, i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase separate by exchanging the solvent with a miscible non-solvent. In another embodiment, spray layered active agents of the present invention particles are filled in a capsule.

An exemplary process for manufacturing the spray layered the active agents of the present invention is the fluidized bed spraying process. The active agents of the present invention suspensions or the active agents of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agents of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agents of the present invention particles or the active agents of the present invention complex particles comprise about 30 to 70 wt % of the active agents of the present invention based on the total weight of the particles.

In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 1 mg (or at least 10 mg or at least 20 mg) of the active agents of the present invention and has a total weight of less than 800 mg (or less than 700 mg). The capsule may contain a plurality of the active agents of the present invention-containing beads, for example, spray layered beads. In some embodiments, the beads are 12-25% the active agents of the present invention by weight. In some embodiments, some or all of the active agents of the present invention containing beads are coated with a coating comprising 6 to 15% (or 8 to 12%) of the total bead weight. Optimization work typically involves lower loading levels and the beads constitute 30 to 60% of the finished bead weight. The capsule may contain a granulated composition, wherein the granulated composition comprises the active agents of the present invention.

The capsule may provide pulsatile release of the active agents of the present invention oral dosage form. Such formulations may comprise: (a) a first dosage unit comprising a compound of Formula I that is released substantially immediately following oral administration of the dosage form to a patient; (b) a second dosage unit comprising a compound of Formula I that is released approximately 2 to 6 hours following administration of the dosage form to a patient. For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In other embodiments, the pulsatile release capsule contains a plurality of beads formulated for modified release and the at least one agent of the present invention is, for example, spray granulated for immediate release.

In some embodiments, the release of the active agents of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric-coated particles and immediate release particles of the present invention beads are filled into a soft gelatin capsule. Additional suitable excipients may also be filled with the coated particles in the capsule. The uncoated particles release the active agent of the present invention immediately upon administration while the coated particles do not release the active agent of the present invention until these particles reach the intestine. By controlling the ratios of the coated and uncoated particles, desirable pulsatile release profiles also may be obtained. In some embodiments, the ratios between the uncoated and the coated particles are e.g., 20/80, or 30/70, or 40/60, or 50/50, w/w to obtain desirable release.

In certain embodiments, spray layered active agents of the present invention can be compressed into tablets with commonly used pharmaceutical excipients. Any appropriate apparatus for forming the coating can be used to make the enteric coated tablets, e.g., fluidized bed coating using a Wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms,” 1990.

In certain embodiments, the spray layered active agents of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agents of the present invention formulation into the gastrointestinal fluid. In other embodiments, the spray layered active agents of the present invention particles or spray layered active agents complex particles with enteric coatings described above and one or more excipients are dry blended and compressed into a mass, such as a tablet.

In certain embodiments, a pulsatile release of the active agent of the present invention formulation comprises a first dosage unit comprising a formulation made from the active agent of the present invention containing granules made from a spray drying or spray granulated procedure or a formulation made from the active agent of the present invention complex containing granules made from a spray drying or spray granulated procedure without enteric or sustained-release coatings and a second dosage unit comprising spray layered the active agent of the present invention particles or spray layered the active agent of the present invention complex particles with enteric or sustained-release coatings. In one embodiment, the active agent is wet or dry blended and compressed into a mass to make a pulsatile release tablet.

In certain embodiments, binding, lubricating and disintegrating agents are blended (wet or dry) to the spray layered active agents of the present invention to make a compressible blend. The dosage units containing a compound of Formula I and the dosage units containing the other pharmacological agent are compressed separately and then compressed together to form a bilayer tablet. In yet another embodiment, the dosage unit containing the other pharmacological agent is in the form of an overcoat and completely covers the second dosage unit containing a compound of Formula I. In yet another embodiment, the dosage unit containing a compound of Formula I is in the form of an overcoat and completely covers the second dosage unit containing the other pharmacological agent.

In certain embodiments, ingredients (including or not including the active agent) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, e.g., via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789, 5,340,591, 5,456,923, 5,700,485, 5,723,269, and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

The pharmacological agents that make up the combination therapy disclosed herein may be a combined dosage form or in separate dosage forms intended for substantially simultaneous administration. The pharmacological agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from a few minutes to several hours, depending upon the properties of each pharmacological agent, such as potency, solubility, bioavailability, plasma half-life and kinetic profile of the pharmacological agent. Circadian variation of the target molecule concentration may also determine the optimal dose interval. For example, structures of Formula I may be administered while the other pharmacological agent is being administered (concurrent administration) or may be administered before or after other pharmacological agent is administered (sequential administration).

By way of non-limiting example, the following formulations may be used in the methods of the present invention. In the below examples the active agent (T-1, T-2, or another referenced species of the present invention) can be replaced with a different compound of the present invention.

### Formulation of Hard Gelatin Capsules

For example, hard gelatin capsules containing the following ingredients can be prepared:

| Ingredient | Quantity (mg/capsule) |
|---|---|
| A compound of Formula I | 30.0 |
| Starch | 205.0 |
| Alpha lipoic acid | 100.0 |
| Magnesium stearate | 5.0 |

The above ingredients are mixed and filled into hard gelatin capsules in 340 mg quantities.

| Ingredient | Quantity (mg/capsule) |
|---|---|
| Compound 1 | 2.0 |
| Starch | 233.0 |
| Alpha lipoic acid | 100.0 |
| Magnesium stearate | 5.0 |

The above ingredients are mixed and filled into hard gelatin capsules in 340 mg quantities.

### Formulation of Tablets

For example, a tablet containing the following ingredients can be prepared:

| Ingredient | Quantity (mg/tablet) |
|---|---|
| A compound of Formula I | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

| Ingredient | Quantity (mg/tablet) |
|---|---|
| Compound 2 | 12.0 |
| Cellulose, microcrystalline | 213.0 |
| Colloidal silicon dioxide | 10.0 |
| Stearic acid | 5.0 |

The components are blended and compressed to form tablets, each weighing 240 mg.

### Formulation of Dry Powder for Insufflation

For example, a dry powder containing the following ingredients can be prepared:

| Ingredient | Weight % |
|---|---|
| A compound of Formula I | 5 |
| Lactose | 95 |

The active mixture is mixed with the lactose and the mixture is added to a dry powder inhaling-appliance.

| Ingredient | Weight % |
|---|---|
| Compound 1 | 5 |
| Lactose | 95 |

The active mixture is mixed with the lactose and the mixture is added to a dry powder inhaling appliance.
Formulation of Tablets with Multiple Active Agents
For example, a tablet containing the following ingredients can be prepared:

| Ingredient | Quantity (mg/tablet) |
|---|---|
| A compound of Formula I | 20.0 |
| Another active agent | 10.0 |
| Starch | 45.0 |
| Microcrystalline cellulose | 35.0 |
| Polyvinylpyrrolidone (as 10% solution in | 4.0 |
| water) | |
| Sodium carboxymethyl starch | 4.5 |
| Magnesium stearate | 0.5 |
| Talc | 1.0 |

The active ingredients, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60′ C and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

| Ingredient | Quantity (mg/tablet) |
|---|---|
| Compound 3 | 20.0 |
| Compound 1 | 10.0 |
| Starch | 45.0 |
| Microcrystalline cellulose | 35.0 |
| Polyvinylpyrrolidone (as 10% solution in | 4.0 |
| water) | |
| Sodium carboxymethyl starch | 4.5 |
| Magnesium stearate | 0.5 |
| Talc | 1.0 |

The active ingredients, starch and cellulose are passed through a No. 20 mesh U.S. sieve and mixed thoroughly. The solution of polyvinylpyrroli done is mixed with the resultant powders, which are then passed through a 16 mesh U.S. sieve. The granules so produced are dried at 50-60′ C and passed through a 16 mesh U.S. sieve. The sodium carboxymethyl starch, magnesium stearate, and talc, previously passed through a No. 30 mesh U.S. sieve, are added to the granules which, after mixing, are compressed on a tablet machine to yield tablets each weighing 120 mg.
Formulation of Capsules with Multiple Active Agents
For example, a capsule containing the following ingredients can be prepared:

| Ingredient | Quantity (mg/capsule) |
|---|---|
| A compound of Formula I (R-enantiomer) | 10.0 |
| Another active agent | 30.0 |
| Starch | 109.0 |
| Magnesium stearate | 1.0 |

The active ingredients, cellulose, starch, an magnesium stearate are bended, passe through a No. 20 mesh U.S. sieve, and filled into hard gelatin capsules in 150 mg quantities.

| Ingredient | Quantity (mg/capsule) |
|---|---|
| Compound 1 | 10.0 |
| Starch | 139.0 |
| Magnesium stearate | 1.0 |

The active ingredients, cellulose, starch, and magnesium stearate are blended, passed through a No. 20 mesh U.S. sieve, and filled into hard gelatin capsules in 150 mg quantities.
Formulation of suppositories
For example, a suppository containing the following ingredients can be prepared:

| Ingredient | Quantity (mg) |
|---|---|
| T-5 (R-enantiomer) | 25.0 |
| Saturated fatty acid glycerides | 2000.0 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

| Ingredient | Quantity (mg) |
|---|---|
| Compound 4 | 25.0 |
| Saturated fatty acid glycerides | 2000.0 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation of Suspension

For example, a suspension containing the following ingredients can be prepared:

| Ingredient | Amount | |
|---|---|---|
| A compound of Formula I (R-enantiomer) | 50.0 | mg |
| Xanthan gum | 4.0 | mg |
| Sodium carboxymethyl cellulose (11%) | 50.0 | mg |
| Microcrystalline cellulose (89%) | 50 | mg |
| Sucrose | 1.75 | g |
| Sodium benzoate | 10.0 | mg |

| | |
|---|---|
| Flavor and Color | q.v. |
| Purified water | To 5.0 ml |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

| Ingredient | Amount | |
|---|---|---|
| Compound 3 | 50.0 | mg |
| Xanthan gum | 4.0 | mg |
| Sodium carboxymethyl cellulose (11%) | 50.0 | mg |
| Microcrystalline cellulose (89%) | 50 | mg |
| Sucrose | 1.75 | g |
| Sodium benzoate | 10.0 | mg |

| | |
|---|---|
| Flavor and Color | q.v. |
| Purified water | To 5.0 ml |

The active ingredient, sucrose an cant an gum are (blended) passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation of Capsules

For example, a capsule containing the following ingredients can be prepared:

| Ingredient | Amount (mg/capsule) |
|---|---|
| A compound of Formula I | 100.0 |
| Starch | 407.0 |
| Magnesium stearate | 3.0 |

The active ingredient, cellulose, starch, and magnesium stearate are blended, passed through a No. 20 mesh U.S. sieve, and filled into hard gelatin capsules in 510 mg quantities.

| Ingredient | Amount (mg/capsule) |
|---|---|
| Compound 1 | 10.0 |
| Starch | 497.0 |
| Magnesium stearate | 3.0 |

The active ingredient, cellulose, starch, and magnesium stearate are blended, passed through a No. 20 mesh U.S. sieve, and filled into hard gelatin capsules in 510 mg quantities.

### Formulation of Intravenous Solution

For example, an intravenous solution containing the following ingredients can be prepared:

| Ingredient | Amount | |
|---|---|---|
| A compound of Formula I | 250.0 | mg |
| Isotonic saline | 1000 | ml |

| Ingredient | Amount | |
|---|---|---|
| Compound 1 | 250.0 | mg |
| Isotonic saline | 1000 | ml |

### Formulation of Topical Form

For example, a topical form containing the following ingredients can be prepared:

| Ingredient | Amount (g) |
|---|---|
| A compound of Formula I | 10.0 |
| Emulsifying Wax | 30.0 |
| Liquid Paraffin | 20.0 |
| White Soft Paraffin | 100 |

The white soft paraffin is heated until molten. The liquid paraffin and emulsifying wax are incorporated and stirred until dissolved. The active ingredient is added and stirring is continued until dispersed. The mixture is then cooled until solid.

| Ingredient | Amount (g) |
|---|---|
| Compound 3 | 10.0 |
| Emulsifying Wax | 30.0 |
| Liquid Paraffin | 20.0 |
| White Soft Paraffin | 100 |

The white soft paraffin is heated until molten. The liquid paraffin and emulsifying wax are incorporated and stirred until dissolved. The active ingredient is added and stirring is continued until dispersed. The mixture is then cooled until solid.

### Formulation of Sublingual or Buccal Tablets

For example, a sublingual or buccal tablet containing the following ingredients can be prepared:

| Ingredient | Amount (mg/tablet) |
|---|---|
| A compound of Formula I | 20.0 |
| Glycerol | 210.5 |
| Water | 143.0 |
| Sodium Citrate | 4.5 |
| Polyvinyl Alcohol | 26.5 |
| Polyvinylpyrrolidone | 15.5 |

The glycerol, water, sodium citrate, polyvinyl alcohol, and polyvinylpyrroli done are admixed together by continuous stirring and maintaining the temperature at about 90′ C. When the polymers have gone into solution, the solution is cooled to about 50-55′ C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

| Ingredient | Amount (mg/tablet) |
|---|---|
| Compound 2 | 20.0 |
| Glycerol | 210.5 |
| Water | 143.0 |
| Sodium Citrate | 4.5 |
| Polyvinyl Alcohol | 26.5 |
| Polyvinylpyrrolidone | 15.5 |

The glycerol, water, sodium citrate, polyvinyl alcohol, and polyvinylpyrroli done are admixed togetherby continuous stirring and maintainingthetemperature at about 90° C. Whenthepolymers have gone into solution, the solution is cooled to about 50-55′ C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.
Formulation of Capsules with Three or More Active Agents
For example, a capsule containing the following ingredients can be prepared:

| Ingredient | Quantity (mg/capsule) |
|---|---|
| A compound of Formula I | 10.0 |
| Another active agent | 30.0 |
| Amphetamine sulfate | 5.0 |
| Starch | 109.0 |
| Magnesium stearate | 1.0 |

The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| Compound 1 | 10.0 |
| Compound 2 | 30.0 |
| Amphetamine sulfate | 5.0 |
| Starch | 109.0 |
| Magnesium stearate | 1.0 |

The active ingredients, cellulose, starch, and magnesium stearate are blended, passed through a No. 20 mesh U.S. sieve, and filled into hard gelatin capsules in 155 mg quantities.

| Ingredient | Quantity (mg/capsule) |
|---|---|
| Compound 4 | 30.0 |
| Compound 1 | 2.0 |
| Psilocybin | 20.0 |
| Starch | 94.0 |
| Magnesium stearate | 1.0 |

The active ingredients, cellulose, starch, and magnesium stearate are blended, passed through a No. 20 mesh U.S. sieve, and filled into hard gelatin capsules in 147 mg quantities.
Formulation of Capsules with Four or More Active Agents
For example, a capsule containing the following ingredients can be prepared:

| Ingredient | Quantity (mg/capsule) |
|---|---|
| A compound of Formula I (70% R- | 30.0 |
| enantiomer) | |
| Another compound of Formula I (70% S- | 10.0 |
| enantiomer) | |
| Psilocybin | 2.0 |
| Alpha lipoic acid | 40.0 |
| Starch | 72.0 |
| Magnesium stearate | 1.0 |

The active ingredients, cellulose, starch, and magnesium stearate are blended, passed through a No. 20 mesh U.S. sieve, and filled into hard gelatin capsules in 155 mg quantities.

### Formulation of Liquid for Vaporization

For example, a liquid formulation containing the following ingredients can be prepared:

| Ingredient | Quantity (units) | |
|---|---|---|
| A compound of Formula I (R-enantiomer) | 500 | mg |
| Propylene Glycol | 2 | ml |
| Glycerin | 2 | ml |

The active mixture is mixed and added to a liquid vaporization appliance.

| Ingredient | Quantity (units) | |
|---|---|---|
| Compound 3 | 500 | mg |
| Propylene Glycol | 2 | ml |
| Glycerin | 2 | ml |

The active mixture is mixed and added to a liquid vaporization appliance.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. Indirect techniques, which are generally preferred, usually involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

It should be readily appreciated that the above formulation examples are illustrative only. Accordingly, it should be understood that reference to particular structure(s) is likewise illustrative, and the structure(s) in any Example may be substituted by other structure(s) of the invention. Likewise, any of the other active compounds (e.g., amphetamine sulfate in Example 17, or psilocybin hydrochloride in Example 18) may be substituted by a different other active compound, as may be the inactive compounds. Moreover, for any of structures of Formula I or for any other active compounds of the invention, substitution of the compound by its prodrug, free base, salt, or hydrochloride salt shall be understood to provide merely an alternative embodiment still within the scope of the invention. Further, compositions within the scope of the invention should be understood to be open-ended and may include additional active or inactive compounds and ingredients.

The type of formulation employed for the administration of the compounds employed in the methods of the present invention generally may be dictated by the compound(s) employed, the type of pharmacokinetic profile desired from the route of administration and the compound(s), and the state of the patient.

### Dosage Regimes

The compounds or pharmaceutically acceptable formulations of the present invention can be administered to the host in any amount, and with any frequency, that achieves the goals of the invention as used by the healthcare provider, or otherwise by the host in need thereof, typically a human, as necessary or desired.

In certain embodiments, the composition as described herein is provided only in a controlled counseling session, and administered only once, or perhaps 2, 3, 4, or 5 or more times in repeated counseling sessions to address a mental disorder as described herein.

In other embodiments, the composition as described herein is provided outside of a controlled counseling session, and perhaps self-administered, as needed to perhaps 2, 3, 4, or 5 or more times in to address a mental disorder as described herein.

In other embodiments, the composition of the present invention may be administered on a routine basis for mental wellbeing or for entactogenic treatment.

The compounds of the current invention can be administered in a variety of doses, routes of administration, and dosing regimens, based on the indication and needs of the patient. Non-limiting examples of therapeutic use include discrete psychotherapeutic sessions, ad libitum use for treatment of episodic disorders, and ongoing use for treatment of subchronic and chronic disorders.

### Psychotherapeutic Sessions

For some indications, the medicine is taken in discrete psychotherapy or other beneficial sessions. It is anticipated that these sessions will typically be separated by more than 5 half-lives of the medicine and, for most patients, will typically occur only 1 to 5 times each year.

For these sessions, it will typically be desirable to induce clearly perceptible entactogenic effects that will facilitate fast therapeutic progress. Non-exhaustive examples of oral doses of medicine that produce clearly perceptible entactogenic effects for exemplary purposes for any of the compounds described herein include (using compounds for illustrative purposes only): about to about 800 mg, about 10 to about 750 mg, about 10 to about 700 mg, about 10 to about 650 mg, about 10 to about 600 mg, about 10 to about 550 mg, about 10 to about 500 mg, about 10 to about 450 mg, about 10 to about 400 mg, about 10 to about 350 mg, about 10 to about 300 mg, about 10 to about 250 mg, about 10 to about 200 mg, about 10 to about 150 mg, about 10 to about 100 mg, about 10 to about 50 mg, of a compound of Formula I. Other non-limiting examples of embodiments include about 50 to about 800 mg, about 100 to about 800 mg, about 150 to about 800 mg, about 200 to about 800 mg, about 250 to about 800 mg, about 300 to about 800 mg, about 350 to about 800 mg, about 400 to about 800 mg, about 450 to about 800 mg, about 500 to about 800 mg, of a compound of Formula I.

It is anticipated that the medicine would be taken once or, more rarely, two or three times in a single therapeutic session. In these cases, it is common for each subsequent dose to be half of the previous dose or lower. Multiple doses within a session typically occur because either the patient's sensitivity to the medicine was unknown and too low of an initial dose was employed or because the patient is experiencing a productive session and it is desirable to extend the duration of therapeutic effects. Controlled release preparations may be used to lengthen the duration of therapeutic effects from a single administration of the medicine. In cases where multiple administrations are used in a session, it is anticipated that individual doses will be lower so that plasma concentrations remain within a desired therapeutic range.

Non-limiting, non-exhaustive examples of indications that may benefit from psychotherapeutic sessions include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders. Also included as exemplary situations where an individual would benefit from a psychotherapeutic session are situations from a reduction of neuroticism or psychological defensiveness, an increase in openness to experience, an increase in creativity, or an increase in decision-making ability.

### Ad Libitum Use for Treatment of Episodic Disorders

For some indications, such as social anxiety, where the patient has need for relief from episodic occurrence of a disorder, it is anticipated that the medicine would be taken as needed but that uses should be separated by more than 5 half-lives of the medicine to avoid bioaccumulation and formation of tolerance.

For treating episodic disorders, clearly perceptible entactogenic effects are often not desirable, as they can impair some aspects of functioning. Non-exhaustive examples of oral doses of medicine for any of the compounds described herein include (using compounds for illustrative purposes only) that produce subtle, barely perceptible therapeutic effects include: about 2 to about 800 mg, about 2 to about 750 mg, about 2 to about 700 mg, about 2 to about 650 mg, about 2 to about 600 mg, about 2 to about 550 mg, about 2 to about 500 mg, about 2 to about 450 mg, about 2 to about 400 mg, about 2 to about 350 mg, about 2 to about 300 mg, about 2 to about 250 mg, about 2 to about 200 mg, about 2 to about 150 mg, about 2 to about 100 mg, about 2 to about 50 mg, of a compound of Formula I. Other non-limiting examples of embodiments include about 50 to about 800 mg, about 100 to about 800 mg, about 150 to about 800 mg, about 200 to about 800 mg, about 250 to about 800 mg, about 300 to about 800 mg, about 350 to about 800 mg, about 400 to about 800 mg, about 450 to about 800 mg, about 500 to about 800 mg of a compound of Formula I.

Non-limiting, non-exhaustive examples of indications that may benefit from episodic treatment include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders, provided that clinically significant signs and symptoms worsen episodically or in predictable contexts.

### Ongoing Use for Treatment of Subchronic and Chronic Disorders

For some indications, such as substance use disorders, inflammatory conditions, and neurological indications, including treatment of stroke, brain trauma, dementia, and neurodegenerative diseases, where the patient has need for ongoing treatment, it is anticipated that the medicine would be taken daily, twice daily, or three times per day. With some indications (subchronic disorders), such as treatment of stroke or traumatic brain injury, it is anticipated that treatment duration will be time-limited and dosing will be tapered when the patient has recovered. An example dose taper regimen is a reduction in dose of 10% of the original dose per week for nine weeks. With other, chronic disorders, such as dementia, it is anticipated that treatment will be continued as long as the patient continues to receive clinically significant benefits.

For treating subchronic and chronic disorders, clearly perceptible entactogenic effects are often not desirable. Non-exhaustive examples of oral doses of medicine for any of the compounds described herein include (using compounds for illustrative purposes only) that produce subtle, barely perceptible therapeutic effects with ongoing dosing include: about 2 to about 800 mg, about 2 to about 750 mg, about 2 to about 700 mg, about 2 to about 650 mg, about 2 to about 600 mg, about 2 to about 550 mg, about 2 to about 500 mg, about 2 to about 450 mg, about 2 to about 400 mg, about 2 to about 350 mg, about 2 to about 300 mg, about 2 to about 250 mg, about 2 to about 200 mg, about 2 to about 150 mg, about 2 to about 100 mg, about 2 to about 50 mg, of a compound of Formula I. Other non-limiting examples of embodiments include about 50 to about 800 mg, about 100 to about 800 mg, about 150 to about 800 mg, about 200 to about 800 mg, about 250 to about 800 mg, about 300 to about 800 mg, about 350 to about 800 mg, about 400 to about 800 mg, about 450 to about 800 mg, about 500 to about 800 mg, of a compound of Formula I.

Non-limiting, non-exhaustive examples of subchronic and chronic disorders that may benefit from regular treatment include migraine, headaches (e.g., cluster headache), neurodegenerative disorders, Alzheimer's disease, Parkinson's disease, schizophrenia, stroke, traumatic brain injury, phantom limb syndrome, and other conditions where increasing neuronal plasticity is desirable.

### Synthetic Approaches for Compounds of the Present Invention

Methods for synthesis of the compounds described herein and/or starting materials are either described in the art or will be readily apparent to the skilled artisan in view of general references well-known in the art (see, e.g., Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the compounds of the invention. In general, the approaches used for similar compounds (Ghinea & Dinica. 2016. Scope of Selective Heterocycles from Organic and Pharmaceutical Perspective, 115-142; Ramalakshmi et al. 20201. Der PharmaChemica, 2021, 13(2): 60-69; Shulgin & Shulgin. 1992. P¹HKAL. A chemical love story, Transform Press, Berkeley CA; Glennon et al. 1986. Journal of medicinal chemistry, 29(2), 194-199; Nichols et al. 1991. Journal of medicinal chemistry, 34(1), 276-281; Kedrowski et al. 2007. Organic Letters, 9(17), 3205-3207; Heravi & Zadsirjan. 2016. Current Organic Synthesis, 13(6), 780-833; Keri et al. 2017. European journal of medicinal chemistry, 138, 1002-1033; Perez-Silanes et al. 2001. Journal of Heterocyclic Chemistry, 38(5), 1025-1030; and references therein), such adaptation being that known and understood to those of ordinary skill.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched (having either more (R)-enantiomer than (S)-enantiomer, or more (S)-enantiomer than (R)-enantiomer), racemic mixture, diastereomerically pure, diastereomerically enriched, or a stereoisomeric mixture. Enantiomerically enriched compounds may have an enantiomeric excess of one enantiomer of at least 60%, at least 65%, at least 70%, at least 75%, at least 80%, at least 85%, at least 90%, at least 95%, at least 98%, at least 99%, or at least 99.5%.

In addition, it is understood that in any compound described herein having one or more double bond(s) generating geometrical isomers that can be defined as E or Z, each double bond may independently be E or Z a mixture thereof.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

Stereoisomers may include enantiomers, diastereomers, racemic mixtures, and combinations thereof. Such stereoisomers can be prepared and separated using conventional techniques, either by reacting enantiomeric starting materials, or by separating isomers of compounds disclosed herein.

Isomers may include geometric isomers. Examples of geometric isomers include cis isomers or trans isomers across a double bond. Other isomers are contemplated among the compounds of the present disclosure. The isomers may be used either in pure form or in admixture with other isomers of the structures of Formula I described herein.

Various methods are known in the art for preparing optically active forms and determining activity. Such methods include standard tests described herein and other similar tests which are well known in the art. Examples of methods that can be used to obtain optical isomers of the compounds according to the present disclosure include the following:
i) physical separation of crystals whereby macroscopic crystals of the individual enantiomers are manually separated. This technique may particularly be used if crystals of the separate enantiomers exist (i.e., the material is a conglomerate), and the crystals are visually distinct;
ii) simultaneous crystallization whereby the individual enantiomers are separately crystallized from a solution of the racemate, possible only if the latter is a conglomerate in the solid state;
iii) enzymatic resolutions whereby partial or complete separation of a racemate by virtue of differing rates of reaction for the enantiomers with an enzyme;
iv) enzymatic asymmetric synthesis, a synthetic technique whereby at least one step of the synthesis uses an enzymatic reaction to obtain an enantiomerically pure or enriched synthetic precursor of the desired enantiomer;
v) chemical asymmetric synthesis whereby the desired enantiomer is synthesized from an achiral precursor under conditions that produce asymmetry (i.e., chirality) in the product, which may be achieved using chiral catalysts or chiral auxiliaries;
vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors whereby the desired enantiomer is obtained from non-chiral starting materials and where the stereochemical integrity is not or is only minimally compromised over the course of the synthesis;
x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography whereby the racemate is volatilized and enantiomers are separated by virtue of their differing interactions in the gaseous mobile phase with a column containing a fixed non-racemic chiral adsorbent phase;
xii) extraction with chiral solvents whereby the enantiomers are separated by virtue of preferential dissolution of one enantiomer into a particular chiral solvent; and
xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane, which allows only one enantiomer of the racemate to pass through.

### Example 1: Synthesis Schemes

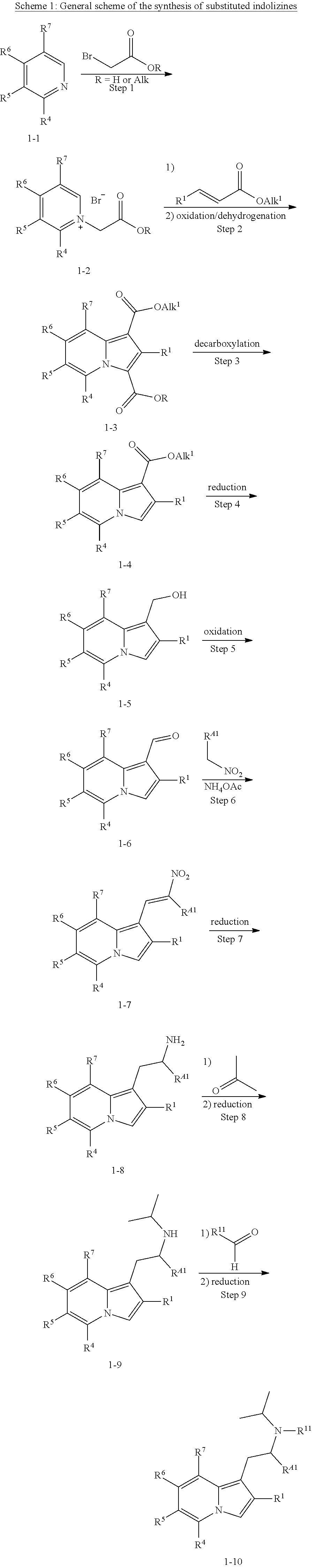

In certain aspects the compound 1-10 is prepared from pyridinium salt 1-2 as a building block for the construction of indolizine system. Under basic conditions, methylene group between pyridinium nitrogen and carbonyl in compound 1-2 is deprotonated resulting in pyridinium ylide (not shown), which reacts with alpha-beta-unsaturated carboxylate (alkene) according to the 1,3-cycloaddition reaction scheme. Subsequent oxidation (dehydrogenation) in step 2 is required for the formation of indolizine system to give compound 1-3. Further reactions, such as decarboxylation, sequence of reductions and oxidations, include transformations of substituents attached to the five-membered ring in indolizine.

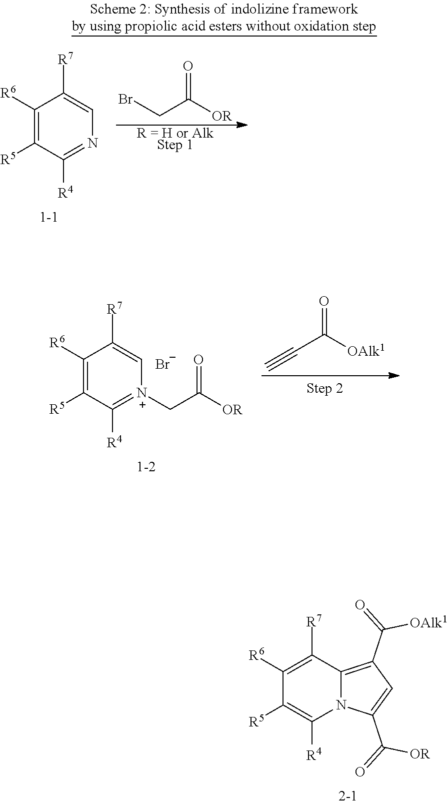

This synthesis is akin to the synthesis described in Scheme 1, with the only difference being the usage of propiolic acid esters (alkyne) as a dipolarophile in the 1,3-cycloaddition reaction (step 2) with pyridinium ylide generated from salt 1-2. In such a case, subsequent dehydrogenation is not required, and the reaction directly affords indolizine 2-1, which may be further used in subsequent chemical transformations.

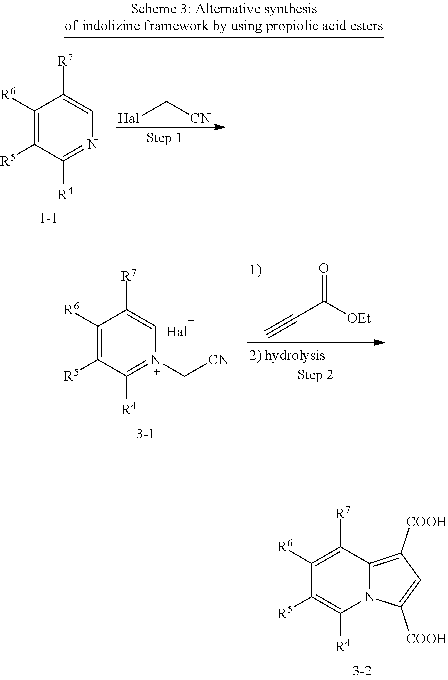

According to this synthetic scheme, pyridinium salt 3-1 is formed from haloacetonitrile and later used in the same reaction sequence as illustrated in Scheme 2.

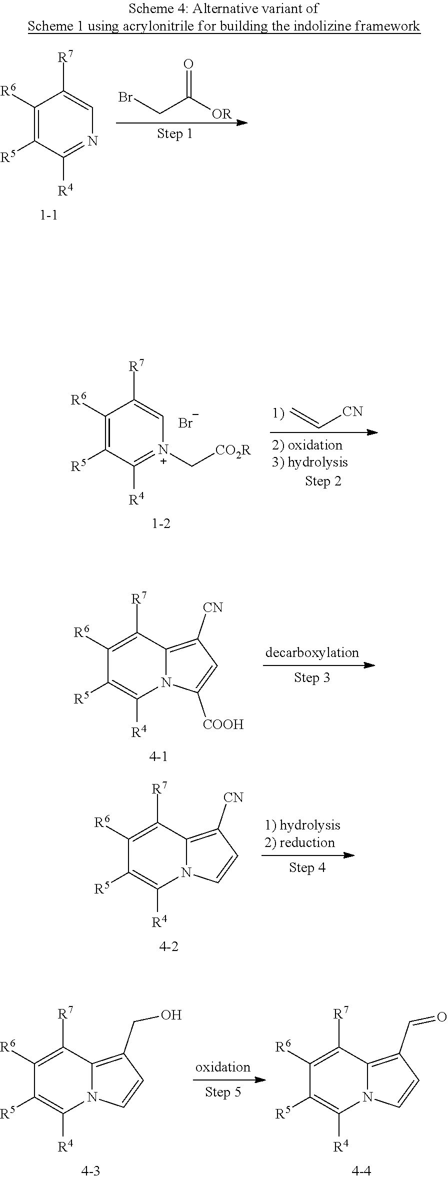

In this reaction scheme, which is a variation of Scheme 1, pyridinium ylide generated from salt 1-2 is subjected to the reaction with acrylonitrile as a dipolarophile. Further transformations may include reactions with substituents attached to the indolizine heterocycle.

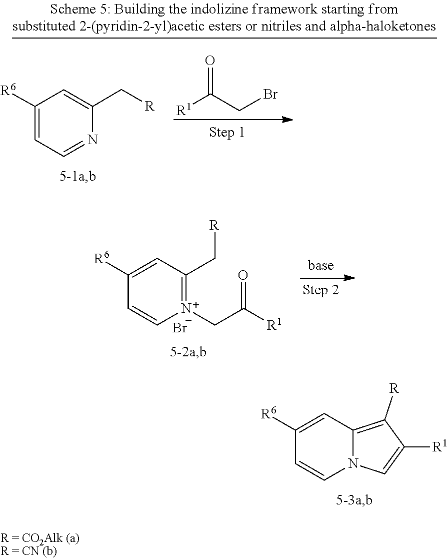

According to this synthetic route, N-alkylation of 2-substituted pyridine 5-1a,b with an α-haloketone gives the quaternary pyridinium salts 5-2a,b, which can undergo the ring closure reaction under basic conditions (step 2) to give the corresponding indolizine 5-3a,b. This reaction proceeds as a result of deprotonation of methylene unit between pyridine ring and group R followed by an intramolecular aldol type condensation.

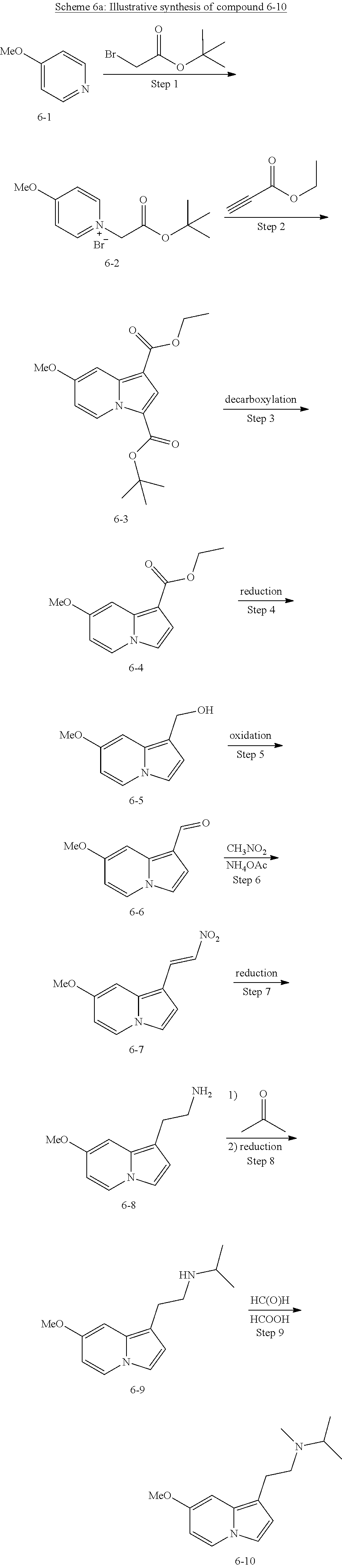

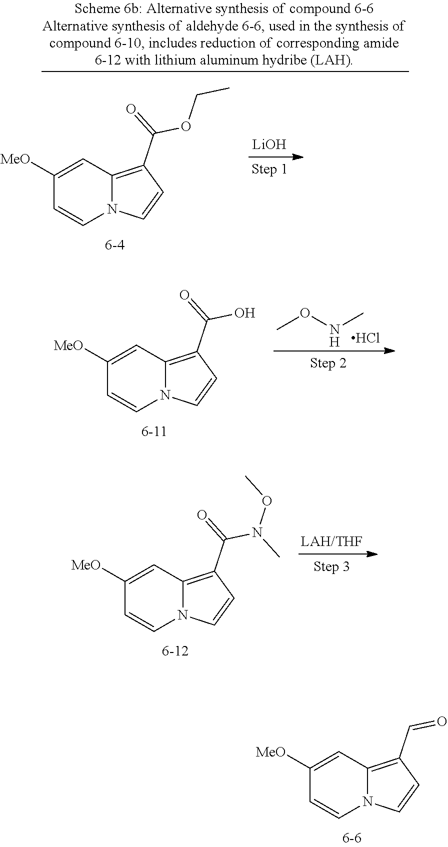

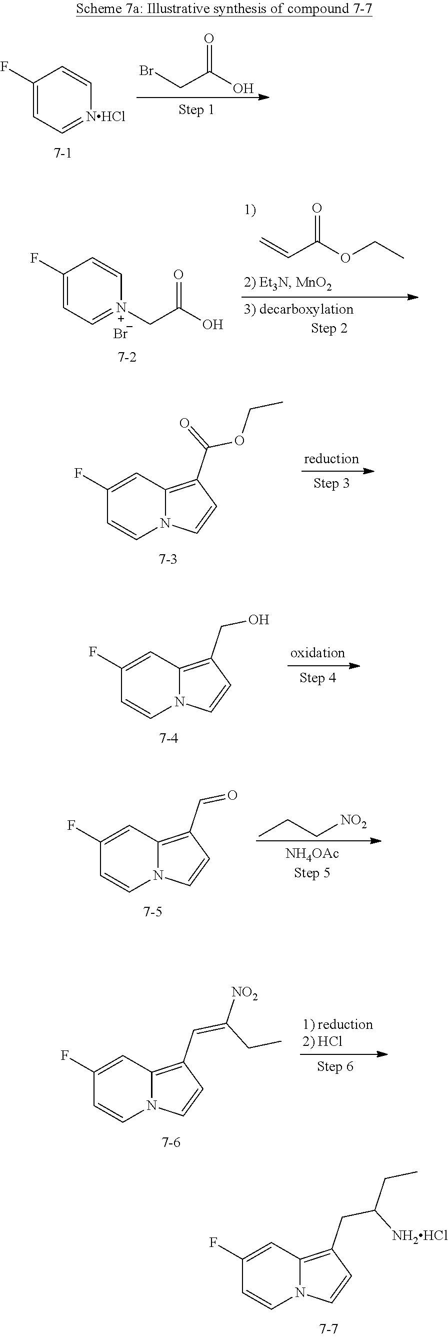

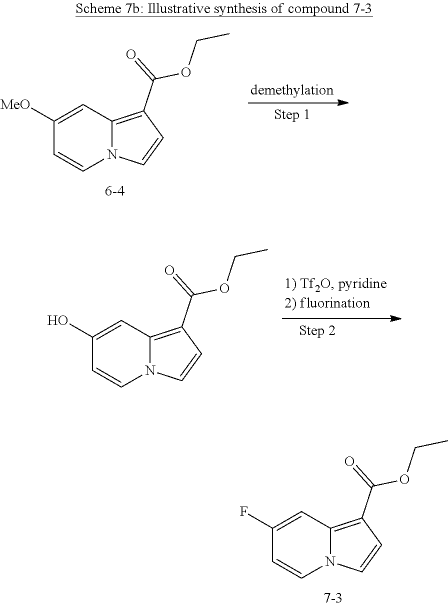 [SMILES: CCOC(=O)c1ccn2ccc(OC)cc12 ; CCOC(=O)c1ccn2ccc(O)cc12 ; CCOC(=O)c1ccn2ccc(F)cc12]

Intermediate compound 7-3, used in the synthesis of compound 7-7, may be also prepared from corresponding methoxy analogue 6-4 through demethylation (which may be performed by using dodecanethiol, pyridinium hydrochloride, boron tribromide as demethylation agents) in the first step, followed by the synthesis of triflate derivative from corresponding phenol intermediate, followed by the exchange of triflate group for fluorine to furnish compound 7-3 in step 2.

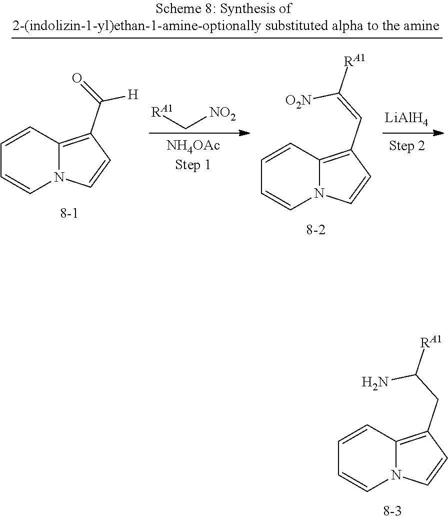

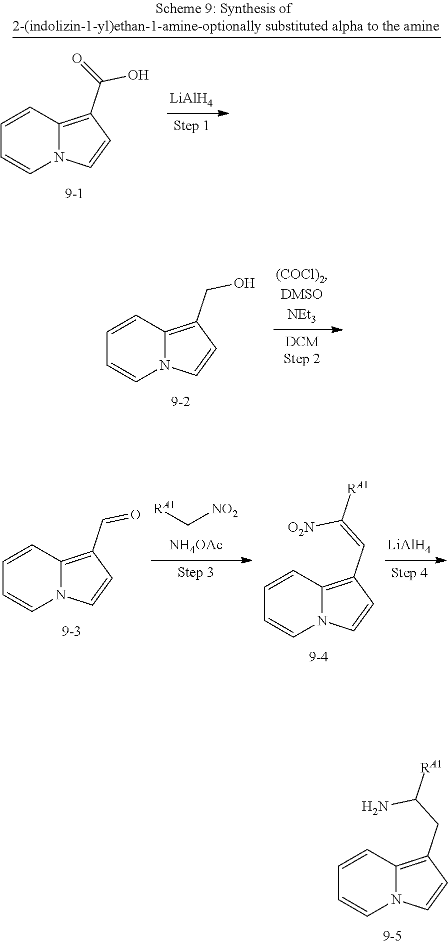

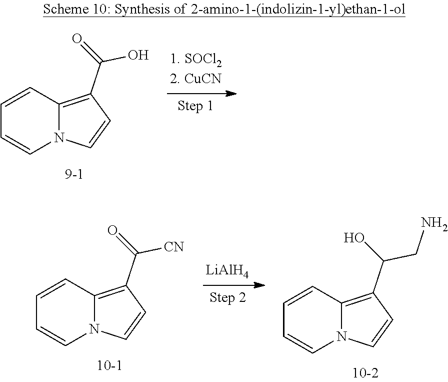 [SMILES: O=C(O)c1ccn2ccccc12 ; N#CC(=O)c1ccn2ccccc12 ; NCC(O)c1ccn2ccccc12]

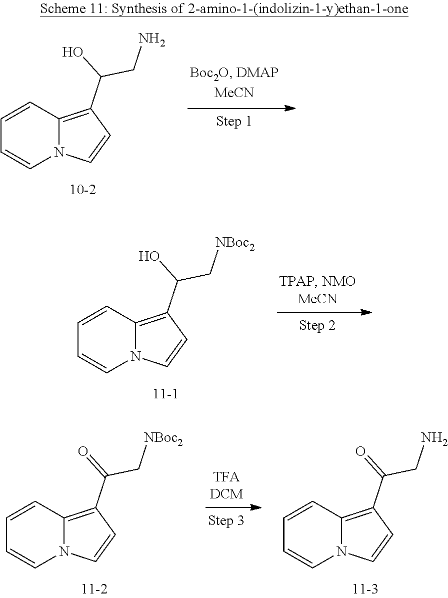

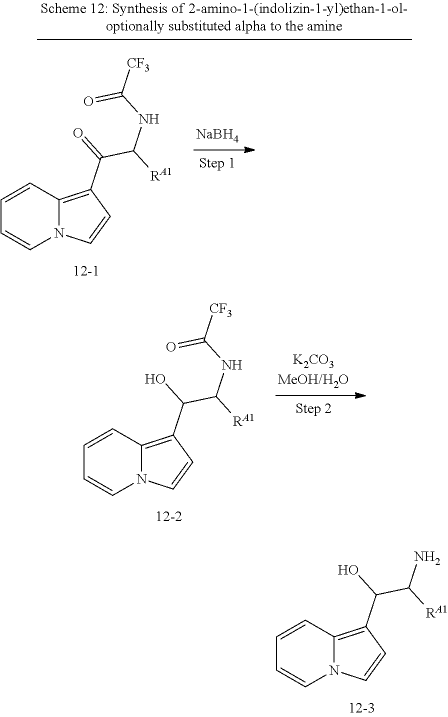

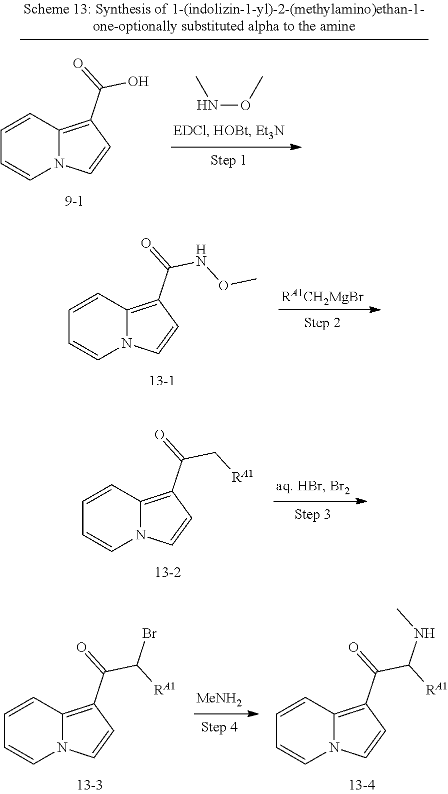

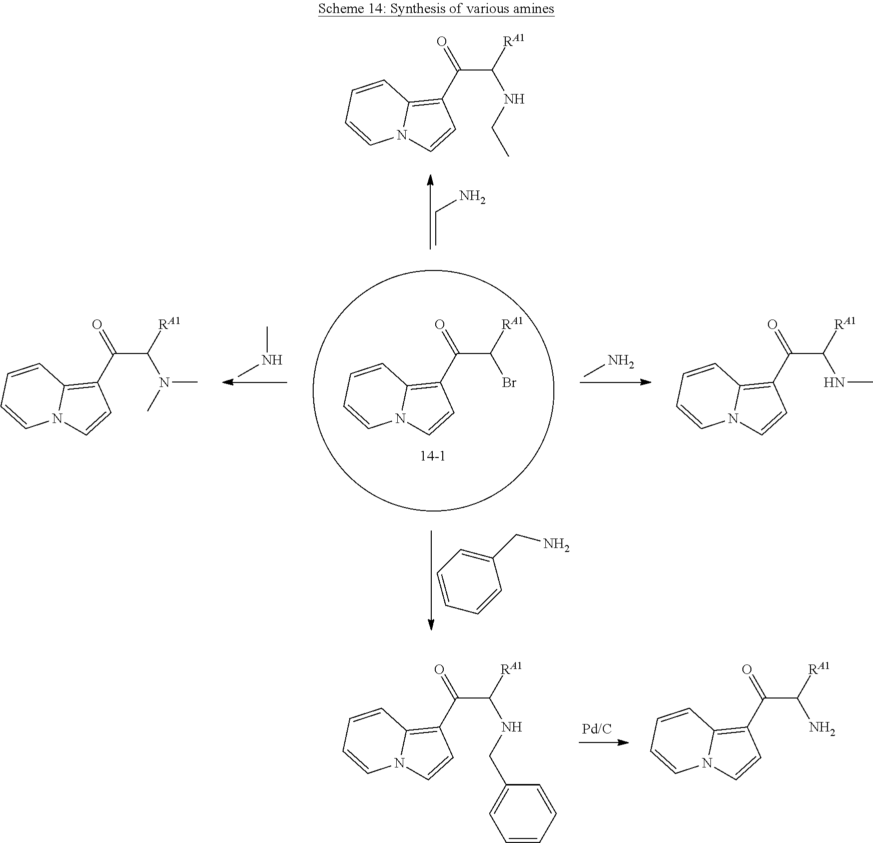

The chiral compounds of the present invention can be separated using the various chiral separation techniques discussed herein and otherwise known such as chiral HPLC. In other embodiments a compound is provided that is a diastereomer and the diastereomers can be separated using conventional techniques and then enantiomers can be further purified if desired.

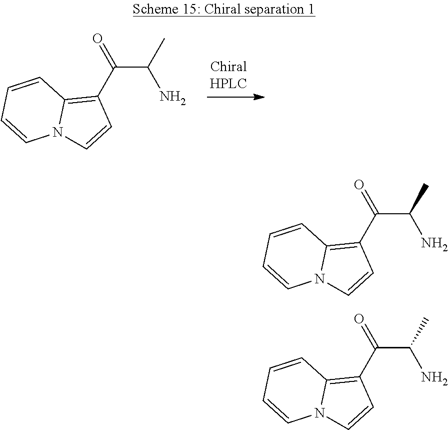

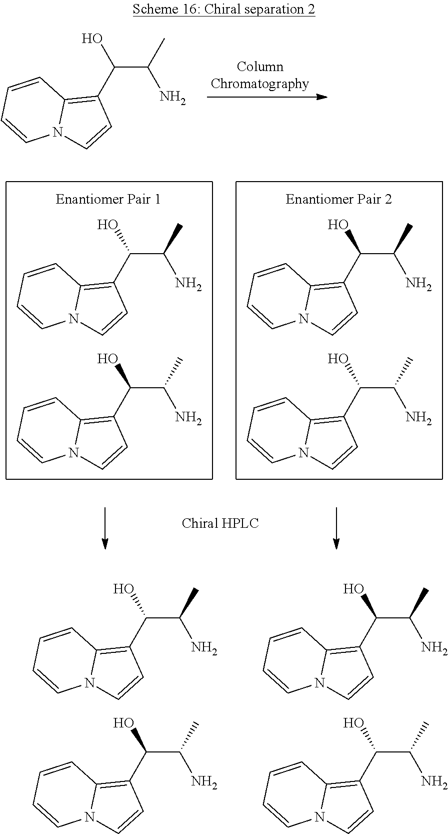

In other embodiments the chirality of the molecule can be set or enhanced by choice of synthetic conditions. For example, chiral reducing agents are well known and using bulky or small reducing agents can effect stereochemical selectivity (see Schemes 19, 20). Alternatively other chiral reagents can be employed (see Schemes 18, 21).

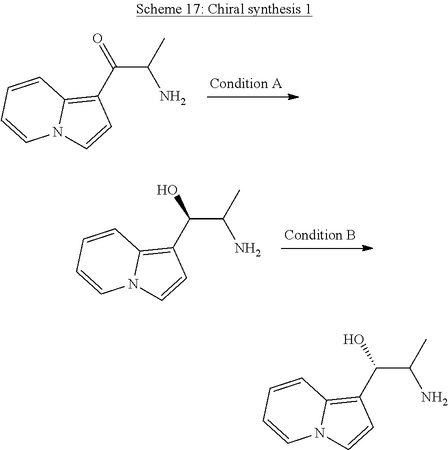

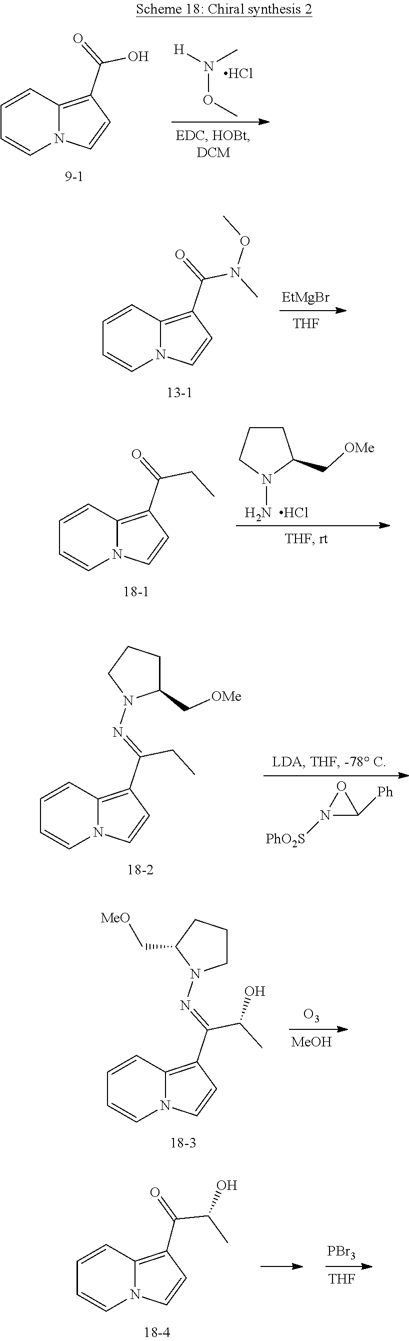

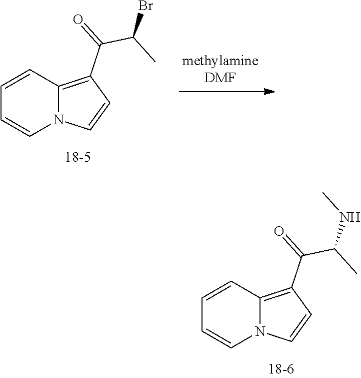

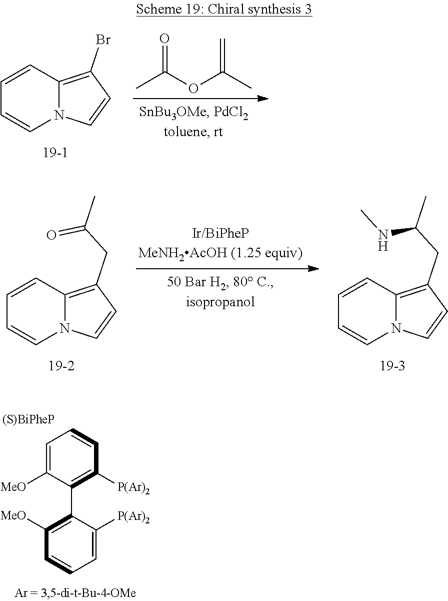

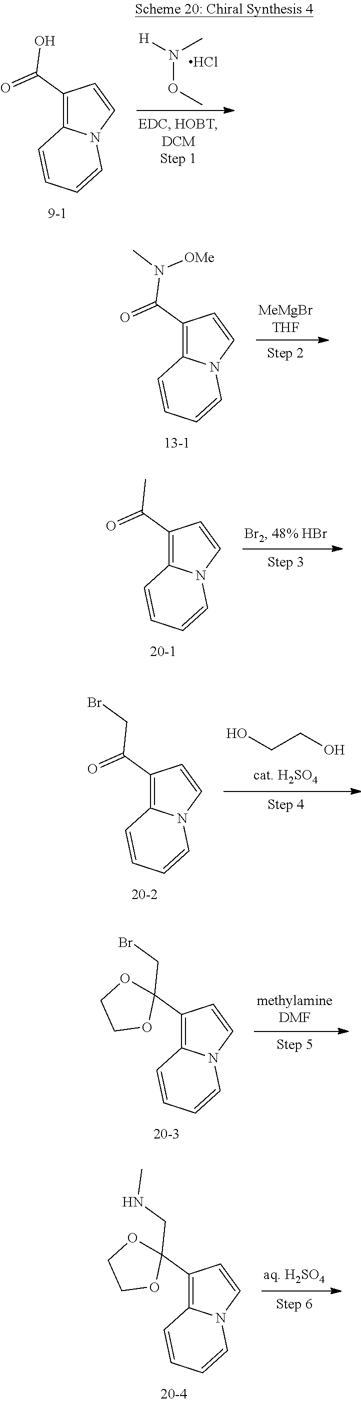

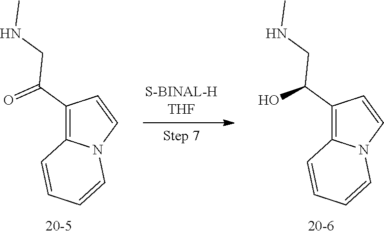

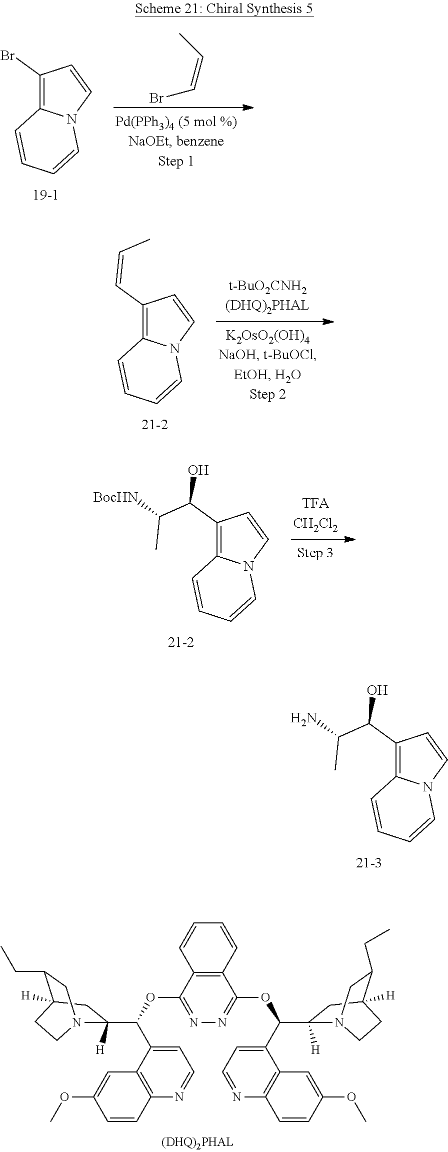

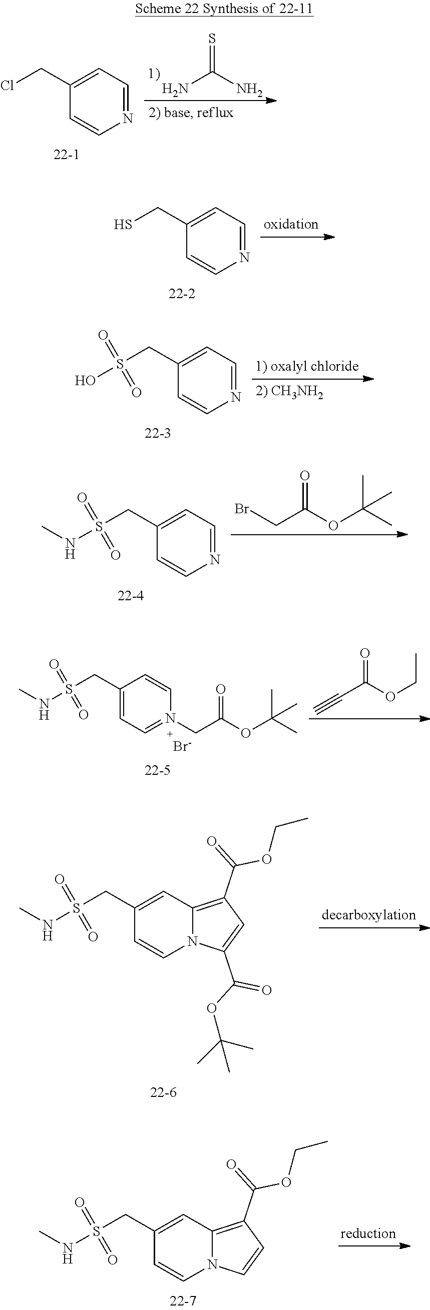

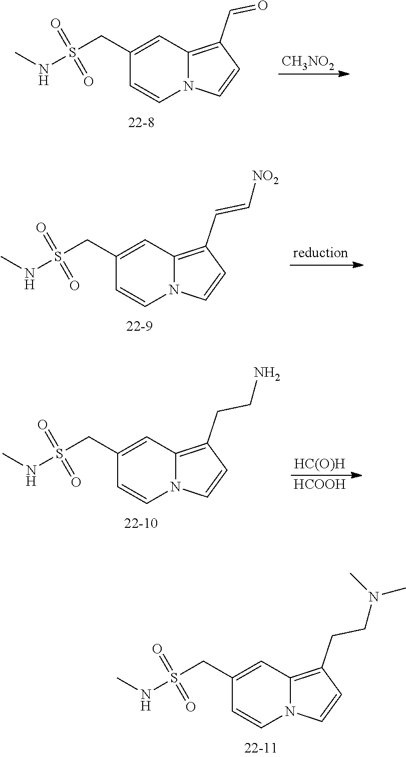

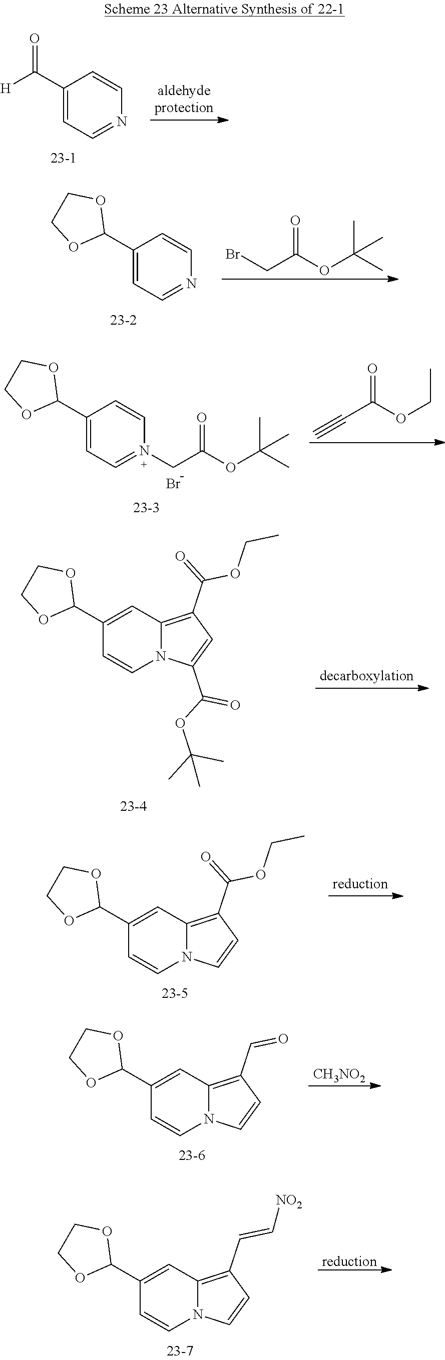

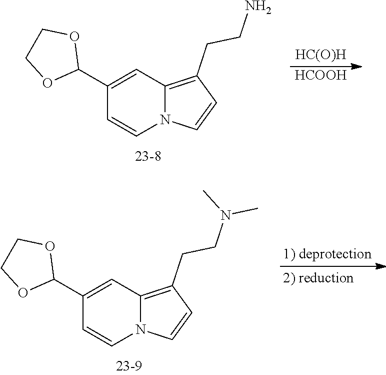 [SMILES: NCCc1ccn2ccc(C3OCCO3)cc12 ; "HCOOH" ; "HC(O)H" ; CN(C)CCc1ccn2ccc(C3OCCO3)cc12]

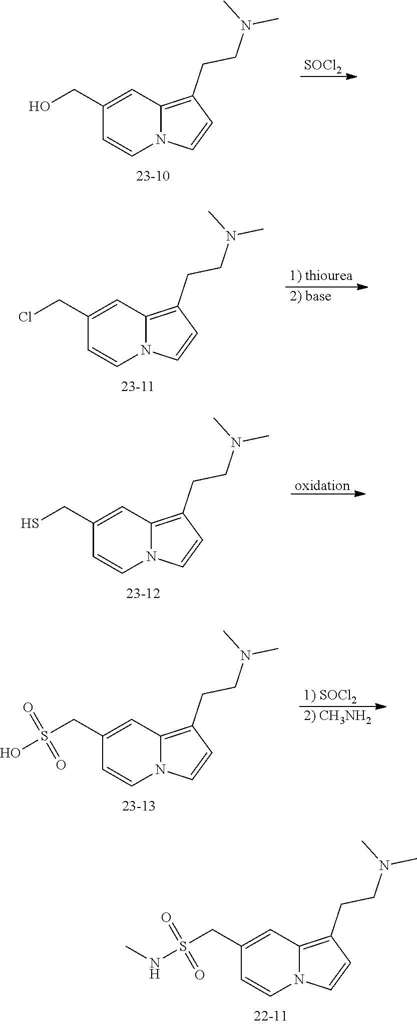

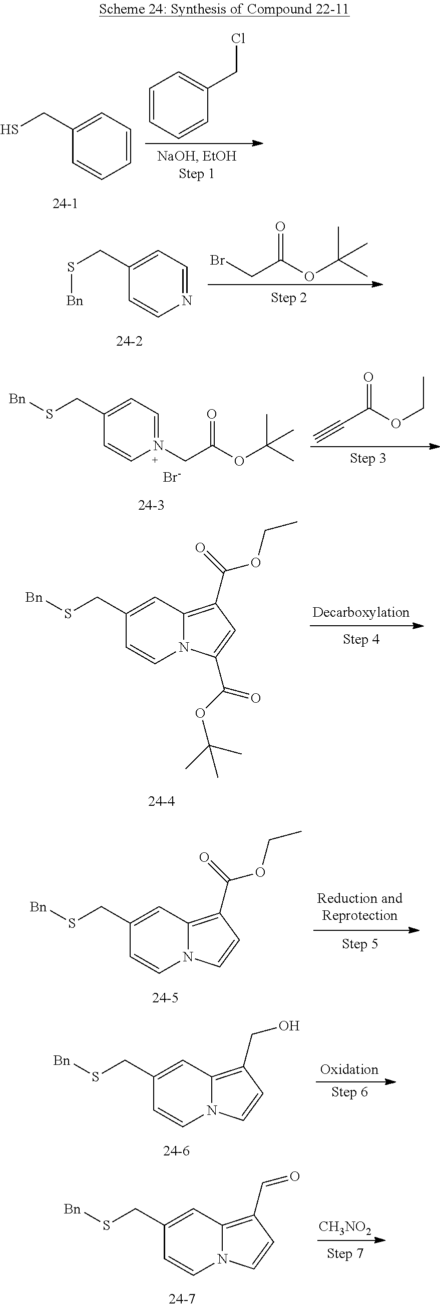

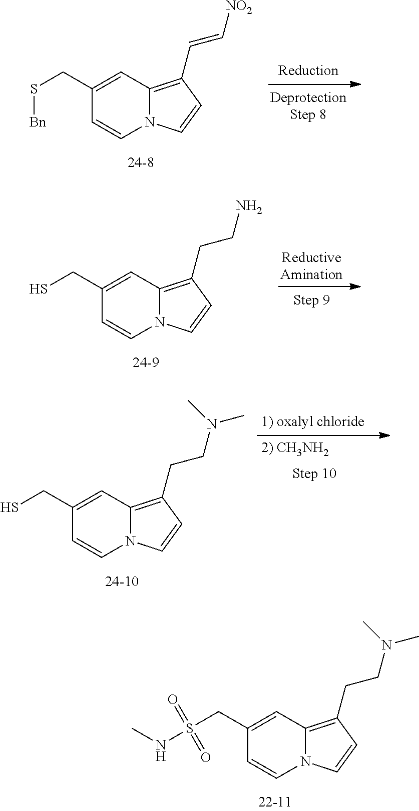

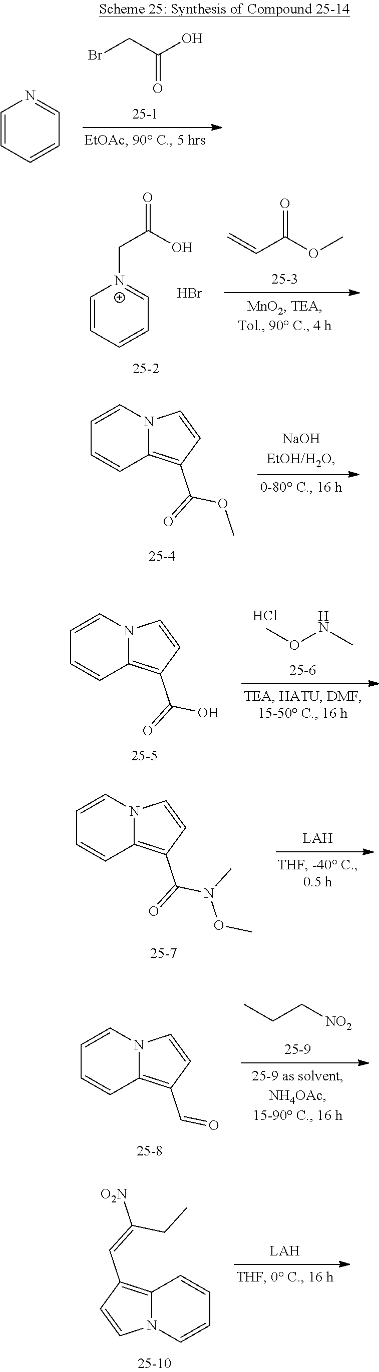

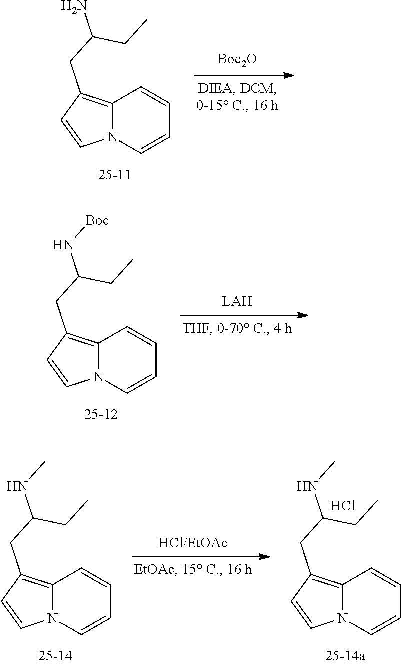

### Synthesis of 1-(carboxymethyl)pyridin-1-ium HBr salt (25-2)

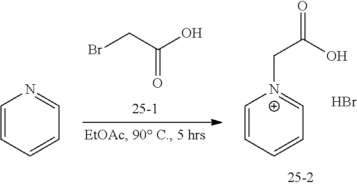 [SMILES: c1ccncc1 ; O=C(O)CBr ; O=C(O)C[n+]1ccccc1 ; Br]

To a solution of pyridine (50 g, 632.12 mmol) in CH₃CN (500 mL) was added 2-bromoacetic acid (87.84 g, 632.12 mmol) at 20° C. The mixture was stirred at 90° C. for 16 h. The reaction mixture was filtered. The filter cake was concentrated under reduced pressure to give 1-(carboxymethyl)pyridin-1-ium HBr salt (122 g, crude) as white solid. LCMS, m/z=138.4 [M+H]+¹H NMR (400 MHz, D₂O): δ 8.8 (d, J=5.6 Hz, 2H), 8.4 (t, J=8.0 Hz, 1H), 8.0 (t, J=7.2 Hz, 2H), 5.49 (s, 2H).

### Synthesis of methylindolizine-1-carboxylate (25-4)

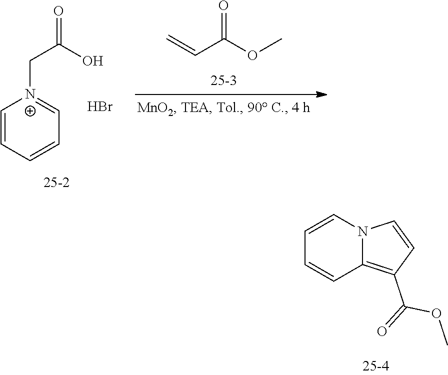

To a solution of 1-(carboxymethyl) pyridin-1-ium HBr salt (120 g, 273.9 mmol) in Tol. (1200 mL) was added MnO₂ (142.86 g, 821.7 mmol), TEA (55.44 g, 273.9 mmol) and methyl acrylate (141.48 g, 821.7 mmol) at 0° C. The mixture was stirred at 90° C. for 16 h. The mixture was quenched by addition water (1000 mL) at 0° C. The mixture was filtered. The filtrate was extracted with EtOAc (3×300 mL). The combined organic layers were washed with brine (1000 mL), dried over anhydrous sodium sulfate, filtered and concentrated under reduced pressure to give a residue. The residue was purified by silica gel column chromatography (PE:EtOAc=1:0 to 0:1) to give methyl indolizine-1-carboxylate (7.1 g, 7%) as yellow solid (PE:EtOAc=3:1, Rf=0.57). LCMS, m/z=176.3 [M+H]^{+ 1}H NMR (400 MHz, CDCl3): δ 8.17 (d, J=9.2 Hz, 1H), 7.99 (d, J=7.2 Hz, 1H), 7.26-7.20 (m, 2H), 7.08-6.98 (m, 1H), 6.69 (t, J=6.8 Hz, 1H), 3.90 (s, 3H).

### Synthesis of indolizine-1-carboxylic acid (25-5)

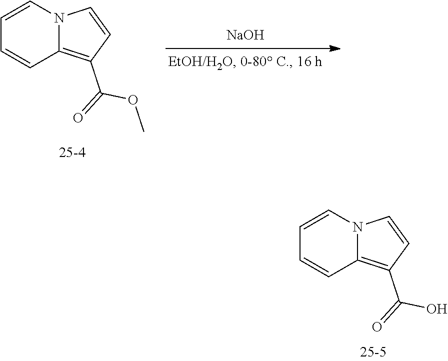

To a solution of methyl indolizine-1-carboxylate (7.1 g, 40.53 mmol) in EtOH (60 mL) was added a solution of NaOH (4.86 g, 121.59 mmol) in H₂O (12 mL) at 0° C. The mixture was heated to 80° C. and stirred for 16 h. The reaction mixture was concentrated under reduced pressure and then adjusted to pH=4 by addition of aq.HCl (1 M) and filtered. The filter cake was concentrated under reduced pressure to give indolizine-1-carboxylic acid (6.2 g, crude) as white solid. LCMS, m/z=162.2 [M+H]^{+ 1}H NMR (400 MHz, DMSO): δ 12.05-11.60 (m, 1H), 8.44 (d, J=7.2 Hz, 1H), 8.03 (d, J=8.8 Hz, 1H), 7.57 (d, J=3.2 Hz, 1H), 7.11-7.07 (m, 2H), 6.81 (dt, J=1.2, 6.8 Hz, 1H).

### Synthesis of N-methoxy-N-methylindolizine-1-carboxamide (25-7)

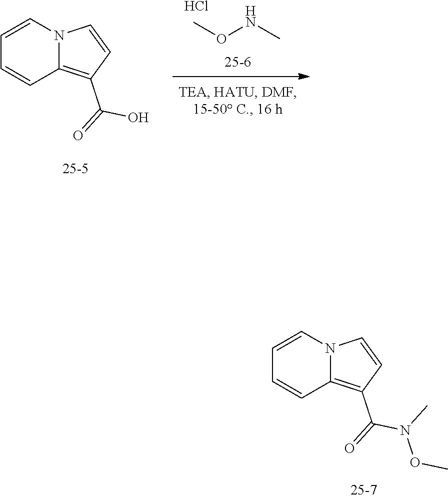

To a solution of indolizine-1-carboxylic acid (6 g, 37.24 mmol) in THE (60 mL) was added N-methoxymethanamine HCl salt (7.62 g, 74.48 mmol), EDCI (10.7 g, 55.84 mmol), DIEA (14.44 g, 111.7 mmol) and HOBt (7.54 g, 55.84 mmol) at 0° C. The mixture was stirred at 30° C. for 12 h. The mixture was quenched by H₂O (60 mL), extracted with EtOAc (3×60 mL). The combined organic layers were washed by brine (3×20 mL), dried over Na₂SO₄, filtered and concentrated under reduced pressure. The residue was purified by silica gel column chromatography (PE:EtOAc=1:0 to 0:1) to give N-methoxy-N-methyl-indolizine-1-carboxamide (7 g, crude) as white solid (PE:EtOAc=1:1, Rf=0.52). LCMS, m/z=205.0 [M+H]^{+ 1}H NMR (400 MHz, CDCl₃): δ 8.39 (d, J=9.2 Hz, 1H), 7.95 (d, J=6.8 Hz, 1H), 7.32 (d, J=3.2 Hz, 1H), 7.21 (d, J=3.2 Hz, 1H), 6.97 (ddd, J=1.2, 6.8, 9.2 Hz, 1H), 6.65 (dt, J=1.2, 6.8 Hz, 1H), 3.70 (s, 3H), 3.37 (s, 3H).

### Synthesis of indolizine-1-carbaldehyde (25-8)

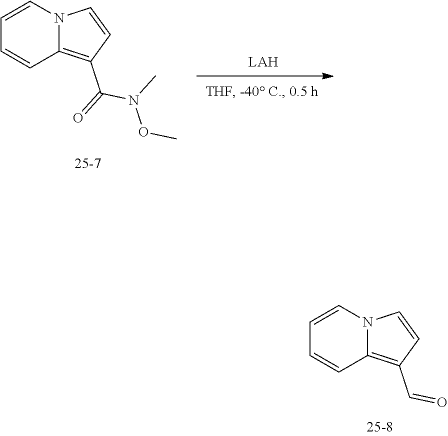

To a solution of N-methoxy-N-methylindolizine-1-carboxamide (4 g, 19.59 mmol) in THF (40 5 mL) was added LiAlH₄ (2.5 M in THF, 3.92 mL, 9.80 mmol) at −40° C. under N2. The mixture was stirred at −40° C.-0° C. for 2 h. Na2SO4.10H₂O (20 g) was added to the reaction mixture at 0° C., and then stirred at 0° C. for 10 min. The mixture was filtered and concentrated under reduced pressure to give indolizine-1-carbaldehyde (3.2 g, crude) as light yellow oil. LCMS, m/z=146.2 [M+H]^{+ 1}H NMR (400 MHz, CDCl₃): δ 9.93 (s, 1H), 8.15 (br d, J=8.8 Hz, 1H), 7.98 (td, J=1.2, 6.8 Hz, 1H), 7.19 (s, 1H), 7.14-7.11 (m, 1H), 7.10-7.09 (m, 1H), 6.75 (dt, J=1.2, 6.8 Hz, 1H),

### Synthesis of (E)-1-(2-nitrobut-1-en-1-yl)indolizine (25-10)

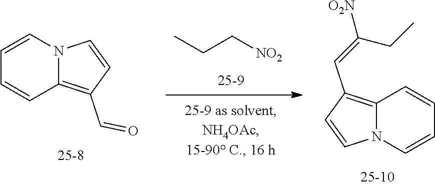

To a solution of indolizine-1-carbaldehyde (3 g, 12.60 mmol) in 1-nitropropane (29.94 g, 336.06 mmol, 30 mL) was added NH₄OAc (972 mg, 25.22 mmol) at 15° C. The mixture was stirred at 90° C. for 16 h. The reaction mixture was filtered and concentrated under reduced pressure to give a residue. The residue was purified by silica gel column chromatography (Petroleum ether:Ethyl acetate=1:0 to 1:1) to give (E)-1-(2-nitrobut-1-en-1-yl)indolizine (1.3 g, 41%) as orange solid (PE:EtOAc=10:1, Rf=0.5). LCMS, m/z=217.2 [M+H]^{+ 1}H NMR (400 MHz, CDCl₃): δ 8.52 (s, 1H), 8.02 (d, J=6.8 Hz, 1H), 7.76 (d, J=9.2 Hz, 1H), 7.43 (d, J=3.2 Hz, 1H), 7.09 (d, J=3.2 Hz, 1H), 7.09-7.05 (m, 1H), 6.77 (dt, J=0.8, 6.8 Hz, 1H), 3.04 (q, J=7.6 Hz, 2H), 1.30 (t, J=7.2 Hz, 3H).

### Synthesis of 1-(indolizin-1-yl)butan-2-amine (25-11)

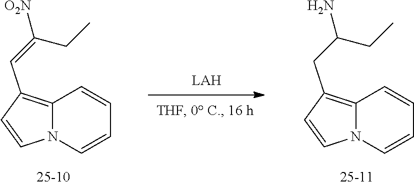 [SMILES: CC/C(=C\c1ccn2ccccc12)[N+](=O)[O-] ; CCC(N)Cc1ccn2ccccc12]

To a solution of (E)-1-(2-nitrobut-1-en-1-yl)indolizine (1.3 g, 6.01 mmol) in THF (10 mL) was added LiAlH₄ (2.5 M in THF, 7.21 mL, 18.03 mmol) at 0° C. under N2. The mixture was stirred at 0° C.-15° C. for 16 h. Na2SO4.10H₂O (15 g) was added to the reaction mixture at 0° C., and then stirred at 0° C. for 10 min. The mixture was filtered and then the filter liquor was concentrated under reduced pressure to give 1-indolizin-1-ylbutan-2-amine (1.5 g, crude) as black oil. LCMS, m/z=189.3 [M+H]+

### Synthesis of tert-butyl (1-(indolizin-1-yl)butan-2-yl)carbamate (Severity of depression, anxiety, and trauma-related)

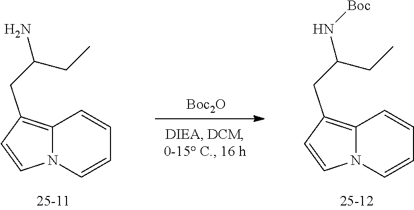 [SMILES: "Boc2O" ; CCC(N)Cc1ccn2ccccc12 ; CCC(Cc1ccn2ccccc12)NC(=O)OC(C)(C)C]

To a solution of 1-(indolizin-1-yl)butan-2-amine (1.5 g, 7.97 mmol) in DCM (15 mL) was added DIEA (3.09 g, 23.90 mmol, 4.16 mL) and Boc₂O (1.74 g, 7.97 mmol) at 0° C. under N2. The mixture was stirred at 15° C. for 2 h. The reaction mixture was quenched by addition water (15 mL), and then extracted with EtOAc (3×5 mL). The combined organic layers were washed with brine (10 mL), dried over Na2SO4, filtered and concentrated under reduced pressure to give a residue. The residue was purified by silica gel column chromatography (PE:EtOAc=1:0 to 1:1) to give tert-butyl (1-(indolizin-1-yl)butan-2-yl)carbamate (1.1 g, 48%) as a black solid. (PE:EtOAc=10:1, Rf=0.6). LCMS, m/z=289.2 [M+H]^{+ 1}H NMR (400 MHz, CDCl₃): δ 7.84 (d, J=7.2 Hz, 1H), 7.33 (br d, J=9.2 Hz, 1H), 7.24 (d, J=2.4 Hz, 1H), 6.63-6.57 (m, 2H), 6.42 (t, J=6.4 Hz, 1H), 4.39-4.30 (m, 1H), 3.76 (br s, 1H), 2.93 (d, J=5.2 Hz, 2H), 1.61-1.51 (m, 2H), 1.44 (s, 9H), 0.94 (t, J=7.2 Hz, 3H)

### Synthesis of 1-(indolizin-1-yl)-N-methylbutan-2-amine (25-14)

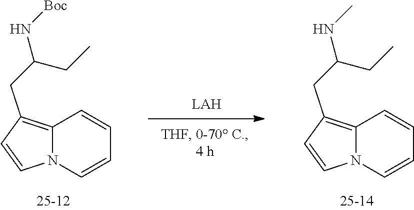 [SMILES: CCC(Cc1ccn2ccccc12)NC(=O)OC(C)(C)C ; CCC(Cc1ccn2ccccc12)NC]

To a solution of tert-butyl (1-(indolizin-1-yl)butan-2-yl)carbamate (320 mg, 1.11 mmol) in THF (3 mL) was added LiAlH₄ (2.5 M in THF, 2.22 mL, 5.55 mmol) at 0° C. under N2. The mixture was stirred at 70° C. for 2 h. Na2SO4.10H₂O (7 g) was added to the reaction mixture at 0° C., and then stirred at 0° C. for 10 min. The mixture was filtered. The filter liquor was concentrated under reduced pressure to give 1-indolizin-1-yl-N-methyl-butan-2-amine (200 mg, 89%) as light yellow oil. LCMS, m/z=203.2 [M+H]⁺

### Synthesis of 1-(indolizin-1-yl)-N-methylbutan-2-amine HCl salt (25-14 HCl)

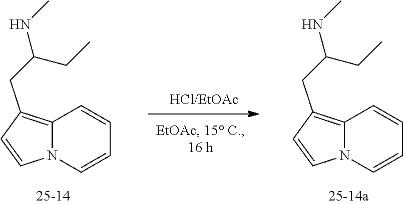

To a solution of 1-(indolizin-1-yl)-N-methylbutan-2-amine (200 mg, 0.989 mmol) in EtOAc (2 mL) was added HCl/EtOAc (4 M, 2 mL) at 15° C. The mixture was stirred at 15° C. for 16 h. The reaction mixture was concentrated under reduced pressure to give a residue. The crude product was triturated with EtOAc (1 mL) at 15° C. for 30 min to give 1-(indolizin-1-yl)-N-methylbutan-2-amine HCl salt (155 mg, 66%) as brown solid. LCMS, m/z=203.2 [M+H]^{+ 1}H NMR (400 MHz, CDCl₃): δ 9.2-8.99 (m, 2H), 8.18 (br d, J=7.2 Hz, 1H), 7.51-7.49 (m, 2H), 6.70 (d, J=2.0 Hz, 1H), 6.66-6.62 (m, 1H), 6.49-6.46 (m, 1H), 3.29-3.16 (m, 2H), 2.98-2.95 (m, 1H), 2.52 (t, J=5.6 Hz, 3H), 1.60-1.53 (m, 2H), 0.90 (t, J=7.6 Hz, 3H).

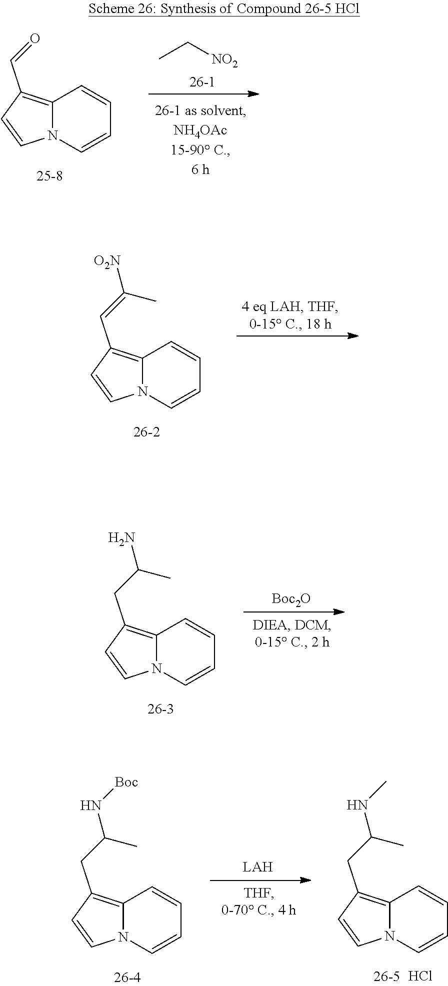

Synthesis of (E)-1-(2-nitroprop-1-en-1-yl)indolizine (26-2): To a solution of indolizine-1-carbaldehyde (2 g, 13.78 mmol) in 1-nitroethane (23.64 g, 314.92 mmol, 22.47 mL) was added NH₄OAc (2.12 g, 27.56 mmol) at 15° C. The mixture was stirred at 90° C. for 6 h. The reaction mixture was quenched by addition water (30 mL), extracted with EtOAc (3×10 mL). The combined organic layers were washed by brine (3×10 mL), dried over Na₂SO₄, filtered and concentrated under reduced pressure to give a residue. The residue was purified by silica gel column chromatography (PE:EtOAc=100:1 to 85:15) to give 1-[(E)-2-nitroprop-1-enyl]indolizine (550 mg, 20%) as blue solid. LCMS, m/z=203.2 [M+H]^{+ 1}H NMR (400 MHz, CDCl₃): δ 8.57 (s, 1H), 8.02 (d, J=6.8 Hz, 1H), 7.76 (d, J=9.2 Hz, 1H), 7.43 (d, J=3.2 Hz, 1H), 7.12 (d, J=3.2 Hz, 1H), 7.07 (dt, J=0.8, 6.8, 9.2 Hz, 1H), 6.77 (dt, J=0.8, 6.8 Hz, 1H), 2.58 (s, 3H).

Synthesis of 1-indolizin-1-ylpropan-2-amine (26-3): To a solution of 1-[(E)-2-nitroprop-1-enyl]indolizine (550 mg, 2.72 mmol) in THF (6 mL) was added LAH (2.5 M in THF, 3.26 mL) at 0° C. under N2. The mixture was stirred at 15° C. for 18 h. The reaction mixture was quenched by addition Na₂SO₄·10H₂O (8 g), and then filtered and concentrated under reduced pressure to give 1-indolizin-1-ylpropan-2-amine (800 mg, crude) as orange oil. LCMS, m/z=175.2 [M+H]^{+ 1}H NMR (400 MHz, CDCl₃): δ 7.85 (d, J=7.2 Hz, 1H), 7.34 (d, J=9.2 Hz, 1H), 7.25 (d, J=2.4 Hz, 1H), 6.65 (d, J=2.4 Hz, 1H), 6.58 (dd, J=6.4 8.4 Hz, 1H), 6.42-6.40 (m, 1H), 3.25-3.18 (m, 1H), 2.85 (dd, J=5.2, 14.0 Hz, 1H), 2.70-2.66 (m, 1H), 1.15 (d, J=6.0 Hz, 3H).

Synthesis of tert-butyl (1-(indolizin-1-yl)propan-2-yl)carbamate (26-4): To a solution of 1-indolizin-1-ylpropan-2-amine (800 mg, 4.59 mmol) in DCM (8 mL) was added Boc₂O (1.00 g, 4.59 mmol, 1.05 mL) and DIEA (1.78 g, 13.77 mmol, 2.40 mL) at 15° C. The mixture was stirred at 15° C. for 2 h. The mixture was filtered and then quenched by H₂O (20 mL), extracted with DCM (3×10 mL). The combined organic layers were washed by brine (3×10 mL), dried over Na₂SO₄, filtered and concentrated under reduced pressure. The residue was purified by silica gel column chromatography (PE:EtOAc=100:1 to 85:15) to give tert-butyl (1-(indolizin-1-yl)propan-2-yl)carbamate (320 mg, 25%) as white solid. LCMS, m/z=275.2 [M+H]^{+ 1}H NMR (400 MHz, CDCl₃): δ 7.86-7.84 (m, 1H), 7.34 (br d, J=9.2 Hz, 1H), 7.24 (d, J=2.4 Hz, 1H), 6.63 (d, J=2.4 Hz, 1H), 6.59 (dt, J=0.8, 6.4, 9.2 Hz, 1H), 6.42-6.41 (m, 1H), 4.41 (br s, 1H), 3.94 (br s, 1H), 2.97-2.86 (m, 2H), 1.45 (s, 9H), 1.10 (d, J=6.4 Hz, 3H).

Synthesis of 1-(indolizin-1-yl)-N-methylpropan-2-amine HCl salt (26-5 HCl): To a solution of tert-butyl (1-(indolizin-1-yl)propan-2-yl)carbamate (100 mg, 0.36 mmol) in THE (1 mL) was added LAH (2.5 M in THF, 0.73 mL) at 0° C. The mixture was stirred at 70° C. for 2 h, Na₂SO₄·10H₂O (4 g) was added to the reaction mixture at 0° C., and then stirred at 0° C. for 10 min.

The mixture was filtered and concentrated under reduced pressure to give a residue. To a solution of 1-indolizin-1-yl-N-methyl-propan-2-amine (50 mg, 0.26 mmol) in HCl/EtOAc (4 M, 1 mL) at 15° C. The mixture was stirred at 15° C. for 12 h. The mixture was concentrated under reduced pressure to give a residue. The crude product was triturated with EtOAc (1 mL) at 15° C. for 1 h to give 1-indolizin-1-yl-N-methyl-propan-2-amine HCl salt (48 mg, 80%) as yellow solid. LCMS, m/z=189.3 [M+H]^{+ 1}H NMR (400 MHz, DMSO): δ 8.93-8.81 (m, 2H), 8.20 (d, J=6.8 Hz, 1H), 7.52-7.47 (m, 2H), 6.68-6.64 (m, 2H), 6.51-6.50 (m, 1H), 3.29-3.19 (m, 2H), 2.84 (dd, J=9.2, 13.6 Hz, 1H), 2.56 (t, J=5.6 Hz, 3H), 1.13 (d, J=6.4 Hz, 3H).

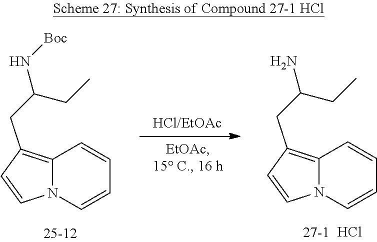 [SMILES: CCC(Cc1ccn2ccccc12)NC(=O)OC(C)(C)C ; "HCl/EtOAc" ; CCC(N)Cc1ccn2ccccc12 ; Cl]

To a solution of tert-butyl (1-(indolizin-1-yl)butan-2-yl)carbamate (80 mg, 0.277 mmol) in EtOAc (1 mL) was added HCl/EtOAc (4 M, 1 mL) at 15° C. The mixture was stirred at 15° C. for 3 h. The reaction mixture was concentrated under reduced pressure to give a residue. The crude product was triturated with EtOAc (1 mL) at 15° C. for 30 min to give 1-indolizin-1-ylbutan-2-amine HCl salt (39 mg, 63%) as black solid. LCMS, m/z=189.2 [M+H]^{+ 1}H NMR (400 MHz, DMSO): δ 8.19 (br d, J=6.8 Hz, 1H), 8.05 (br s, 3H), 7.53-7.40 (m, 2H), 6.69-6.65 (m, 2H), 6.49 (t, J=6.4 Hz, 1H), 3.17 (br s, 1H), 3.10-3.03 (m, 1H), 2.95-2.89 (m, 1H), 1.57-1.49 (m, 2H), 0.92 (t, J=7.6 Hz, 3H).

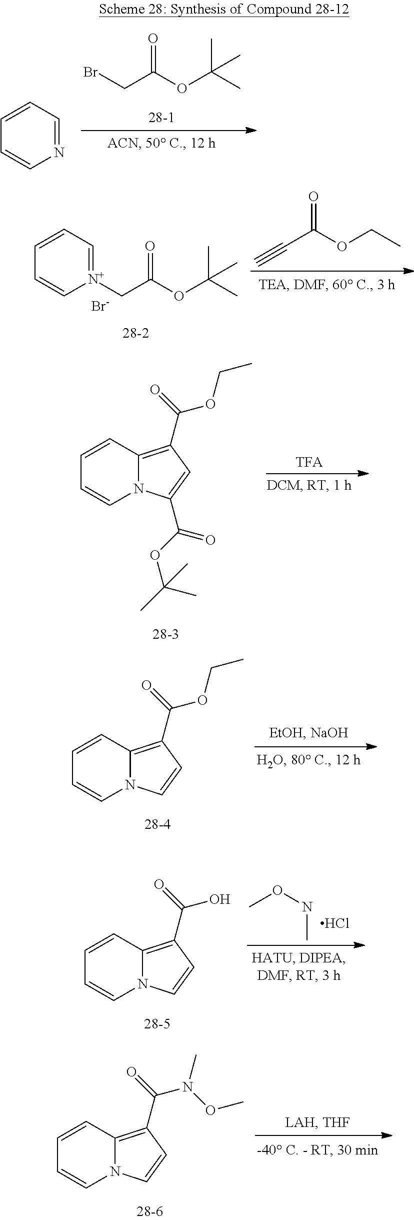

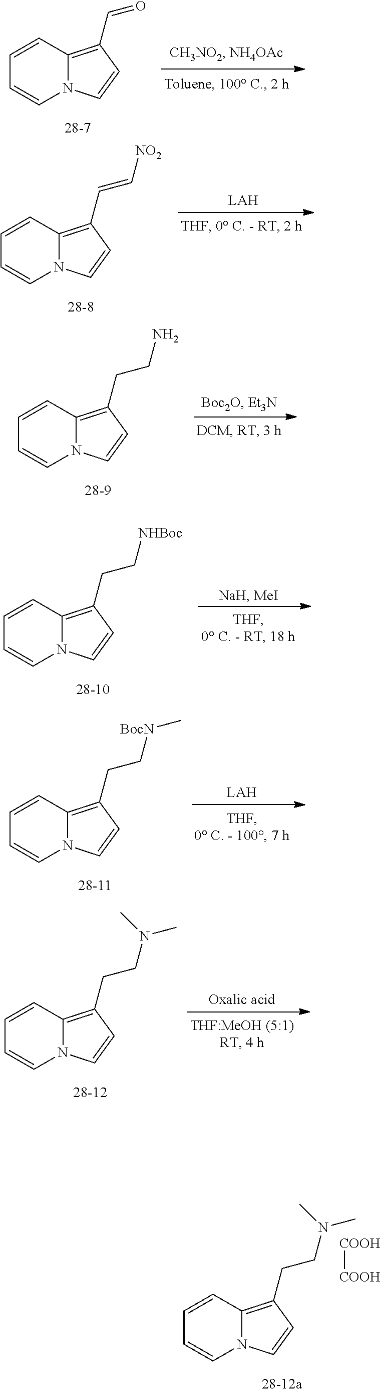

### Synthesis of 1-(2-(tert-butoxy)-2-oxoethyl) pyridin-1-ium bromide (28-2)

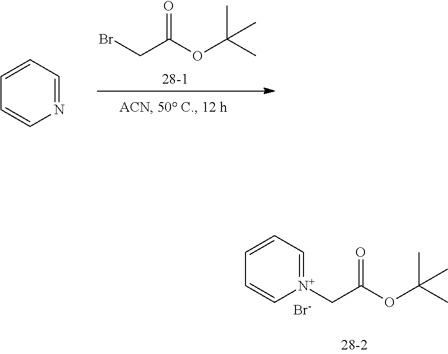

To a stirred solution of pyridine (10.0 g, 126.422 mmol, 1.0 equiv.) in dry ACN (150 mL) was added tert-butyl bromoacetate (28-1) (18.676 mL, 126.422 mmol, 1.0 equiv.) at RT. The resulting reaction mixture was stirred at 50° C. for 12 h. After completion of the reaction [Monitored by TLC, mobile Phase 50% EtOAc-Hex], excess solvent was evaporated to get the crude compound. The crude was washed with diethyl ether to afford the pure compound 1-(2-(tert-butoxy)-2-oxoethyl) pyridin-1-ium bromide (28-2) as a colorless sticky liquid; Yield: (20 g, 81.44%). ¹H NMR (400 MHz, DMSO-d6) δ 9.05 (d, J=5.16 Hz, 2H), 8.72 (t, J=7.32, 7.84 Hz, 1H), 8.25 (t, J=6.56, 6.96 Hz, 2H), 5.57 (s, 2H), 1.46 (s, 9H).

### Synthesis of 3-(tert-butyl) 1-ethyl indolizine-1,3-dicarboxylate (28-3)

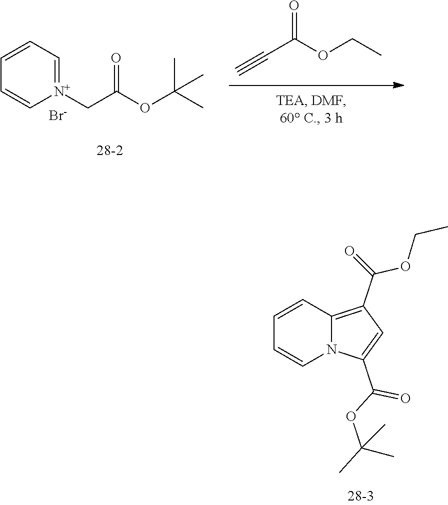

To a stirred solution of 1-(2-(tert-butoxy)-2-oxoethyl) pyridin-1-ium bromide (28-2) (20 g, 72.95 mmol, 1.0 equiv.) in DMF (150 mL) was added TEA (30.5 mL, 218.85 mmol, 3.0 equiv.) and ethyl propiolate (8.13 mL, 80.245 mmol, 1.1 equiv.) at RT. The resulting reaction mixture was stirred at 60° C. for 3 h. After completion of the reaction [Monitored by TLC, mobile Phase 10% EtOAc-Hex], it was diluted with ethyl acetate (1000 mL), washed 2-3 times with cold water and dried over magnesium sulphate and concentrated under reduced pressure to get the crude compound. The crude was purified by silica gel (100-200 mesh) column chromatography eluted with 0-5% ethyl acetate in hexane to afford 3-(tert-butyl) 1-ethyl indolizine-1,3-dicarboxylate (28-3) as a white solid; Yield: (10 g, 47.38%). ¹H NMR (400 MHz, CDCl₃) δ 9.50 (d, J=7.12 Hz, 1H), 8.30 (d, J=9.0 Hz, 1H), 7.88 (s, 1H), 7.28-7.24 (m, 1H), 6.94-6.90 (m, 1H), 4.39-4.34 (q, 2H), 1.60 (s, 9H), 1.42 (t, J=7.16, 7.12 Hz, 3H). LCMS: Rt 4.11 min. MS (ES) C₁₆H₁₉NO₄ requires 289, found 290 [M+H]⁺.

### Synthesis of ethyl indolizine-1-carboxylate (28-4)

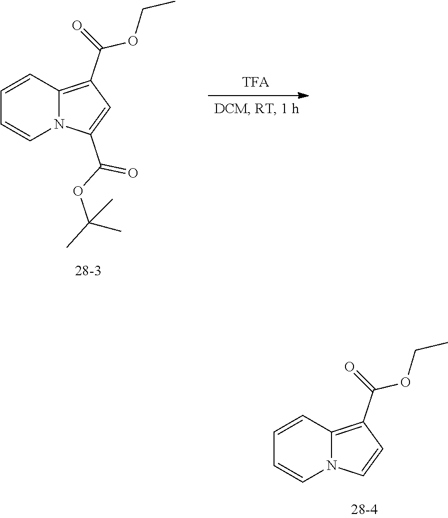 [SMILES: CCOC(=O)c1cc(C(=O)OC(C)(C)C)n2ccccc12 ; "TFA" ; CCOC(=O)c1ccn2ccccc12]

To a stirred solution of 3-(tert-butyl) 1-ethyl indolizine-1,3-dicarboxylate (28-3) (10.0 g, 31.348 mmol, 1.0 equiv.) in DCM (100 mL) was added TFA (24.14 mL, 313.48 mmol, 10.0 equiv.) at 0° C. The resulting reaction mixture was stirred at RT for 2 h. After 2h, TLC monitoring showed (˜15%-20%) SM (28-3), (˜70%-75%) desired product and (˜10%-15%) polar spot [mobile Phase 30% EtOAc-Hex], the reaction mixture was quenched with saturated sodium bicarbonate solution (200 mL) and extracted with DCM (200 mL). Then dried over magnesium sulphate and concentrated under reduced pressure. The crude compound was purified by silica gel (100-200 mesh) column chromatography eluted with 15-20% ethyl acetate in hexane to afford ethyl indolizine-1-carboxylate (28-4) as a colorless sticky liquid; Yield: (2.5 g, 42.15%). ¹H NMR (400 MHz, CDCl₃) δ 8.17 (d, J=9.0 Hz, 1H), 7.98 (d, J=6.84 Hz, 1H), 7.24-7.20 (m, 2H), 7.03 (t, J=7.04, 8.44 Hz, 1H), 6.69 (t, J=6.72, 6.28 Hz, 1H), 4.38-4.32 (q, 2H), 1.40 (t, J=7.12, 7.04 Hz, 3H). LCMS: Rt 3.21 min. MS (ES) C₁₁H₁₁NO₂ requires 189, found 190 [M+H]⁺.

### Synthesis of indolizine-1-carboxylic acid (28-5)

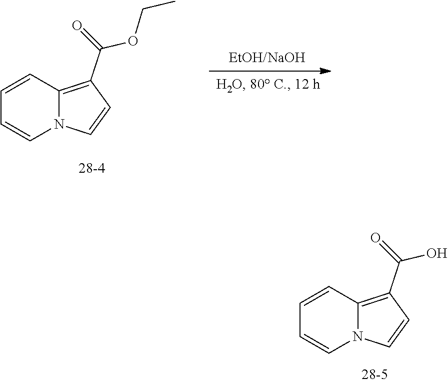

To a stirred solution of ethyl indolizine-1-carboxylate (28-4) (7.5 g, 39.638 mmol, 1.0 equiv.) in absolute ethanol (80.0 mL) was added aqueous solution of sodium hydroxide (80 mL, 198.192 mmol, 5.0 equiv.) at RT. The resulting reaction mixture was stirred at 80° C. for 12 h. After the completion [Monitored by TLC and LCMS, mobile Phase 30% EtOAc-Hex], excess solvent was evaporated and acidified with 1(N) HCL in ice cooling condition and extracted with DCM (2×500 mL). Dried over anhydrous sodium sulphate and concentrated under vacuum to afford indolizine-1-carboxylic acid (28-5) as an off white solid; Yield: (5.0 g, 78.27%). ¹H NMR (400 MHz, DMSO-d6) δ 11.85 (s, 1H), 8.44 (d, J=6.88 Hz, 1H), 8.03 (d, J=9.0 Hz, 1H), 7.57 (d, J=2.68 Hz, 1H), 7.12-7.08 (m, 2H), 6.82 (t, J=6.24, 6.56 Hz, 3H). LCMS: Rt 1.38 min. MS (ES) C₉H₇NO₂ requires 161, found 162 [M+H]⁺.

### Synthesis of N-methoxy-N-methylindolizine-1-carboxamide (28-6)

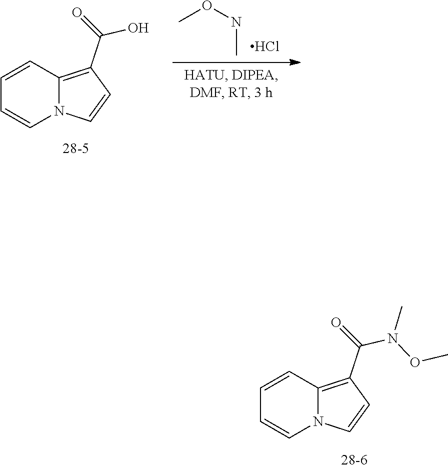

To a stirred solution of indolizine-1-carboxylic acid (28-5) (5.0 g, 31.056 mmol, 1.0 equiv.) in DMF (80.0 mL) under nitrogen atmosphere DIPEA (16.3 mL, 93.168 mmol, 3.0 equiv.) and HATU (17.71 g, 46.584 mmol, 1.5 equiv.) was added at RT and allowed to stir at RT for 15 min. Then N, O-dimethylhydroxylamine hydrochloride (7.57 g, 77.64 mmol, 2.5 equiv.) was added to the reaction mixture and the resulting reaction mixture was stirred at RT for 3 h. After completion [monitored by TLC, mobile Phase 50% EtOAc-hexane], the reaction mixture was diluted with ethyl acetate (500 mL), washed 2-3 times with cold water (200 mL) and dried over magnesium sulphate then concentrated under reduced pressure to get the crude compound. The crude compound was purified by combi flash column chromatography, eluted with 20% ethyl acetate in hexane to afford N-methoxy-N-methylindolizine-1-carboxamide (28-6) as an off white solid; Yield: (4.7 g, 74.1%). ¹H NMR (400 MHz, CDCl₃) δ 8.39 (d, J=9.2 Hz, 2H), 7.96 (d, J=6.88 Hz, 1H), 7.32 (d, J=2.16 Hz, 1H), 7.21-7.20 (m, 1H), 6.99-6.95 (m, 1H), 6.67-6.64 (m, 1H), 3.71 (s, 3H), 3.38 (s, 3H). LCMS: Rt 2.81 min. MS (ES) C₁₁H₁₂N₂O₂ requires 204, found 205 [M+H]⁺.

### Synthesis of indolizine-1-carbaldehyde (28-7)

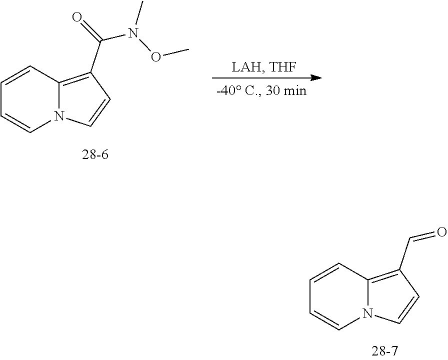

To a stirred solution of N-methoxy-N-methylindolizine-1-carboxamide (28-6) (2.3 g, 11.262 mmol, 1.0 equiv.) in dry THE (25.0 mL) was added LAH (1m in THF) (22 mL, 22.524 mmol, 2.0 equiv.) at −40° C. The resulting reaction mixture was stirred at −40° C. to RT for 30 min. After completion [monitored by TLC, mobile Phase 30% EtOAc-hexane], the reaction mixture was quenched with sodium sulphate decahydrate, filtered off and washed with DCM (100 mL). Then dried over sodium sulphate and concentrated under reduced pressure to obtain the crude indolizine-1-carbaldehyde (28-7) (1.6 g) as a colorless sticky liquid, which was forwarded to the next step without further purification. LCMS: Rt 2.25 min. MS (ES) C₉H₇NO requires 145, found 146 [M+H]⁺.

### Synthesis of (E)-1-(2-nitrovinyl) indolizine (28-8)

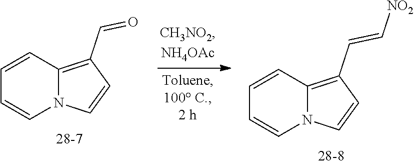 [SMILES: O=Cc1ccn2ccccc12 ; O=[N+]([O-])/C=C/c1ccn2ccccc12]

To a stirred solution of indolizine-1-carbaldehyde (28-7) (crude) (1.6 g, 11.022 mmol, 1.0 equiv.) in dry toluene (60.0 mL) was added Nitromethane (30.0 mL) and NH₄OAc (680 mg, 8.818 mmol, 0.8 equiv.) at RT. The resulting reaction mixture was stirred at 100° C. for 2 h. After completion [monitored by TLC, mobile Phase 40% EtOAc in hexane], the excess solvent was evaporated under reduced pressure to obtain the crude. The crude compound was purified by combi flash column chromatography eluted with 100% DCM to afford (E)-1-(2-nitrovinyl) indolizine (28-8) as a yellow solid; Yield: (0.75 g, 36.16%). ¹H NMR (400 MHz, DMSO-d6) δ 8.49-8.47 (m, 2H), 8.44 (s, 1H), 8.09 (d, J=8.8 Hz, 1H), 7.95 (d, J=12.4 Hz, 1H), 7.75 (d, J=2.4 Hz, 1H), 7.39 (d, J=2.8 Hz, 1H), 7.24 (t, J=7.2, 8.0 Hz, 1H), 6.94 (t, J=6.8, 6.0 Hz, 1H). LCMS: Rt 3.09 min. MS (ES) C₁₀H₈N₂O₂, requires 188, found 189 [M+H]⁺.

### Synthesis of 2-(3aH-inden-1-yl) ethan-1-amine (28-9)

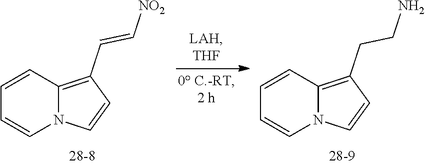 [SMILES: O=[N+]([O-])/C=C/c1ccn2ccccc12 ; NCCc1ccn2ccccc12]

To a stirred solution of 1-(2-nitrovinyl) indolizine (28-8) (800 mg, 188.19 mmol, 1.0 equiv.) in dry THF (20 mL) was added LAH (1 m in THF) (12.7 mL, 12.753 mmol, 3.0 equiv.) at 0° C. The resulting reaction mixture was stirred at RT for 2 h. After completion [monitored by TLC, mobile Phase 40% EtOAc-hexane], the reaction mixture was quenched with sodium sulphate decahydrate, filtered off and washed with 5% MeOH in DCM (100 mL). Then dried over sodium sulphate and concentrated under reduced pressure to get the crude 2-(3aH-inden-1-yl) ethan-1-amine (28-9) (800 mg) as a colorless sticky liquid, which was forwarded to the next step without further purification.

### Synthesis of tert-butyl (2-(indolizin-1-yl) ethyl) carbamate (28-10)

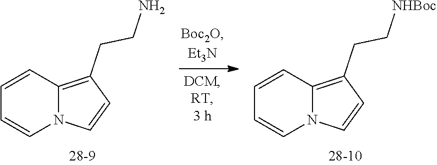 [SMILES: NCCc1ccn2ccccc12 ; CC(C)(C)OC(=O)NCCc1ccn2ccccc12]

To a stirred solution of 2-(3aH-inden-1-yl) ethan-1-amine (28-9) (crude) (800 mg, 4.993 mmol, 1.0 equiv.) in DCM (15 mL) was added TEA (1.4 mL, 9.986 mmol, 2.0 equiv.) and Boc anhydride (2.3 mL, 9.986 mmol, 2.0 equiv.) at 0° C. and the resulting reaction mixture was stirred at RT for 3 h. After completion [monitored by TLC, mobile Phase 20% EtOAc-hexane] the reaction mixture was diluted with DCM (100 mL) and washed with water (50 mL), followed by NaCl (50 mL) solution. The organic layer was dried over sodium sulphate and concentrated under reduced pressure to get the crude. The crude compound was purified by combi flash column chromatography, eluted with 10% ethyl acetate in hexane to afford tert-butyl (2-(indolizin-1-yl) ethyl) carbamate (28-10) as a colorless sticky liquid; Yield: (400 mg, 30.77%). ¹H NMR (400 MHz, DMSO-d6) δ 8.14 (d, J=6.56 Hz, 1H), 7.42 (bs, 1H), 7.34 (d, J=8.8 Hz, 1H), 6.83 (m, 1H), 6.60-6.56 (m, 2H), 6.45 (t, J=6.64 Hz, 1H), 3.11-3.09 (m, 2H), 2.80-2.76 (t, J=7.76, 7.2 Hz, 2H), 1.37 (s, 9H). LCMS: Rt 3.51 min. MS (ES) C₁₅H₂₀N₂O₂, requires 260, found 261 [M+H]⁺.

### Synthesis of tert-butyl (2-(indolizin-1-yl) ethyl) (methyl) carbamate (28-11)

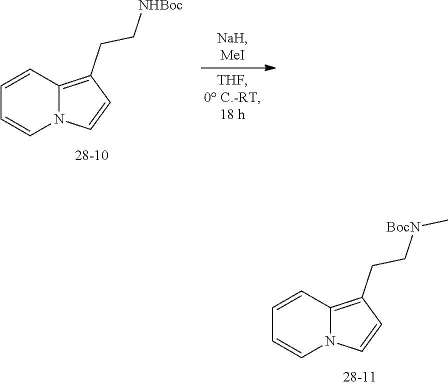 [SMILES: CC(C)(C)OC(=O)NCCc1ccn2ccccc12 ; CN(CCc1ccn2ccccc12)C(=O)OC(C)(C)C]

To stirred solution of tert-butyl (2-(indolizin-1-yl) ethyl) carbamate (28-10) (850 mg, 3.269 mmol, 1.0 equiv.) in dry THF (20.0 mL) was added (60%) NaH (260 mg, 6.538 mmol, 2.0 equiv.) and Mel (0.82 mL, 13.077 mmol, 4.0 equiv.) at 0° C. in a seal-tube vessel and the resulting reaction mixture was stirred at 0° C.-RT for 18 h. After completion (Monitoring by TLC, 20% EA in Hex), the reaction mixture was quenched with cold water (50 mL) and extracted with ethyl acetate (200 mL) and washed with NaCl solution then dried over magnesium sulphate and concentrated under reduced pressure to get the crude. The crude compound was purified by silica gel (100-200 mesh) column flash chromatography, eluted with 5%-8% ethyl acetate in hexane to afford tert-butyl (2-(indolizin-1-yl) ethyl) (methyl) carbamate (28-11) as a colorless sticky liquid; Yield: (450 mg, 50.17%). ¹H NMR (400 MHz, DMSO-d6) δ 8.15 (d, J=6.88 Hz, 1H), 7.43 (d, J=2.24 Hz, 1H), 7.36 (d, J=8.6 Hz, 1H), 6.61-6.57 (m, 2H), 6.45 (t, J=6.72, 6.4 Hz, 1H), 3.35 (m, 2H), 2.88-2.84 (m, 2H), 2.76 (s, 3H), 1.38-1.27 (m, 9H). LCMS: Rt 3.78 min. MS (ES) C₁₆H₂₂N₂O₂, requires 274, found 275 [M+H]⁺.

### Synthesis of 2-(indolizin-1-yl)-N, N-dimethylethan-1-amine (28-12)

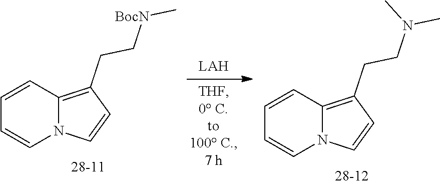 [SMILES: CN(CCc1ccn2ccccc12)C(=O)OC(C)(C)C ; CN(C)CCc1ccn2ccccc12]

To stirred solution of tert-butyl (2-(indolizin-1-yl) ethyl) (methyl) carbamate (28-11) (530 mg, 1.596 mmol, 1.0 equiv.) in dry THF (10.0 mL) was added drop wise LAH (1 m in THF) (24.0 mL, 23.946 mmol, 15.0 equiv.) at 0° C. and the resulting reaction mixture was stirred at 100° C. for 7h. After completion [monitored by TLC, mobile Phase 10% MeOH in DCM], the reaction mixture was quenched with sodium sulphate decahydrate, filtered off and washed with 5% MeOH in DCM (100 mL). Then dried over sodium sulphate and concentrated under reduced pressure to obtained crude. The crude compound was purified by combi column chromatography eluted with 1%-5% [(1% TEA in MeOH) in DCM] to afford 2-(indolizin-1-yl)-N,N-dimethylethan-1-amine (28-12) as a colorless sticky liquid; Yield: (260 mg, 86.51%). ¹H NMR (400 MHz, DMSO-d6) δ 8.13 (d, J=6.8 Hz, 1H), 7.41 (bs, 1H), 7.37 (d, J=9.2 Hz, 1H), 6.61 (bs, 1H), 6.58 (t, J=6.8 Hz, 1H), 6.44 (t, J=6.4, Hz, 1H), 2.81-2.77 (m, 2H), 2.45-2.38 (m, 2H), 2.19 (s, 6H). LCMS: Rt 1.73 min. MS (ES) C₁₂H₁₆N₂ requires 188, found 189 [M+H]⁺.

### Synthesis of 2-(indolizin-1-yl)-N, N-dimethylethan-1-amine oxalate (28-12a or 28-12 oxalate)

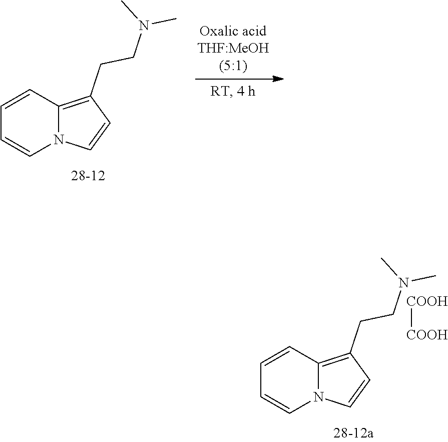

To a stirred solution of 2-(indolizin-1-yl)-N, N-dimethylethan-1-amine (28-12) (260 mg, 1.381 mmol, 1.0 equiv.) in THF:MeOH (5:1) (3.6 mL) was added oxalic acid (250 mg, 2.762 mmol, 2.0 equiv.) at RT. The resultant reaction mixture was stirred at RT for 4 h. After complete consumption of SM [monitored by TLC, mobile Phase 40% MeOH-DCM], the excess solvent was evaporated under reduced pressure to get the crude and the residue was washed with 5% MeOH in diethyl ether (20 mL×2) and dried to afford 2-(indolizin-1-yl)-N, N-dimethylethan-1-amine oxalate (28-12a) as a light green solid; Yield: (200 mg, 77%). ¹H NMR (400 MHz, DMSO-d6) δ 8.19 (d, J=6.8 Hz, 1H), 7.48-7.46 (m, 2H), 6.67-6.63 (m, 2H), 6.50 (t, J=2.64 Hz, 1H), 3.22-3.21 (m, 2H), 3.09-3.07 (m, 2H), 2.80 (s, 6H). LCMS: Rt 1.73 min. MS (ES) C₁₄H₁₈N₂O₄ requires 188, found 189 [M+H]⁺. HPLC: Rt 5.83 min. Purity (λ 220 nm): 99.23%.

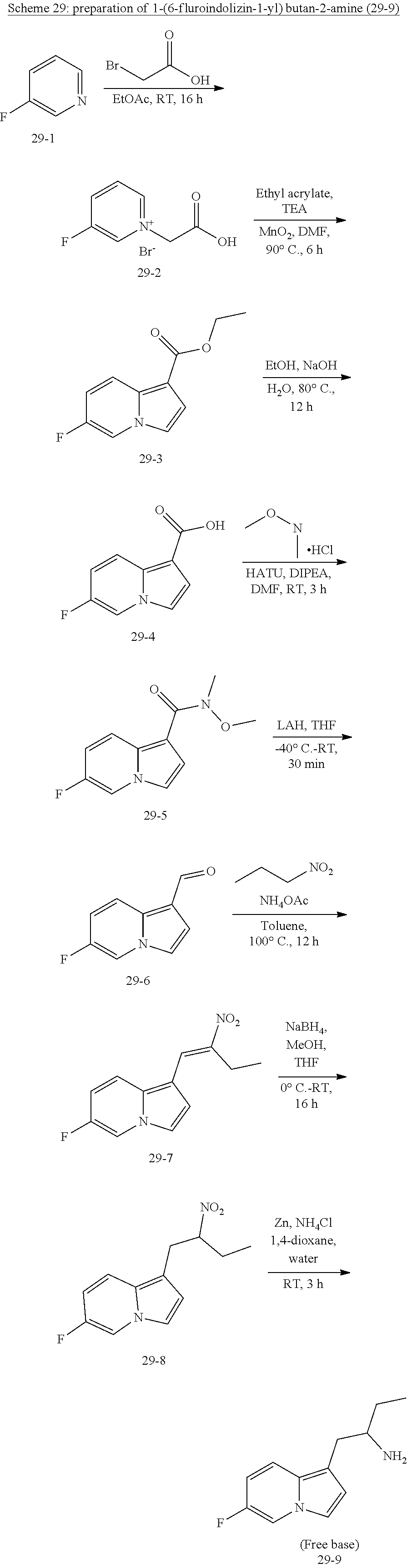

### Synthesis of 1-(carboxymethyl)-3-fluoropyridine-1-ium bromide (29-2)

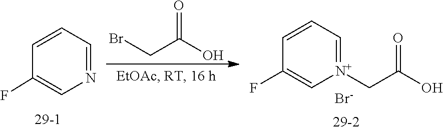

To a stirred solution of 3-fluoropyridine (29-1) (20 g, 206.186 mmol, 1.0 equiv.) in dry Ethyl Acetate (200 mL) was added bromoacetic acid (28.649 g, 206.186 mmol, 1.0 equiv.) at RT and the resulting reaction mixture was allowed to stir at room temperature for 16h. Upon completion, monitored by TLC (50% EA in Hexane), the reaction mixture was evaporated under reduced pressure to get the crude which was washed with diethyl ether to afford the 1-(carboxymethyl)-3-fluoropyridine-1-ium bromide (29-2) as an off white solid (22 g, 68%). ¹H NMR (400 MHz, DMSO-d6) δ 9.44 (s, 1H), 9.01 (d, J=5.96 Hz, 1H), 8.79-8.75 (m, 1H), 8.37-8.31 (m, 1H), 5.57 (s, 2H). LCMS: Rt 0.21 min. MS (ES) C7H7FNO2+ requires 156, found 157 [M+H]⁺.

### Synthesis of Ethyl 6-fluoroindolizine-1-carboxylate (29-3)

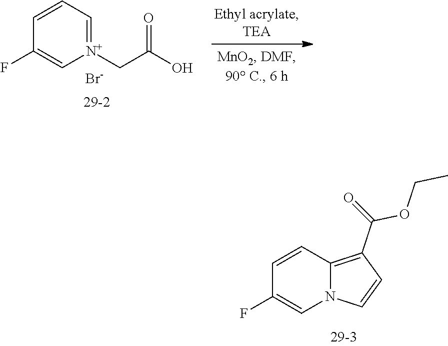

To a stirred solution of 1-(carboxymethyl)-3-fluoropyridine-1-ium bromide (29-2) (20 g, 84.66 mmol, 1.0 equiv.) in dry DMF (200 mL) was added Triethylamine (21.417 mL, 211.649 mmol, 2.5 equiv.) followed by Ethyl acrylate (25.428 g, 253.979 mmol, 3.0 equiv.), MnO₂ (29.441 g, 338.639 mmol, 4.0 equiv.) at RT and the resulting reaction mixture was allowed to stir at 90° C. for 6 h under N2 atmosphere. Upon completion, monitored by TLC (20% EA in Hexane), the reaction mixture was filtered through celite bed, the organic part was extracted with ethyl acetate, repeatedly washed with cold water (2×100 mL). The organic layer was collected and evaporated under reduced pressure to get the crude which was purified by column chromatography using 100-200 mesh size silica and 0-6% EA in Hexane to afford the Ethyl 6-fluoroindolizine-1-carboxylate (29-3) as light brown liquid (900 mg, 7%). ¹H NMR (400 MHz, DMSO-d₆) δ 8.71 (d, J=2.88 Hz, 1H), 8.06-8.02 (m, 1H), 7.60 (d, J=2.64 Hz, 1H), 7.26-7.21 (m, 1H), 7.18 (d, J=2.64 Hz, 1H), 4.28-4.23 (q, 2H), 1.31 (t, J=7.16 Hz, 3H). LCMS. Rt 3.38 min. MS (ES) C11H1OFNO2 requires 207, found 208 [M+H]⁺.

### Synthesis of 6-fluoroindolizine-1-carboxylate (29-4)

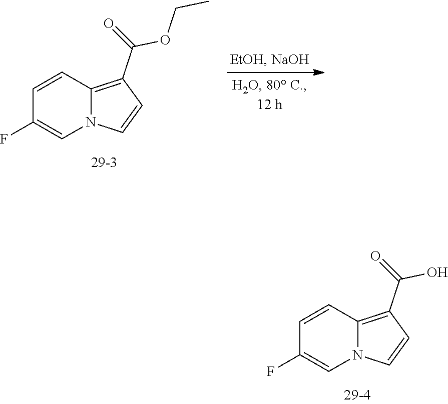

To a stirred solution of Ethyl 6-fluoroindolizine-1-carboxylate (29-3) (1.2 g, 5.792 mmol, 1.0 equiv.) in Ethanol (12 mL) was added NaOH (1.158 g, 28.958 mmol, 5.0 equiv.) in water (6 mL) at 0° C. and the resulting reaction mixture was allowed to stir for 12h. Upon completion, monitored by TLC (50% EA in Hexane), the reaction mixture was acidified with 2(N) HCl, the organic part was extracted with DCM, dried over sodium sulfate, and evaporated under reduced pressure to get the crude, which was washed with pentane to afford the 6-fluoroindolizine-1-carboxylate (29-4) as off white solid (900 mg, 86%.)¹H NMR (400 MHz, DMSO-d₆) δ 12.03 (s, 1H), 8.68-8.67 (m, 1H), 8.07-8.03 (m, 1H), 7.58 (d, J=2.84, 1H), 7.21-7.14 (m, 2H). LCMS: Rt 1.85 min. MS (ES) C9H6FNO2 requires 179, found 180 [M+H]⁺.

### Synthesis of 6-fluoro-N-methoxy-N-methylindolizine-1-carboxamide (29-5)

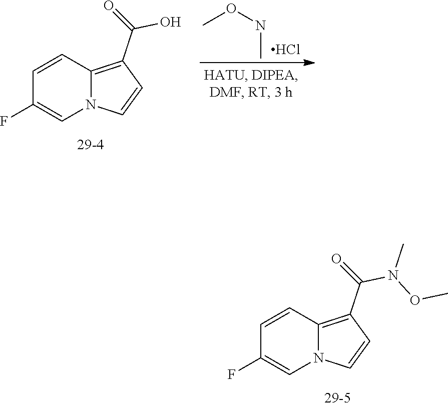

To a stirred solution of 6-fluoroindolizine-1-carboxylate (29-4) (1.1 g, 6.14 mmol, 1.0 equiv.) in DMF (10 mL) was added DIPEA (3.209 mL, 18.42 mmol, 3.0 equiv.) and HATU (3.502 g, 9.21 mmol, 1.5 equiv.) then the reaction mixture was allowed to stir at room temperature under N2 atmosphere for 1h. Then N—O-Dimethylhydroxylamine hydrochloride (1.797 g, 18.42 mmoL, 3.0 equiv.) was added to the reaction mixture and it was allowed to stir for 12 h at the same temperature. Upon completion, monitored by TLC (40% EA in Hexane), water was added to the reaction mixture and the organic part was extracted with ethyl acetate, and washed with cold water (2×100 mL). The organic layer was collected and dried over sodium sulfate, evaporated under reduced pressure to get the crude which was purified by combi-flash chromatography using 0-20% EA in Hexane to afford the 6-fluoro-N-methoxy-N-methylindolizine-1-carboxamide (29-5) as light yellow liquid (1.1 gm, 80%). ¹H NMR (400 MHz, DMSO-d₆) δ 8.66 (d, J=2.68 Hz, 1H), 8.29-8.25 (m, 1H), 7.60 (d, J=2.24 Hz, 1H), 7.29 (d, J=2.2 Hz, 1H), 7.16 (t, J=8.24, 8.56 Hz, 1H), 3.68 (s, 3H), 3.24 (s, 3H). LCMS: Rt 2.90 min. MS (ES) C11H11FN₂O₂ requires 222, found 223 [M+H]⁺.

### Synthesis of 6-fluoroindolizine-1-carbaldehyde (29-6)

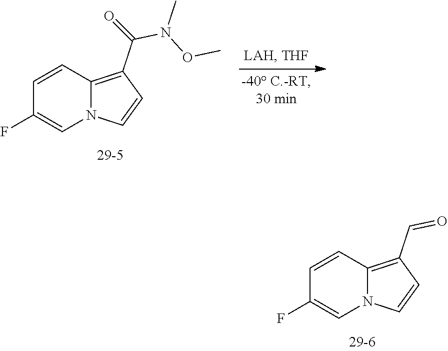 [SMILES: CON(C)C(=O)c1ccn2cc(F)ccc12 ; O=Cc1ccn2cc(F)ccc12]

To a stirred solution of 6-fluoro-N-methoxy-N-methylindolizine-1-carboxamide (29-5) (1 g, 4.5 mmol, 1.0 equiv.) in dry THE (10 mL) was added LAH (1M in THF) (9 mL, 9.0 mmol, 2.0 equiv.) at −40° C. under N2 atmosphere and then the reaction mixture was allowed to stir at room temperature for 15 mins. Upon completion, monitored by TLC (40% EA in Hexane), sodium sulfate decahydrate was added portion-wise to the reaction mixture at 0° C. After quenching, the reaction mixture was filtered through a celite bed, and washed with THF. The filtrate part was evaporated under reduced pressure to get the crude 6-fluoroindolizine-1-carbaldehyde (29-6) as a light yellow liquid, which was forwarded to the next step without further purification. LCMS: Rt 2.61 min. MS (ES) C9H6FNO requires 163, found 163.8 [M+H]⁺.

### Synthesis of (E)-6-fluoro-1-(2-nitrobut-1-en-1-yl) indolizine (29-7)

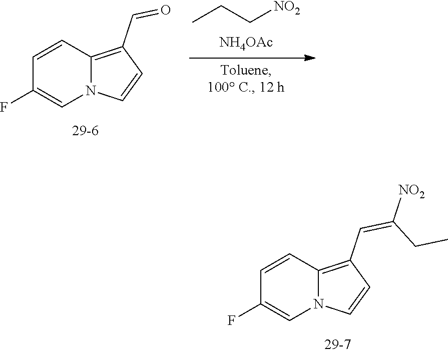

To a stirred solution of crude 6-fluoroindolizine-1-carbaldehyde (29-6) (750 mg, 4.597 mmol, 1.0 equiv.) in Toluene (20 mL) was added Nitro propane (6 mL, 68.955 mmol, 15.0 equiv.) followed by NH₄OAc (354.337 mg, 4.597 mmol, 1.0 equiv.) in a sealed RB flask and the resulting reaction mixture was allowed to stir at 100° C. for 12h. Upon completion, monitored by TLC (20% EA in Hexane), the reaction mixture was evaporated under reduced pressure to get the crude which was purified by combi-flash chromatography using 0-10% EA in Hexane to afford the (E)-6-fluoro-1-(2-nitrobut-1-en-1-yl) indolizine (29-7) as a red solid (150 mg, 14%). ¹H NMR (400 MHz, DMSO-d₆) δ 8.72-8.70 (m, 1H), 8.43 (s, 1H), 8.09-8.05 (m, 1H), 7.83 (d, J=3 Hz, 1H), 7.25-7.20 (m, 2H), 2.97-2.91 (q, 2H), 1.19 (t, J=7.32, 7.44 Hz, 3H). LCMS: Rt 3.55 min. MS (ES) C12H11FN₂O₂ requires 234, found 235 [M+H]⁺.

### Synthesis of 6-fluoro-1-(2-nitrobutyl) indolizine (29-8)

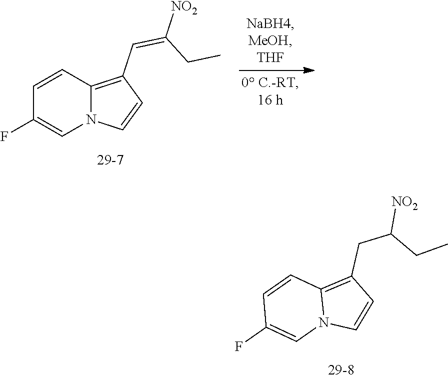 [SMILES: CC/C(=C\c1ccn2cc(F)ccc12)[N+](=O)[O-] ; CCC(Cc1ccn2cc(F)ccc12)[N+](=O)[O-]]

To a stirred solution of (E)-6-fluoro-1-(2-nitrobut-1-en-1-yl) indolizine (29-7) (150 mg, 0.641 mmol, 1.0 equiv.) in MeOH (1 mL) and THE (1 mL) was added NaBH₄ (84.875 mg, 2.244 mmol, 3.5 equiv.) at 0° C. under N2 atmosphere and the resulting reaction mixture was allowed to stir at RT for 16h. Upon completion, monitored by TLC (20% EA in Hexane), the reaction mixture was quenched with cold water, the mixture was evaporated, the organic part was extracted with ethyl acetate. The organic layer was collected and dried over sodium sulfate then evaporated under reduced pressure to get the crude which was purified by combi-flash chromatography using 0-6% EA in Hexane to afford the 6-fluoro-1-(2-nitrobutyl) indolizine (29-8) as light yellow gummy liquid (80 mg, 52%). ¹H NMR (400 MHz, DMSO-d₆) δ 8.38 (d, J=5.08 Hz, 1H), 7.52-7.48 (m, 1H), 7.46 (d, J=2.2 Hz, 1H), 6.75 (t, J=9.68, 8.32 Hz, 1H), 6.62 (d, J=2.04 Hz, 1H), 4.78-4.74 (m, 1H), 3.24-3.19 (m, 2H), 1.91-1.85 (m, 2H), 0.90 (t, J=7.36 Hz, 3H) LCMS: Rt 3.56 min. MS (ES) C12H13FN₂O₂ requires 236, found 236.8 [M+H]⁺.

### Synthesis of 1-(6-fluroindolizin-1-yl) butan-2-amine (29-9, Free base)

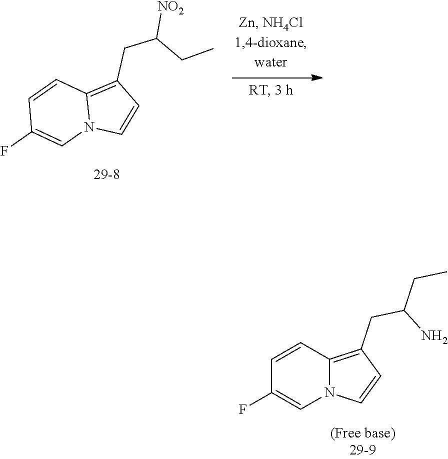 [SMILES: CCC(Cc1ccn2cc(F)ccc12)[N+](=O)[O-] ; CCC(N)Cc1ccn2cc(F)ccc12]

To a stirred solution of 6-fluoro-1-(2-nitrobutyl) indolizine (29-8) (100 mg, 0.424 mmol, 1.0 equiv.) in 1,4-Dioxane (2.0 mL) and water (0.5 mL) was added NH₄C₁ (113.347 mg, 2.119 mmol, 5.0 equiv.) followed by Zn (138.517 mg, 2.119 mmol, 5.0 equiv.) at 0° C. under N2 atmosphere and the resulting reaction mixture was stirred at RT for 3h. Upon completion, monitored by TLC (10% MeOH in DCM), the reaction mixture was filtered through celite bed using DCM. The filtrate part was evaporated under reduced pressure to get the crude which was purified by reverse phase HPLC to afford the 1-(6-fluroindolizin-1-yl) butan-2-amine (29-9, free base) as brown gummy liquid (40 mg, 46%) as free base. ¹H NMR (400 MHz, DMSO-d₆) δ 8.35 (d, J=4.4 Hz, 1H), 7.46-7.42 (m, 2H), 6.66-6.63 (m, 2H), 2.73-2.66 (m, 2H), 2.61-2.55 (m, 1H), 1.41-1.36 (m, 2H), 1.21-1.14 (m, 2H), 0.89 (t, J=7.20, 7.40 Hz, 3H). LCMS: Rt 2.09 min. MS (ES) C12H15FN2 requires 206, found 207 [M+H]⁺. HPLC: Rt 6.23 min. Purity (λ 210 nm): 98.79%.

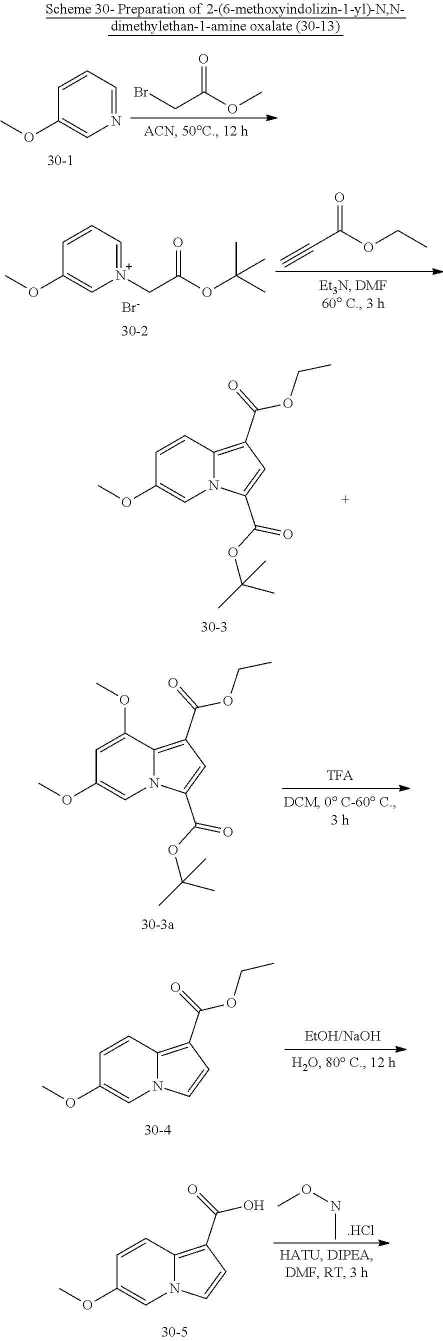

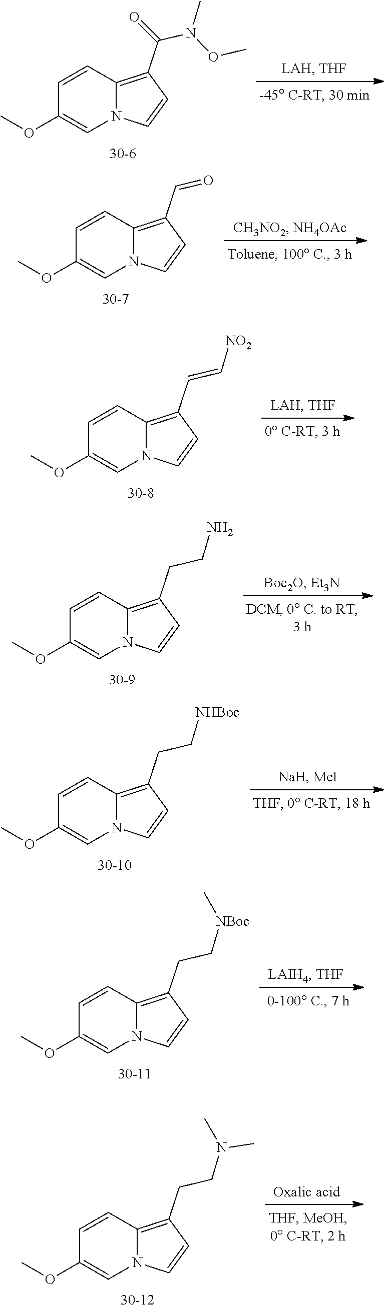

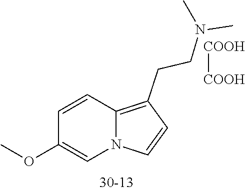

### Synthesis of 1-(2-(tert-butoxy)-2-oxoethyl)-3-methoxypyridin-1-ium bromide (30-2)

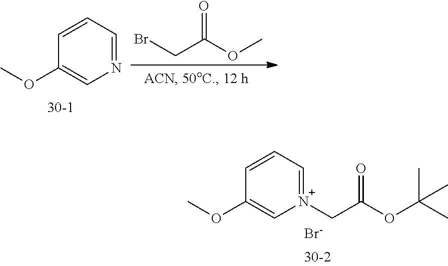

To a stirred solution of 3-methoxy pyridine (30-1) (30 g, 274.901 mmol, 1.0 equiv.) in dry ACN (400 mL) was added tert-butyl bromoacetate (40.6 mL, 274.901 mmol, 1.0 equiv.) at RT. The resulting reaction mixture was stirred at 50° C. for 12 h. After completion [Monitored by TLC, mobile Phase 50% EtOAc-Hex], the excess solvent was evaporated. The crude was washed with diethyl ether to afford the pure compound 1-(2-(tert-butoxy)-2-oxoethyl)-3-methoxypyridin-1-ium bromide (30-2) as an off-white solid; Yield: (60 g, 97.3%). ¹H NMR (400 MHz, DMSO-d₆) δ 9.02 (s, 1H), 8.71 (d, J=5.88 Hz, 1H), 8.36 (dd, J=2.28, 8.68 Hz, 1H), 8.18 (dd, J=5.92, 9.52 Hz, 1H), 5.56 (s, 2H), 4.00 (s, 3H), 1.46 (s, 9H).

### Synthesis of 3-(tert-butyl) 1-ethyl 6-methoxyindolizine-1,3-dicarboxylate (30-3)

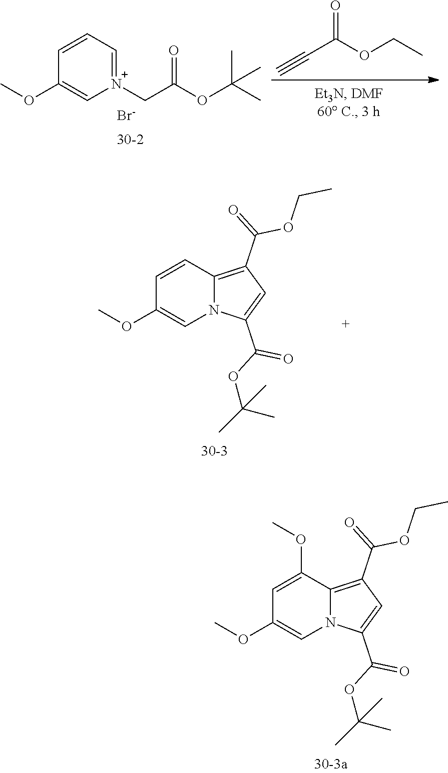

To a stirred solution of 1-(2-(tert-butoxy)-2-oxoethyl)-3-methoxypyridin-1-ium bromide (30-2) (60 g, 199.336 mmol, 1.0 equiv.) in DMF (400 mL) was added Et₃N (84.0 mL, 598.007 mmol, 3.0 equiv.) and ethyl propiolate (24 mL, 219.269 mmol, 1.1 equiv.) at RT. The resulting reaction mixture was stirred at 60° C. for 3 h. After completion [Monitored by TLC, mobile Phase 10% EtOAc-Hex], the reaction mixture was diluted with ethyl acetate (1000 mL), washed 2-3 times with cold water and dried over magnesium sulfate and concentrated under reduced pressure to get the crude compound. The crude was purified by silica gel (100-200 mesh) column chromatography eluted with 0-5% ethyl acetate in hexane to afford 3-(tert-butyl) 1-ethyl 6-methoxyindolizine-1,3-dicarboxylate (30-3) (9 g) as a white solid and (30-3a) (20 g) as an off-white solid which was used for the synthesis of 31-10. ¹H NMR [(30-3)] (400 MHz, DMSO-d₆) δ 9.03 (d, J=1.84 Hz, 1H), 8.10 (d, J=9.72 Hz, 1H), 7.62 (s, 1H), 7.26 (dd, J=2.08, 9.68 Hz, 1H), 4.30 (q, 2H), 3.84 (s, 3H), 1.57 (s, 9H), 1.34 (t, J=7.12, 7.08 Hz, 3H). LCMS: Rt 2.31 min. MS (ES) C₁₇H₂₁NO₅ requires 319, found 320 [M+H]⁺. ¹H NMR [Undesired isomer (30-3a)] (400 MHz, DMSO-d₆) δ 9.08 (d, J=6.64 Hz, 1H), 7.61 (s, 1H), 7.07 (t, J=6.96, 6.88 Hz, 1H), 6.85 (d, J=7.48 Hz, 1H), 4.25 (q, 2H), 3.89 (s, 3H), 1.56 (s, 9H), 1.31 (t, J=6.44, 6.76 Hz, 3H). LCMS: Rt 2.14 min. MS (ES) C₁₇H₂₁NO₅ requires 319, found 320 [M+H]⁺.

### Synthesis of ethyl 6-methoxyindolizine-1-carboxylate (30-4)

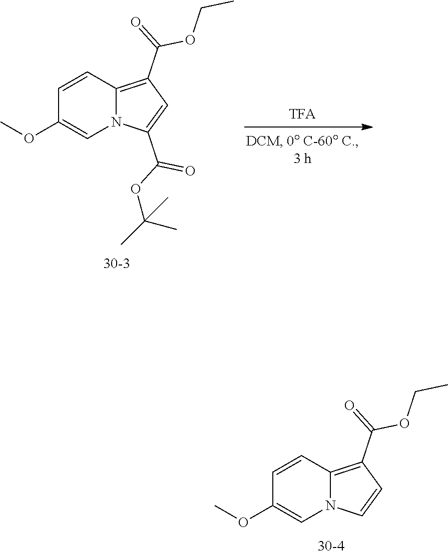 [SMILES: CCOC(=O)c1cc(C(=O)OC(C)(C)C)n2cc(OC)ccc12 ; CCOC(=O)c1ccn2cc(OC)ccc12]

To a stirred solution of 3-(tert-butyl) 1-ethyl 6-methoxyindolizine-1,3-dicarboxylate (30-3) (9 g, 28.182 mmol, 1.0 equiv.) in DCM (200 mL) was added TFA (22 mL, 281.822 mmol, 10.0 equiv.) at 0° C. The resulting reaction mixture was stirred at 0° C.-60° C. for 3 h. After completion [Monitored by TLC, mobile Phase 20% EtOAc-Hex], the reaction mixture was quenched with saturated sodium bicarbonate solution (150 mL) and extracted with DCM (200 mL). Then dried over sodium sulfate and concentrated under reduced pressure. The crude compound was purified by silica gel (100-200 mesh) column chromatography eluted with 15-20% ethyl acetate in hexane to afford ethyl 6-methoxyindolizine-1-carboxylate (30-4) as a colorless sticky liquid; Yield: (2.5 g, 40.5%). ¹H NMR (400 MHz, DMSO-d₆) δ 8.20 (s, 1H), 7.93 (d, J=9.72 Hz, 1H), 7.51 (d, J=2.8 Hz, 1H), 7.06 (d, J=2.76 Hz, 1H), 6.97 (dd, J=1.88, 9.76 Hz, 1H), 4.26 (q, 2H), 3.79 (s, 3H), 1.31 (t, J=7.12, 7.00 Hz, 3H). LCMS: Rt 1.82 min. MS (ES) C₁₂H₁₃NO₃ requires 219, found 220 [M+H]⁺.

### Synthesis of 6-methoxyindolizine-1-carboxylic acid (30-5)

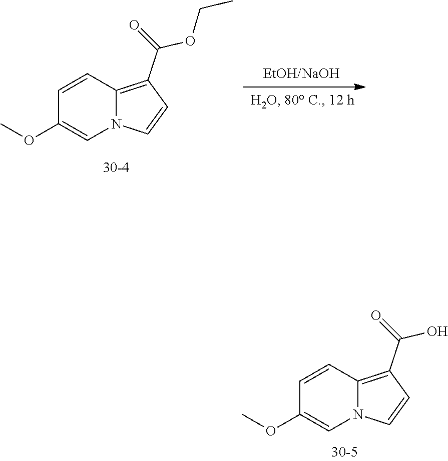 [SMILES: CCOC(=O)c1ccn2cc(OC)ccc12 ; "EtOH/NaOH" ; COc1ccc2c(C(=O)O)ccn2c1]

To a stirred solution of ethyl 6-methoxyindolizine-1-carboxylate (30-4) (2.5 g, 11.403 mmol, 1.0 equiv.) in absolute ethanol (50 mL) was added aqueous solution of sodium hydroxide (50 mL, 57.015 mmol, 5.0 equiv.) at RT. The resulting reaction mixture was stirred at 80° C. for 12 h. After completion [Monitored by TLC and LCMS, mobile Phase 30% EtOAc-Hex], the excess solvent was evaporated and acidified with 1(N) HCL in ice cooling condition and extracted with DCM (2×500 mL). The organic layer was collected and dried over anhydrous sodium sulfate and concentrated under vacuum to afford 6-methoxyindolizine-1-carboxylic acid (30-5) as an off-white solid; Yield: (2 g, 92%). ¹H NMR (400 MHz, DMSO-d₆) δ 11.79 (s, 1H), 8.17 (d, J=1.2 Hz, 1H), 7.93 (d, J=9.6 Hz, 1H), 7.48 (d, J=2.8 Hz, 1H), 7.03 (d, J=2.8 Hz, 1H), 6.92 (dd, J=2.00, 10 Hz, 1H), 3.77 (s, 3H). LCMS: Rt 1.30 min. MS (ES) C₁₀H₉NO₃ requires 191, found 192 [M+H]⁺.

### Synthesis of N,6-dimethoxy-N-methylindolizine-1-carboxamide (30-6)

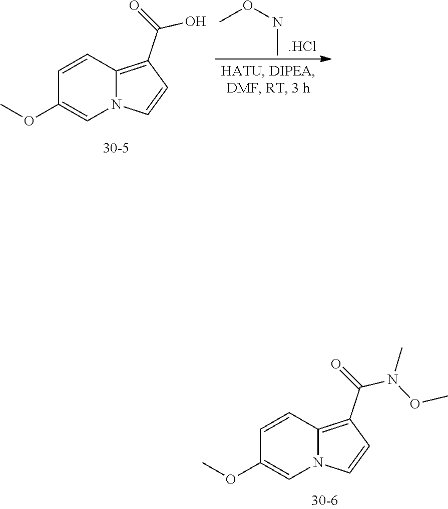

To a stirred solution of 6-methoxyindolizine-1-carboxylic acid (30-5) (2 g, 10.461 mmol, 1.0 equiv.) in DMF (50 mL) under nitrogen atmosphere DIPEA (5.5 mL, 31.382 mmol, 3.0 equiv.) and HATU (6 g, 15.691 mmol, 1.5 equiv.) was added at RT and allowed to stir at same temperature for 15 min. Then N, O-dimethylhydroxylamine hydrochloride (3.1 g, 31.382 mmol, 3.0 equiv.) was added to the reaction mixture and the resulting reaction mixture was stirred at RT for 3 h. After completion [monitored by TLC, mobile Phase 50% EtOAc-hexane], the reaction mixture was diluted with ethyl acetate (500 mL), and washed with water (500 ml). The organic layer was collected and dried over magnesium sulfate and then concentrated under reduced pressure to get the crude. The crude compound was purified by combi column chromatography eluted with 20% ethyl acetate in hexane to afford N,6-dimethoxy-N-methylindolizine-1-carboxamide (30-6) as an off-white solid; Yield: (2.1 g, 85.7%). ¹H NMR (400 MHz, DMSO-d₆) δ 8.16-8.13 (m, 2H), 7.49 (d, J=2.72 Hz, 1H), 7.17 (d, J=2.8 Hz, 1H), 6.88 (dd, J=1.96, 9.84 Hz, 1H), 3.77 (s, 3H), 3.69 (s, 3H), 3.23 (s, 3H). LCMS: Rt 1.59 min. MS (ES) C₁₂H₁₄N₂O₃ requires 234, found 235 [M+H]⁺.

### Synthesis of 6-methoxyindolizine-1-carbaldehyde (30-7)

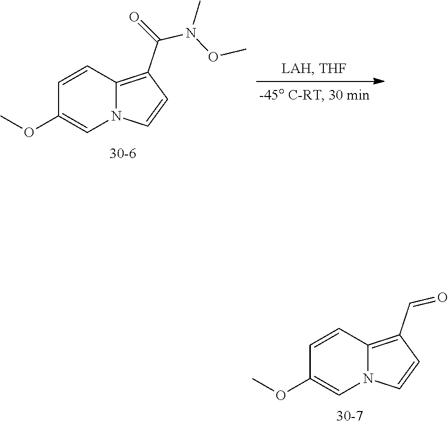

To a stirred solution of N,6-dimethoxy-N-methylindolizine-1-carboxamide (30-6) (2.4 g, 10.245 mmol, 1.0 equiv.) in dry THE (40 mL) was added LAH (2.0 M in THF) (10.24 mL, 20.49 mmol, 2.0 equiv.) at −40° C. The resulting reaction mixture was stirred at −45° C. to RT for 30 min. After completion [monitored by TLC, mobile Phase 30% EtOAc-hexane], the reaction mixture was quenched with sodium sulfate decahydrate, then filtered off and washed with THE and DCM (100 mL). The organic layer was collected and dried over sodium sulfate and concentrated under reduced pressure to obtain the crude 6-methoxyindolizine-1-carbaldehyde (30-7) (1.6 g) as a colorless sticky liquid, which was forwarded to the next step without purification.LCMS: Rt 1.48 min. MS (ES) C₁₀H₉NO₂ requires 175, found 176 [M+H]⁺.

### Synthesis of (E)-6-methoxy-1-(2-nitrovinyl) indolizine (30-8)

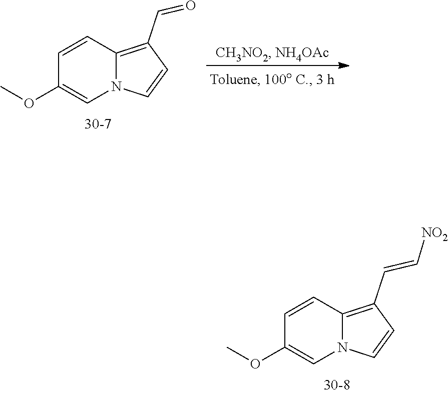 [SMILES: COc1ccc2c(C=O)ccn2c1 ; COc1ccc2c(/C=C/[N+](=O)[O-])ccn2c1]

To a stirred solution of 6-methoxyindolizine-1-carbaldehyde (30-7) (crude) (1.6 g, 9.133 mmol, 1.0 equiv.) in dry toluene (40 mL) was added Nitromethane (20 mL) and NH₄OAc (565 mg, 7.306 mmol, 0.8 equiv.) at RT. The resulting reaction mixture was stirred at 100° C. for 2 h. After completion [monitored by TLC, mobile Phase 40% EtOAc in hexane], the excess solvent was evaporated under reduced pressure to obtain the crude, which was purified by combi column chromatography eluted with 100% DCM to afford (E)-6-methoxy-1-(2-nitrovinyl) indolizine (30-8) as a yellow solid; Yield: (600 mg, 30.11%). ¹H NMR (400 MHz, DMSO-d₆) δ 8.42 (d, J=12.8 Hz, 1H), 8.24 (s, 1H), 8.03 (d, J=9.64 Hz, 1H), 7.91 (d, J=12.84 Hz, 1H), 7.65 (d, J=2.88 Hz, 1H), 7.32 (d, J=2.92 Hz, 1H), 7.03 (dd, J=1.44, 9.56 Hz, 1H), 3.79 (s, 3H). LCMS: Rt 1.77 min. MS (ES) C¹¹H₁₀N₂O₃, requires 218, found 219 [M+H]⁺.

### Synthesis of 2-(6-methoxyindolizin-1-yl) ethan-1-amine (30-9)

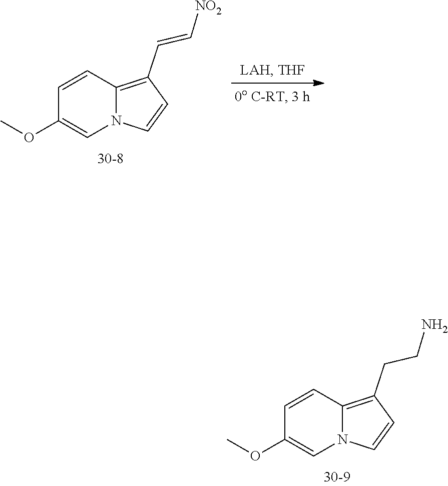 [SMILES: COc1ccc2c(/C=C/[N+](=O)[O-])ccn2c1 ; COc1ccc2c(CCN)ccn2c1]

To a stirred solution of (E)-6-methoxy-1-(2-nitrovinyl) indolizine (30-8) (800 mg, 3.666 mmol, 1.0 equiv.) in dry THE (40 mL) was added LAH (2.0 M in THF) (5.5 mL, 10.999 mmol, 3.0 equiv.) at 0° C. The resulting reaction mixture was stirred at RT for 2 h. After completion [monitored by TLC, mobile Phase 40% EtOAc-hexane], the reaction mixture was quenched with sodium sulfate decahydrate, filtered off, and washed with 5% MeOH in DCM (100 mL). Collected the organic layer and dried over sodium sulfate then concentrated under reduced pressure to obtain the crude 2-(6-methoxyindolizin-1-yl) ethan-1-amine (30-9) (650 mg) as a colorless sticky liquid, which was forwarded to the next step without purification. ¹H NMR (400 MHz, DMSO-d₆) δ 7.84 (s, 1H), 7.34 (d, J=2.12 Hz, 1H), 7.31 (d, J=9.64 Hz, 1H), 6.49 (d, J=2.12 Hz, 1H), 6.40 (dd, J=1.76, 9.72 Hz, 1H), 3.69 (s, 3H), 2.91-2.70 (m, 4H). LCMS: Rt 1.29 min. MS (ES) C₁₁H₁₄N₂O, requires 190, found 191 [M+H]⁺.

### Synthesis of tert-butyl (2-(6-methoxyindolizin-1-yl) ethyl) carbamate (30-10)

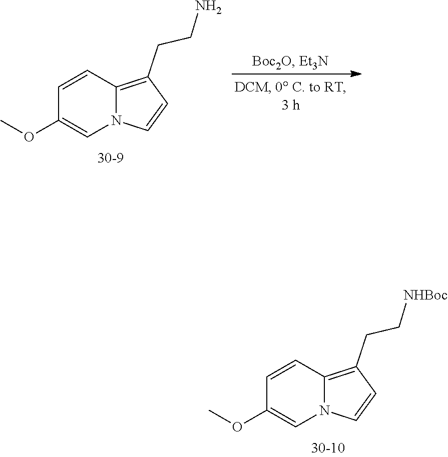 [SMILES: COc1ccc2c(CCN)ccn2c1 ; COc1ccc2c(CCNC(=O)OC(C)(C)C)ccn2c1]

To a stirred solution of 2-(6-methoxyindolizin-1-yl) ethan-1-amine (30-9) (crude) (650 mg, 4.057 mmol, 1.0 equiv.) in DCM (20 mL) was added Et₃N (1.2 mL, 8.114 mmol, 2.0 equiv.) and Boc anhydride (1.8 mL, 8.114 mmol, 2.0 equiv.) at 0° C. and the resulting reaction mixture was stirred at RT for 3 h. After completion [monitored by TLC, mobile Phase 20% EtOAc-hexane], the reaction mixture was diluted with DCM (100 mL) and washed with water (50 mL), followed by NaCl (50 mL) solution. The organic layer was collected and dried over sodium sulfate and concentrated under reduced pressure to get the crude, which was purified by combi column chromatography eluted with 10% ethyl acetate in hexane to afford tert-butyl (2-(6-methoxyindolizin-1-yl) ethyl) carbamate (30-10) as a colorless sticky liquid; Yield: (540 mg, 45.84%). ¹H NMR (400 MHz, DMSO-d₆) δ 7.85 (d, J=1.36 Hz, 1H), 7.34 (d, J=2.32 Hz, 1H), 7.27 (d, J=9.64 Hz, 1H), 6.83-6.81 (m, 1H), 6.49 (d, J=2.16 Hz, 1H), 6.42 (dd, J=1.92, 9.64 Hz, 1H), 3.69 (s, 3H), 3.11-3.05 (m, 2H), 2.76-2.73 (m, 2H), 1.36 (s, 9H). LCMS: Rt 1.97 min. MS (ES) C₁₆H₂₂N₂O₃, requires 290, found 291 [M+H]⁺.

### Synthesis of tert-butyl (2-(6-methoxyindolizin-1-yl) ethyl) (methyl)carbamate (12)

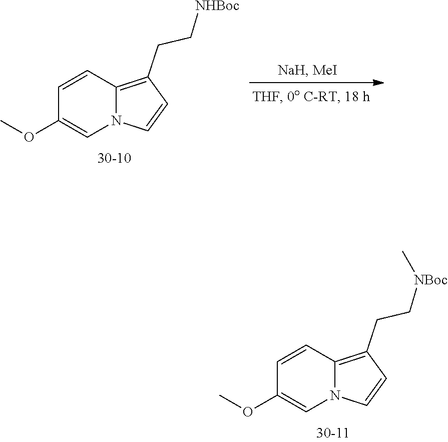 [SMILES: COc1ccc2c(CCNC(=O)OC(C)(C)C)ccn2c1 ; COc1ccc2c(CCN(C)C(=O)OC(C)(C)C)ccn2c1]

To stirred solution of tert-butyl (2-(6-methoxyindolizin-1-yl) ethyl) carbamate (30-10) (340 mg, 1.171 mmol, 1.0 equiv.) in dry THF (20 mL) was added (60%) NaH (140 mg, 3.513 mmol, 3.0 equiv.) and Mel (0.3 mL, 4.685 mmol, 4.0 equiv.) at 0° C. in the seal-tube vessel and the resulting reaction mixture was stirred at 0° C.-RT for 18 h. After completion (Monitoring by TLC,20% EA in Hex), the reaction mixture was quenched with cold water (50 mL) and extracted with ethyl acetate (200 mL) followed by NaCl solution. The organic layer was collected and dried over magnesium sulfate and then concentrated under reduced pressure to get the crude. The crude compound was purified by silica gel (100-200 mesh) column chromatography eluted with 5%-8% ethyl acetate in hexane to afford tert-butyl (2-(6-methoxyindolizin-1-yl) ethyl) (methyl)carbamate (30-11) as a colorless sticky liquid; Yield: (270 mg, 50.17%). ¹H NMR (400 MHz, DMSO-d₆) δ 7.86 (s, 1H), 7.35 (d, J=2.20 Hz, 1H), 7.29 (d, J=9.2 Hz, 1H), 6.47 (bs, 1H), 6.43 (dd, J=1.84, 9.68 Hz, 1H), 3.69 (s, 3H), 3.32 (m, 2H), 2.84 (t, J=7.24, 6.92 Hz, 2H), 2.74 (s, 3H), 1.38-1.28 (m, 9H). LCMS: Rt 2.10 min. MS (ES) C₁₇H₂₄N₂O₃, requires 304, found 305 [M+H]⁺.

### Synthesis of 2-(6-methoxyindolizin-1-yl)-N,N-dimethylethan-1-amine (30-12)

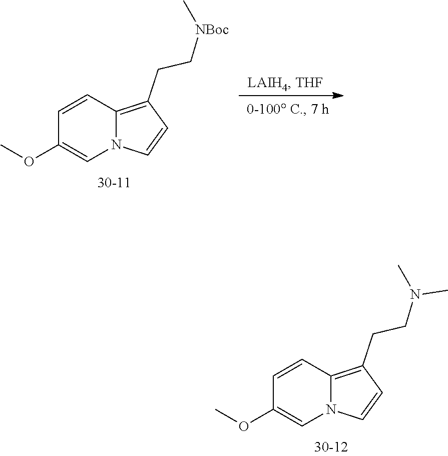

To stirred solution of tert-butyl (2-(6-methoxyindolizin-1-yl) ethyl) (methyl)carbamate (30-11) (270 mg, 0.887 mmol, 1.0 equiv.) in dry THE (15 mL) was added drop wise LAH (2.0 M in THF) (1.6 mL, 3.2 mmol, 3.5 equiv.) at 0° C. and the resulting reaction mixture was stirred at 100° C. for 7 h. After completion [monitored by TLC, mobile Phase 10% MeOH in DCM], the reaction mixture was quenched with sodium sulfate decahydrate, then filtered off and washed with 5% MeOH in DCM (100 mL). The organic layer was collected and dried over sodium sulfate and concentrated under reduced pressure to obtain the crude. The crude compound was purified by combi column chromatography eluted with 0%-5% [(1% Et₃N in MeOH) in DCM] to afford 2-(6-methoxyindolizin-1-yl)-N,N-dimethylethan-1-amine (30-12) as a colorless sticky liquid; Yield: (170 mg, 88%). ¹H NMR (400 MHz, DMSO-d₆) δ 7.84 (d, J=1.32 Hz, 1H), 7.33-7.28 (m, 2H), 6.50 (d, J=2.28 Hz, 1H), 6.40 (dd, J=1.92, 9.68 Hz, 1H), 3.69 (s, 3H), 2.78-2.74 (m, 2H), 2.43-2.39 (m, 2H), 2.18 (s, 6H). LCMS: Rt 1.86 min. MS (ES) C₁₃H₁₈N₂O requires 218, found 219 [M+H]⁺.

### Synthesis of 2-(6-methoxyindolizin-1-yl)-N,N-dimethylethan-1-amine oxalate (30-13)

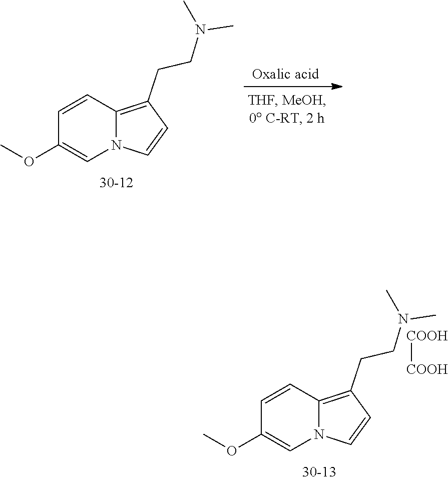

To a stirred solution of 2-(6-methoxyindolizin-1-yl)-N,N-dimethylethan-1-amine (30-12) (410 mg, 1.878 mmol, 1.0 equiv.) in THF:MeOH (5:1) (12 mL) was added oxalic acid (340 mg, 3.756 mmol, 2.0 equiv.) at RT. The resultant reaction mixture was transparent and stirred at RT for 4h. After 30 min solid ppt formation was observed and completed [monitored by TLC, mobile Phase 40% MeOH-DCM], filtered and the excess solvent was evaporated under reduced pressure to obtain the crude compound, and the residue was washed with 5% MeOH in diethyl ether (20 mL×2) and dried to afford 2-(6-methoxyindolizin-1-yl)-N,N-dimethylethan-1-amine oxalate (30-13) as a green solid as oxalate salt; Yield: (200 mg, 77%). Formation of oxalate salt was confirmed by carbon NMR spectra. ¹H NMR (400 MHz, DMSO-d₆) δ 7.90 (s, 1H), 7.41-7.39 (m, 2H), 6.57 (d, J=2.08 Hz, 1H), 6.50 (dd, J=1.60, 9.60 Hz, 1H), 3.70 (s, 3H), 3.23-3.19 (m, 2H), 3.06-3.02 (m, 2H), 2.80 (s, 6H). LCMS: Rt 1.84 min. MS (ES) C₁₅H₂₀N205 requires 218, found 219 [M+H]⁺.

HPLC: Rt 6.06 min. Purity (λ 240 nm): 97.83%.

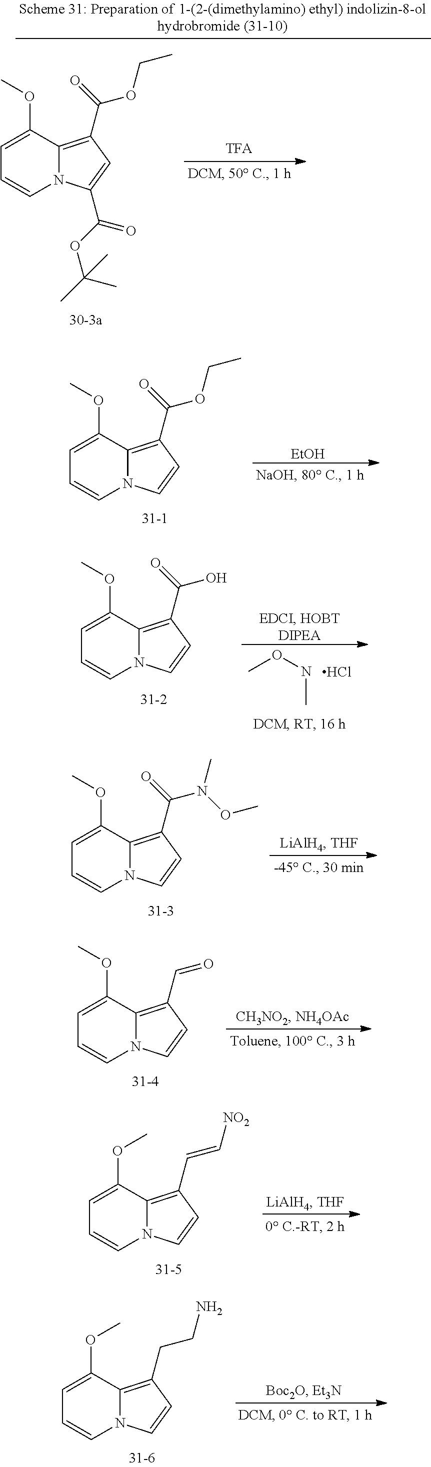

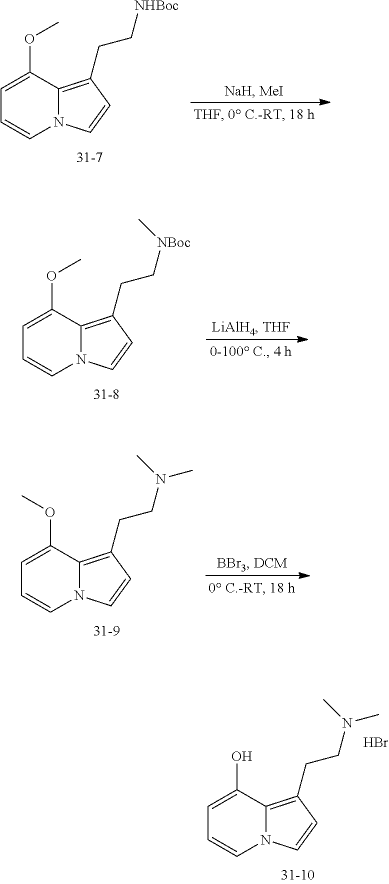

### Synthesis of ethyl 8-methoxyindolizine-1-carboxylate (31-1)

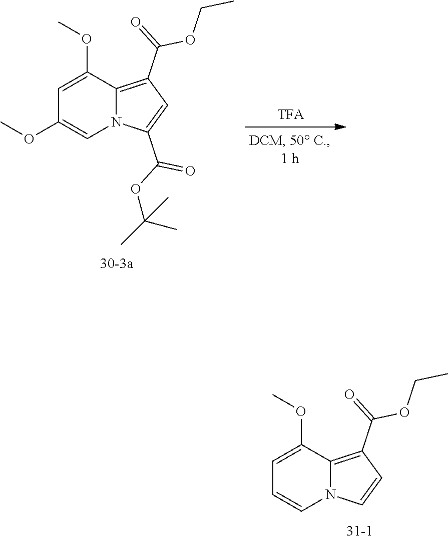 [SMILES: CCOC(=O)c1cc(C(=O)OC(C)(C)C)n2cc(OC)cc(OC)c12 ; CCOC(=O)c1ccn2cccc(OC)c12]

To a stirred solution of 3-(tert-butyl) 1-ethyl 8-methoxyindolizine-1,3-dicarboxylate (30-3a) (420 mg, 1.315 mmol, 1.0 equiv.) in DCM (10 mL) was added TFA (1.0 mL, 13.151 mmol, 10.0 equiv.) at 0° C. The resulting reaction mixture was stirred at 50° C. for 1 h. After completion [Monitored by TLC, mobile Phase 20% EtOAc-Hex], the reaction mixture was quenched with saturated sodium bicarbonate solution (100 mL) and extracted with DCM (100 mL). Then dried over sodium sulfate, and concentrated under reduced pressure to get the crude. The crude compound was purified by silica gel (100-200 mesh) column chromatography eluted with 15-20% ethyl acetate in hexane to afford ethyl 8-methoxyindolizine-1-carboxylate (31-1) as an off-white solid; Yield: (120 mg, 41.6%). ¹H NMR (400 MHz, CDCl₃) δ 7.62 (d, J=6.84 Hz, 1H), 7.18 (s, 2H), 6.59 (t, J=7.08, 7.20 Hz, 1H), 6.29 (d, J=7.56 Hz, 1H), 4.34-4.29 (q, 2H), 3.95 (s, 3H), 1.38 (t, J=7.08, 7.04 Hz, 3H). LCMS: Rt 1.55 min. MS (ES) C₁₂H₁₃NO₃ requires 219, found 220 [M+H]⁺.

### Synthesis of 8-methoxyindolizine-1-carboxylic acid (31-2)

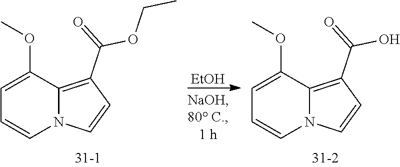 [SMILES: "EtOH" ; CCOC(=O)c1ccn2cccc(OC)c12 ; COc1cccn2ccc(C(=O)O)c12]

To a stirred solution of ethyl 8-methoxyindolizine-1-carboxylate (31-1) (400 mg, 1.826 mmol, 1.0 equiv.) in absolute ethanol (5.0 mL) was added aqueous solution of sodium hydroxide (10 mL, 18.257 mmol, 10.0 equiv.) at RT. The resulting reaction mixture was stirred at 80° C. for 1h. After completion [Monitored by TLC and LCMS, mobile Phase 30% EtOAc-Hex], the excess solvent was evaporated and acidified with a saturated solution of citric acid (50 mL) and extracted with DCM (2×500 mL). Dried over anhydrous sodium sulfate and concentrated under vacuum to afford 8-methoxyindolizine-1-carboxylic acid (31-2) as an off-white solid; Yield: (300 mg, 86%).

¹H NMR (400 MHz, DMSO-d₆) δ 11.47 (s, 1H), 8.07 (d, J=6.64 Hz, 1H), 7.57 (d, J=2.76 Hz, 1H), 7.05 (d, J=2.68 Hz, 1H), 6.74 (t, J=7.28, 6.96 Hz, 1H), 6.52 (d, J=7.56 Hz, 1H), 3.89 (s, 3H). LCMS: Rt 1.27 min. MS (ES) C₁₀H₉NO₃ requires 191, found 192 [M+H]⁺.

### Synthesis of N,8-dimethoxy-N-methylindolizine-1-carboxamide (31-3)

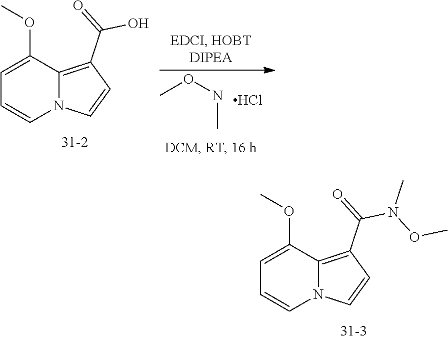

To a stirred solution of 8-methoxyindolizine-1-carboxylic acid (31-2) (2.2 g, 11.507 mmol, 1.0 equiv.) in DCM (50 mL) under nitrogen atmosphere DIPEA (10.1 mL, 57.534 mmol, 5.0 equiv.), EDC.HCl (2.5 g, 12.658 mmol, 1.1 equiv.) and HOBt (2.4 g, 17.26 mmol, 1.5 equiv.) was added at RT and allowed to stir at the same temperature for 15 min. Then N, O-dimethylhydroxylamine hydrochloride (3.4 g, 34.521 mmol, 3.0 equiv.) was added to the reaction mixture and the resulting reaction mixture was stirred at RT for 16 h. After completion [monitored by TLC, mobile Phase 50% EtOAc-hexane], the reaction mixture was diluted with ethyl acetate (500 mL), washed with water (500 mL). The organic layer was collected and dried over magnesium sulfate and concentrated under reduced pressure to get the crude. The crude compound was purified by combi column chromatography eluted with 30%-40% ethyl acetate in hexane to afford N,8-dimethoxy-N-methylindolizine-1-carboxamide (31-3) as an off white solid; Yield: (2 g, 74%). ¹H NMR (400 MHz, CDCl₃) δ 7.57 (d, J=6.8 Hz, 1H), 7.20 (d, J=2.76 Hz, 1H), 6.80 (d, J=2.68 Hz, 1H), 6.47 (t, J=7.12, 7.12 Hz, 1H), 6.09 (d, J=7.40 Hz, 1H), 3.87 (s, 3H), 3.63 (s, 3H), 3.25 (s, 3H).

### Synthesis of 8-methoxyindolizine-1-carbaldehyde (31-4)

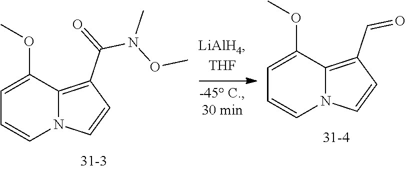 [SMILES: COc1cccn2ccc(C(=O)N(C)OC)c12 ; COc1cccn2ccc(C=O)c12]

To a stirred solution of N,8-dimethoxy-N-methylindolizine-1-carboxamide (31-3) (2 g, 8.538 mmol, 1.0 equiv.) in dry THE (40 mL) was added LAH (2 M in THF) (8.5 mL, 17.075 mmol, 2.0 equiv.) at −40° C. The resulting reaction mixture was stirred at −45° C. to RT for 30 min. After completion [monitored by TLC, mobile Phase 30% EtOAc-hexane], the reaction mixture was quenched with sodium sulfate decahydrate, filtered off, and washed with DCM (100 mL). The organic layer was collected and dried over sodium sulfate and concentrated under reduced pressure to get the crude 8-methoxyindolizine-1-carbaldehyde (31-4) (1.4 g) as an off-white solid, which was forwarded to the next step without purification. LCMS: Rt 2.62 min. MS (ES) C₁₀H₉NO₂ requires 175, found 176 [M+H]⁺.

### Synthesis of (E)-8-methoxy-1-(2-nitrovinyl) indolizine (31-5)

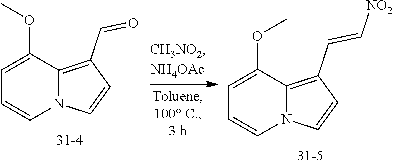 [SMILES: COc1cccn2ccc(C=O)c12 ; COc1cccn2ccc(/C=C/[N+](=O)[O-])c12]

To a stirred solution of 8-methoxyindolizine-1-carbaldehyde (31-4) (1.4 g, 7.991 mmol, 1.0 equiv.) in dry toluene (40 mL) was added Nitromethane (20 mL) and NH₄OAc (495 mg, 6.393 mmol, 0.8 equiv.) at RT. The resulting reaction mixture was stirred at 100° C. for 3 h. After completion [monitored by TLC, mobile Phase 40% EtOAc in hexane], the excess solvent was evaporated under reduced pressure to get the crude. The crude compound was purified by combi column chromatography eluted with 100% DCM to afford (E)-8-methoxy-1-(2-nitrovinyl) indolizine (31-5) as a yellow solid; Yield: (800 mg, 46%). ¹H NMR (400 MHz, CDCl₃) δ 8.91 (d, J=13.2 Hz, 1H), 7.65 (d, J=6.64 Hz, 1H), 7.51 (d, J=13.24 Hz, 1H), 7.28 (d, J=2.8 Hz, 1H), 6.94 (d, J=2.76 Hz, 1H), 6.66 (t, J=7.12, 7.12 Hz, 1H), 6.40 (d, J=7.6 Hz, 1H), 4.02 (s, 3H). LCMS: Rt 3.29 min.

MS (ES) C₁₁H₁₀N203, requires 218, found 219 [M+H]⁺.

### Synthesis of 2-(8-methoxyindolizin-1-yl) ethan-1-amine (31-6)

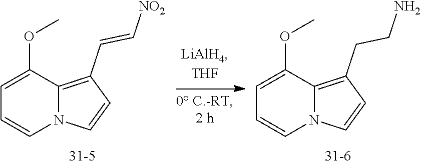 [SMILES: COc1cccn2ccc(/C=C/[N+](=O)[O-])c12 ; COc1cccn2ccc(CCN)c12]

To a stirred solution of (E)-8-methoxy-1-(2-nitrovinyl) indolizine (31-5) (200 mg, 0.917 mmol, 1.0 equiv.) in dry THE (15 mL) was added LAH (2.0 M in THF) (1.4 mL, 2.75 mmol, 3.0 equiv.) at 0° C. The resulting reaction mixture was stirred at RT for 2 h. After completion [monitored by TLC, mobile Phase 50% EtOAc-hexane], the reaction mixture was quenched with sodium sulfate decahydrate, filtered off, and washed with 5% MeOH in DCM (100 mL). The organic layer was collected and dried over sodium sulfate and concentrated under reduced pressure to obtain the crude 2-(8-methoxyindolizin-1-yl) ethan-1-amine (31-6) (170 mg) as a colorless sticky liquid, which was forwarded to the next step without purification. LCMS: Rt 2.05 min. MS (ES) C₁₁H₁₄N₂O, requires 190, found 191 [M+H]⁺.

### Synthesis of tert-butyl (2-(8-methoxyindolizin-1-yl)ethyl)carbamate (31-7)

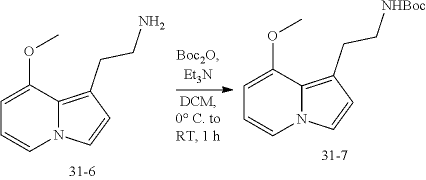 [SMILES: COc1cccn2ccc(CCN)c12 ; COc1cccn2ccc(CCNC(=O)OC(C)(C)C)c12]

To a stirred solution of 2-(8-methoxyindolizin-1-yl) ethan-1-amine (31-6) (190 mg, 1.789 mmol, 1.0 equiv.) in DCM (10 mL) was added Et₃N (0.25 mL, 1.789 mmol, 2.0 equiv.) and Boc anhydride (0.41 mL, 1.789 mmol, 2.0 equiv.) at 0° C. and the resulting reaction mixture was stirred at RT for 3 h. After completion [monitored by TLC, mobile Phase 20% EtOAc-hexane] the reaction mixture was diluted with DCM (100 mL) and washed with water (50 mL), followed by NaCl (50 mL) solution. The organic layer was collected and dried over sodium sulfate and concentrated under reduced pressure to get the crude. The crude compound was purified by combi column chromatography eluted with 10% ethyl acetate in hexane to afford tert-butyl (2-(8-methoxyindolizin-1-yl) ethyl) carbamate (31-7) as a colorless sticky liquid; Yield: (140 mg, 54%).

¹H NMR (400 MHz, DMSO-d₆) δ 7.76 (d, J=6.84 Hz, 1H), 7.36 (d, J=2.52 Hz, 1H), 6.76 (bs, 1H), 6.46 (d, J=2.28 Hz, 1H), 6.35 (t, J=7.08, 7.04 Hz, 1H), 5.97 (d, J=7.32 Hz, 1H), 3.83 (s, 3H), 3.14-3.09 (m, 2H), 2.95-2.92 (m, 2H), 1.36-1.31 (m, 9H). LCMS: Rt 2.02 min. MS (ES) C₁₆H₂₂N₂O₃, requires 290, found 291 [M+H]⁺.

### Synthesis of tert-butyl (2-(8-methoxyindolizin-1-yl) ethyl) (methyl)carbamate (31-8)

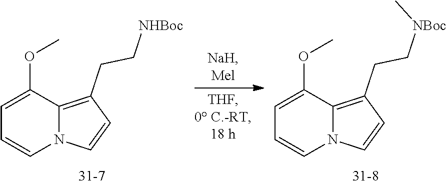 [SMILES: COc1cccn2ccc(CCNC(=O)OC(C)(C)C)c12 ; COc1cccn2ccc(CCN(C)C(=O)OC(C)(C)C)c12]

To stirred solution of tert-butyl (2-(8-methoxyindolizin-1-yl) ethyl) carbamate (31-7) (140 mg, 0.482 mmol, 1.0 equiv.) in dry THF (10 mL) was added (60%) NaH (60 mg, 1.447 mmol, 3.0 equiv.) and Mel (0.15 mL, 2.411 mmol, 5.0 equiv.) at 0° C. in a seal-tube vessel and the resulting reaction mixture was stirred at 0° C. to RT for 18 h. After completion (Monitoring by TLC, 20% EA in Hex), the reaction mixture was quenched with cold water (50 mL) and extracted with ethyl acetate (200 mL), then washed with NaCl solution. The organic layer was collected and dried over magnesium sulfate and concentrated under reduced pressure to get the crude. The crude compound was purified by combi-column chromatography, eluted with 5% ethyl acetate in hexane to afford tert-butyl (2-(8-methoxyindolizin-1-yl) ethyl) (methyl)carbamate (31-8) as a colorless sticky liquid; Yield: (120 mg, 81%). ¹H NMR (400 MHz, DMSO-d₆) δ 7.77 (d, J=6.76 Hz, 1H), 7.36 (d, J=1.52 Hz, 1H), 6.42 (bm, 1H), 6.36 (t, J=6.84, 7.00 Hz, 1H), 5.99 (d, J=7.24 Hz, 1H), 3.84 (s, 3H), 3.35-3.30 (m, 2H), 3.00 (t, J=6.76, 6.76 Hz, 2H), 2.76 (s, 3H), 1.36-1.20 (m, 9H). LCMS: Rt 2.19 min. MS (ES) C₁₇H₂₄N₂O₃, requires 304, found 305 [M+H]⁺.

### Synthesis of 2-(8-methoxyindolizin-1-yl)-N,N-dimethylethan-1-amine (31-9)

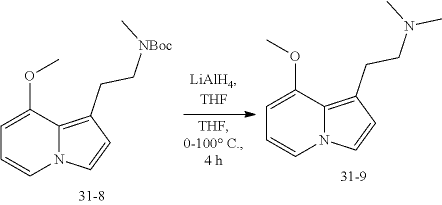 [SMILES: COc1cccn2ccc(CCN(C)C(=O)OC(C)(C)C)c12 ; COc1cccn2ccc(CCN(C)C)c12]

To stirred solution of tert-butyl (2-(8-methoxyindolizin-1-yl) ethyl) (methyl)carbamate (31-8) (120 mg, 0.394 mmol, 1.0 equiv.) in dry THF (10 mL) was added dropwise LAH (2 M in THF) (0.7 mL, 1.38 mmol, 3.5 equiv.) at 0° C. and the resulting reaction mixture was stirred at 100° C. for 7 h. After completion [monitored by TLC, mobile Phase 10% MeOH in DCM], the reaction mixture was quenched with sodium sulfate decahydrate, filtered off, and washed with 5% MeOH in DCM (100 mL). The organic layer was collected and then dried over sodium sulfate and concentrated under reduced pressure to get the crude. The crude compound was purified by combi column chromatography eluted with 0-5% [MeOH in DCM] to afford 2-(8-methoxyindolizin-1-yl)-N,N-dimethylethan-1-amine (31-9) as a colorless sticky liquid; Yield: (50 mg, 59%). ¹H NMR (400 MHz, DMSO-d₆) δ 7.75 (d, J=6.84 Hz, 1H), 7.35 (d, J=2.2 Hz, 1H), 6.47 (d, J=1.96 Hz, 1H), 6.34 (t, J=7.08, 7.04 Hz, 1H), 5.96 (d, J=7.24 Hz, 1H), 3.82 (s, 3H), 2.97 (t, J=7.64, 8.12 Hz, 2H), 2.42 (t, J=8.08, 7.72 Hz, 2H), 2.17 (s, 6H). LCMS: Rt 1.42 min. MS (ES) C₁₃H₁₈N₂O requires 218, found 219 [M+H]⁺.

### Synthesis of 1-(2-(dimethylamino) ethyl) indolizin-8-ol hydrobromide (31-10)

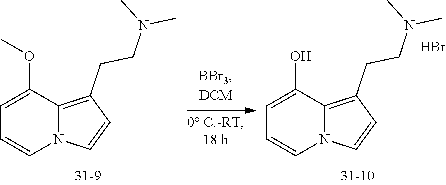 [SMILES: COc1cccn2ccc(CCN(C)C)c12 ; CN(C)CCc1ccn2cccc(O)c12 ; Br]

To stirred solution of 2-(8-methoxyindolizin-1-yl)-N,N-dimethylethan-1-amine (31-9) (90 mg, 0.412 mmol, 1.0 equiv.) in dry DCM (20 mL) was added dropwise BBr₃ (1.0 M in DCM) (4.1 mL, 4.123 mmol, 10.0 equiv.) at 0° C. and the resulting reaction mixture was stirred at RT for 18 h. After completion [monitored by LC-MS], the reaction mixture was cooled to 0° C., quenched with methanol, and concentrated under reduced pressure to get the crude. The crude was washed with diethyl ethyl ether and methanol, and dried to afford 1-(2-(dimethylamino)ethyl)indolizin-8-ol hydrobromide (31-10) as a green solid; Yield: (50 mg, 77%). ¹H NMR (400 MHz, DMSO-d₆) δ 10.09 (s, 1H), 9.25 (bs, 1H), 7.71 (d, J=6.84 Hz, 1H), 7.41 (d, J=2.4 Hz, 1H), 6.54 (d, J=2.24 Hz, 1H), 6.32 (t, J=6.96, 7.04 Hz, 1H), 5.94 (d, J=7.08 Hz, 1H), 3.26 (s, 4H), 2.82 (s, 6H). LCMS: Rt 2.64 min. MS (ES) C₁₂H₁₇BrN₂O requires 204, found 205 [M+H]⁺. HPLC: Rt 5.83 min. Purity (λ 220 nm): 96.45%.

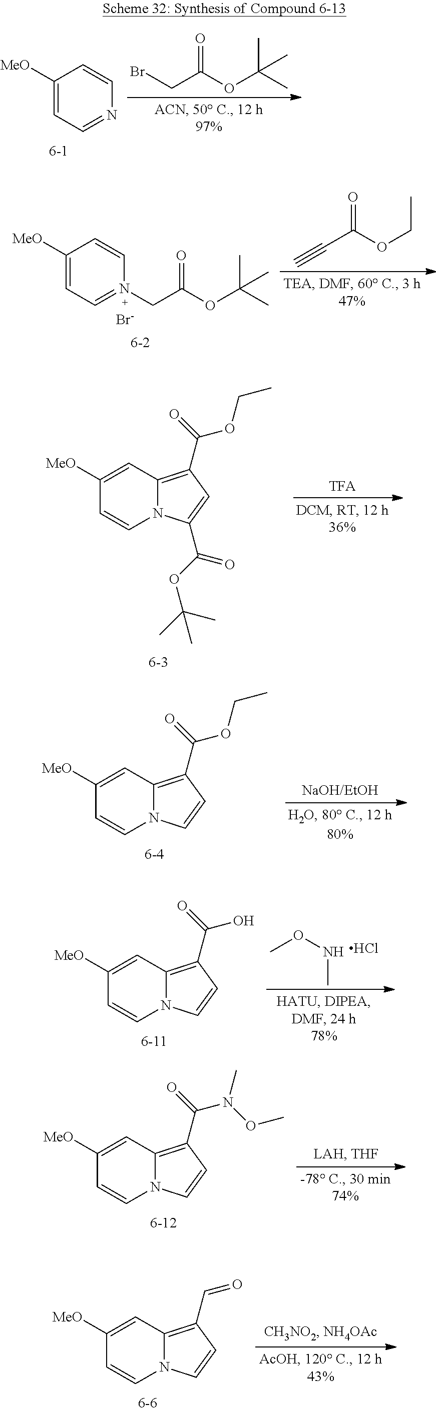

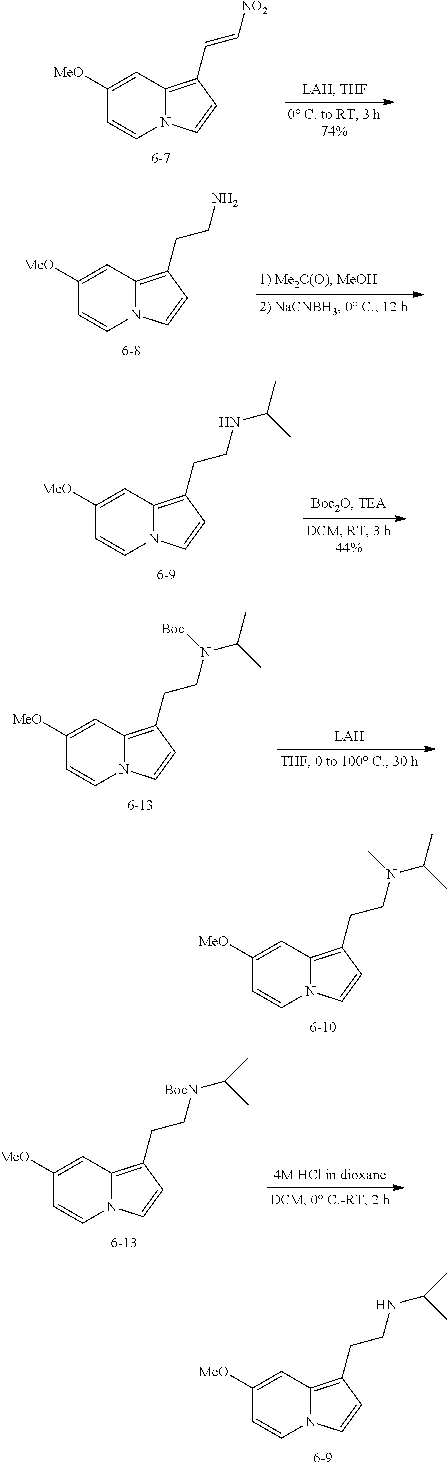

### Synthesis of 1-(2-(tert-butoxy)-2-oxoethyl)-4-methoxypyridin-1-ium bromide (6-2)

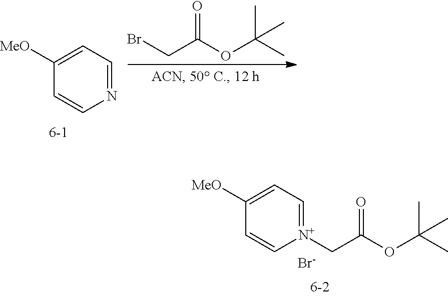

To a stirred solution of 4-methoxypyridine (1) (5.0 g, 45.82 mmol, 1 equiv) in dry ACN (100 mL) was added tert-butyl bromoacetate (6.76 mL, 45.82 mmol, 1 equiv). The reaction mixture was stirred at 50° C. for 12 h. The reaction was monitored by TLC. After completion of the reaction, it was concentrated under vacuum and became solid, then the solid was washed with diethyl ether to afford the pure compound 1-(2-(tert-butoxy)-2-oxoethyl)-4-methoxypyridin-1-ium bromide (6-2); Yield: (10.0 g, 97%); as white solid. ¹H NMR (400 MHz, DMSO-d₆) δ 8.83 (d, J=7.16 Hz, 2H), 7.71 (d, J=7.16 Hz, 2H), 5.37 (s, 2H), 4.12 (s, 3H), 1.45 (s, 9H). LCMS: Rt 1.34 min. MS (ES) C₁₂H₁₈BrNO₃ requires 224, found 225 [M+H]⁺.

### Synthesis of 1-ethyl 3-isopropyl 7-methoxyindolizine-1, 3-dicarboxylate (6-4)

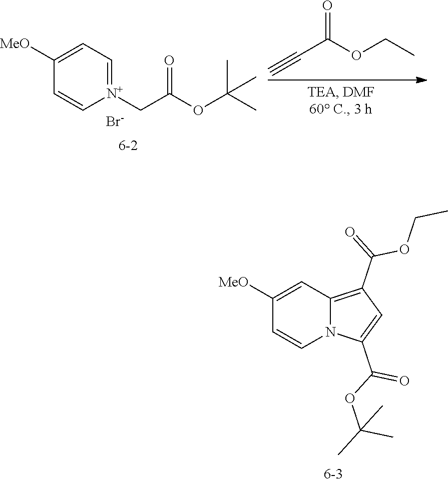

To a stirred solution of 1-(2-(tert-butoxy)-2-oxoethyl)-4-methoxypyridin-1-ium bromide (6-2) (10 g, 33.22 mmol, 1 equiv) in dry DMF (100 mL) was added TEA (13.89 mL, 99.67 mmol, 3 equiv), and ethyl propiolate (3.66 mL, 33.22 mmol, 1 equiv). Then the reaction mixture was stirred at 60° C. for 3h. Completion of the reaction was monitored by TLC (10% EA in Hexane). Upon completion, the reaction mixture was extracted with EA twice (2×200 mL) and ice cold water followed by brine solution. Combined organic layer was dried over anhydrous sodium sulphate, solvent was removed under vacuum and purified by silica gel column chromatography using ethyl acetate/hexane (10:90 v/v) as eluent to afford 1-ethyl 3-isopropyl 7-methoxyindolizine-1,3-dicarboxylate (6-3) as light white solid (5.0 g, 47%). ¹H NMR (400 MHz, CDCl₃) δ 9.34 (d, J=7.72 Hz, 1H), 7.77 (s, 1H), 7.64 (d, J=2.52 Hz, 1H), 6.62-6.59 (m, 1H), 4.37-4.32 (q, 2H), 3.90 (s, 3H), 1.59 (s, 9H), 1.41 (t, J=7.04, 7.16 Hz, 3H). LCMS: Rt 2.27 min. MS (ES) C₁₆H₁₉NO₅ requires 319, found 320 [M+H]⁺.

### Synthesis of ethyl 7-methoxyindolizine-1-carboxylate (6-4)

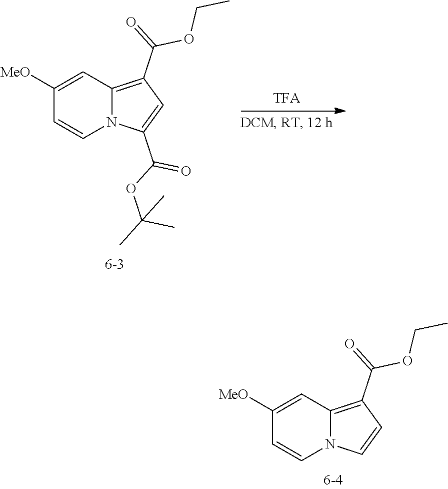 [SMILES: CCOC(=O)c1cc(C(=O)OC(C)(C)C)n2ccc(OC)cc12 ; CCOC(=O)c1ccn2ccc(OC)cc12]

To a stirred solution of 1-ethyl 3-isopropyl 7-methoxyindolizine-1, 3-dicarboxylate (6-3) (5.0 g, 15.66 mmol, 1 equiv) in dry DCM (80 mL) was added TFA (12.06 mL, 156.57 mmol, 10 equiv) at 0° C. The reaction mixture was stirred at RT for 12h. Upon completion, monitored by TLC (20% EA in Hexane), the reaction mixture was diluted with ice cold sodium bicarbonate solution and extracted twice with DCM (2×200 mL). Combined organic layer was dried over anhydrous sodium sulphate, solvent was removed under vacuum and crude material was purified by gravity column to afford ethyl 7-methoxyindilozine-1-carboxylate (6-4) as sticky gum (1.25 g, 36.4%). ¹H NMR (400 MHz, CDCl₃) δ 7.82 (d, J=7.36 Hz, 1H), 7.50 (d, J=2.56 Hz, 1H), 7.10 (d, J=2.88 Hz, 1H), 7.01 (d, J=3 Hz, 1H), 6.43-6.40 (dd, J=2.64, 2.56 Hz, 1H), 4.35-4.30 (q, 2H), 3.88 (s, 3H), 1.39 (t, J=2.32, 4.84 Hz, 3H). LCMS: Rt 3.19 min. MS (ES) C₁₂H₁₃NO₃ requires 219, found 220 [M+H]⁺.

### Synthesis of 7-methoxyindolizine-1-carboxylic acid (6-11)

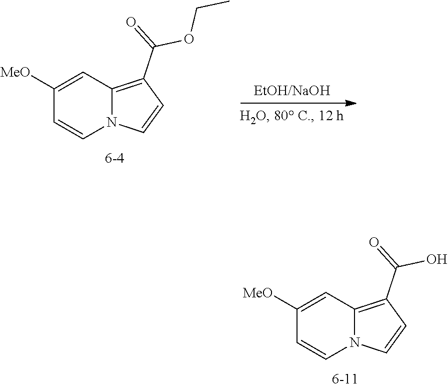 [SMILES: CCOC(=O)c1ccn2ccc(OC)cc12 ; COc1ccn2ccc(C(=O)O)c2c1]

To a stirred solution of ethyl 7-methoxyindilozine-1-carboxylate (6-4) (2.0 g, 9.12 mmol, 1 equiv) in absolute ethanol was added aqueous solution of sodium hydroxide (3.65 mL, 91.22 mmol, 10 equiv). Then the reaction mixture was stirred at 80° C. for 12 h. After completion of the reaction, it was evaporated and diluted with water and washed with DCM to remove non-polar impurities. Then the aqueous part was neutralized with 2(N) HCl and again extracted with DCM. The organic part was concentrated to afford 7-methoxyindolizine-1-carboxylic acid (6-11) as off white solid (1.5 g, 86%). ¹H NMR (400 MHz, DMSO-d₆) δ 11.64 (s, 1H), 8.34 (d, J=7.4 Hz, 1H), 7.36-7.34 (m, 2H), 6.95 (d, J=3 Hz, 1H), 6.56-6.54 (dd, J=2.6 Hz, 1H), 3.83 (s, 3H). LCMS. Rt 1.53 min.

MS (ES) C₁₀H₉NO₃ requires 191, found 192 [M+H]⁺.

### Synthesis of N, 7-dimethoxy-N-methylindolizine-1-carboxamide (6-12)

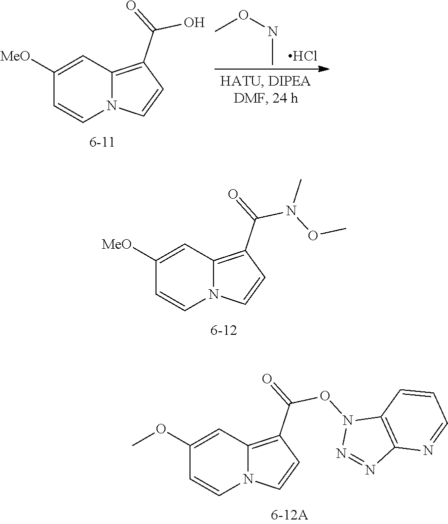

To a stirred solution of 7-methoxyindolizine-1-carboxylic acid (6-11) (2.0 g, 10.46 mmol, 1 equiv) in DMF (30 mL) under nitrogen atmosphere DIPEA (5.47 mL, 31.38 mmol, 3 equiv) and HATU (5.97 g, 15.69 mmol, 1.5 equiv) was added and allowed to stir at RT for 15 min. Then N, O -dimethylhydroxylamine hydrochloride (1.12 g, 11.51 mmol, 1.1 equiv) was added to the resulting reaction mixture and was allowed to stir at room temperature for 24h. Completion of the reaction was monitored by TLC (40% EA in Hexane). Upon completion, the reaction mixture was extracted with EtOAc twice (2×200 mL) and work up with cold water followed by brine solution. Combined organic layer was dried over anhydrous sodium sulphate, solvent was removed under vacuum. Then the whole crude (2.5 g) was dissolved in THE (30 mL) followed by the addition of DIPEA (7 mL, 40.45 mmol, 5 equiv) and N, O-dimethylhydroxylamine hydrochloride (2.37 g, 24.27 mmol, 3 equiv) and stirred it at 70° C. for 12h. Upon completion, the reaction mixture was extracted with EtOAc twice (2×200 mL) and work up with cold water followed by brine solution. Combined organic layer was dried over anhydrous sodium sulphate, solvent was removed under vacuum and purified by silica gel column chromatography to afford N,7-dimethoxy-N-methylindolizine-1-carboxamide (6-12) as off white solid (1.2 g, 79%). ¹H NMR (400 MHz, CDCl₃) δ 7.82-7.78 (m, 2H), 7.23 (d, J=2.72 Hz, 1H), 7.03 (d, J=2.76 Hz, 1H), 6.43-6.40 (dd, J=2.24, 2.20 Hz, 1H), 3.86 (s, 3H), 3.72 (s, 3H), 3.36 (s, 3H). LCMS: Rt 1.69 min. MS (ES) C₁₂H₁₄N₂O₃ requires 234, found 235 [M+H]⁺.

### Synthesis of 7-methoxyindolizine-1-carbaldehyde (6-6)

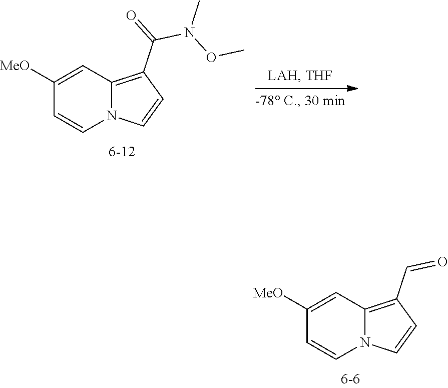 [SMILES: COc1ccn2ccc(C(=O)N(C)OC)c2c1 ; COc1ccn2ccc(C=O)c2c1]

To a stirred solution of N,7-dimethoxy-N-methylindolizine-1-carboxamide (6-12) (450 mg, 1.92 mmol, 1 equiv) in dry THE (15 mL) was added LAH (3.84 ml, 3.84 mmol, 2 equiv) at −78° c. for 30 min. New non polar spot was formed monitored by TLC (40% EA in Hexane). Upon completion, the reaction mixture was quenched with sodium sulphate decahydrate and the formed solid was filtered off, washed with DCM. The organic part was concentrated and crude material was purified by combi-flash column chromatography to afford 7-methoxyindolizine-1-carbaldehyde (6-6) as sticky gum (220 mg, 65.4%). ¹H NMR (400 MHz, CDCl₃) δ 9.85 (s, 1H), 7.88 (d, J=7.28 Hz, 1H), 7.62 (bs, 1H), 7.05 (s, 2H), 6.56-6.53 (dd, J=2.44, 2.24 Hz, 1H), 3.90 (s, 3H). LCMS: Rt 1.53 min. MS (ES) C₁₀H₉NO₂ requires 175, found 176 [M+H]⁺.

### Synthesis of (E)-7-methoxy-1-(2-nitrovinyl)indolizine (6-7)

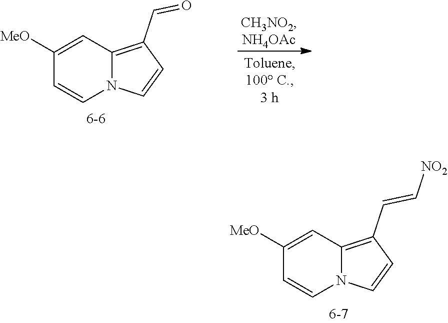 [SMILES: COc1ccn2ccc(C=O)c2c1 ; COc1ccn2ccc(/C=C/[N+](=O)[O-])c2c1]

To a stirred solution of 7-methoxyindolizine-1-carbaldehyde (6-6) (550 mg, 3.14 mmol, 1 equiv) in dry toluene (15 mL) was added Nitromethane (2.55 mL, 47.14 mmol, 15 equiv) and NH₄OAc (157.46 mg, 2.04 mmol, 0.65 equiv) at room temperature and then rise the temperature and stirred at 100° C. for 3h. Upon completion of reaction (monitored by TLC, 40% EA in Hexane), the reaction mixture was concentrated and purified by silica gel column chromatography using Methanol/DCM as eluent to afford (E)-7-methoxy-1-(2-nitrovinyl)indolizine (6-7) as yellow solid (300 mg, 43.74%). ¹H NMR (400 MHz, CDCl₃) δ 8.37 (d, J=12.84 Hz, 1H), 7.84 (d, J=7.36 Hz, 1H), 7.49 (d, J=12.8 Hz, 1H), 7.16 (d, J=3.04 Hz, 1H), 6.92-6.87 (dd, J=1.88, 3 Hz, 2H), 6.51-6.48 (dd, J=2.32 Hz, 1H), 3.91 (s, 3H). LCMS: Rt 3.17 min. MS (ES) Ci¹H₁₀N203, requires 218, found 219 [M+H]⁺.

### Synthesis of 2-(7-methoxyindolizin-1-yl) ethan-1-amine (6-8)

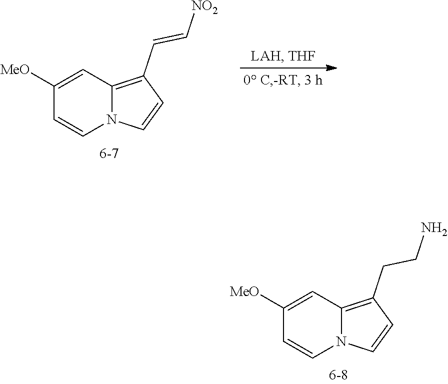 [SMILES: COc1ccn2ccc(/C=C/[N+](=O)[O-])c2c1 ; COc1ccn2ccc(CCN)c2c1]

To a stirred solution of (E)-7-methoxy-1-(2-nitrovinyl) indolizine (6-7) (400 mg, 1.83 mmol, 1 equiv) in dry THE (10 mL) was added LAH (5.5 mL, 5.5 mmol, 3 equiv) at 0° C. Then the reaction mixture was stirred at room temperature for 3h. Upon completion of reaction (monitored by LC-MS), the reaction mixture was quenched with sodium sulphate decahydrate and solid was formed which was filtered off and washed with THF. The organic part was concentrated to afford (2-(7-methoxyindolizin-1-yl) ethan-1-amine (6-8) as sticky gum (300 mg, 86%). Crude compound used in next step without purification. LCMS: Rt 2.06 min. MS (ES) C₁₁H₁₄N₂O, requires 190, found 191 [M+H]⁺.

### Synthesis of N-(2-(7-methoxyindolizin-1-yl) ethyl) propan-2-amine (6-9)

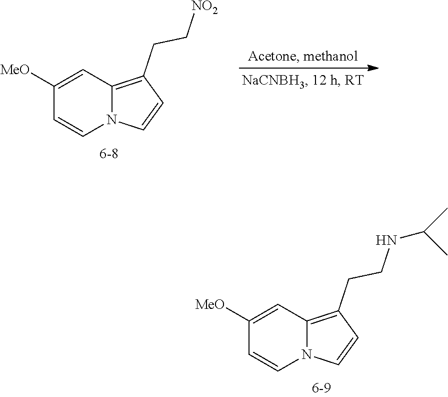 [SMILES: COc1ccn2ccc(CC[N+](=O)[O-])c2c1 ; COc1ccn2ccc(CCNC(C)C)c2c1]

To a stirred solution of 2-(7-methoxyindolizin-1-yl) ethan-1-amine (6-8) (300 mg, 1.58 mmol, 1.0 equiv) in methanol (10 mL) was added acetone (457.32 mg, 7.89 mmol, 5.0 equiv) at room temperature. After 10 min sodium cyano borohydride (198.2 mg, 3.15 mmol, 2.0 equiv) was added to the reaction mixture at 0° C. Then the reaction mixture was stirred at room temperature for 12h. After completion of the reaction (monitored by TLC and LCMS) the mixture was extracted with 5% MeOH/DCM and washed with water. The combined organic part was concentrated to afford N-(2-(7-methoxyindolizin-1-yl) ethyl) propan-2-amine (6-9) as sticky gum (300 mg, 81.89%). Crude compound used in next step without purification. LCMS: Rt 2.41 min. MS (ES) C₁₄H₂₀N20, requires 232, found 233 [M+H]⁺.

### Synthesis of tert-butyl isopropyl (2-(7-methoxyindolizin-1-yl) ethyl) carbamate (6-13)

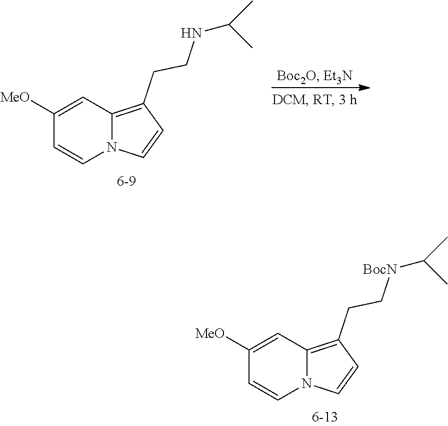 [SMILES: COc1ccn2ccc(CCNC(C)C)c2c1 ; COc1ccn2ccc(CCN(C(=O)OC(C)(C)C)C(C)C)c2c1]

To a stirred solution of N-(2-(7-methoxyindolizin-1-yl)ethyl)propan-2-amine (6-9) (300 mg, 0.90 mmol, 1 equiv) in dry DCM (15 mL) was added TEA (0.4 mL, 2.71 mmol, 3 equiv) and Boc anhydride (320 mg, 2.71 mmol, 3 equiv). Then the reaction mixture was stirred at room temperature for 2h. Upon completion of reaction (monitored by TLC, 20% EA in Hexane), the reaction mixture was extracted with DCM (2×200 mL) and washed with water. The organic part was concentrated and crude material was purified by combi-flash column chromatography using ethyl acetate/hexane (10:90 v/v) as eluent to afford tert-butyl isopropyl (2-(7-methoxyindolizin-1-yl) ethyl) carbamate (6-13) as sticky gum (140 mg, 46.6%). ¹H NMR (400 MHz, CDCl₃) δ 7.69 (d, J=7.36 Hz, 1H), 7.04 (s, 1H), 6.54 (bs, 2H), 6.16 (d, J=4.96 Hz, 1H), 4.34 (bs, 1H), 3.78 (s, 3H), 3.20 (bs, 2H), 2.88 (t, J=8.20, 7.28 Hz, 2H), 1.51 (s, 9H), 1.13 (d, J=6.76 Hz, 6H). LCMS: Rt 3.93 min. MS (ES) C₁₉H₂₈N₂O₃, requires 332, found 333 [M+H]⁺.

### Synthesis of N-(2-(7-methoxyindolizin-1-yl) ethyl-N-methylpropan-2-amine (6-10)

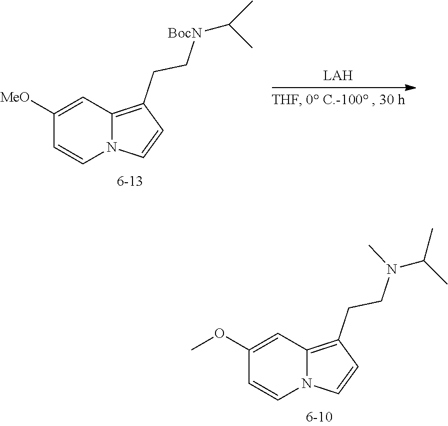 [SMILES: COc1ccn2ccc(CCN(C(=O)OC(C)(C)C)C(C)C)c2c1 ; COc1ccn2ccc(CCN(C)C(C)C)c2c1]

To a stirred solution of tert-butyl isopropyl (2-(7-methoxyindolizin-1-yl) ethyl)carbamate (6-13) (70 mg, 0.21 mmol, 1 equiv) in dry THF (5 mL) was added LAH (0.63 mL, 0.63 mmol, 3 equiv) at 0° C. Then the reaction mixture was stirred at 100° C. for 30 h. After completion of reaction (confirmed by LCMS), the reaction mixture was quenched with sodium sulphate decahydrate and the solid was formed which was filtered off through sintered and washed with THF. Finally the organic part was concentrated and the crude material was purified by combi-flash column chromatography to afford N-(2-(7-methoxyindolizin-1-yl) ethyl)-N-methylpropan-2-amine (6-10) as sticky brown solid (20 mg, 38%). ¹H NMR (400 MHz, DMSO-d₆) δ 8.03 (d, J=7.48 Hz, 1H), 7.21 (d, J=2.64 Hz, 1H), 6.61 (d, J=2.24 Hz, 1H), 6.50 (d, J=2.6 Hz, 1H), 6.18-6.15 (dd, J=2.44, 2.48 Hz, 1H), 3.73 (s, 3H), 2.86-2.82 (m, 1H), 2.73-2.66 (m, 2H), 2.54-2.52 (m, 2H), 2.20 (s, 3H), 0.95 (d, J=6.52 Hz, 6H). LCMS: Rt 2.91 min. MS (ES) C₁₅H₂₂N₂O requires 246, found 247 [M+H]⁺. HPLC: Rt 4.294 min. Purity (λ 220 nm): 93.05%.

### Synthesis of N-(2-(7-methoxyindolizin-1-yl) ethyl) propan-2-amine (6-9)

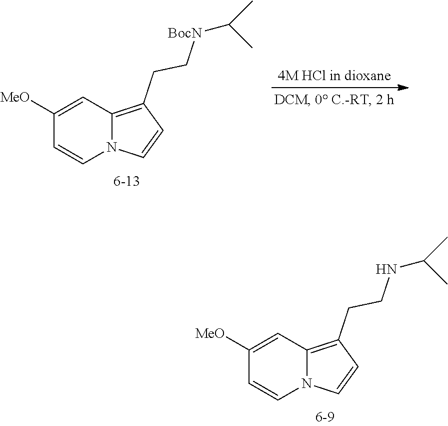 [SMILES: COc1ccn2ccc(CCN(C(=O)OC(C)(C)C)C(C)C)c2c1 ; COc1ccn2ccc(CCNC(C)C)c2c1]

To a stirred solution of tert-butyl isopropyl (2-(7-methoxyindolizin-1-yl) ethyl)carbamate (6-13) (50.0 mg, 0.151 mmol, 1.0 equiv) in dry DCM (2.0 mL) at 0° C., 4M HCl in 1, 4 dioxane (2.0 mL) was added to it and the resultant reaction mixture was stirred at room temperature for 2 h. After completion (monitoring by TLC), the solvent was removed in vacuum and the residue was dissolved in DCM (80 mL) and neutralized by saturated solution of NaHCO₃ (20 mL). Combined organic layer was collected, dried over sodium sulphate and evaporated to get the crude residue which was purified by combi-flash column chromatography using 5% MeOH (1% in TEA) in DCM to afford N-(2-(7-methoxyindolizin-1-yl)ethyl)propan-2-amine (6-9) as colorless sticky gum (16 mg, 45%). ¹H NMR (400 MHz, DMSO) δ 8.03 (d, J=7.52 Hz, 1H), 7.22 (d, J=2.6 Hz, 1H), 6.63 (d, J=2.44 Hz, 1H), 6.50 (d, J=2.64 Hz, 1H), 6.18-6.16 (dd, J=2.52 Hz, 1H), 3.73 (s, 3H), 3.50-3.47 (m, 1H), 2.76-2.66 (m, 4H), 0.96 (d, J=6.16 Hz, 6H). LCMS: Rt 2.83 min. MS (ES) C₁₄H₂₀N20 requires 232, found 233 [M+H]⁺. HPLC: Rt 7.326 min. Purity (λ 240 nm): 95.53%.

### Additional Synthetic Schemes

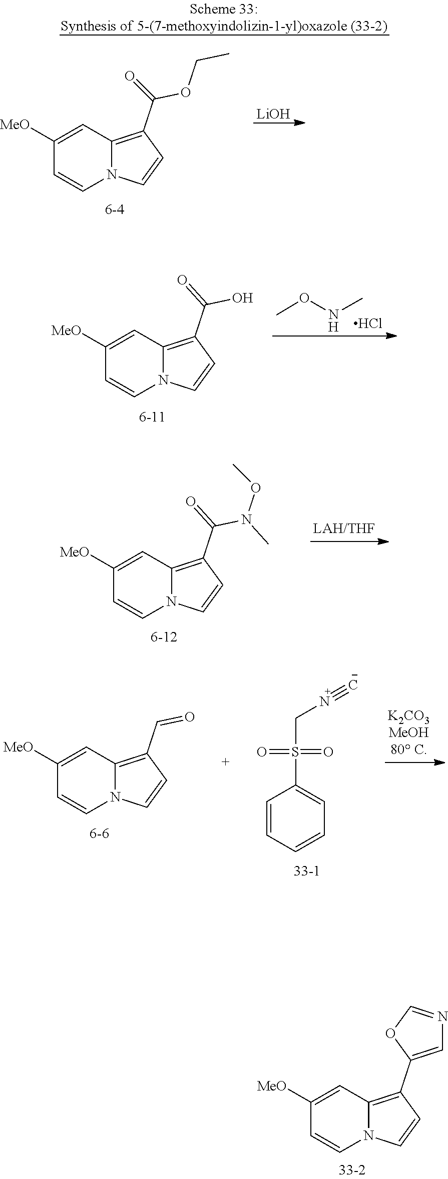

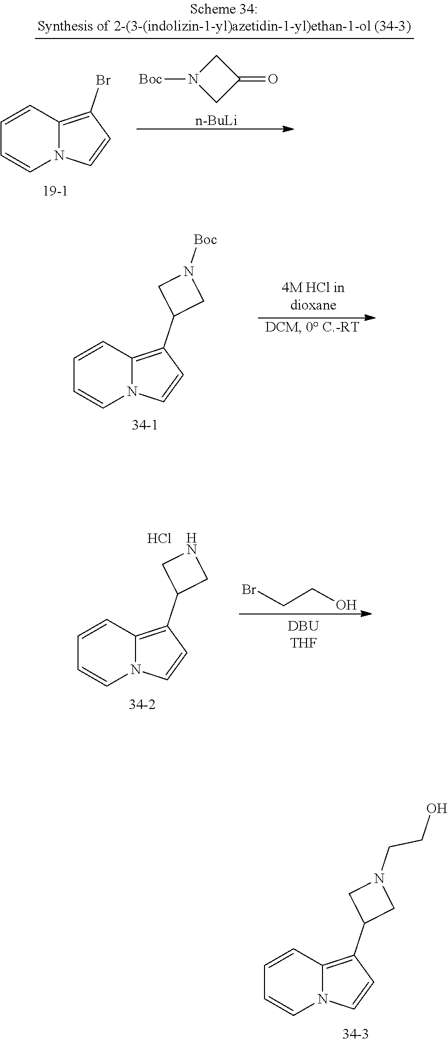

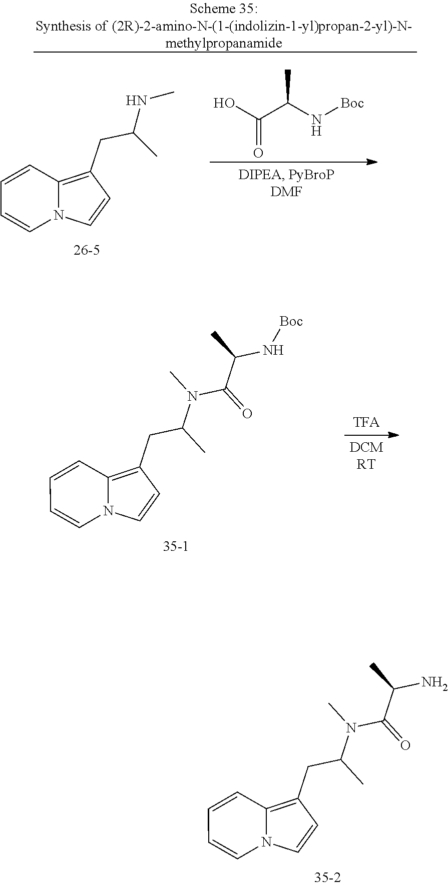 [SMILES: CNC(C)Cc1ccn2ccccc12 ; C[C@@H](NC(=O)OC(C)(C)C)C(=O)O ; CC(Cc1ccn2ccccc12)N(C)C(=O)[C@@H](C)NC(=O)OC(C)(C)C ; CC(Cc1ccn2ccccc12)N(C)C(=O)[C@@H](C)N]

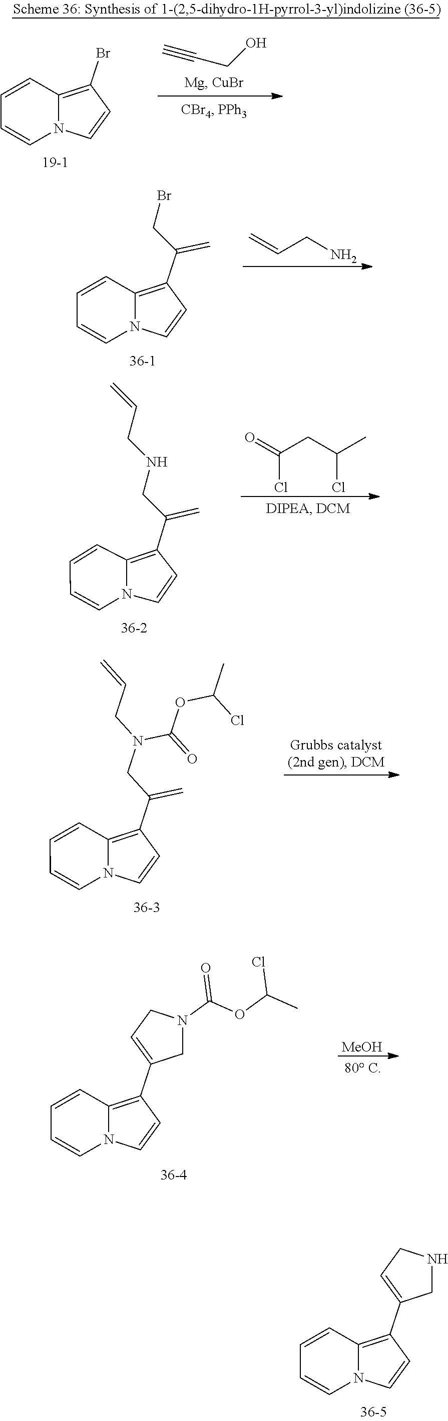

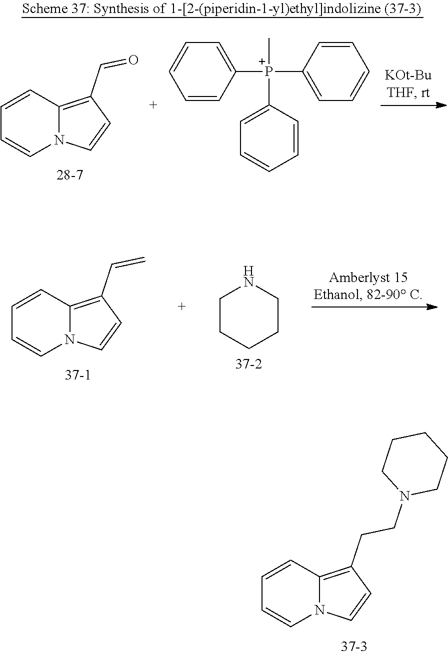 [SMILES: O=Cc1ccn2ccccc12 ; C[P+](c1ccccc1)(c1ccccc1)c1ccccc1 ; C=Cc1ccn2ccccc12 ; C1CCNCC1 ; c1ccn2ccc(CCN3CCCCC3)c2c1]

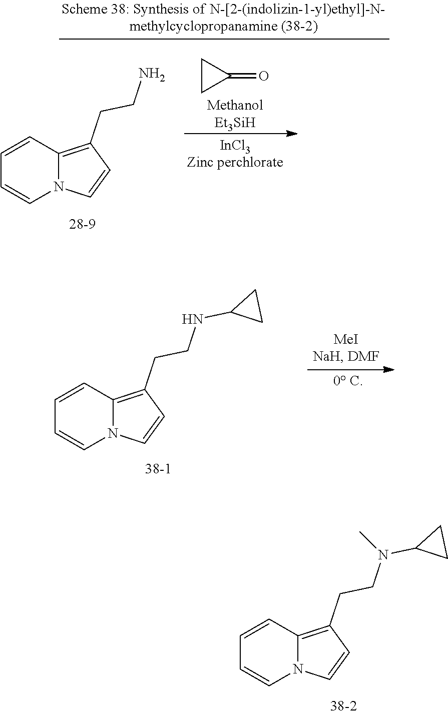

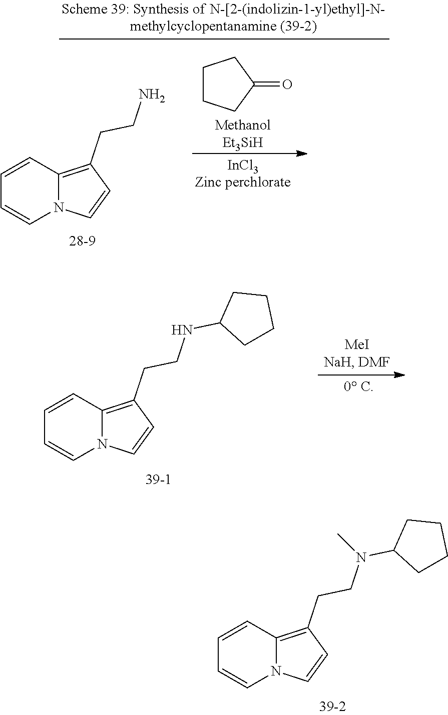 [SMILES: NCCc1ccn2ccccc12 ; O=C1CCCC1 ; c1ccn2ccc(CCNC3CCCC3)c2c1 ; CN(CCc1ccn2ccccc12)C1CCCC1]

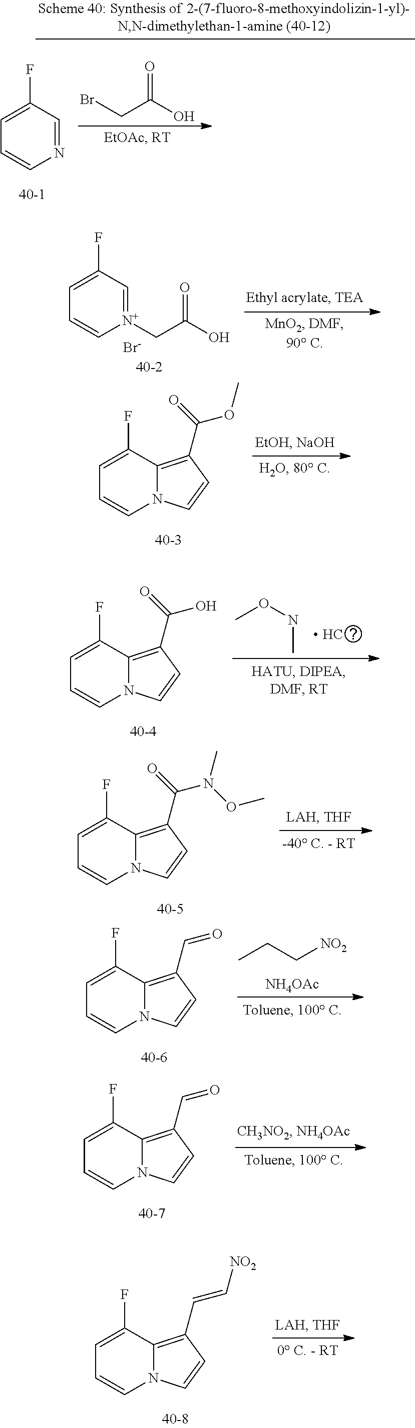

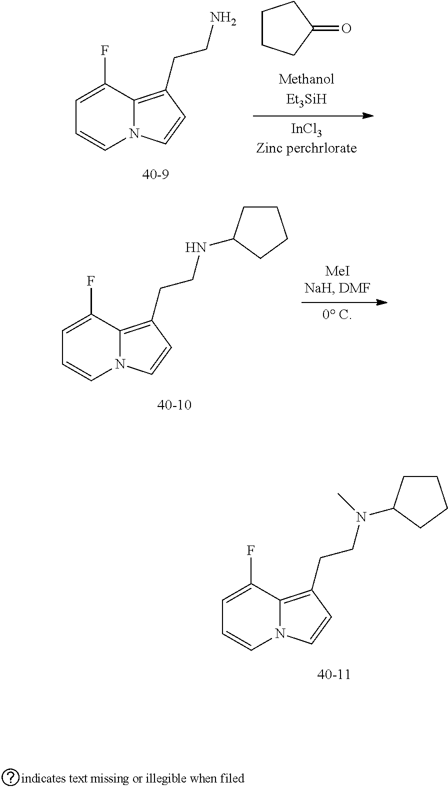 [SMILES: NCCc1ccn2cccc(F)c12 ; O=C1CCCC1 ; Fc1cccn2ccc(CCNC3CCCC3)c12 ; CN(CCc1ccn2cccc(F)c12)C1CCCC1]

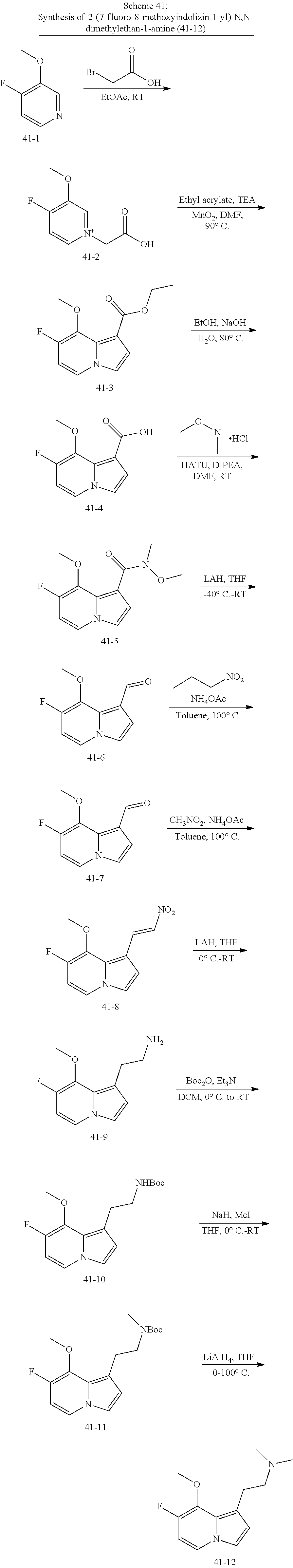

### Example 2: Evaluation of Therapeutic Properties

The clinical and therapeutic effects of compounds that increase extracellular monoamine neurotransmitters are thought to be correlated with their relative tendencies to increase serotonin and dopamine. Liechti and colleagues have proposed that new psychoactive drugs can be classified based on their DAT/SERT inhibition ratios, defined as 1/IC50 at DAT divided by 1/IC50 at SERT (e.g., Luethi and Liechti. 2020. Archives of toxicology, 94(4), pp. 1085-1133). These authors use IC₅₀ measuring uptake inhibition rather than EC₅₀ measuring neurotransmitter release, presumably because drugs that release neurotransmitter also have measurable effects in uptake inhibition assays, producing a metric that can accommodate both reuptake inhibitors and releasers.

In the classification system of Liechti and colleagues, DAT/SERT IC₅₀ ratios>1 are thought to predict psychostimulant effects and compounds with this profile have potential value in treating attention deficit hyperactivity disorder (ADHD) and stimulant use disorders. Example compounds with this profile include dextroamphetamine and methylphenidate (Ritalin, Concerta).

In contrast, serotonin release and a DAT/SERT IC₅₀ ratio of 0.01-0.1 is said to result in a psychoactive drug profile similar to that of MDMA, which includes feelings of emotional openness, authenticity, and decreased neuroticism. MDMA is an experimental adjunct to psychotherapy that shows great potential for treating PTSD and substance use disorders. It may also be able to generally accelerate progress in psychotherapy and aid emotional decision making. MDMA has a reported DAT/SERT IC₅₀ ratio of 0.08 (Simmler and Liechti, New Psychoactive Substances, pp. 143-164).

Compounds with intermediate DAT/SERT IC₅₀ ratios (between 0.1 and 1) appear to sometimes have antidepressant-like or nootropic (cognitive enhancement) qualities and have been proposed as antidepressants, cognitive enhancers, or treatments for substance use disorders. For example, 4-bromomethcathinone (4-BMC, Brephedrone; IUPAC: 1-(4-bromophenyl)-2-(methylamino)propan-1-one) does not have typical psychostimulant effects and has been proposed as a potential antidepressant (Foley and Cozzi. 2003. Drug development research, 60(4), pp. 252-260). The different therapeutic profiles of these intermediate compounds are believed to be at least partially the result of serotonin inhibiting and modifying the stimulating effects of dopamine (Kimmel et al. 2009. Pharmacology Biochemistry and Behavior, 94(2), pp. 278-284; Suyama et al. 2019. Psychopharmacology, 236(3), pp.1057-1066; Wee et al. 2005. Journal of Pharmacology and Experimental Therapeutics, 313(2), pp. 848-854).

One caveat to Liechti's classification system is that compounds that release neurotransmitter may be misclassified if their relative abilities to release dopamine and serotonin are substantially different from their relative abilities to inhibit uptake of dopamine and serotonin. Compounds that appear misclassified in this manner include 3,4,-methylenedioxyethylamphetamine (MDEA; IUPAC [1-(2H-1,3-benzodioxol-5-yl)propan-2-yl](ethyl)amine), which has a reported DAT/SERT IC₅₀ ratio of 3.2 (Simmler et al. 2013. British journal of pharmacology, 168(2), pp. 458-470) but is also reported to have MDMA-like effects in humans (e.g., Hermle et al. 1993. Neuropsychopharmacology, 8(2), pp. 171-176).

Such releasing compounds may be alternatively classified according to their DAT/SERT EC₅₀ ratios, where MDEA has been reported as 0.76 (Rothman et al. 2012. Journal of Pharmacology and Experimental Therapeutics, 341(1), pp.251-262). In this release-based system, MDMA-like therapeutic effects appear present at ratios below 2, with compounds having DAT/SERT EC₅₀ ratios between 2 and 5 having diminished but often still noticeable MDMA-like effects. These intermediate compounds may prove useful for treating ADHD, substance use disorders, and other conditions in individuals who experience significant anxiety from approved psychostimulant pharmacotherapies such as d-amphetamine. Similar to the IC₅₀ system, compounds with higher DAT/SERT EC₅₀ ratios are potential treatments for ADHD and psychostimulant use disorders.

Although MDMA has significant therapeutic potential, it has a number of features that limit its clinical use and may make it contraindicated for some patients. This includes its moderate abuse liability (likely related to its ability to increase extracellular dopamine), acute hypertensive effects (likely related to its norepinephrine release), variable inter-individual metabolism that includes inhibition of the liver enzyme CYP2D6 (increasing risk of drug-drug interactions), potential to induce hyponatremia in women, oxidative stress (likely related to its extensive, though variable, metabolism and formation of reactive metabolites), ability to produce decreases in SERT density after high doses, diminishing therapeutic benefits with repeated use; and a hangover-like after-effects including poor mood and lowered energy. There is therefore a need for additional pharmacologic agents that have similar therapeutic properties while having different pharmacological profiles compared to MDMA.

Compounds that increase extracellular dopamine also often increase extracellular norepinephrine to a similar or greatest extent. For example, d-methamphetamine has a reported DAT/NET EC₅₀ ratio of 0.5, while d-amphetamine has a ratio of 0.9 (Rothman et al. 2001. Synapse, 39(1), pp. 32-41), indicating both are more potent at increasing norepinephrine than dopamine. Differences in the relative balance of dopamine and norepinephrine increases can yield compounds with valuable therapeutic profiles. Norepinephrine increases contribute to cognitive improvements in ADHD but, in excess, can also lead to cardiovascular changes. Dopamine similarly modulates impulsive action but, in excess, can produce compounds with high abuse liability. Nonetheless, individuals with histories of substance abuse who have desensitized dopamine receptors can benefit from compounds that adequately stimulate these receptors. Thus, there is a need for novel treatment compounds that differently balance therapeutic benefits against cardiovascular and abuse liability side effects.

As previously noted, increases in extracellular serotonin and direct stimulation of serotonin receptors present ways for compounds (or compound combinations) to decrease off-target effects and increase select therapeutic effects. For example, compounds that release dopamine and/or norepinephrine and also stimulate 5-HT_{1A} or 5-HT_{1B} receptors can provide fast-acting therapeutic effects on mood and attention while decreasing social anxiety. Similarly, compounds that stimulate 5-HT_{2A} receptors while increasing extracellular neurotransmitter can provide the therapeutic benefits of 5-HT_{2A} agonists while having predictable positive effects on mood that decrease the need for clinical monitoring.

### Example 3: Enantiomeric Separation of Racemic Compounds of the Present Invention

Enantiomers of the present invention can be separated as described herein in the presence or absence of a protecting group. For example, when a compound of the present invention has an amino or hydroxyl substituent a chiral auxiliary or achiral protecting group can be installed on the amino or hydroxyl substituent to facilitate separation or enrichment of its enantiomers. This group can then be removed using conventional methods after separation.

### Example 4: Serum Serotonin Concentrations to Index Drug Interactions with the

Serotonin Transporter (SERT, SLC6A4) Serum serotonin can be measured using High Performance Liquid Chromatography and Fluorescence Detection. Venipuncture collects at least 1 mL of sample, which is spun with serum frozen to below −20° C. within 2 hours of collection. For active compounds, assay results will show increases in serum serotonin, indicating that the compound is a releaser of serotonin.

### Example 5: Human Serotonin Transporter (SERT, SLC6A4) Functional Antagonist Uptake Assay

Human recombinant serotonin transporter expressed in HEK-293 cells are plated. Test compound and/or vehicle is preincubated with cells (1×10E5/ml) in modified Tris-HEPES buffer pH 7.1 for 20 minutes at 25° C. and 65 nM. [3H]Serotonin is then added for an additional 15 minute incubation period. Bound cells are filtered and counted to determine [3H]Serotonin uptake. Compounds are screened at concentrations from 10 to 0.001 μM or similar. Reduction of [3H]Serotonin uptake relative to 1 μM fluoxetine indicates inhibitory activity.

### Example 6: Monoamine Transporter Uptake and Release Assays

An alternative, invasive method of measuring compound interactions with the serotonin, dopamine, or norepinephrine transporter can be conducted according to the methods of Solis et al (2017. Neuropsychopharmacology, 42(10), 1950-1961) and Rothman and Baumann (Partilla et al. 2016. In: Bonisch S, Sitte HH (eds) Neurotransmitter Transporters Springer; New York, pp 41-52).

Male Sprague-Dawley rats (Charles River, Kingston, NY, USA) are used for the synaptosome assays. Rats are group-housed with free access to food and water, under a 12 h light/dark cycle with lights on at 0700 h. Rats are euthanized by CO2 narcosis, and synaptosomes prepared from brains using standard procedures (Rothman, R. B., & Baumann, M. H. (2003). Monoamine transporters and psychostimulant drugs. European journal of pharmacology, 479(1-3), 23-40). Transporter uptake and release assays are performed as described previously (Solis et al. (2017). N-Alkylated analogs of 4-methylamphetamine (4-MA) differentially affect monoamine transporters and abuse liability. Neuropsychopharmacology, 42(10), 1950-1961). In brief, synaptosomes are prepared from caudate tissue for dopamine transporter (DAT) assays, and from whole brain minus caudate and cerebellum for norepinephrine transporter (NET) and serotonin (5-HT) transporter (SERT) assays.

For uptake inhibition assays, 5 nM [3H]dopamine, [3H]norepinephrine, or [3H]5-HT are used for DAT, NET, or SERT assays respectively. To optimize uptake for a single transporter, unlabeled blockers are included to prevent the uptake of [3H]transmitter by competing transporters. Uptake inhibition is initiated by incubating synaptosomes with various doses of test compound and [3H]transmitter in Krebs-phosphate buffer. Uptake assays are terminated by rapid vacuum filtration and retained radioactivity is quantified with liquid scintillation counting (Baumann et al. (2013). Powerful cocaine-like actions of 3, 4-methylenedioxypyrovalerone (MDPV), a principal constituent of psychoactive ‘bath salts’ products. Neuropsychopharmacology, 38(4), 552-562).

For release assays, 9 nM [3H]MPP+ is used as the radiolabeled substrate for DAT and NET, whereas 5 nM [3H]5-HT is used for SERT. Alternatively [3H]dopamine and [3H]norepinephrine may be used for DAT and NET assays, respectively. All buffers used in the release assay contain 1 μM reserpine to block vesicular uptake of substrates. The selectivity of release assays is optimized for a single transporter by including unlabeled blockers to prevent the uptake of [3H]MPP+ or [3H]5-HT by competing transporters. Synaptosomes are preloaded with radiolabeled substrate in Krebs-phosphate buffer for 1 h to reach steady state. Release assays are initiated by incubating preloaded synaptosomes with various concentrations of the test drug. Release is terminated by vacuum filtration and retained radioactivity quantified by liquid scintillation counting.

Effects of test drugs on release are expressed as a percent of maximal release, with maximal release (i.e., 100% Emax) defined as the release produced by tyramine at doses that evoke the efflux of all ‘releasable’ tritium by synaptosomes (10 μM tyramine for DAT and NET assay conditions, and 100 μM tyramine for SERT assay conditions). Effects of test drugs on uptake inhibition and release are analyzed by nonlinear regression. Dose-response values for the uptake inhibition and release are fit to the equation, Y(x)=Ymin+(Ymax−Ymin)/(1+10exp[(log P50−logx)]× n), where x is the concentration of the compound tested, Y(x) is the response measured, Ymax is the maximal response, P50 is either IC50 (the concentration that yields half-maximal uptake inhibition response) or EC₅₀ (the concentration that yields half-maximal release), and n is the Hill slope parameter. EC50s for release of less than 10 uM, but often less than 1 uM, are usually considered indicative of substrate-type releasers.

### Example 7: Marble Burying Measure of Decreased Anxiety and Neuroticism

The marble burying test is a model of neophobia, anxiety, and obsessive-compulsive behavior. Moreover, it has been proposed to have predictive validity for the screening of novel antidepressants and anxiolytics. It is well established to be sensitive to the effects of SSRIs as well as serotonin releasers such as fenfluramine and MDMA (De Brouwer et al., Cognitive, Affective, and Behavioral Neuroscience, 2019, 19(1), 1-39).

The test involves the placement of a standardized number of marbles gently onto the surface of a layer of bedding material within a testing arena. Mice are then introduced into the arena for a standardized amount of time and allowed to explore the environment. The outcome measure of the test is the number of marbles covered, as scored by automatic scoring software or blinded observers. General locomotor activity, often operationalized as total distance traveled, is often used as a control measure. A compound that attenuates anxiety, neuroticism, or obsessive-compulsive behavior decreases marble burying. A compound of the present invention is given to mice and decreases in marble burying, indicates an acute decrease in anxiety and neuroticism.

### Example 8: Neuroplasticity Assay in Primary Cortical Neurons

Compounds of the current invention can be considered psychoplastogens, that is, small molecules that are able to induce rapid neuroplasticity (Olson, 2018, Journal of experimental neuroscience, 12, 1179069518800508). One exemplary method for measuring this, a neurite outgrowth assay conducted in murine primary cortical neurons, is provided below. Other methods are well known in the literature (e.g. Olson, 2018, Journal of experimental neuroscience, 12, 1179069518800508; Ly et al. Cell reports 23, no. 11 (2018): 3170-3182; and references therein).

Primary cortical neurons are prepared from timed pregnant wild-type C57BL/6JRccHsd mice at E18. Animals are sacrificed and embryos are dissected in Calcium and Magnesium free Hanks Balanced Salt Solution (CMF-HBSS) containing 15 mM HEPES and 10 mM NaHCO₃, pH 7.2. Embryos are decapitated, skin and skull gently removed and hemispheres are separated. After removing meninges and brain stem, the hippocampi are isolated, chopped with a sterile razor blade in Chop solution (Hibernate-E without Calcium containing 2% B-27) and digested in 2 mg/mL papain (Worthington) dissolved in Hibernate-E without Calcium for 30 minutes (±5 min) at 30° C. Hippocampi are triturated for 10-15 times with a fire-polished silanized Pasteur pipette in Hibernate-E without Calcium containing 2% B-27, 0.01% DNaseI, 1 mg/mL BSA, and 1 mg/mL Ovomucoid Inhibitor. Undispersed pieces are allowed to settle by gravity for 1 min and the supernatant is centrifuged for 3 min at 228 g. The pellet is resuspended in Hibernate-E containing 2% B-27, 0.01% DNaseI, 1 mg/ml BSA, 1 mg/mL Ovomucoid Inhibitor and diluted with Hibernate-E containing 2% B-27. After the second centrifugation step (3 min at 228 g), the pellet is resuspended in nutrition medium (Neurobasal, 2% B-27, 0.5 mM glutamine, 1% Penicillin-Streptomycin).

Cells are counted in a hemacytometer and seeded in nutrition medium on poly-D-lysine pre-coated 96-well plates at a density of 2.6×104 cells/well. Cells are cultured at 37° C.; 95% humidity and 5% CO2. All wells are handled the same way.

The experiment is performed in adequate technical replicates for all groups, for example five replicates.

On the day of preparation (DIV1), mouse cortical neurons are seeded on poly-D-lysine pre-coated 96-well plates at a density of 2.6×104 cells per well.

On DIV2, cells are treated with test compounds at concentrations selected based on their EC50 at SERT release or 5-HT receptor agonism for three different time points (4 h, 8 h and 24 h), followed by a complete medium change. Additionally, cells are treated with 40 ng/mL of a positive control (Fibroblast growth factor, FGF) or vehicle control (VC) for 48 h.

The experiment is carried out with several, for example five, technical replicates per condition, vehicle treated cells serve as control.

Treated primary neurons are fixed on DIV4 by addition of equal volume 4% paraformaldehyde (PFA) to the medium at room temperature (RT) for 30 minutes.

Cells are rinsed two times with PBS and are permeabilized with 0.1% Triton X-100 in PBS for 30 minutes at RT. Next, cells are blocked for 90 min at RT with 20% horse serum, 0.1% Triton X-100 in PBS.

Then, samples are incubated with the primary antibody against Beta Tubulin Isotype III at 4° C. overnight.

Next day, cells are further incubated for another 30 min at RT. After three washing steps with PBS, cells are incubated with a fluorescently labelled secondary antibody and DAPI (nucleus) for 1.5 hours at RT in the darkness. Cells are again rinsed four times with PBS and imaged with the Cytation 5 Multimode reader (BioTek). From each well, images are taken at 10×magnification.

Digital images from cortical neurons are analyzed for the following parameter using a software-supported automatic quantification method: Number of neurites, number of branches, total length of neurites and length of the longest neurite. Analysis is performed using HCA-Vision software or similar standard software.

Basic statistical analysis is performed. If appropriate, data are presented as mean±standard error of mean (SEM) and group differences are evaluated by e.g. one or two-way ANOVA or T-test. EC50 may be calculated as described elsewhere.

### Example 9: Evaluation of Entactogenic Effect of Decreased Neuroticism

The entactogenic effect of decreased neuroticism can be measured as a decrease in social anxiety using the Brief Fear of Negative Evaluation-revised (BFNE) (Carleton et al., 2006, Depression and Anxiety, 23(5), 297-303; Leary, 1983, Personality and Social Psychology bulletin, 9(3), 371-375). This 12-item Likert scale questionnaire measures apprehension and distress due to concerns about being judged disparagingly or with hostility by others. Ratings use a five-point Likert scale with the lowest, middle, and highest values labeled with “much less than normal,” “normal,” and “much more than normal.” The BFNE can be administered before and repeatedly during therapeutic drug effects. Participants are instructed to answer how they have been feeling for the past hour, or otherwise during the effect of the drug. Baseline-subtracted responses are typically used in statistical models.

### Example 10: Evaluation of Entactogenic Effect of Authenticity

The entactogenic effect of authenticity can be measured using the Authenticity Inventory (Kernis & Goldman. 2006. Advances in experimental social psychology, 38, 283-357) as modified by Baggott et al (Journal of Psychopharmacology 2016, 30.4: 378-87). Administration and scoring of the instrument is almost identical to that of the BFNE. The Authenticity Inventory consists of the following items, which are each rated on a 1-5 scale, with select items reverse scored as specified by Kernis & Goldman:
I am confused about my feelings.
I feel that I would pretend to enjoy something when in actuality I really didn't.
For better or worse, I am aware of who I truly am.
I understand why I believe the things I do about myself
I want the people with whom I am close to understand my strengths.
I actively understand which of my self-aspects fit together to form my core or true self.
I am very uncomfortable objectively considering my limitations and shortcomings.
I feel that I would use my silence or head-nodding to convey agreement with someone else's statement or position even though I really disagreed.
I have a very good understanding of why I do the things I do.
I am willing to change myself for others if the reward is desirable enough.
I would find it easy to pretend to be something other than my true self.
I want people with whom I am close to understand my weaknesses.
I find it difficult to critically assess myself. (unchanged)
I am not in touch with my deepest thoughts and feelings.
I feel that I would make it a point to express to those I am close with how much I truly care for them.
I have difficulty accepting my personal faults, so I try to cast them in a more positive way.
I feel that I idealize the people close to me rather than objectively see them as they truly are.
If asked, people I am close to could accurately describe what kind of person I am.
I prefer to ignore my darkest thoughts and feelings.
I am aware of times when I am not being my true self.
I am able to distinguish the self-aspects that are important to my core or true self from those that are unimportant.
People close to me would be shocked or surprised if they discovered what I am keeping inside me.
It is important for me to understand the needs and desires of those with whom I am close.
I want people close to me to understand the real me, rather than just my public persona or “image”.
I could act in a manner that is consistent with my personally held values, even if others criticized me or rejected me for doing so.
If a close other and I were in disagreement, I would rather ignore the issue than constructively work it out.
I feel that I would do things that I don't want to do merely to avoid disappointing people.
My behavior expresses my values.
I actively attempt to understand myself as well as possible.
I feel that I'd rather feel good about myself than objectively assess my personal limitations and shortcomings.
My behavior expresses my personal needs and desires.
I have on a “false face” for others to see.
I feel that I would spend a lot of energy pursuing goals that are very important to other people even though they are unimportant to me.
I am not in touch with what is important to me.
I try to block out any unpleasant feelings I have about myself.
I question whether I really know what I want to accomplish in my lifetime.
I am overly critical about myself.
I am in touch with my motives and desires.
I feel that I would deny the validity of any compliments that I receive.
I place a good deal of importance on people close to me understanding who I truly am.
I find it difficult to embrace and feel good about the things I have accomplished.
If someone pointed out or focused on one of my shortcomings, I would quickly try to block it out of my mind and forget it.
The people close to me could count on me being who I am, regardless of what setting we were in.
My openness and honesty in close relationships are extremely important to me.
I am willing to endure negative consequences by expressing my true beliefs about things.

### Example 11: Evaluation of Side Effects of Entactogens and Related Drugs

Adverse effects of an entactogen include formation of tolerance to entactogens, headache, difficulty concentrating, lack of appetite, lack of energy, and decreased mood. In addition to these mild toxicities, MDMA is associated with a number of more severe toxicities, including but not limited to acute and chronic cardiovascular changes, hepatotoxicity, hyperthermic syndromes, hyponatremia, and neurotoxicity (see the MDMA Investigator's Brochure, 13th Edition: Mar. 22, 2021, and references therein, available from the sponsor of MDMA clinical trials at MAPS.org).

Acute physiological changes can be measured in humans with standard clinical methods (blood pressure cuffs, 3-lead EKG, tympanic or oral temperature, serum sodium, etc), with measures usually collected before and at scheduled intervals after an entactogen. For example, measures may be collected before, 0.5, 0.75, 1, 1.5, 2, 2.5, 3, 4, 5, 6, and 8 hours after an entactogen. Maximum change from baseline and area-under-the-effects-versus-time-curve may be used as summary measures and statistically compared to a placebo control condition.

To measure adverse symptoms, patients can be asked to complete a self-report symptom questionnaire, such as the Subjective Drug Effects Questionnaire (SDEQ) or List of Complaints. The SDEQ is a 272-item self-report instrument measuring perceptual, mood, and somatic changes caused by drugs including hallucinogens like LSD (Katz et al. 1968. J Abnorm Psychology 73:1-14). It has also been used to measure the therapeutic and adverse effects of MDMA (Harris et al. 2002. Psychopharmacology, 162(4), 396-405). The List of Complaints is a 66-item questionnaire that measures physical and general discomfort and is sensitive to entactogen-related complaints (e.g., Vizeli & Liechti. 2017. Journal of Psychopharmacology, 31(5), 576-588).

Alternatively, individual items can be taken from the SDEQ or List of Complaints in order to create more focused questionnaires and reduce the burden of filling out time-consuming paperwork on participants. To measure tolerance formation, a global measure of the intensity of therapeutic effects can be used, such as the question “on a scale from 0 to 100 where 0 is no ‘good drug effect’ and 100 is the most ‘good drug effect’ you have ever felt, how would you rate this drug experience?”

In some embodiments, the questionnaire will be administered approximately 7 hours after a patient takes MDMA or another entactogen (with instructions to answer for the time since taking the entactogen) and then daily (with instructions to answer for the last 24 hours) for up to 96 hours after the entactogen was taken. Decreases in adverse effects of a compound compared to MDMA can be shown by comparing the intensity (for the tolerance question) or prevalence (for other symptom questions) of effects that occur. Prevalence of adverse effects including formation of tolerance to entactogens, headache, difficulty concentrating, lack of appetite, lack of energy, and decreased mood may be decreased by approximately 10%, 20%, 30%, 40%, 50%, 60%, 70%, 80%, 90%, 99%, or 100%.

As an alternative to measuring side effects of entactogens in clinical trials, preclinical studies in rodents may also be used. Appropriate tasks and behaviors that may be used to measure side effects include physiological measures (heart rate, blood pressure, body temperature), the modified Irwin procedure or functional observational battery (Irwin, Psychopharmacologia, 13, 222-257, 1968), and locomotor activity (such as distance traveled, rearing frequency, and rearing duration; Piper et al., J Pharmacol Exp Ther, 317, 838-849, 2006). In these studies, an entactogen is administered at different doses (including a vehicle only placebo) to different groups of animals and measures are made at scheduled times before and after administration. For example, 0, 1.5, 3, 15, and 30 mg/kg of a compound may be administered intraperitoneally and measures made before and 15, 30, 60, 120 and 180 minutes and 12, 24, 36, and 48 hours after administration of the test substance.

### Example 12: Dopamine Release Assay

[1-(indolizin-1-yl)propan-2-yl](methyl)amine (1ZP2MA) HCl salt (26-5 HCl), 1-(indolizin-1-yl)butan-2-amine (1ZB2A) HCl salt (27-1 HCl), and [1-(indolizin-1-yl)butan-2-yl](methyl)amine (1ZB2MA) HCl salt (25-14 HCl) were assayed for dopamine release in rat synaptosomes. Testing concentrations were 10 μM, followed by 4-fold serial dilution, with a total of eight tested concentrations (from 10 to 0.0001 μM). Methods were as described in Example 6 with 30 nM [³H]DA used at a 1 hr incubation to preload synaptosomes. Tyramine (150 μM initial concentration) was used a positive control. Measurements were repeated twice. Concentration-response functions were fit once for each compound via the drmo function in the R package drc, using a four-parameter log-logistic model.

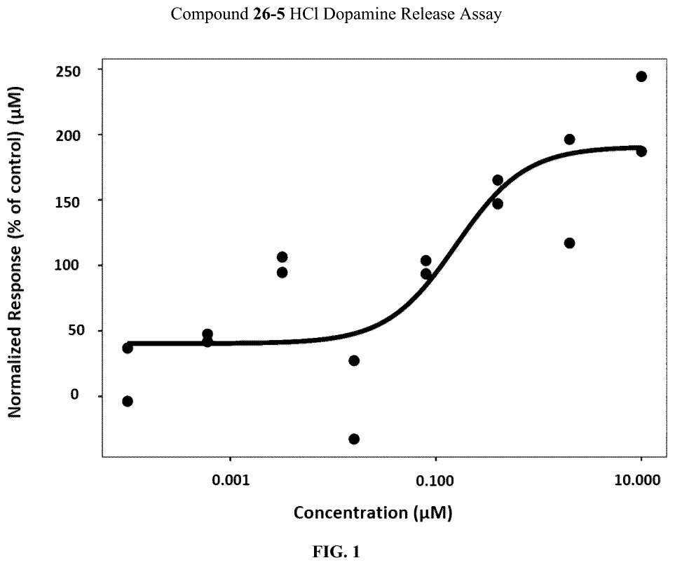

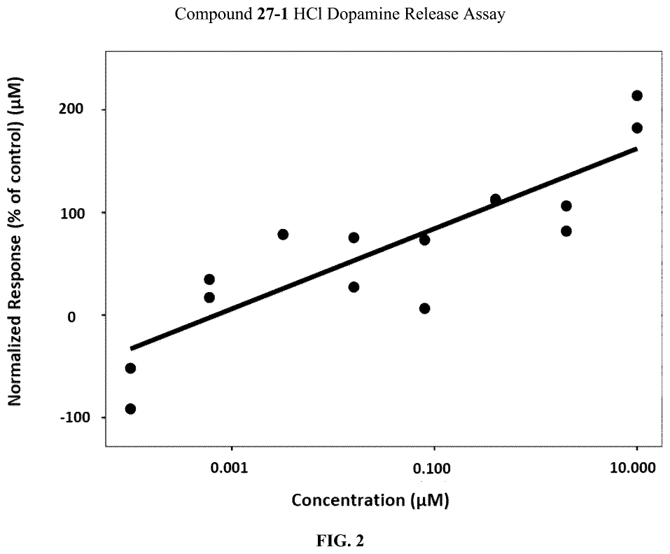

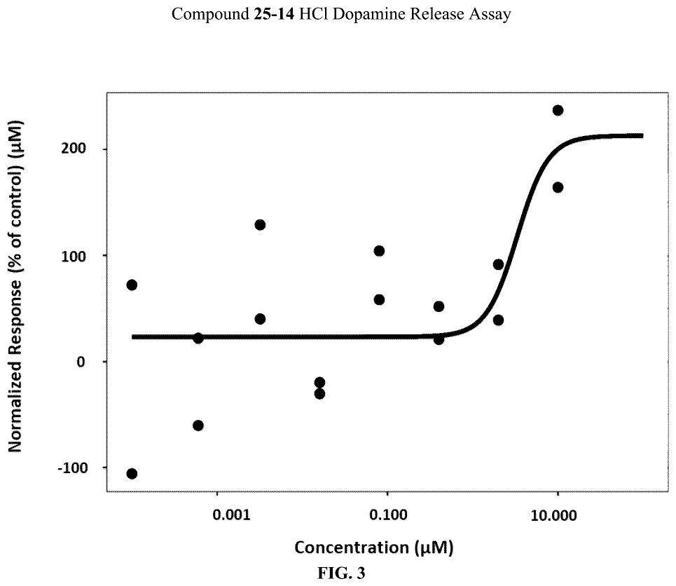

All three compounds showed ability to release dopamine, with EC50s for 26-5, 27-1, and 25-14 estimated as 0.0619, 21.8, 2.71 μM, respectively (FIGS. 1, 2, and 3 respectively).

### Example 13: Rodent Head-Twitch Response (HTR) Assay of Psychedelic Effects

In order to assay for in vivo psychedelic effects, the rodent head-twitch response (HTR) is commonly used. The HTR is a rhythmic rotational head movement that resembles the shaking movements of a wet dog attempting to dry itself and that occurs in mice and rats in response to 5-HT_{2A}receptor activation. Head movement during the HTR is rhythmic and occurs within a specific frequency range (around 90 Hz in mice). The HTR is correlated with tendency to produce psychedelic effects in humans (Halberstadt et al 2020, doi:10.1016/j.neuropharm.2019.107933). Accordingly, the HTR is considered “a reliable proxy for psychedelic drug activity in humans” (Glatfelter et al. 2022 doi:10.1021/acsptsci.1c00237).

HTR is typically assessed in mice for approximately 20 min beginning from 0 to 30 minutes after administration of a 5-HT_{2A} agonist or control. (Exact timing will depend on the pharmacokinetics of the compound being assessed.) HTR can be assessed in real time by a trained observer, by recording video for later assessment, or by using automated techniques (Glatfelter et al. 2022 doi:10.1021/acsptsci.1c00237; Halberstadt 2020, doi:10.1038/s41598-020-65264-x). Total twitches per session is used as the primary outcome measure.

### Example 14: Measuring Antidepressant-Like Effects in Humans

Severity of depression, anxiety, and trauma-related disorders can be measured with a variety of self-report and observer-rated scales (e.g., Bardhoshi et al 2016 doi:10.1002/jcad.12075; Fried et al. 2022 doi:10.1038/s44159-022-00050-2; Fried 2017 doi:10.1016/j.jad.2016.10.019; Glaesmer et al 2015, doi:10.1016/j.psychres.2015.07.010; Kazlauskas et al 2017 doi:10.1159/000484415; Steel et al 2012 doi:10.1016/j.injury.2010.11.045), such as the Hamilton Rating Scale for Depression, the Beck Depression Inventory, the Centre for Epidemiological Studies Depression Scale, Edinburgh Postnatal Depression Scale, the Clinician-Administered PTSD Scale, the Impact of Event Scale-Revised, the Adjustment Disorder New Module, the International Adjustment Disorder Questionnaire, Social Interaction Anxiety Scale, the Social Phobia Scale, the State-Trait Anxiety Inventory, the Generalized Anxiety Disorder Test-7, the Perceived Stress Scale, the Positive Negative Affect Scale, the Posttraumatic Stress Diagnostic Scale, the Clinician-Administered PSTD Scale, the PTSD Checklist, and the Structured Interview for PTSD. Typically, to show a therapeutic improvement, one or more measures (i.e., instruments, scales, subscales, or single items) are administered at least once, but preferably more than once, to establish a baseline before treatment. Subsequently, a treatment is initiated with additional administration(s) of the same instrument to detect potential changes. Changes from pre- to post-treatment are statistically analyzed to establish that apparent improvements are not due to chance fluctuations. Representative symptoms and signs of depressive, anxiety, and trauma-related disorders that may improve with treatment include: Early insomnia, Middle insomnia, Late insomnia, Hypersomnia, Sad mood, Anxious/fearful mood, Panic/phobic symptoms, Irritable mood, Mood reactivity, Diurnal variation, Grief, Appetite decrease, Appetite increase, Weight decrease, Weight increase, Concentration problems, Indecisiveness, Guilt, Worthlessness, Pessimism/hopeful about future, Suicidal ideation, Loss of interest, Loss of pleasure, Tiredness, fatigue, Loss of energy, Decreased libido, Psychomotor retardation, Psychomotor agitation, Demoralization, Rumination or preoccupation, Somatic complaints (e.g., pain, headaches, heaviness of limbs), Sympathetic arousal (e.g., palpitations,tremors, tinnitus, chest pain), Gastrointestinal problems (e.g., constipation, diarrhea), Interpersonal sensitivity, Leaden paralysis, Past failure (e.g., “I am a total failure as a person”), Punishment feelings, Self-dislike, Self-criticalness (e.g., blaming oneself for bad things that happen), Crying, Lonely, “Everything I do is an effort”, “Talked less than usual”, “People were unfriendly”, “People disliked me”, “Bothered by things that usually don't bother me”, “Feeling as good as other people”, Feeling Happy, “I am useful and needed”, “My life is pretty full”, Inner tension, Inability to feel, Hypochondriasis, and Loss of insight.

### Example 15: In Vitro Activity Studies

Compound 28-12 oxalate ([2-(indolizin-1-yl)ethyl]dimethylamine oxalate) was evaluated for activity at 47 target sites at ten concentrations up to 30 μM, with EC50 or IC50 determined whenever possible.

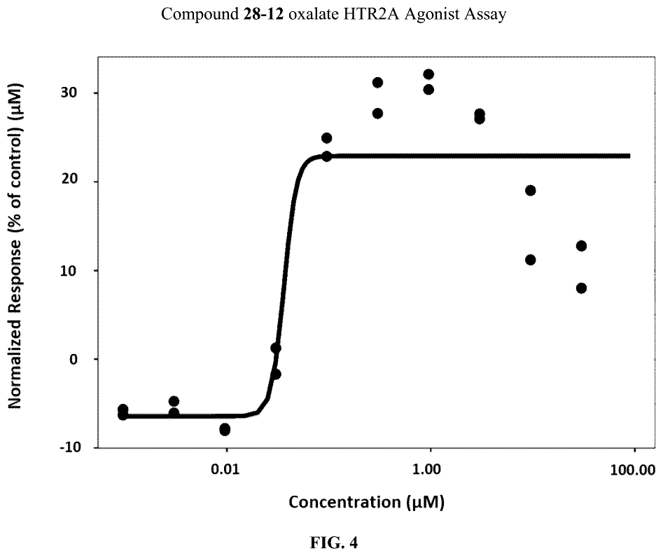

Compound 28-12 oxalate showed surprising activity at 5-HT_{2A}R. The 5-HT_{2A}R agonist activity of tryptamine-based psychedelics is thought to rely, to a great extent, on the indole nitrogen. Because the nitrogen of the indolizine is less available for the salt bridge interaction that occurs between psychedelic tryptamine's indole nitrogen and a serine on the 5-HT_{2A}receptor, compound 28-12 oxalate was not expected to retain activity. Surprisingly, compound 28-12 oxalate showed high potency and partial agonist effects at 5-HT_{2A}R. The EC50 was estimated as approximately 52.25 nM, while the maximum effect was approximately 30% of the response produced by 5-HT (FIG. 4). These partial agonist effects suggest the unexpected use of compound 28-12 oxalate and other indolizine derivatives as modulators of 5-HT_{2A}R activity in situations where hallucinatory effects would be undesirable.

Compound 28-12 oxalate showed relatively few interactions with other potential binding sites, with agonism of 5-HT_{1B} receptors (EC50 of approximately 143.32 nM) being the only interaction with sub-micromolar potency. This further supports the use of compound 28-12 oxalate and other compounds of Formula I for conditions that include signs or symptoms related to headache, inflammation, stress sensitivity, addiction and reward system dysfunction, mood, anxiety, and aggression.

| | | Compound 28-12 oxalate | |
|---|---|---|---|
| | | EC₅₀ (μM) | IC₅₀ (μM) |
| | | Agonist/ | Antag./Blocker/ |
| Assay Target | Assay Type | Opener | Inhibitor |

Serotonergic Receptors

| | | | |
|---|---|---|---|
| 5-HT1B | cAMP | 0.143 | >30 |
| 5-HT2A | Calcium Flux | 0.052 | 1.083 |
| 5-HT2B | Calcium Flux | >30 | 3.283 |
| 5-HT3A | Ion Channel | >30 | 7.816 |
| 5-HT1A | cAMP | >30 | >30 |

Other GPCRs

| | | | |
|---|---|---|---|
| ADRA1A | Calcium Flux | >30 | 2.309 |
| ADRA2A | cAMP | >30 | >30 |
| ADRB2 | cAMP | >30 | 10.171 |
| HRH1 | Calcium Flux | >30 | 9.269 |
| ADORA2A | Calcium Flux | >30 | >30 |
| ADRB1 | cAMP | >30 | >30 |
| AVPR1A | Calcium Flux | >30 | >30 |
| CCKAR | Calcium Flux | >30 | >30 |
| CHRM1 | Calcium Flux | >30 | >30 |
| CHRM2 | cAMP | >30 | >30 |
| CHRM3 | Calcium Flux | >30 | >30 |
| CNR1 | cAMP | >30 | >30 |
| CNR2 | cAMP | >30 | >30 |
| DRD1 | cAMP | >30 | >30 |
| DRD2S | cAMP | >30 | 22.272 |
| EDNRA | Calcium Flux | >30 | >30 |
| HRH2 | cAMP | >30 | >30 |
| OPRD1 | cAMP | >30 | >30 |
| OPRK1 | cAMP | >30 | >30 |
| OPRM1 | cAMP | >30 | >30 |

Other Ion Channels

| | | | |
|---|---|---|---|
| nAChR(a4/b2) | Ion Channel | >30 | >30 |
| NAV1.5 | Ion Channel | | >30 |
| CAV1.2 | Ion Channel | | >30 |
| GABAA | Ion Channel | >30 | >30 |
| hERG | Ion Channel | | >30 |
| KvLQT1/minK | Ion Channel | >30 | >30 |
| NMDAR (1A/2B) | Ion Channel | >30 | >30 |

Monoamine Transporters

| | | |
|---|---|---|
| DAT | Transporter | >30 |
| NET | Transporter | >30 |
| SERT | Transporter | >30 |

Enzymes

| | | |
|---|---|---|
| MAOA | Enzymatic | 7.252 |
| AChE | Enzymatic | >30 |
| COX1 | Enzymatic | 5.131 |
| COX2 | Enzymatic | >30 |
| PDE3A | Enzymatic | >30 |
| PDE4D2 | Enzymatic | >30 |

Kinases

| | | | |
|---|---|---|---|
| ROCK1 | Binding | | >30 |
| INSR | Binding | | >30 |
| AR | NHR Nuclear | >30 | >30 |
| | Translocation | | |
| GR | NHR Protein | >30 | 17.080 |
| | Interaction | | |
| LCK | Binding | | >30 |
| VEGFR2 | Binding | | >30 |

### In Vitro Assay Methods

Concentrations of test compounds were 0.000954855, 0.003017342, 0.0095348, 0.030129971, 0.095210709, 0.300865859, 0.950736105, 3.004326105, 9.493670464, and 30 μM.

### A) CAMP Secondary Messenger Assays

CAMP secondary messenger assays used cell lines that stably expressed non-tagged GPCRs. Hit Hunter® CAMP assays monitored the activation of a GPCR via Gi and Gs secondary messenger signaling in a homogenous, non-imaging assay format using Enzyme Fragment Complementation (EFC) with B-galactosidase (B-gal) as the functional endpoint. For the assay system, exogenously introduced Enzyme Donor (ED) fused to cAMP (ED-cAMP) competes with endogenously generated cAMP for binding to an anti-cAMP-specific antibody. Active 3-gal is formed by complementation of exogenous Enzyme Acceptor (EA) to any unbound ED-cAMP. Active enzyme can then convert a chemiluminescent substrate, generating an output signal detectable on a standard microplate reader.

### Assay Design

### Cell Handling

1. cAMP Hunter cell lines were expanded from freezer stocks according to standard procedures.

2. Cells were seeded in a total volume of 20 μL into white walled, 384-well microplates and incubated at 37° C. for the appropriate time prior to testing.

3. cAMP modulation was determined using the DiscoverX HitHunter cAMP XS+ assay.

### Gs Agonist Format

1. For agonist determination, cells were incubated with sample to induce response.

2. Media was aspirated from cells and replaced with 15 μL 2:1 HBSS/l0 mM Hepes: cAMP XS+Ab reagent.

3. Intermediate dilution of sample stocks was performed to generate 4× sample in assay buffer.

4. 5 μL of 4× sample was added to cells and incubated at 37° C. or room temperature for 30 or 60 minutes.

### Gi Agonist Format

1. For agonist determination, cells were incubated with sample in the presence of EC₅₀ forskolin to induce response.

2. Media was aspirated from cells and replaced with 15 μL 2:1 HBSS/10 MM Hepes: cAMP XS+Ab reagent.

3. Intermediate dilution of sample stocks was performed to generate 4× sample in assay buffer containing 4× EC₅₀ forskolin.

4. 5 μL of 4× sample was added to cells and incubated at 37° C. or room temperature for 30 or 60 minutes.

### Antagonist Format

1. For antagonist determination, cells were pre-incubated with sample followed by agonist challenge at the EC80 concentration.

2. Media was aspirated from cells and replaced with 10 μL 1:1 HBSS/Hepes: cAMP XS+Ab reagent.

3. 5 μL of 4× compound was added to the cells and incubated at 37° C. or room temperature for 30 minutes.

4. 5 μL of 4× EC₅₀ agonist was added to cells and incubated at 37° C. or room temperature for 30 or 60 minutes. For Gi coupled GPCRs, EC₅₀ forskolin was included.

### Signal Detection

1. After appropriate compound incubation, assay signal was generated through incubation with 20 μL cAMP XS+ED/CL lysis cocktail for one hour followed by incubation with 20 μL cAMP XS+EA reagent for three hours at room temperature.

2. Microplates were read following signal generation with PerkinElmer Envision instrument for chemiluminescent signal detection.

### Data Analysis

1. Compound activity was analyzed using CBIS data analysis suite (ChemInnovation, CA).

2. For Gs agonist mode assays, percentage activity was calculated using the following formula: % Activity=100%×(mean RLU of test sample−mean RLU of vehicle control)/(mean RLU of MAX control−mean RLU of vehicle control).

3. For Gs antagonist mode assays, percentage inhibition was calculated using the following formula: % Inhibition=100%×(1−(mean RLU of test sample−mean RLU of vehicle control)/(mean RLU of EC₅₀ control−mean RLU of vehicle control)).

4. For Gi agonist mode assays, percentage activity was calculated using the following formula: % Activity=100%×(1−(mean RLU of test sample−mean RLU of MAX control)/(mean RLU of vehicle control−mean RLU of MAX control)).

5. For Gi antagonist or negative allosteric mode assays, percentage inhibition was calculated using the following formula: % Inhibition=100%×(mean RLU of test sample−mean RLU of EC₅₀ control)/(mean RLU of forskolin positive control−mean RLU of EC₅₀ control).

For Primary screens, percent response was capped at 0% or 100% where calculated percent response returned a negative value or a value greater than 100, respectively.

### B) Calcium Flux Secondary Messenger Assays

The Calcium No Wash^{PLUS} assay was used to monitor GPCR activity via Gq secondary messenger signaling in a live cell, non-imaging assay format. Calcium mobilization in PathHunter® cell lines or other cell lines stably expressing Gq-coupled GPCRs was monitored using calcium-sensitive dye loaded into cells. GPCR activation by a compound resulted in the release of calcium from intracellular stores and an increase in dye fluorescence that was measured in real-time.

### Assay Design

### Cell Handling

1. Cell lines were expanded from freezer stocks according to standard procedures.

2. Cells (10,000 cells/well) were seeded in a total volume of 50 μL (200 cells/L) into black-walled, clear-bottom, Poly-D-lysine coated 384-well microplates and incubated at 37° C. for the appropriate time prior to testing.

### Dye Loading

1. Assays were performed in 1× Dye Loading Buffer consisting of 1× Dye (DiscoverX, Calcium No Wash PLUS kit, Catalog No. 90-0091), 1× Additive A and 2.5 mM Probenecid in HBSS/20 mM Hepes. Probenecid was prepared fresh.

2. Cells were loaded with dye prior to testing. Media was aspirated from cells and replaced with 25 μL Dye Loading Buffer.

3. Cells were incubated for 45 minutes at 37° C. and then 20 minutes at room temperature.

### Agonist Format

1. For agonist determination, cells were incubated with sample to induce response.

2. After dye loading, cells were removed from the incubator and 25 μL of 2× compound in HBSS/20 mM Hepes was added using a FLIPR Tetra (MDS).

3. Compound agonist activity was measured on a FLIPR Tetra. Calcium mobilization was monitored for 2 minutes with a 5 second baseline read.

### Antagonist Format

1. For antagonist determination, cells were pre-incubated with sample followed by agonist challenge at the EC₅₀ concentration.

2. After dye loading, cells were removed from the incubator and 25 μL 2× sample was added. Cells were incubated for 30 minutes at room temperature in the dark to equilibrate plate temperature.

3. After incubation, antagonist determination was initiated with addition of 25 μL 1× compound with 3× EC₅₀ agonist using FLIPR

4. Compound antagonist activity was measured on a FLIPR Tetra (MDS). Calcium mobilization was monitored for 2 minutes with a 5 second baseline read.

### Data Analysis

1. FLIPR read—Area under the curve was calculated for the entire two minute read.

2. Compound activity was analyzed using CBIS data analysis suite (ChemInnovation, CA).

3. For agonist mode assays, percentage activity was calculated as: % Activity=100% x (mean RFU of test sample−mean RFU of vehicle control)/(mean MAX RFU control ligand -mean RFU of vehicle control).

4. For antagonist mode assays, percentage inhibition was calculated as: % Inhibition=100%×(1−(mean RFU of test sample−mean RFU of vehicle control)/(mean RFU of EC80 control−mean RFU of vehicle control)).

For Primary screens, percent response was capped at 0% or 100% where calculated percent response returned a negative value or a value greater than 100, respectively.

### C) Nuclear Hormone Receptor Assays

PathHunter® NHR Protein Interaction (NHR Pro) and Nuclear Translocation (NHR NT) assays monitored the activation of specific nuclear hormone receptors in a homogenous, non-imaging assay format using Enzyme Fragment Complementation (EFC). The NHR Pro assay is based on detection of protein-protein interactions between an activated, full length NHR protein and a nuclear fusion protein containing Steroid Receptor Co-activator Peptide (SRCP) domains with one or more canonical LXXLL interaction motifs.

The NHR was tagged with the ProLink™ (PK) component of the DiscoverX EFC assay system, and the SRCP domain was fused to the Enzyme Acceptor component (EA) expressed in the nucleus. When bound by ligand, the NHR migrates to the nucleus and recruites the SRCP domain, whereby complementation occurs, generating a unit of active β-galactosidase (p-gal) and production of chemiluminescent signal upon the addition of PathHunter detection reagents.

The NHR NT assay monitored movement of an NHR between the cytoplasmic and nuclear compartments. The receptor was tagged with the ProLabel™ (PL) component of the EFC assay system, and EA was fused to a nuclear location sequence that restricted the expression of EA to the nucleus. Migration of the NHR to the nucleus resulted in complementation with EA generating a unit of active B-gal and production of a chemiluminescent signal upon the addition of Path Hunter detection reagents.

### Assay Design

### Cell Handling

1. PathHunter NHR cell lines were expanded from freezer stocks according to standard procedures.

2. Cells were seeded in a total volume of 20 μL into white walled, 384-well microplates and incubated at 37° C. for the appropriate time prior to testing. Assay media contained charcoal-dextran filtered serum to reduce the level of hormones present.

### Agonist Format

1. For agonist determination, cells were incubated with sample to induce response.

2. Intermediate dilution of sample stocks was performed to generate 5× sample in assay buffer.

3. 5 μL of 5× sample was added to cells and incubated at 37° C. or room temperature for 3-16 hours.

### Antagonist Format

1. For antagonist determination, cells were pre-incubated with antagonist followed by agonist challenge at the EC₅₀ concentration.

2. Intermediate dilution of sample stocks was performed to generate 5× sample in assay buffer.

3. 5 μL of 5× sample was added to cells and incubated at 37° C. or room temperature for 60 minutes. Vehicle concentration was 1%.

4. 5 μL of 6× EC80 agonist in assay buffer was added to the cells and incubated at 37° C. or room temperature for 3-16 hours.

### Signal Detection

1. Assay signal was generated through a single addition of 12.5 or 15 μL (50% v/v) of PathHunter Detection reagent cocktail, followed by a one hour incubation at room temperature.

2. Microplates were read following signal generation with a PerkinElmer Envision instrument for chemiluminescent signal detection.

### Data Analysis

1. Compound activity was analyzed using CBIS data analysis suite (ChemInnovation, CA).

2. For agonist mode assays, percentage activity was calculated as:

% Activity=100%×(mean RLU of test sample−mean RLU of vehicle control)/(mean MAX control ligand−mean RLU of vehicle control).

3. For antagonist mode assays, percentage inhibition was calculated as:

% Inhibition=100%×(1−(mean RLU of test sample−mean RLU of vehicle control)/(mean RLU of EC80 control−mean RLU of vehicle control)).

4. Note that for select assays, the ligand response produces a decrease in receptor activity (inverse agonist with a constitutively active target). For those assays inverse agonist activity was calculated as: % Inverse Agonist Activity=100%×((mean RLU of vehicle control−mean RLU of test sample)/(mean RLU of vehicle control−mean RLU of MAX control)).

For Primary screens, percent response was capped at 0% or 100% where calculated percent response returned a negative value or a value greater than 100, respectively.

### D) KINOMEscan® Assays

Kinase activity was measured using the KINOMEscan screening platform, which employs a site-directed competition binding assay to quantitatively measure interactions between test compounds and the kinases. Compounds that bind the kinase active site and directly (sterically) or indirectly (allosterically) prevent kinase binding to the immobilized ligand, will reduce the amount of kinase captured on the solid support (A and B). Conversely, test molecules that do not bind the kinase have no effect on the amount of kinase captured on the solid support (C). Screening “hits” were identified by measuring the amount of kinase captured in test versus control samples by using a quantitative, precise and ultra-sensitive qPCR method that detects the associated DNA label (D). In a similar manner, dissociation constants (Kds) for test compound-kinase interactions were calculated by measuring the amount of kinase captured on the solid support as a function of the test compound concentration.

### Assay Design

### Protein Expression

For most assays, kinase-tagged T7 phage strains were grown in par allel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 m) to remove cell debris. The remaining kinases were produced in HEK-293 cells and subsequently tagged with DNA for qPCR detection.

### Capture Ligand Production

Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding.

### Binding Reaction Assembly

Binding reactions were assembled by combining kinases, liganded affinity beads, and test compounds in 1× binding buffer (20% SeaBlock, 0.17×PBS, 0.05% Tween 20, 6 mM DTT). All reactions were performed in polypropylene 384-well plates in a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Signal Detection

The kinase concentration in the eluates was measured by qPCR. qPCR reactions were assembled by adding 2.5 μL of kinase eluate to 7.5 μL of qPCR master mix containing 0.15 μM amplicon primers and 0.15 μM amplicon probe. The qPCR protocol consisted of a 10 minute hot start at 950 C, followed by 35 cycles of 95° C. for 15 seconds, 60° C. for 1 minute.

### Data Analysis

### Percent Response Calculation

$$100 * ( test compound signal - positive control signal ) / ( negative compound signal - positive control signal )$$

where
   Test compound=compound submitted by Customer
   Negative control=DMSO (100% Ctrl)
   Positive control=control compound (0% Ctrl)
      Percent of Control was converted to Percent Response with the conversion: Percent Response=(100—Percent Control).
      For Primary screens, percent response was capped at 0% or 100% where calculated percent response returned a negative value or a value greater than 100, respectively.

### Binding Constants (Kds)

Binding constants (Kds) were calculated with a standard dose response curve using the Hill equation with Hill Slope set to -1. Curves were fitted using a non-linear least square fit with the Levenberg-Marquardt algorithm.

### E) Monoamine Transporter Uptake Assays

The Neurotransmitter Transporter Uptake Assay Kit from Molecular Devices was used as a homogeneous fluorescence based assay for the detection of dopamine, norepinephrine or serotonin transporter activity in cells expressing these transporters. The kit employs a fluorescent substrate that mimics the biogenic amine neurotransmitters that are taken into the cell through these specific transporters, resulting in increased intracellular fluorescence intensity.

It should be noted that fluorescence based assays for the detection of dopamine, norepinephrine or serotonin transporter activity have poor sensitivity for compounds that are substrates for these monoamine transporters. We therefore separately measured interactions with these transporters using two additional types of assays: an antagonist radioligand assay of inhibition of the human 5-HT transporter (hSERT) expressed in CHO cells (Tatsumi, M. et al. (1999), Eur. J. Pharmacol., 368: 277-283) and an assay measuring release of [³H] Serotonin or [³H] dopamine, respectively, from cells stably expressing SERT or DAT. While the former is sensitive to classic reuptake inhibition, the latter can detect he effects of substrates, which also induce release.

### F) Potassium Assay

The FLIPR Potassium Assay Kit from Molecular Devices was used for ion channel assays. This approach exploited the permeability of thallium ions (Tl+) through both voltage and ligand-gated potassium (K+) channels. A highly-sensitive Tl+ indicator dye produced a bright fluorescent signal upon the binding to Tl+ conducted through potassium channels. The intensity of the Tl+ signal was proportional to the number of potassium channels in the open state and therefore provided a functional indication of the potassium channel activities. In addition, a masking dye was included to reduce background fluorescence for improved signal/noise ratio.

### Assay Design

### Cell Handling

1. Cell lines were expanded from freezer stocks according to standard procedures.

2. Cells were seeded in a total volume of 20 μL into black-walled, clear-bottom, Poly-D-lysine coated 384-well microplates and incubated at 37° C. for the appropriate time prior to testing.

### Dye Loading

1. Assays were performed in 1× Dye Loading Buffer consisting of 1× Dye, and 2.5 mM Probenecid when applicable. Probenecid was prepared fresh.

2. Cells were loaded with dye prior to testing.

3. Cells were incubated for 30-60 minutes at 37° C.

### Agonist/Opener Format

1. For agonist determination, cells were incubated with sample to induce response.

2. Intermediate dilution of sample stocks was performed to generate 2-5× sample in assay buffer.

3. 10-25 μL of 2-5× sample was added to cells and incubated at 37° C. or room temperature for 30 minutes.

### Antagonist/Blocker Format

1. For antagonist determination, cells were pre-incubated with sample.

2. Intermediate dilution of sample stocks was performed to generate 2-5× sample in assay buffer.

3. After dye loading, cells were removed from the incubator and 10-25 μL 2-5× sample was added to cells in the presence of EC80 agonist when appropriate. Cells were incubated for 30 minutes at room temperature in the dark to equilibrate plate temperature.

### Signal Detection

1. Compound activity was measured on a FLIPR Tetra (MDS).

### Data Analysis

1. Compound activity was analyzed using CBIS data analysis suite (ChemInnovation, CA).

2. For agonist mode assays, percentage activity was calculated using the following formula:

$$\% Inhibition = 100 \% \times ( 1 - ( mean RLU of test sample - mean RLU of vehicle control ) / ( mean RLU of EC 80 control - mean RLU of vehicle control ) ) .$$

3. For antagonist percentage inhibition was calculated using the following formula:

$$\% Activity = 100 \% \times ( mean RLU of test sample - mean RLU of vehicle control ) / ( mean MAX control ligand - mean RLU of vehicle control ) .$$

For Primary screens, percent response was capped at 0% or 100% where calculated percent response returned a negative value or a value greater than 100, respectively.

### G) Membrane Potential Assay

The FLIPR® Membrane Potential Assay Kit was used which employs a fluorescent indicator dye in combination with a quencher to reflect real-time membrane potential changes associated with ion channel activation and ion transporter proteins. Unlike traditional dyes such as DiBAC, the FLIPR Membrane Potential Assay Kit detects bidirectional ion fluxes so both variable and control conditions can be monitored within a single experiment. Specific assay steps and reference compounds are given below.

### H) Calcium Assays

The DiscoveRx Calcium NWPLUS Assay Kit was used for detection of changes in intracellular calcium. Cells expressing a receptor of interest that signals through calcium were pre-loaded with a calcium sensitive dye and then treated with compound. Upon stimulation, the receptor signaled release of intracellular calcium, which resulted in an increase of dye fluorescence. Signal was measured on a fluorescent plate reader equipped with fluidic handling capable of detecting rapid changes in fluorescence upon compound stimulation. Specific assay steps and reference compounds are given below.

### Assay Design: Transporter Assays

### Cell Handling

1. Cell lines were expanded from freezer stocks according to standard procedures.

2. Cells were seeded in a total volume of 25 μL into black-walled, clear-bottom, Poly-D-lysine coated 384-well microplates and incubated at 37° C. for the appropriate time prior to testing.

### Blocker/Antagonist Format

1. After cell plating and incubation, media was removed and 25 μL of 1× compound in 1× HBSS/0.1% BSA was added.

2. Compounds were incubated with cells at 37° C. for 30 minutes.

### Dye Loading

1. Assays were performed in 1× Dye Loading Buffer consisting of 1× Dye, 1×HBSS/20 mM Hepes.

2. After compound incubation, 25 μL of 1× dye was added to wells.

3. Cells were incubated for 30-60 minutes at 37° C.

### Signal Detection

1. After dye incubation, microplates were transferred to a PerkinElmer Envision instrument for fluorescence signal detection.

### Data Analysis

1. Compound activity was analyzed using CBIS data analysis suite (ChemInnovation, CA).

2. For blocker mode assays, percentage inhibition was calculated using the following formula: % Inhibition=100%×(1−(mean RLU of test sample−mean RLU of vehicle control)/(mean RLU of positive control−mean RLU of vehicle control)).

For Primary screens, percent response was capped at 0% or 100% where calculated percent response returned a negative value or a value greater than 100, respectively.

### I) Enzymatic Assays

Enzymatic assays determined enzymatic activity by measuring either the consumption of substrate or production of product over time. Different detection methods were used in each enzymatic assay to measure the concentrations of substrates and products, including spectrophotometric, fluorometric, and luminescent readouts.

### Assay Design

### Enzyme Preparations

Enzyme preparations were sourced from various vendors: AChE (R&D Systems), COX1 and COX2 (BPS Bioscience), MAOA (Sigma), PDE3A and PDE4D2 (Signal Chem).

### Enzyme Activity Assays

1. Enzymatic assays determine the enzymatic activity by measuring either the consumption of substrate or production of product over time. Different detection methods were used in each enzymatic assay to measure the concentrations of value greater than 100, respectively. substrates and products.

2. ACHE: Enzyme and test compound were preincubated for 15 minutes at room temp before substrate addition. Acetylthiocholine and DTNB were added and incubated at room temperature for 30 minutes. Signal was detected by measuring absorbance at 405 nm.

3. COX1 & COX2: Enzyme stocks were diluted in Assay Buffer (40 mM Tris-HCl, 1× PBS, 0.5 mM Phenol, 0.01% Tween-20+100 nM Hematin) and allowed to equilibrate with compounds at room temperature for 30 minutes (binding incubation). Arachidonic acid (1.7 μM) and Ampliflu Red (2.5 μM) were prepared and dispensed into a reaction plate. Plates were read immediately on a fluorimeter with the emission detection at 590 nm and excitation wavelength 544 nm.

4. MAOA: Enzyme and test compound were preincubated for 15 minutes at 37° C. before substrate addition. The reaction was initiated by addition of kynuramine and incubated at 37° C. for 30 minutes. The reaction was terminated by addition of NaOH. The amount of 4-hydroquioline formed was determined through spectrofluorimetric readout with the emission detection at 380 nm and excitation wavelength 310 nm.

5. PDE3A & PDE4D2: Enzyme and test compound were preincu bated for 15 minutes at room temp before substrate addition. cAMP substrate (at a concentration equal to EC₅₀) was added and incubated at room temperature for 30 minutes. Enzyme reaction was terminated by addition of 9 mM IBMX. Signal was detected using the HitHunter® cAMP detection kit.

### Signal Detection

1. For each assay, microplates were transferred to a PerkinElmer Envision instrument and readout as described.

### Data Analysis

1. Compound activity was analyzed using CBIS data analysis suite (ChemInnovation, CA).

2. For enzyme activity assays, percentage inhibition was calculated using the following formula: % Inhibition=100%×(1−(mean RLU of test sample−mean RLU of vehicle control)/(mean RLU of positive control−mean RLU of vehicle control)).

For Primary screens, percent response was capped at 0% or 100% where calculated percent response returned a negative value or a value greater than 100, respectively.

Positive Controls (Reference Compounds) used in Assays

| | | | | Result | Result |
|---|---|---|---|---|---|
| Target | Abbreviation | Mode | Reference Compound | Type | (μM) |
| 5-Hydroxytryptamine | 5-HTR1A | Agonist | Serotonin Hydrochloride | EC50 | 0.004 |
| (Serotonin) Receptor 1A | | | | | |
| 5-Hydroxytryptamine | 5-HTR1A | Antagonist | Spiperone | IC50 | 0.105 |
| (Serotonin) Receptor 1A | | | | | |
| 5-Hydroxytryptamine | 5-HTR1B | Agonist | Serotonin Hydrochloride | EC50 | 2.00E−04 |
| (Serotonin) Receptor 1B | | | | | |
| 5-Hydroxytryptamine | 5-HTR1B | Antagonist | SB 224289 | IC50 | 0.006 |
| (Serotonin) Receptor 1B | | | | | |
| 5-Hydroxytryptamine | 5-HTR2A | Agonist | Serotonin Hydrochloride | EC50 | 0.003 |
| (Serotonin) Receptor 2A | | | | | |
| 5-Hydroxytryptamine | 5-HTR2A | Antagonist | Altanserin | IC50 | 0.016 |
| (Serotonin) Receptor 2A | | | | | |
| 5-Hydroxytryptamine | 5-HTR2B | Agonist | Serotonin Hydrochloride | EC50 | 0.004 |
| (Serotonin) Receptor 2B | | | | | |
| 5-Hydroxytryptamine | 5-HTR2B | Antagonist | LY 272015 | IC50 | 3.00E−04 |
| (Serotonin) Receptor 2B | | | | | |
| 5-Hydroxytryptamine | 5-HTR3A | Blocker | Bemesetron | IC50 | 0.003 |
| (Serotonin) Receptor 3A | | | | | |
| 5-Hydroxytryptamine | 5-HTR3A | Opener | Serotonin Hydrochloride | EC50 | 0.367 |
| (Serotonin) Receptor 3A | | | | | |
| Acetylcholinesterase | ACHE | Inhibitor | Physostigmine | IC50 | 0.037 |
| Adenosine Receptor A2A | ADORA2A | Antagonist | SCH 442416 | IC50 | 0.080 |
| Adenosine Receptor A2A | ADORA2A | Agonist | NECA | EC50 | 0.0178 |
| Adrenergic Receptor α₁A | ADRA1A | Agonist | A 61603 Hydrobromide | EC50 | 9.00E−05 |
| Adrenergic Receptor α₁A | ADRA1A | Antagonist | Tamsulosin | IC50 | 0.001 |
| Adrenergic Receptor α₂A | ADRA2A | Agonist | UK 14304 | EC50 | 6.00E−05 |
| Adrenergic Receptor α₂A | ADRA2A | Antagonist | Yohimbine | IC50 | 0.005 |
| Adrenergic Receptor β1 | ADRB1 | Agonist | (−)-Isoproterenol | EC50 | 0.002 |
| Adrenergic Receptor β1 | ADRB1 | Antagonist | Betaxolol | IC50 | 0.003 |
| Adrenergic Receptor β2 | ADRB2 | Agonist | (−)-Isoproterenol | EC50 | 3.00E−04 |
| Adrenergic Receptor β2 | ADRB2 | Antagonist | ICI 118,551 hydrochloride | IC50 | 0.0006 |
| Nuclear Hormone | AR | Agonist | 6a-Fluorotestosterone | EC50 | 0.002 |
| Androgen Receptor | | | | | |
| Nuclear Hormone | AR | Antagonist | Geldanamycin | IC50 | 0.094 |
| Androgen Receptor | | | | | |
| Arginine Vasopressin | AVPR1A | Agonist | [Arg⁸]-Vasopressin | EC50 | 0.0004 |
| Receptor 1A | | | | | |
| Arginine Vasopressin | AVPR1A | Antagonist | SR 49059 | IC50 | 0.002 |
| Receptor 1A | | | | | |
| Voltage-gated L-type | CAV1.2 | Blocker | Isradipine | IC50 | 0.017 |
| calcium channel | | | | | |
| Cholecystokinin Receptor | CCKAR | Agonist | (Tyr[SO₃H]₂₇)Cholecystokinin | EC50 | 1.00E−04 |
| A | | | fragment 26-33 Amide | | |
| Cholecystokinin Receptor | CCKAR | Antagonist | SR 27897 | IC50 | 0.037 |
| A | | | | | |
| Muscarinic acetylcholine | CHRM1 | Agonist | Acetylcholine chloride | EC50 | 0.016 |
| Receptor M1 | | | | | |
| Muscarinic acetylcholine | CHRM1 | Antagonist | Atropine | IC50 | 0.003 |
| Receptor M1 | | | | | |
| Muscarinic acetylcholine | CHRM2 | Agonist | Acetylcholine chloride | EC50 | 0.025 |
| Receptor M2 | | | | | |
| Muscarinic acetylcholine | CHRM2 | Antagonist | Atropine | IC50 | 0.004 |
| Receptor M2 | | | | | |
| Muscarinic acetylcholine | CHRM3 | Agonist | Acetylcholine chloride | EC50 | 0.040 |
| Receptor M3 | | | | | |
| Muscarinic acetylcholine | CHRM3 | Antagonist | Atropine | IC50 | 0.002 |
| Receptor M3 | | | | | |
| Cannabinoid Receptor 1 | CNR1 | Agonist | CP 55940 | EC50 | 4.00E−05 |
| Cannabinoid Receptor 1 | CNR1 | Antagonist | AM 251 | IC50 | 0.003 |
| Cannabinoid Receptor 2 | CNR2 | Agonist | CP 55940 | EC50 | 0.0002 |
| Cannabinoid Receptor 2 | CNR2 | Antagonist | SR 144528 | IC50 | 0.035 |
| Cyclooxygenase 1 | COX1 | Inhibitor | Indomethacin | IC50 | 0.033 |
| Cyclooxygenase 2 | COX2 | Inhibitor | NS-398 | IC50 | 0.022 |
| Dopamine transporter | DAT | Blocker | GBR 12909 | IC50 | 0.015 |
| Dopamine Receptor D1 | DRD1 | Agonist | Dopamine | EC50 | 0.086 |
| Dopamine Receptor D1 | DRD1 | Antagonist | SCH 39166 | IC50 | 0.0009 |
| Dopamine Receptor D2 | DRD2S | Agonist | Dopamine | EC50 | 0.001 |
| Dopamine Receptor D2 | DRD2S | Antagonist | Risperidone | IC50 | 0.002 |
| Endothelin Receptor Type | EDNRA | Agonist | Endothelin 1 | EC50 | 0.001 |
| A | | | | | |
| Endothelin Receptor Type | EDNRA | Antagonist | BMS 182874 | IC50 | 1.107 |
| A | | | | | |
| Gamma-aminobutyric acid | GABAA | Blocker | Picrotoxin | IC50 | 2.778 |
| Receptor A | | | | | |
| Gamma-aminobutyric acid | GABAA | Opener | GABA | EC50 | 6.358 |
| Receptor A | | | | | |
| Nuclear Hormone | GR | Agonist | Dexamethasone | EC50 | 0.05 |
| Glucocorticoid Receptor | | | | | |
| Nuclear Hormone | GR | Antagonist | Mifepristone | IC50 | 0.072 |
| Glucocorticoid Receptor | | | | | |
| Nuclear Hormone | HERG | Blocker | Astemizole | IC50 | 0.222 |
| Glucocorticoid Receptor | | | | | |
| Histamine Receptor H1 | HRH1 | Agonist | Histamine | EC50 | 0.032 |
| Histamine Receptor H1 | HRH1 | Antagonist | Mepyramine | IC50 | 0.005 |
| Histamine Receptor H2 | HRH2 | Agonist | Histamine | EC50 | 0.264 |
| Histamine Receptor H2 | HRH2 | Antagonist | Tiotidine | IC50 | 0.139 |
| Insulin Receptor (tyrosine | INSR | Inhibitor | BMS-754807 | IC50 | 0.0005 |
| kinase) | | | | | |
| Kv11.1, the alpha subunit | KvLQT1/minK | Blocker | XE 991 | IC50 | 1.668 |
| of a potassium ion channel | | | | | |
| Kv11.1, the alpha subunit | KvLQT1/mink | Opener | ML-277 | EC50 | 2.306 |
| of a potassium ion channel | | | | | |
| Lymphocyte Cell-Specific | LCK | Inhibitor | Gleevec | IC50 | 13.361 |
| Protein-Tyrosine Kinase | | | | | |
| (Src family) | | | | | |
| Monoamine oxidase type A | MAOA | Inhibitor | Clorgyline | IC50 | 0.004 |
| Nicotinic acetylcholine | nACHR(a4/b2) | Blocker | Dihydro-AY-erythroidine | IC50 | 0.682 |
| Receptor α4 β2 | | | | | |
| Nicotinic acetylcholine | nACHR(a4/b2) | Opener | (−)-Nicotine | EC50 | 2.374 |
| Receptor α4 β2 | | | | | |
| A tetrodotoxin-resistant | NAV1.5 | Blocker | Lidocaine | IC50 | 32.05 |
| voltage-gated sodium | | | | | |
| channel N-methyl-D- | | | | | |
| aspartate (NMDA) | | | | | |
| Glutamate | | | | | |
| Norepinephrine transporter | NET | Blocker | Desipramine | IC50 | 0.013 |
| N-methyl-D-aspartate | NMDAR | Blocker | (+)-MK 801 maleate | IC50 | 0.039 |
| (NMDA) Glutamate | (1A/2B) | | | | |
| Receptor 1A/2B | | | | | |
| N-methyl-D-aspartate | NMDAR | Opener | L-Glutamic Acid | EC50 | 0.413 |
| (NMDA) Glutamate | (1A/2B) | | | | |
| Receptor 1A/2B | | | | | |
| Opioid Receptor Delta 1 | OPRD1 | Agonist | DADLE | EC50 | 0.0001 |
| Opioid Receptor Delta 1 | OPRD1 | Antagonist | Naltriben | IC50 | 0.0004 |
| Opioid Receptor Kappa 1 | OPRK1 | Agonist | Dynorphin A (1-17) | EC50 | 0.012 |
| Opioid Receptor Kappa 1 | OPRK1 | Antagonist | nor-Binaltorphimine | IC50 | 0.007 |
| Opioid Receptor Mu | OPRM1 | Agonist | DAMGO | EC50 | 0.002 |
| Opioid Receptor Mu | OPRM1 | Antagonist | Naloxone | IC50 | 0.006 |
| cGMP-inhibited cyclic | PDE3A | Inhibitor | Cilostamide | IC50 | 0.056 |
| nucleotide | | | | | |
| phosphodiesterase 3A | | | | | |
| cAMP-specific 3′,5′-cyclic | PDE4D2 | Inhibitor | Cilomilast | IC50 | 0.017 |
| phospho-diesterase | | | | | |
| Catecholamine | | | | | |
| Transporters | | | | | |
| Rho Associated Coiled- | ROCK1 | Inhibitor | Staurosporine | IC50 | 8.00E−05 |
| Coil Containing Protein | | | | | |
| Kinase 1 (serine-threonine | | | | | |
| kinase) | | | | | |
| Serotonin transporter | SERT | Blocker | Clomipramine | IC50 | 0.002 |
| Vascular endothelial | VEGFR2 | Inhibitor | SU-11248 | IC50 | 0.0002 |
| growth factor receptor 2 | | | | | |
| (KDR tyrosine kinase) | | | | | |

Each patent, publication, and non-patent literature cited in the application is hereby incorporated by reference in its entirety as if each was incorporated by reference individually.

Where reference is made to patents, publications, non-patent literature, and other sources of information, it is for the general purpose of providing context for discussing features of the present invention. Accordingly, unless specifically stated otherwise, reference is not to be construed as an admission that the document or underlying information, in any jurisdiction, is prior art, or forms part of the common general knowledge in the art.

I claim:

1. A compound of Formula: 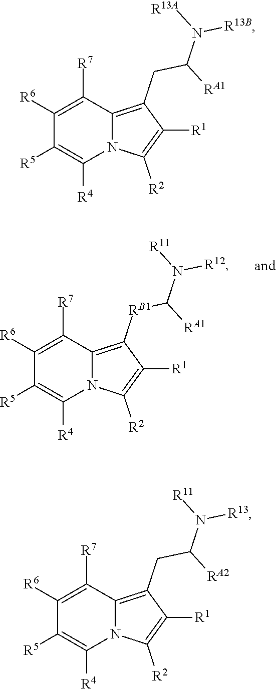
or a pharmaceutically acceptable salt or salt mixture thereof;
wherein:
R^{A1} is hydrogen, —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R^{A2} is —CH₃, —CH₂X, —CHX₂, —CX₃, —CH₂CH₃, —CH₂CH₂X, —CH₂ CHX₂, —CH₂CX₃, —CH₂OH, or —CH₂CH₂OH;
R^{B1} is 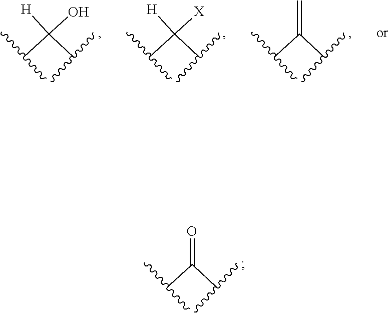
R¹, R², R⁴, R⁵ R⁶, and R⁷ are independently selected from the group consisting of hydrogen, F, Cl, alkyl, haloalkyl, —OP(O)(OR⁹)₂, —SR⁹, —NR⁹R¹⁰, alkenyl, alkynyl, aminoalkyl, —S(O)₂R¹⁷, -alkyl-S(O)₂R¹⁷, —NR⁹S(O)₂R¹⁷, OCH₃, and —NR⁹S(O)₂R¹⁷;
R⁹ and R¹⁰ are independently selected at each instance from the group consisting of hydrogen, alkyl, and haloalkyl;
R¹¹ is hydrogen, —(C₁-C₆)alkyl, —CH₂CH₂X, —CH₂ CHX₂, —CH₂CX₃, or —CH₂CH₂OH;
R¹² is hydrogen, —(C₁-C₆)alkyl, —CH₂CH₂X, —CH₂ CHX₂, —CH₂CX₃, or —CH₂CH₂OH;
R¹³ is —(C₁-C₆)alkyl, —CH₂CH₂X, —CH₂ CHX₂, —CH₂CX₃, —CH₂CH₂OH, or hydroxy;
R^{13A} is —(C₁-C₆)alkyl, —CH₂CH₂X, —CH₂ CHX₂, —CH₂CX₃, or —CH₂CH₂OH;
R^{13B} is —(C₃-C₆)alkyl, —CH₂CH₂X, —CH₂ CHX₂, —CH₂CX₃, or —CH₂CH₂OH;
R¹⁷ is alkyl, haloalkyl, —NR⁹R¹⁰, or —OR⁹; and
X at each instance is independently selected from F and Cl.

2. The compound of claim 1, wherein the compound is of formula: 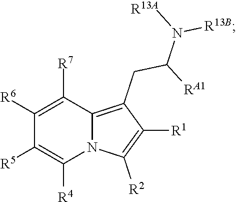
or a pharmaceutically acceptable salt or salt mixture thereof.

3. The compound of claim 2, wherein R^{A1} is hydrogen.

4. The compound of claim 2, wherein R^{A1} is methyl.

5. The compound of claim 2, wherein R^{A1} is ethyl.

6. The compound of claim 1, wherein the compound is 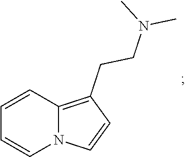
or a pharmaceutically acceptable salt thereof.

7. The compound of claim 1, wherein R¹¹ is hydrogen or CH₃.

8. The compound of claim 7, wherein the compound is of formula: 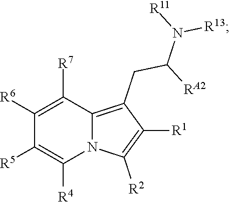
or a pharmaceutically acceptable salt or salt mixture thereof.

9. The compound of claim 8, wherein R^{A2} is methyl.

10. The compound of claim 8, wherein R^{A2} is ethyl.

11. The compound of claim 7, wherein the compound is of formula: 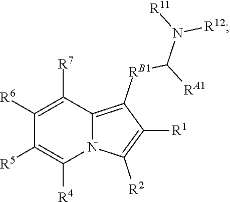
or a pharmaceutically acceptable salt or salt mixture thereof.

12. The compound of claim 1, wherein the compound is of formula: 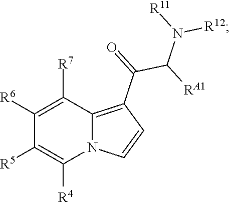
or a pharmaceutically acceptable salt or salt mixture thereof.

13. The compound of claim 12, wherein R^{A1} is hydrogen.

14. The compound of claim 12, wherein R^{A1} is methyl.

15. The compound of claim 12, wherein R^{A1} is ethyl.

16. The compound of claim 1, wherein R¹ and R² are hydrogen.

17. The compound of claim 16, wherein R⁴ and R⁷ are hydrogen.

18. The compound of claim 1, wherein the compound is: 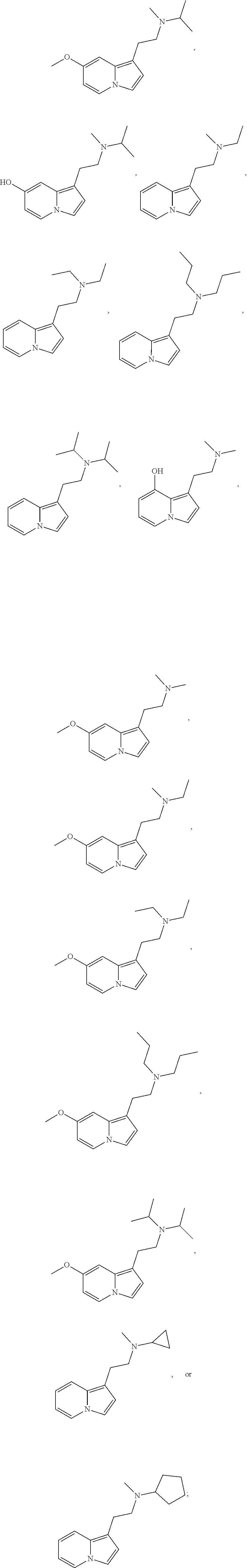
or a pharmaceutically acceptable salt or salt mixture thereof.

19. A pharmaceutical composition comprising an effective amount of a compound of claim 1 or a pharmaceutically acceptable salt or salt mixture thereof and a pharmaceutically acceptable carrier or excipient.

20. A method for treating a central nervous system disorder comprising administering an effective amount of a compound of claim 1 or a pharmaceutically acceptable salt or salt mixture thereof to a human in need thereof.
